(19) United States
(12) Patent Application Publication
(10) Pub. No.: US 2024/0226299 A1
(43) Pub. Date: Jul. 11, 2024

(54) CELLULAR PRODUCTION OF PURE IRON OXIDE NANOPARTICLES

(71) Applicant: NANOBACTERIE, Paris (FR)

(72) Inventor: Edouard ALPHANDERY, Paris (FR)

(73) Assignee: NANOBACTERIE, Paris (FR)

(21) Appl. No.: 18/340,297

(22) Filed: Jun. 23, 2023

Related U.S. Application Data
(63) Continuation-in-part of application No. 16/589,227, filed on Oct. 1, 2019, now Pat. No. 11,732,277.

(30) Foreign Application Priority Data
Oct. 2, 2018 (FR) 1801033
Oct. 2, 2018 (FR) 1801034

(51) Int. Cl. A61K 41/00; A61K 9/48; A61K 33/06; A61K 33/24; A61K 33/241; A61K 33/30; A61K 33/32; A61K 33/34

(52) U.S. Cl. CPC A61K 41/0052; A61K 9/4816; A61K 33/06; A61K 33/24; A61K 33/241; A61K 33/30; A61K 33/32; A61K 33/34

(57) ABSTRACT

High purity magnetosomes produced by magnetotactic bacteria, wherein the metallic composition of the magnetosome is characterized by a high level of iron purity, preferentially not achieved through a chemical synthesis. The high purity magnetosomes are further characterized by having a content of Cd, Pb, Ni, Co, Al, Mn, Zn, Li, and/or Cu.

### FIELD

The field of the invention is that of biological production of nanoparticles comprising a low quantity of impurities.

### BACKGROUND

Bacteria producing nanoparticle(s), such as magnetotactic bacteria, are known to accumulate impurities in their crystallized structure. For example, when magnetotactic bacteria are grown in the presence of cobalt, they produce magnetosomes comprising iron oxide and cobalt (S. Staniland et al, Nature nanotechnology, V. 3, P. 158 (2008)). For medical applications, it is desired that nanoparticle(s) contain(s) a low level of toxic impurities such as cobalt.

### SUMMARY

The invention relates to a method for producing high purity iron oxide nanoparticles using nanoparticle-producing cells, comprising:
a) A pre-growth step comprising amplifying the nanoparticle-producing cell(s) in a pre-growth and/or fed-batch medium/media preferentially such that nanoparticle-producing cell(s) produce(s) essentially no nanoparticles, and
b) A growth step comprising amplifying the nanoparticle-producing cell(s) originating from the pre-growth step in a growth and/or fed-batch medium/media preferentially such that nanoparticle-producing cell(s) produce nanoparticles,
   wherein the pre-growth and/or growth and/or fed-batch medium/media comprise(s), per kilogram or liter of pre-growth and/or growth and/or fed-batch medium/media:
i) no more than 5·10⁵, 5·10³, 50, 5, 0.5, 0.005, 0.0005, 0.00005 or 5·10⁻¹⁰ gram of yeast extract, and/or
ii) no more than 10⁵, 10³, 10, 1, 0.1, 0.001, 10⁻⁵ or 10⁻¹⁰ gram of CMR agent preferentially selected from the group consisting of boric acid and nitrilotriacetic acid,
   wherein preferentially the fed-batch medium when it is present is a medium that supplements the pre-growth and/or growth medium/media,
   wherein preferentially more nanoparticles are produced in the growth step than in the pre-growth step, preferentially by a factor of at least 0, 0.1, 0.5, 2, 5, 10, 10³, 10⁵ or 10¹⁰, where this factor is preferentially Q₂/Q₁, where Q₁ and Q₂ are preferentially the quantities of nanoparticles produced in/during the pre-growth and growth steps or sub-steps, respectively, and/or
   wherein preferentially the pre-growth and/or growth and/or fed-batch medium/media comprise(s) at least another compound, which is preferentially involved in bacterial growth and/or magnetosome production or synthesis, wherein preferentially the other compound is a source of carbon, nitrogen, calcium, vitamin, oxygen, iron, phosphate, phosphorous, and/or magnesium.

### BRIEF DESCRIPTION OF THE FIGURES

FIG. 1: TGA-DSC analysis of whole magnetotactic bacteria and magnetosomes extracted from magnetotactic bacteria according to condition 2 of lysis. (a), The variation of the percentage in weight as a function of temperature as well as the derivative of this variation as a function of temperature for a sample comprising 3 mg of lyophilized magnetotactic bacteria when it is heated between 20° C. and 600° C. at a rate of 6° C./min. (b), Heat flow in mW as a function of temperature produced by a sample comprising 3 mg of lyophilized magnetotactic bacteria when it is heated between 20° C. and 600° C. at a rate of 6° C./min. (c), The variation of the percentage in weight as a function of temperature as well as the derivative of this variation as a function of temperature for a sample comprising 3 mg of lyophilized magnetosomes extracted from whole bacteria according to condition 2 of lysis when the sample is heated between 20° C. and 600° C. at a rate of 6° C./min. (d), Heat flow in mW as a function of temperature produced by a sample comprising 3 mg of lyophilized magnetosomes extracted from whole bacteria according to condition 2 of lysis when the sample is heated between 20° C. and 600° C. at a rate of 6° C./min. Concerning (a) and (c), the y axis can be replaced by the percentage in mass, leading to the same plots.

FIG. 2: TGA-DSC analysis of magnetosomes extracted from magnetotactic bacteria according to condition 3 and of chemically synthesized SIGMA nanoparticles. (a), Variation of the percentage in weight as a function of temperature as well as the derivative of this variation as a function of temperature for a sample comprising 3 mg of lyophilized magnetosomes extracted from magnetotactic bacteria according to condition 3. (b), Heat flow in mW as a function of temperature produced by a sample comprising 3 mg of lyophilized magnetosomes extracted from magnetotactic bacteria according to condition 3. (c), Variation of the percentage in weight as a function of temperature as well as the derivative of this variation as a function of temperature for a sample comprising 3 mg of lyophilized SIGMA nanoparticles. (d), Heat flow in mW as a function of temperature produced by a sample comprising 3 mg of lyophilized SIGMA nanoparticles. Concerning (a) and (c), the y axis can be replaced by the percentage in mass, leading to the same plots.

FIG. 3: An illustrative example of use of the method according to the invention, following a series of pre-growth step(s) 1, i, and i+1, where the pre-growth step is preferentially started by inserting nanoparticle-producing cells of the cell bank (typically 10⁷ cells) in the volume of pre-growth step V_{PGS1} (typically ˜50 mL), nanoparticle-producing cells are amplified in this volume typically during ˜7 days, nanoparticle-producing cells are then transferred from V_{PGS1} to V_{PGSi} (V_{PGSi} typically ˜500 mL), nanoparticle-producing cells are amplified in this volume during typically ˜3 days, nanoparticle-producing cells are then transferred from V_{PGSi} to V_{PGSi+1} (V_{PGSi+1} typically ˜5 L), nanoparticle-producing cells are amplified in this volume during typically 3 days up to an OD of typically 1 and are then transferred to V_{GS0} (typically 45 liters) where V_{GS0} is supplemented by a fed-batch medium (typically 5-10 liters) during the growth step of preferentially ˜5 days in the presence of oxygen bubbled through the growth medium to enable bacterial growth up to an OD of typically 5 to 40 and quantity of magnetosomes produced of 5 to 500 mg per liter of growth medium, where the pre-growth medium comprises a limited concentration of iron or iron source (preferentially <2 μM) preferentially to prevent the production of nanoparticles and the growth medium comprises iron or an iron source (preferentially >2 μM) preferentially to promote nanoparticle production.

FIG. 4: Concentrations C₁ and C₂ of the carbon source, nitrogen source and iron source at the beginning and end of pre-growth step.

FIGS. 5 and 6: Concentrations C₁ and C₂ of the carbon source, nitrogen source and iron source at the beginning, 20 hours following the beginning, 40 hours following the beginning and more than 40 hours following the beginning of the growth step.

### DETAILED DESCRIPTION

In one embodiment of the invention, the nanoparticle-producing cells are cells, preferentially eukaryotic or prokaryotic ones, that have the faculty or ability to produce or synthesize nanoparticles, preferentially when they are inserted or amplified in a medium that comprises at least one compound that is comprised in the nanoparticle, preferentially at a concentration larger than 10⁻⁶, 1 or 10⁶ μM.

In one embodiment of the invention, nanoparticle-producing cell(s) produce(s) essentially no nanoparticles when these cells produce less than 10⁵⁰, 10¹⁰, 10⁵, 10³, 10, 1, 10⁻¹, 10⁻³ or 10⁻⁵ mg of nanoparticles per liter of pre-growth and/or growth and/or fed-batch medium/media or when the pre-growth and/or growth and/or fed-batch medium/media preferentially comprising nanoparticle-cell(s) comprise(s) less than 10⁵⁰, 10¹⁰, 10⁵, 10³, 10, 1, 10⁻¹, 10⁻³ or 10⁻⁵ mg of nanoparticles per liter of pre-growth and/or growth and/or fed-batch medium/media.

In one embodiment of the invention, nanoparticle-producing cell(s) produce nanoparticles when these cells produce more than 0, 10⁻⁵⁰, 10⁻²⁰, 10⁻¹⁰, 10⁻⁵, 10⁻¹, 1, 10, 10³ or 10⁵ mg of nanoparticles per liter of pre-growth and/or growth and/or fed-batch medium/media or when the pre-growth and/or growth and/or fed-batch medium/media preferentially comprising nanoparticle-cell(s) comprise(s) more than 0, 10⁻⁵⁰, 10⁻²⁰, 10⁻¹⁰, 10⁻⁵, 10⁻¹, 1, 10, 10³ or 10⁵ mg of nanoparticles per liter of pre-growth and/or growth and/or fed-batch medium/media.

In one embodiment of the invention, the quantity of nanoparticles produced by the nanoparticle cells in the pre-growth step is equal to Q₂−Q₁, where Q₁ and Q₂ are the quantities of nanoparticles produced at times t₁ and t₂ of the pre-growth step, where t₂ is larger than t₁, preferentially t₂/t₁ is larger than 1, 2, 5, 10 or 10³, preferentially t₂ is the end of the pre-growth step and t₁ is the beginning of the pre-growth step.

In one embodiment of the invention, the quantity of nanoparticles produced by the nanoparticle cells in the growth step is equal to Q′₂-Q′₁, where Q′₁ and Q′₂ are the quantities of nanoparticles produced at times t′₁ and t′₂ of the growth step, where t′₂ is larger than t′₁, preferentially t′₂/t′₁ is larger than 1, 2, 5, 10 or 10³, preferentially t′₂ is the end of the growth step and t′₁ is the beginning of the growth step.

Preferentially, Q′₂-Q′₁ is larger than Q₂−Q₁, preferentially by a factor of at least 0, 10⁻¹⁰, 10⁻¹, 1, 5, 10, 10³ or 10⁵.

Preferentially, the total quantity of nanoparticles produced by the nanoparticle-producing cells is equal to Qₜₒₜₐₗ=Q′₂−Q′₁+Q₂−Q₁.

In some cases, Qₜₒₜₐₗ can be larger than 10⁻⁵⁰, 10⁻¹⁰, 10⁻⁵, 10⁻³, 10⁻¹, 0, 1, 5, 10 or 100 mg of nanoparticles, preferentially of iron comprised in nanoparticles, preferentially per liter of pre-growth and/or growth medium/media.

In some other cases, Qₜₒₜₐₗ can be lower than 10⁵⁰, 10¹⁰, 10⁵, 10³, 100, 50, 10 or 1 mg of nanoparticles, preferentially of iron comprised in nanoparticles, preferentially per liter of pre-growth and/or growth medium/media.

In one embodiment of the invention, the yeast extract is one element selected from the group consisting of: i) whole yeast extract, ii) a medium comprising more than 1, 10, 50 or 90% of compounds from the whole yeast extract, and iii) a chemical equivalent of the yeast extract.

In one embodiment of the invention, the peptone is one element selected from the group consisting of: i) whole peptone, ii) a medium comprising more than 1, 10, 50 or 90% of compounds from the whole peptone, and iii) a chemical equivalent of peptone.

The invention relates to a method for producing high purity iron oxide nanoparticles using nanoparticle-producing cells, preferentially comprising a pre-growth step comprising amplifying the nanoparticle-producing cells preferentially in a pre-growth medium while preferentially producing essentially no nanoparticles, which is preferentially followed by a growth step, comprising amplifying the nanoparticle-producing cells preferentially originating from the pre-growth step preferentially in a growth medium while producing nanoparticles, wherein preferentially the growth step differs from the pre-growth step by at least one property selected from the group consisting of:
i) a ratio C_{FeGS}/C_{FePGS} that is larger than 0, 10⁻⁵, 10⁻³, 1, 10, 10³ or 10⁵, where C_{FeGS} and C_{FePGS} are concentrations in iron or iron source of the growth medium and pre-growth medium, respectively,
ii) a ratio C_{CGS}/C_{CPGS} that is larger than 0, 10⁻⁵, 10⁻³, 1, 10, 10³ or 10⁵, where C_{CGS} and C_{CPGS} are the concentrations in carbon or carbon source of the growth medium and pre-growth medium, respectively,
iii) a ratio C_{NGS}/C_{NPGS} that is larger than 0, 10⁻⁵, 10⁻³, 1, 10, 10³ or 10⁵, where C_{NGS} and C_{NPGS} are the concentrations in nitrogen or nitrogen source of the growth medium and pre-growth medium, respectively,
iv) a ratio ΔₚH_{GS}/ΔpH_{PGS} that is lower than 0, 10¹⁰, 10⁵, 10³, 10², 1, 0.5 or 0.1, where Δ_{pHGS} and ΔpH_{PGS} are the pH variations of the growth medium and pre-growth medium, respectively,
v) a ratio Q_{GGS}/Q_{GPGS} that is larger than 0, 10¹⁰, 10⁵, 10³, 10², 1, 0.5 or 0.1, where Q_{GGS} and Q_{GPGS} are quantities of gas, oxygen or air brought in or bubbled through the growth medium and pre-growth medium, respectively,
vi) a ratio N_{SSGS}/N_{SSPGS} that is lower than 0, 10⁻⁵, 10⁻³, 1, 10, 10³ or 10⁵, where N_{SSGS} and N_{SSPGS} are numbers of sub-steps of the growth step and numbers of sub-steps of the pre-growth step, respectively, where two sub-steps are separated by each other by a transfer of nanoparticle-producing cells from a first sub-step to a second sub-step, and
vii) the growth medium is supplemented by a fed-batch medium, whereas the pre-growth medium is not supplemented by such medium, and/or
wherein preferentially the pre-growth, growth, and/or fed-batch medium/media does/do not comprise:
I) at least one compound or assembly of compounds comprised in or originating from yeast extract selected in the group consisting of:
   I.1) more than 0, 10⁻¹⁰, 10⁻⁵, 10⁻¹, 1, 10³ or 10⁵ gram per liter of pre-growth and/or growth and/or fed-batch medium/media of protein,
   I.2) more than 0, 10⁻¹⁰, 10⁻⁶, 10⁻¹, 1, 10³ or 10⁵ gram per liter of pre-growth and/or growth and/or fed-batch medium/media of nucleic acids,
   I.3) more than 0, 10⁻¹⁰, 10⁻⁵, 10⁻¹, 1, 10³ or 10⁵ gram per liter of pre-growth and/or growth and/or fed-batch medium/media of glutathione,
   I.4) more than 0, 10⁻¹⁰, 10⁻⁷, 10⁻¹, 1, 10³ or 10⁵ gram per liter of pre-growth and/or growth and/or fed-batch medium/media of at least one the compounds selected from the group consisting of: dextran, mannan, trehalose, flavoring nucleotide, B vitamins, biotin, and volatile aromatic compounds,
   I.5) more than 0, 10⁻¹⁰, 10⁻⁷, 10⁻¹, 1, 10³ or 10⁵ gram per liter of pre-growth and/or growth and/or fed-batch medium/media of calcium,
   I.6) more than 0, 10⁻¹⁰, 10⁻⁶, 10⁻¹, 1, 10³ or 10⁵ gram per liter of pre-growth and/or growth and/or fed-batch medium/media of phosphorous,
   I.7) more than 0, 10⁻¹⁰, 10⁻⁸, 10⁻¹, 1, 10³ or 10⁵ gram per liter of pre-growth and/or growth and/or fed-batch medium/media of zinc,
   I.8) more than 0, 10⁻¹⁰, 10⁻¹, 1, 10³ or 10⁵ gram per liter of pre-growth and/or growth and/or fed-batch medium/media of chrome,
   I.9) more than 0, 10⁻¹⁰, 10⁻⁷, 10⁻¹, 1, 10³ or 10⁵ gram per liter of pre-growth and/or growth and/or fed-batch medium/media of potassium,
   I.10) more than 0, 10⁻¹⁰, 10⁻¹, 1, 10³ or 10⁵ gram per liter of pre-growth and/or growth and/or fed-batch medium/media of cobalt,
   I.11) more than 0, 10⁻¹⁰, 10⁻⁹, 10⁻¹, 1, 10³ or 10⁵ gram per liter of pre-growth and/or growth and/or fed-batch medium/media of manganese,
   I.12) more than 0, 10⁻¹⁰, 10⁻¹, 1, 10³ or 10⁵ gram per liter of pre-growth and/or growth and/or fed-batch medium/media of strontium,
   I.13) more than 0, 10⁻¹⁰, 10⁻⁷, 10⁻¹, 1, 10³ or 10⁵ gram per liter of pre-growth and/or growth and/or fed-batch medium/media of magnesium,
   I.14) more than 0, 10⁻¹⁰, 10⁻⁴, 10⁻¹, 1, 10³ or 10⁵ gram per liter of pre-growth and/or growth and/or fed-batch medium/media of yeast extract, and
   I.15) more than 0, 1, 5, 10, 15, 20 or 50 different components originating from yeast extract;
II. at least one compound or assembly of compounds comprised in or originating from peptone selected in the group consisting of:
   II.1) more than 0, 10⁻¹⁰, 10⁻⁴, 10⁻¹, 1, 10³ or 10⁵ gram per liter of pre-growth and/or growth and/or fed-batch medium/media of ashes,
   II.2) more than 0, 10⁻¹⁰, 10⁻³, 10⁻¹, 1, 10³ or 10⁵ gram per liter of pre-growth and/or growth and/or fed-batch medium/media of proteins,
   II.3) more than 0, 10⁻¹⁰, 10⁻⁴, 10⁻¹, 1, 10³ or 10⁵ gram per liter of pre-growth and/or growth and/or fed-batch medium/media of sucrose,
   II.4) more than 0, 10⁻¹⁰, 10⁻⁵, 10⁻¹, 1, 10³ or 10⁵ gram per liter of pre-growth and/or growth and/or fed-batch medium/media of raffinose,
   II.5) more than 0, 10⁻¹⁰, 10⁻⁴, 10⁻¹, 1, 10³ or 10⁵ gram per liter of pre-growth and/or growth and/or fed-batch medium/media of neutral detergent fiber,
   II.6) more than 0, 10⁻¹⁰, 10⁻⁵, 10⁻¹, 1, 10³ or 10⁵ gram per liter of pre-growth and/or growth and/or fed-batch medium/media of Ethereal Extract,
   II.7) more than 0, 10⁻¹⁰, 10⁻⁵, 10⁻¹, 1, 10³ or 10⁵ gram per liter of pre-growth and/or growth and/or fed-batch medium/media of peptone, and
   II.8) more than 0, 1, 5, 10, 15, 20 or 50 different components originating from peptone;
III. at least one compound or assembly of compounds comprised in or originating from Wolf's minerals or mineral elixir selected in the group consisting of:
   III.1) more than 0, 10⁻²⁰, 10⁻⁵, 10⁻¹, 1, 10³ or 10⁵ mol per liter of pre-growth and/or growth and/or fed-batch medium/media of nitrilotriacetic acid,
   III.2) more than 0, 10⁻²⁰, 10⁻⁶, 10⁻¹, 1, 10³ or 10⁵ mol per liter of pre-growth and/or growth and/or fed-batch medium/media of magnesium sulfate,
   III.3) more than 0, 10⁻²⁰, 10⁻⁵, 10⁻¹, 1, 10³ or 10⁵ mol per liter of pre-growth and/or growth and/or fed-batch medium/media of sodium chloride,
   III.4) more than 0, 10⁻²⁰, 10⁻⁵, 10⁻¹, 1, 10³ or 10⁵ mol per liter of pre-growth and/or growth and/or fed-batch medium/media of manganese sulfate,
   III.5) more than 0, 10⁻²⁰, 10⁻⁶, 10⁻¹, 1, 10³ or 10⁵ mol per liter of pre-growth and/or growth and/or fed-batch medium/media of ferrous sulfate,
   III.6) more than 0, 10⁻²⁰, 10⁻⁶, 10⁻¹, 1, 10³ or 10⁵ mol per liter of pre-growth and/or growth and/or fed-batch medium/media of cobalt nitrate,
   III.7) more than 0, 10⁻²⁰, 10⁻⁶, 10⁻¹, 1, 10³ or 10⁵ mol per liter of pre-growth and/or growth and/or fed-batch medium/media of calcium chloride,
   III.8) more than 0, 10⁻²⁰, 10⁻⁶, 10⁻¹, 1, 10³ or 10⁵ mol per liter of pre-growth and/or growth and/or fed-batch medium/media of zinc sulfate,
   III.9) more than 0, 10⁻²⁰, 10⁻⁷, 10⁻¹, 1, 10³ or 10⁵ mol per liter of pre-growth and/or growth and/or fed-batch medium/media of copper sulfate,
   III.10) more than 0, 10⁻²⁰, 10⁻⁷, 10⁻¹, 1, 10³ or 10⁵ mol per liter of pre-growth and/or growth and/or fed-batch medium/media of aluminium potassium sulfate originating from Wolf's minerals or mineral elixir,
   III.11) more than 0, 10⁻²⁰, 10⁻⁷, 10⁻¹, 1, 10³ or 10⁵ mol per liter of pre-growth and/or growth and/or fed-batch medium/media of boric acid,
   III.12) more than 0, 10⁻²⁰, 10⁻⁸, 10⁻¹, 1, 10³ or 10⁵ mol per liter of pre-growth and/or growth and/or fed-batch medium/media of sodium molybdate,
   III.13) more than 0, 10⁻²⁰, 10⁻⁹, 10⁻¹, 1, 10³ or 10⁵ mol per liter of pre-growth and/or growth and/or fed-batch medium/media of sodium selenite,
   III.14) more than 0, 10⁻²⁰, 10⁻⁷, 10⁻¹, 1, 10³ or 10⁵ mol per liter of pre-growth and/or growth and/or fed-batch medium/media of sodium tungstate,
   III.15) more than 0, 10⁻²⁰, 10⁻⁷, 10⁻¹, 1, 10³ or 10⁵ mol per liter of pre-growth and/or growth and/or fed-batch medium/media of nickel chloride,
   III.16) more than 0, 10⁻²⁰, 10⁻⁸, 10⁻¹, 1, 10³ or 10⁵ mol per liter of pre-growth and/or growth and/or fed-batch medium/media of Wolf's mineral or mineral elixir, and
   III.17) more than 0, 10 different components of Wolf's mineral or mineral elixir;
IV) more than 0, 10⁻²⁰, 10⁻⁵, 10⁻¹, 1, 10³ or 10⁵ mol per liter of pre-growth and/or growth and/or fed-batch medium/media of EDTA;
and/or
V) at least one compound or assembly of compounds comprised in or originating from Wolf's vitamins selected in the group consisting of:
   V.1) more than 0, 10⁻²⁰, 10⁻⁹, 10⁻¹, 1, 10³ or 10⁵ mol per liter of pre-growth and/or growth and/or fed-batch medium/media of biotin,
   V.2) more than 0, 10⁻²⁰, 10⁻⁸, 10⁻¹, 1, 10³ or 10⁵ mol per liter of pre-growth and/or growth and/or fed-batch medium/media of calcium pantothenate,
   V.3) more than 0, 10⁻²⁰, 10⁻⁹, 10⁻¹, 1, 10³ or 10⁵ mol per liter of pre-growth and/or growth and/or fed-batch medium/media of folic acid,
   V.4) more than 0, 10⁻²⁰, 10⁻⁹, 10⁻¹, 1, 10³ or 10⁵ mol per liter of pre-growth and/or growth and/or fed-batch medium/media of inositol,
   V.5) more than 0, 10⁻²⁰, 10⁻⁸, 10⁻¹, 1, 10³ or 10⁵ mol per liter of pre-growth and/or growth and/or fed-batch medium/media of nicotinic acid,
   V.6) more than 0, 10⁻²⁰, 10⁻⁸, 10⁻¹, 1, 10³ or 10⁵ mol per liter of pre-growth and/or growth and/or fed-batch medium/media of p-Aminobenzoic acid,
   V.7) more than 0, 10⁻²⁰, 10⁻⁹, 10⁻¹, 1, 10³ or 10⁵ mol per liter of pre-growth and/or growth and/or fed-batch medium/media of pyridoxine HCl,
   V.8) more than 0, 10⁻²⁰, 10⁻⁹, 10⁻¹, 1, 10³ or 10⁵ mol per liter of pre-growth and/or growth and/or fed-batch medium/media of riboflavin,
   V.9) more than 0, 10⁻²⁰, 10⁻⁹, 10⁻¹, 1, 10³ or 10⁵ mol per liter of pre-growth and/or growth and/or fed-batch medium/media of Thiamine HCl,
   V.10) more than 0, 10⁻²⁰, 10⁻⁸, 10⁻¹, 1, 10³ or 10⁵ mol per liter of pre-growth and/or growth and/or fed-batch medium/media of thiotic acid,
   V.11) more than 0, 10⁻²⁰, 10⁻⁹, 10⁻¹, 1, 10³ or 10⁵ mol of at least one component of Wolf's vitamin, and
   V.12) more than 0, 1, 5, 10, 15 or 20 different Wolf's vitamin; and/or
      preferentially wherein the pre-growth, growth, and/or fed-batch medium/media does/do not comprise, preferentially as measured per mg or per gram or per milliliter or per liter of pre-growth, growth and/or fed-batch medium/media, more than:
1) 0, 10⁻²⁰, 10⁻¹⁰, 10⁻⁵, 10⁻³ or 1 gram of protein preferentially originating from yeast extract,
2) 0, 10⁻²⁰, 10⁻¹⁰, 10⁻⁶, 10⁻³ or 1 gram of nucleic acids preferentially originating from yeast extract,
3) 0, 10⁻²⁰, 10⁻¹⁰, 10⁻, 10⁻¹ or 1 gram of glutathione preferentially originating from yeast extract,
4) 0, 10⁻²⁰, 10⁻¹⁰, 10⁻⁷, 10⁻¹ or 1 gram of at least one the compounds preferentially originating from yeast extract selected from the group consisting of: dextran, mannan, trehalose, flavoring nucleotide, B vitamins, biotin, and volatile aromatic compounds.
5) 0, 10⁻²⁰, 10⁻¹⁰, 10⁻⁷, 10⁻¹ or 1 gram of calcium preferentially originating from yeast extract,
6) 0, 10⁻²⁰, 10⁻¹⁰, 10⁻⁶, 10⁻¹ or 1 gram of phosphorous preferentially originating from yeast extract,
7) 0, 10⁻²⁰, 10⁻¹⁰, 10⁻⁸, 10⁻¹ or 1 gram of zinc preferentially originating from yeast extract,
8) 0, 10⁻²⁰, 10⁻¹⁰, 10⁻⁵, 10⁻¹ or 1 gram of chrome preferentially originating from yeast extract,
9) 0, 10⁻²⁰, 10⁻¹⁰, 10⁻⁷, 10⁻⁵, 10⁻¹ or 1 gram of potassium preferentially originating from yeast extract,
10) 0, 10⁻²⁰, 10⁻¹⁰, 10⁻⁵, 10⁻¹ or 1 gram of cobalt preferentially originating from yeast extract,
11) 0, 10⁻²⁰, 10⁻⁹, 10⁻⁵, 10⁻¹ or 1 gram of manganese preferentially originating from yeast extract,
12) 0, 10⁻²⁰, 10⁻¹⁰, 10⁻⁵, 10⁻¹ or 1 gram of strontium preferentially originating from yeast extract,
13) 0, 10⁻²⁰, 10⁻¹⁰, 10⁻⁷, 10⁻³, 10⁻¹ or 1 gram of magnesium preferentially originating from yeast extract,
14) 0, 10⁻²⁰, 10⁻¹⁰, 10⁻⁴, 10⁻¹ or 1 gram of yeast extract,
15) 0, 1, 5, 10 or 15 different components originating from yeast extract,
16) 0, 10⁻²⁰, 10⁻¹⁰, 10⁻⁴, 10⁻¹ or 1 gram of ashes preferentially originating from peptone,
17) 0, 10⁻²⁰, 10⁻¹⁰, 10⁻³, 10⁻¹ or 1 gram of proteins preferentially originating from peptone,
18) 0, 10⁻²⁰, 10⁻¹⁰, 10⁻⁴, 10⁻¹ or 1 gram of sucrose preferentially originating from peptone,
19) 0, 10⁻²⁰, 10⁻¹⁰, 10⁻⁵, 10⁻¹ or 1 gram of raffinose preferentially originating from peptone,
20) 0, 10⁻²⁰, 10⁻¹⁰, 10⁻⁴, 10⁻¹ or 1 gram of neutral detergent fiber preferentially originating from peptone,
21) 0, 10⁻²⁰, 10⁻¹⁰, 10⁻⁵, 10⁻¹ or 1 gram of Ethereal Extract preferentially originating from peptone,
22) 0, 10⁻²⁰, 10⁻¹⁰, 10⁻⁵, 10⁻¹ or 1 gram of peptone,
23) 0, 1, 5, 7, 10 or 15 different components originating from peptone,
24) 0, 10⁻²⁰, 10⁻¹⁰, 10⁻⁵, 10⁻¹ or 1 mol of nitrilotriacetic acid preferentially originating from Wolf's minerals or mineral elixir,
25) 0, 10⁻²⁰, 10⁻¹⁰, 10⁶, 10⁻¹ or 1 mol of magnesium sulfate preferentially originating from Wolf's minerals or mineral elixir,
26) 0, 10⁻²⁰, 10⁻¹⁰, 10⁻⁵, 10⁻¹ or 1 mol of sodium chloride preferentially originating from Wolf's minerals or mineral elixir,
27) 0, 10⁻²⁰, 10⁻¹⁰, 10⁻⁵, 10⁻¹ or 1 mol of manganese sulfate preferentially originating from Wolf's minerals or mineral elixir,
28) 0, 10⁻²⁰, 10⁻¹⁰, 10⁻⁶, 10⁻¹ or 1 mol of ferrous sulfate preferentially originating from Wolf's minerals or mineral elixir,
29) 0, 10⁻²⁰, 10⁻¹⁰, 10⁻⁶, 10⁻¹ or 1 mol of cobalt nitrate preferentially originating from Wolf's minerals or mineral elixir,
30) 0, 10⁻²⁰, 10⁻¹⁰, 10⁻⁶, 10⁻¹ or 1 mol of calcium chloride preferentially originating from Wolf's minerals or mineral elixir,
31) 0, 10⁻²⁰, 10⁻¹⁰, 10⁻⁶, 10⁻¹ or 1 mol of zinc sulfate preferentially originating from Wolf's minerals or mineral elixir,
32) 0, 10⁻²⁰, 10⁻¹⁰, 10⁻⁷, 10⁻¹ or 1 mol of copper sulfate preferentially originating from Wolf's minerals or mineral elixir,
33) 0, 10⁻²⁰, 10⁻¹⁰, 10⁻⁷, 10⁻¹ or 1 mol of aluminium potassium sulfate preferentially originating from Wolf's minerals or mineral elixir,
34) 0, 10⁻²⁰, 10⁻¹⁰, 10⁻⁷, 10⁻¹ or 1 mol of boric acid preferentially originating from Wolf's minerals or mineral elixir,
35) 0, 10⁻²⁰, 10⁻¹⁰, 10⁻⁸, 10⁻³, 10⁻¹ or 1 mol of sodium molybdate preferentially originating from Wolf's minerals or mineral elixir,
36) 0, 10⁻²⁰, 10⁻¹⁵, 10⁻⁹, 10⁻³, 10⁻¹ or 1 mol of sodium selenite preferentially originating from Wolf's minerals or mineral elixir,
37) 0, 10⁻²⁰, 10⁻⁷, 10⁻³, 10⁻¹ or 1 mol of sodium tungstate preferentially originating from Wolf's minerals or mineral elixir,
38) 0, 10⁻²⁰, 10⁻⁷, 10⁻³, 10⁻¹ or 1 mol of nickel chloride preferentially originating from Wolf's minerals or mineral elixir,
39) 0, 10⁻²⁰, 10⁻⁸, 10⁻³, 10⁻¹ or 1 mol of Wolf's mineral or mineral elixir,
40) 0, 1, 5, 10 or 20 different components of Wolf's mineral or mineral elixir,
41) 0, 10⁻²⁰, 10⁻¹⁰, 10⁻⁵, 10⁻¹ or 1 mol of EDTA,
42) 0, 10⁻⁵⁰, 10⁻³⁰, 10⁻⁹, 10⁻³, 10⁻¹ or 1 mol of biotin preferentially originating from Wolf's vitamins,
43) 0, 10⁻⁵⁰, 10⁻³⁰, 10⁻⁸, 10⁻³, 10⁻¹ or 1 mol of Calcium pantothenate preferentially originating from Wolf's vitamins,
44) 0, 10⁻⁵⁰, 10⁻³⁰, 10⁻⁹, 10⁻³, 10⁻¹ or 1 mol of folic acid preferentially originating from Wolf's vitamins,
45) 0, 10⁻⁵⁰, 10⁻³⁰, 10⁻⁹, 10⁻³, 10⁻¹ or 1 mol of inositol preferentially originating from Wolf's vitamins,
46) 0, 10⁻⁵⁰, 10⁻³⁰, 10⁻¹⁰, 10⁻⁸, 10⁻³, 10⁻¹ or 1 mol of nicotinic acid preferentially originating from Wolf's vitamins,
47) 0, 10⁻⁵⁰, 10⁻³⁰, 10⁻⁸, 10⁻³, 10⁻¹ or 1 mol of p-Aminobenzoic acid preferentially originating from Wolf's vitamins,
48) 0, 10⁻⁵⁰, 10⁻³⁰, 10⁻⁹, 10⁻³, 10⁻¹ or 1 mol of pyridoxine HCl preferentially originating from Wolf's vitamins,
49) 0, 10⁻⁵⁰, 10⁻³⁰, 10⁻⁹, 10⁻³, 10⁻¹ or 1 mol of riboflavin preferentially originating from Wolf's vitamins,
50) 0, 10⁻⁵⁰, 10⁻³⁰, 10⁻⁹, 10⁻³, 10⁻¹ or 1 mol of Thiamine HCl preferentially originating from Wolf's vitamins,
51) 0, 10⁻⁵⁰, 10⁻³⁰, 10⁻⁸, 10⁻³, 10⁻¹ or 1 mol of thiotic acid preferentially originating from Wolf's vitamins,
52) 0, 10⁻⁵⁰, 10⁻³⁰, 10⁻⁹, 10⁻³, 10⁻¹ or 1 mol of at least one component of Wolf's vitamin,
53) 0, 1, 5, 10 or 20 different Wolf's vitamins,
54) 0, 1, 2, 3, 6, 10 or 100 different vitamins,
55) 0, 10⁻⁵⁰, 10⁻²⁰, 10⁻⁹, 10⁻⁸, 10⁻⁷, 10⁻⁵, 10⁻³, 10⁻¹ or 1 mol of at least one vitamin,
56) 0, 10⁻⁵⁰, 10⁻²⁰, 10⁻¹⁰, 10⁻⁵, 10⁻⁴, 10⁻², 10⁻¹, 1, 10 or 10³ g of yeast extract,
57) 0, 10⁻⁵⁰, 10⁻²⁰, 10⁻⁹, 10⁻³, 10⁻¹, 1, 5 or 10 mol of at least one component of yeast extract,
58) 0, 1, 2, 5, 10 or 100 different components of yeast extract,
59) 0, 10⁻⁵⁰, 10⁻¹⁰, 10⁻⁵, 10⁻¹, 1, 10 or 10³ g of peptone,
60) 0, 10⁻⁵⁰, 10⁻⁹, 10⁻⁵, 10⁻³, 10⁻¹, 1 or 10 mol of at least one component of peptone,
61) 0, 1, 2, 5, 10 or 100 different components of peptone,
62) 0, 1, 2, 5, 10 or 100 different CMR agents,
63) 0, 10⁻⁵⁰, 10⁻⁹, 10⁻⁵, 0.05, 10⁻¹, 1, 10, 10³ or 10⁶ mg of at least one CMR agent,
64) 0, 1, 2, 5, 10 or 100 different chelating agents,
65) 0, 10⁻⁵⁰, 10⁻²⁰, 10⁻⁹, 10⁻⁸, 10⁻³, 10⁻¹, 1, 5, 10 or 10³ mol of at least one chelating agent,
66) 0, 1, 2, 5, 10 or 100 different amino acids,
67) 0, 10⁻⁵⁰, 10⁻¹⁰, 10⁻⁵, 10⁻³, 1, 10, 10³, 10⁵ or 10¹⁰ mg of at least one amino acid,
68) 0, 1, 2, 5, 10 or 100 different toxic or cytotoxic compounds,
69) 0, 10⁻⁵⁰, 10⁻¹⁰, 10⁻⁵, 10⁻³, 10⁻¹, 1, 10, 10³ or 10⁵ mg of at least one toxic or cytotoxic compound,
70) 0, 1, 3 or 7 different heavy metals different from iron,
71) 0, 10⁻⁵⁰, 10⁻¹⁰, 10⁻⁵, 10⁻³, 10⁻¹, 1, 10, 10³ or 10⁵ mg of at least one heavy metal different from iron,
72) 0, 1, 2, 3, 4, 5, 6, 7, 8, 9, 10 metal(s) or chemical element(s) chosen among cadmium, lead, arsenic, mercury, cobalt, vanadium, nickel, lithium, antimony, and copper,
73) 1 mg of cadmium, lead, arsenic, mercury, cobalt, vanadium, nickel, lithium, antimony, and/or copper,
74) 0, 10⁻⁵⁰, 10⁻¹⁰, 0.5, 1, 5, 10, 10³ or 10⁶ mL or 10⁻⁵⁰, 10⁻³⁰, 10⁻¹⁰, 10⁻⁸, 10⁻⁵, 10⁻³, 1, 10, 10³ or 10⁶ mol of Wolf's vitamin,
75) 10⁻⁵⁰, 10⁻²⁰, 10⁻⁹, 10⁻⁵, 10⁻¹, 1, 5, 10, 10³ or 10⁵ mol of at least one component of Wolf's vitamin,
76) 0, 1, 2, 5, 10 or 100 different components of Wolf's vitamin,
77) 0, 10⁻⁵⁰, 10⁻¹⁰, 0.5, 1, 5, 10, 10³ or 10⁶ mL or 10⁻⁵⁰, 10⁻³⁰, 10⁻¹⁰, 10⁻⁸, 10⁻⁵, 10⁻³, 1, 10, 10³ or 10⁶ mol of Wolf's mineral,
78) 10⁻⁵⁰, 10⁻²⁰, 10⁻⁹, 10⁻⁵, 10⁻¹, 1, 5, 10, 10³ or 10⁵ mol of at least one component of Wolf's mineral,
79) 0, 1, 2, 5, 7, 10, 15 or 100 different components of Wolf's mineral,
80) 10⁻⁵⁰, 10⁻²⁰, 10⁻⁹, 10⁻⁵, 10⁻¹, 1, 5, 10, 10³ or 10⁵ mol of mineral elixir,
81) 10⁻⁵⁰, 10⁻²⁰, 10⁻⁹, 10⁻⁵, 10⁻¹, 1, 5, 10, 10³ or 10⁵ mol of at least one component of mineral elixir, and/or
82) 0, 1, 2, 5, 10, 14 or 100 different components of mineral elixir.

The invention also relates to the method according to the invention, wherein the fed-batch medium comprises at least one compound that is selected from the group consisting of: iron, iron source, carbon, carbon source, nitrogen, nitrogen source, and combination thereof and the concentration of the at least one compound in the fed-batch medium is larger than 10⁻⁶ μM, 1 μM, and/or 10⁶ μM.

In one embodiment of the invention, at least one vitamin, component of Wolf's vitamin or vitamin solution is selected from the group consisting of: folic acid, folates, pyridoxine, pyridoxamine, pyridoxal, riboflavin, biotin, thiamine, nicotinic acid, pantothenic acid, vitamin B12, amino benzoic acid, thiotic acid, all-trans-Retinol, Retinals, alternative provitamin A-functioning Carotenoids including all-trans-beta-carotene, Niacin, Niacinamide, Nicotinamide, riboside, cyanocobalamin, hydroxocobalamin, methylcobalamin, adenosylcobalamin, ascorbic acid, cholecalciferol, ergocalciferol, tocopherols, tocotrienols, phylloquinone, menaquinones, vitamin A, vitamin B₁, vitamin B₂, vitamin B₃, vitamin B₅, vitamin B₆, vitamin B₇, vitamin B₉, vitamin B₁₂, vitamin C, vitamin D, vitamin D₂, vitamin D₃, vitamin E, vitamin K, vitamin Vᵢ where V can be any letter from A to Z and i can be any integer between 1 and 100, and derivatives thereof.

In another embodiment of the invention, at least one component of Wolf's mineral is selected from the group consisting of: nitrilotriacetic acid, magnesium sulfate, sodium chloride, manganese sulfate, ferrous sulfate heptahydrate, cobalt nitrate, calcium chloride, zinc sulfate heptahydrate, hydrate copper sulfate, aluminum potassium sulfate dodecahydrate, boric acid, sodium molybdate, sodium selenite, sodium tungstate dihydrate, nickel chloride, and derivatives thereof.

In another embodiment of the invention, at least one component of yeast extract is selected from the group consisting of: i) at least one protein, ii) at least one nucleic acid, iii) at least one functional peptide, iv) glutathione, v) dextran, vi) mannan, vii) trehalose, viii) flavoring nucleotide, ix) B vitamin, x) biotin, x) at least one volatile aromatic compound, xi) calcium, xii) Phosphorus, xiii) Zinc, xiv) Iron, xv) Chromium, xvi) Potassium, xvii) Cobalt, xviii) Manganese, xix) Strontium, xx) Magnesium, and xxi) derivatives thereof.

In another embodiment of the invention, at least one component of Mineral elixir is selected from the group consisting of: nitrilotriacetic acid, MgSO₄, MnSO₄, NaCl, FeSO₄, CoSO₄, CaCl₂, ZnSO₄, CuSO₄, KAl(SO₄)₂, H₃BO₃, Na₂MoO₄, NiCl₂, Na₂SeO₃, and derivatives thereof.

In one embodiment of the invention, it is equivalent to say that the pre-growth, growth, and/or fed-batch medium/media does/do not comprise more than a certain quantity or concentration or number of at least one compound than to say that the pre-growth, growth, and/or fed-batch medium/media comprise(s) less than a certain quantity or concentration or number of at least one compound.

In one embodiment of the invention, the nanoparticle-producing cells are amplified or grown in the pre-growth and/or growth and/or fed-batch medium/media, preferentially in the pre-growth and/or growth medium/media and not in the fed-batch medium.

In one embodiment of the invention, the growth and/or pre-growth medium/media comprise(s) the growth and/or pre-growth medium/media either: i) without/before the fed-batch medium is inserted in the growth and/or pre-growth medium/media or ii) with/after the fed-batch medium is inserted in the growth and/or pre-growth medium/media.

In one embodiment of the invention, the fed-batch medium is the fed-batch medium or medium before it is inserted in the growth and/or pre-growth medium/media.

In another embodiment of the invention, the fed-batch medium is part of the pre-growth and/or growth medium/media after it is inserted in the growth and/or pre-growth medium/media.

In one embodiment of the invention, the parameters C_{FEGS}, C_{FePGS}, C_{CGS}, C_{CPGS}, C_{NGS}, C_{NPGS}, ΔₚH_{GS}, and/or ΔpH_{PGS}, exist or is/are measured in the pre-growth, growth and/or fed-batch medium/media.

In one embodiment of the invention, the parameters Q_{GGS}, Q_{GPGS}, N_{SSGS}, and N_{SSPGS}, as well as the situation during which the growth medium is supplemented by a fed-batch medium, whereas the pre-growth medium is not supplemented by such medium, occur during, at the beginning or at the end of the pre-growth and/or growth step(s).

In one embodiment of the invention, the derivative thereof is a derivative of at least one first compound selected in the group consisting of: i) a second compound differing from the at least one first compound by at least one different atom or functional group, preferentially originating from the at least one first compound after transformation of the at least one first compound, preferentially with at least one atom or functional group in common with the at least one first compound, ii) a non-hydrated form of the at least one first compound, iii) a hydrated form of the at least one first compound, iv) a reduced form of the at least one first compound, v) an oxidized form of the at least one first compound, vi) an acidic form of the at least one first compound, vii) a basic form of the at least one first compound, viii) a crystalline or solid form of the at least one first compound, ix) a soluble or solubilized form of the at least one first compound, and x) a salt of the at least one first compound.

The invention relates to the method according the invention, wherein the growth step differs from the pre-growth step by at least one property selected from the group consisting of:
i) a ratio C_{FeGS}/C_{FePGS} that is larger than 10⁻⁵, 10⁻³, 1, 10, 10³ or 10⁵, where C_{FeGS} and C_{FePGS} are concentrations in iron or iron source of the growth medium and pre-growth medium, respectively,
ii) a ratio C_{CGS}/C_{CPGS} that is larger than 10⁻⁵, 10⁻³, 1, 10, 10³ or 10⁵, where C_{CG}S and C_{CPGS} are the concentrations in carbon or carbon source of the growth medium and pre-growth medium, respectively,
iii) a ratio C_{NGS}/C_{NPGS} that is larger than 10⁻⁵, 10⁻³, 1, 10, 10³ or 10⁵, where C_{NGS} and C_{NPGS} are the concentrations in nitrogen or nitrogen source of the growth medium and pre-growth medium, respectively,
iv) a ratio ΔpH_{GS}/ΔpH_{PGS} that is lower than 10¹⁰, 10⁵, 10³, 10², 1, 0.5 or 0.1, where Δ_{pHGS} and ΔpH_{PGS} are the pH variations of the growth medium and pre-growth medium, respectively,
v) a ratio Q_{GGS}/Q_{CPGS} that is larger than 10¹⁰, 10⁵, 10³, 10², 1, 0.5 or 0.1, where Q_{GGS} and Q_{GPGS} are quantities of gas, oxygen or air brought in or bubbled through the growth medium and pre-growth medium, respectively,
vi) a ratio N_{SSGS}/N_{SSPGS} that is lower than 10⁻⁵, 10⁻³, 1, 10, 10³ or 10⁵, where N_{SSGS} and N_{SSPGS} are numbers of sub-steps of the growth step and numbers of sub-steps of the pre-growth step, respectively, where two sub-steps are separated by each other by a transfer of nanoparticle-producing cells from a first sub-step, preferentially associated to the growth of nanoparticle-producing cells in a first volume, to a second sub-step, preferentially associated to the growth of nanoparticle-producing cells in a second volume, and
vii) the growth medium is supplemented by a fed-batch medium, whereas the pre-growth medium is not supplemented by such medium.

The invention also relates to the method according to the invention, wherein the pre-growth and/or growth and/or fed-batch medium/media comprise(s), preferentially per kilogram or liter of pre-growth and/or growth and/or fed-batch medium/media, less than:
i) 10³% in mass or volume, or 1% in mass or volume, or 10⁻²% in mass or volume, or 5·10⁻³% in mass or volume, or 10³ gram, or 10² gram, or 10 gram, or 10¹⁰ mL, or 10⁵ mL, or 10³ mL, or 10 mL, or 5 mL, or 1 mL, or 0.5 mL, or 10⁻⁵ mL, or 10³ mol, or 10 mol, or 1 mol, or 10⁻⁵ mol, or 10⁻⁸ mol, or 10⁻⁹ mol, or 10⁻¹⁰ mol of vitamins, preferentially Wolf's vitamins, or chemical components selected from the group consisting of: folic acid, folates, pyridoxine, Pyridoxine HCl, pyridoxamine, pyridoxal, riboflavin, biotin, thiamine, thiamine HCl, nicotinic acid, pantothenic acid, calcium pantothenate, inositol, p-Aminobenzoic acid, amino benzoic acid, thiotic acid, all-trans-Retinol, Retinals, alternative provitamin A-functioning Carotenoids including all-trans-beta-carotene, Niacin, Niacinamide, Nicotinamide, riboside, cyanocobalamin, hydroxocobalamin, methylcobalamin, adenosylcobalamin, ascorbic acid, cholecalciferol, ergocalciferol, tocopherols, tocotrienols, phylloquinone, menaquinones, vitamin A, vitamin B1, vitamin B2, vitamin B3, vitamin B5, vitamin B6, vitamin B7, vitamin B9, vitamin B12, vitamin C, vitamin D, vitamin D2, vitamin D3, vitamin E, vitamin K, vitamin Vᵢ where V can be any letter from A to Z and i can be any integer between 1 and 100, and derivatives thereof,
ii) 1, 5, 6, 10 or 20 different vitamins, preferentially Wolf's vitamins, or chemical components, which are selected from the group consisting of: folic acid, folates, pyridoxine, Pyridoxine HCl, pyridoxamine, pyridoxal, riboflavin, biotin, thiamine, thiamine HCl, nicotinic acid, pantothenic acid, calcium pantothenate, inositol, p-Aminobenzoic acid, amino benzoic acid, thiotic acid, all-trans-Retinol, Retinals, alternative provitamin A-functioning Carotenoids including all-trans-beta-carotene, Niacin, Niacinamide, Nicotinamide, riboside, cyanocobalamin, hydroxocobalamin, methylcobalamin, adenosylcobalamin, ascorbic acid, cholecalciferol, ergocalciferol, tocopherols, tocotrienols, phylloquinone, menaquinones, vitamin A, vitamin B1, vitamin B2, vitamin B3, vitamin B5, vitamin B6, vitamin B7, vitamin B9, vitamin B12, vitamin C, vitamin D, vitamin D2, vitamin D3, vitamin E, vitamin K, vitamin Vᵢ where V can be any letter from A to Z and i can be any integer between 1 and 100, and derivatives thereof,
iii) 10³% in mass or volume, or 10% in mass or volume, or 1% in mass or volume, or 10⁻²% in mass or volume, or 10⁻⁵% in mass or volume, or 10³ gram, or 10 gram, or 1 gram, or 10⁵ mL, or 10³ mL, or 10 mL, or 1 mL, or 10⁻³ mL, or 10³ mol, or 10 mol, or 1 mol, or 10⁻³ mol, or 10⁻⁷ mol, or 10⁻⁸ mol, or 10⁻¹⁰ mol of minerals, preferentially Wolf's mineral or mineral elixir, or chemical components selected from the group consisting of: nitrilotriacetic acid, magnesium sulfate, sodium chloride, manganese sulfate, ferrous sulfate, ferrous sulfate heptahydrate, cobalt nitrate, calcium chloride, zinc sulfate, zinc sulfate heptahydrate, copper sulfate, hydrate copper sulfate, aluminum potassium sulfate, aluminum potassium sulfate dodecahydrate, boric acid, sodium molybdate, sodium selenite, sodium tungstate, sodium tungstate dihydrate, nickel chloride, EDTA, MgSO₄, MnSO₄, NaCl, FeSO₄, CoSO₄, CaCl₂, ZnSO₄, CuSO₄, KAl(SO₄)₂, H₃BO₃, Na₂MoO₄, NiCl₂, Na₂SeO₃, and derivatives thereof,
iv) 1, 3, 7 or 10 different components of minerals, preferentially Wolf's mineral or mineral elixir, or chemical components selected from the group consisting of: nitrilotriacetic acid, magnesium sulfate, sodium chloride, manganese sulfate, ferrous sulfate, ferrous sulfate, ferrous sulfate heptahydrate, cobalt nitrate, calcium chloride, zinc sulfate, zinc sulfate heptahydrate, copper sulfate, hydrate copper sulfate, aluminium potassium sulfate, aluminum potassium sulfate dodecahydrate, boric acid, sodium molybdate, sodium selenite, sodium tungstate, sodium tungstate dihydrate, nickel chloride, EDTA, MgSO₄, MnSO₄, NaCl, FeSO₄, CoSO₄, CaCl₂, ZnSO₄, CuSO₄, KAl(SO₄)₂, H₃BO₃, Na₂MoO₄, NiCl₂, Na₂SeO₃, and derivatives thereof,
v) 10⁻⁵⁰ gram, or 10⁻¹⁰ gram, or 10⁻⁵ gram, or 0.005 gram, or 10⁻¹ gram, or 1 gram, or 10 gram, or 10⁵ gram, or 10⁻⁵⁰ M, or 10⁻⁸ M, or 10⁻³ M, or 10⁻¹ M, or 1 M, or 10³ M of at least one component of yeast extract or at least one compound originating from yeast extract selected from the group of compounds consisting of: at least one protein, at least one nucleic acid, at least one functional peptide, glutathione, dextran, mannan, trehalose, flavoring nucleotide, B vitamin, biotin, at least one volatile aromatic compound, calcium, Phosphorus, Zinc, Iron, Chrome or Chromium, Potassium, Cobalt, Manganese, Strontium, Magnesium, and derivatives thereof,
vi) 1, 2, 3, 5, 10, 15, 20 or 50 different components of yeast extract or compounds originating from yeast extract selected from the group consisting of: at least one protein, at least one nucleic acid, at least one functional peptide, glutathione, dextran, mannan, trehalose, flavoring nucleotide, B vitamin, biotin, at least one volatile aromatic compound, calcium, Phosphorus, Zinc, Iron, Chrome or Chromium, Potassium, Cobalt, Manganese, Strontium, Magnesium, and derivatives thereof,
vii) 10⁻⁵⁰ gram, or 10⁻¹⁰ gram, or 10⁻³ gram, or 0.01 gram, or 1 gram, or 5 gram, or 10 gram or 10⁵ gram, or 10⁻⁵⁰ M, or 10⁻²⁰ M, or 10⁻⁸ M, or 10⁻³ M, or 10⁻¹ M, or 1 M, or 10 M, or 10³ M of at least one component of peptone or at least one compound originating from peptone selected from the group consisting of: ashes, proteins, sucrose, stachyose, raffinose, neutral detergent fiber, Ethereal Extract, and derivatives thereof,
viii) 1, 3, 5, 10, 20 or 50 different components of peptone or compounds originating from peptone selected from the group consisting of: ashes, proteins, sucrose, stachyose, raffinose, neutral detergent fiber, Ethereal Extract, and derivatives thereof,
ix) 10⁻⁵⁰, 10⁻¹⁰, 10⁻⁵, 0.001, 10⁻¹, 1, 10, 10³ or 10⁵ gram of EDTA,
x) 10⁻⁵⁰, 10⁻¹⁰, 10⁻⁵, 0.001, 10⁻¹, 1, 10, 10³ or 10⁵ gram of at least one amino acid,
xi) 1, 3, 5, 10, 20 or 50 different amino acids,
xii) 1, 5, 7, 12, 15, 20 or 50 different CMR, toxic or cytotoxic compounds selected from the group consisting of: nitrilotriacetic acid, manganese sulfate, cobalt nitrate, zinc sulfate, copper sulfate, aluminum potassium sulfate, boric acid, sodium molybdate, sodium selenite, sodium tungstate, nickel chloride, and derivatives thereof,
xiii) 1, 2, 5, 10, 50 or 100 different chemical elements or heavy metal selected in the group consisting of: cadmium, lead, arsenic, mercury, cobalt, vanadium, nickel, lithium, antimony, and copper, and derivatives thereof,
xiv) 10⁻⁵⁰, 10⁻¹⁰, 10⁻⁵, 10⁻¹, 1, 10, 10³ or 10⁵ gram of at least one chemical element or heavy metal selected in the group consisting of: cadmium, lead, arsenic, mercury, cobalt, vanadium, nickel, lithium, antimony, and copper, and derivatives thereof,
xv) 10⁻⁵⁰, 10⁻¹⁰, 10⁻⁵, 10⁻¹, 1, 10, 10³ or 10⁵ gram of at least one CMR, toxic or cytotoxic compound selected from the group consisting of: nitrilotriacetic acid, manganese sulfate, cobalt nitrate, zinc sulfate, copper sulfate, aluminum potassium sulfate, boric acid, sodium molybdate, sodium selenite, sodium tungstate, nickel chloride, and any derivative thereof, and/or
xvi) 10⁻⁵⁰, 10⁻¹⁰, 10⁻⁵, 0.01, 10⁻¹, 1, 10, 10³ or 10⁵ gram of peptone.

The invention also relates to the method according to the invention, wherein at least one compound of the pre-growth and/or growth medium/media has a concentration C₂ or a concentration Cₜₒₜₐₗ=C₁+C₂, wherein:
C₁ is a concentration of the at least one compound of the pre-growth and/or growth medium/media not consumed by the nanoparticle-producing cells,
C₂ is a concentration of the at least one compound of the pre-growth and/or growth medium/media consumed by nanoparticle-producing cells,
C₁ and/or C₂ is/are preferentially measured or separated or differentiated by using a method that enables isolating the at least one compound consumed by the nanoparticle-producing cells from the at least one compound not consumed by the nanoparticle-producing cells, such as centrifugation or tangential filtration, and
C₁ and/or C₂ is/are preferentially measured or considered at the beginning, during or at the end of the pre-growth and/or growth step(s).

In one embodiment of the invention, one compound of the pre-growth and/or growth medium/media consumed by nanoparticle-producing cells is one compound that is comprised in the nanoparticle cells, preferentially when nanoparticle-producing cells consume such compound.

In another embodiment of the invention, one compound of the pre-growth and/or growth medium/media not consumed by nanoparticle-producing cells is one compound that is comprised outside of the nanoparticle cells, preferentially when nanoparticle-producing cells do not consume such compound.

In one embodiment, a method is used to separate consumed and non-consumed compounds. This method preferentially separates whole bacteria from the pre-growth and/or growth medium/media that do(does) not comprise such bacteria. This method can be centrifugation or filtration, preferentially tangential filtration, or a method that enables separating whole bacteria from liquid medium, based on a separation between elements of low size and/or low weight comprised in the pre-growth and/or growth medium/media and elements of large size and/or large weight in whole bacteria.

The invention also relates to the method according to the invention, wherein the concentration of at least one compound comprised in the fed-batch medium, preferentially, iron, iron source, carbon, carbon source, nitrogen, and/or nitrogen source, is larger than 10⁻⁶ μM, 1 μM, and/or 10⁶ μM.

The invention also relates to the method according to the invention, wherein the pre-growth, growth, and/or fed-batch medium/media do(does) not comprise the at least one compound at a concentration that affects the growth of nanoparticle-producing cells and/or nanoparticle production, and/or wherein the pre-growth, growth, and/or fed-batch medium/media are(is) substantially free of the at least one compound, wherein the at least one compound is selected from the group consisting of: 1) Wolf's Vitamin or a medium that comprises more than half of the total number of different components of Wolf's vitamin, 2) one component of Wolf's vitamin, 3) folic acid, 4) pyridoxine, 5) riboflavin, 6) biotin, 7) thiamin, 8) nicotinic acid, 9) pantothenic acid, 10) vitamin B₁₂, 11) amino benzoic acid, 12) thiotic acid, 13) Wolf's mineral or a medium that comprises more than half of the total number of different components of Wolf's mineral, 14) nitrilotriacetic acid, 15) magnesium sulfate, 16) sodium chloride, 17) manganese sulfate, 18) ferrous sulfate heptahydrate, 19) cobalt nitrate, 20) calcium chloride, 21) zinc sulfate heptahydrate, 22) hydrate copper sulfate, 23) aluminum potassium sulfate dodecahydrate, 24) boric acid, 25) sodium molybdate, 26) sodium selenite, 27) sodium tungstate dihydrate, 28) yeast extract or a medium that comprises more than half of the total number of different components of yeast extract, 29) equivalent of yeast extract or a medium that comprises more than half of the total number of different components of the equivalent of yeast extract, 30) 1, 2 or 5 protein(s) originating from or comprised in yeast extract, 31) 1, 2 or 5 nucleic acid(s) originating from or comprised in yeast extract, 32) 1, 2 or 5 peptide(s) or functional peptide(s) originating from or comprised in yeast extract, 33) glutathione, 34) dextran, 35) mannan, 36) trehalose, 37) flavoring nucleotide originating from or comprised in yeast extract, 38) B vitamin, 39) biotin, 40) 1, 2 or 5 volatile aromatic compound(s) originating from or comprised in yeast extract, 41) Chromium, 42) Cobalt, 43) Strontium, 44) nickel chloride, 45) or a medium that comprises more than half of the total number of different components of mineral elixir, 46) MnSO₄, 47) NaCl, 48) FeSO₄, 49) CoSO₄, 50) CaCl₂, 51) ZnSO₄, 52) CuSO₄, 53) KAl(SO₄)₂, 54) H₃BO₃, 55) Na₂MoO₄, 56) NiCl₂, 57) Na₂SeO₃, 58) peptone or a medium that comprises more than half of the total number of different components of peptone, 59) one component of peptone, 60) 1, 2 or 5 protein(s) originating from or comprised in peptone, 61) a sugar originating from or comprised in peptone, 62) one amino acid originating from or comprised in peptone, 63) ashes originating from or comprised in peptone, 64) one fiber originating from or comprised in peptone, 65) one CMR agent, 66) boric acid, 67) one amino acid, 68) alanine, 69) arginine, 70) asparagine, 71) aspartic acid, 72) cysteine, 73) glutamine, 74) glutamic acid, 75) glycine, 76) histidine, 77) isoleucine, 78) leucine, 79) lysine, 80) methionine, 81) phenylalanine, 82) proline, 83) serine, 84) threonine, 85) tryptophan, 86) tyrosine, 87) valine, 88) one cytotoxic or toxic compound, 89) manganese sulfate, 90) copper sulfate, 91) aluminum potassium sulfate, 92) boric acid, 93) sodium tungstate, 94) one heavy metal different from iron, 95) Titanium, 96) Vanadium, 97) Manganese, 98) Nickel, 99) Copper, 100) Zinc, 101) Gallium, 102) Germanium, 103) Arsenic, 104) Zirconium, 105) Niobium, 106) Molybdenum, 107) Technetium, 108) Ruthenium, 109) Rhodium, 110) Palladium, 111) Silver, 112) Cadmium, 113) Indium, 114) Tin, 115) Tellurium, 116) Lutetium, 117) Hafnium, 118) Tantalum, 119) Tungsten, 120) Rhenium, 121) Osmium, 122) Iridium, 123) Platinum, 125) Gold, 126) Mercury, 127) Thallium, 128) Lead, 129) Bismuth, 130) Polonium, 131) Astatine, 132) Lanthanum, 133) Cerium, 134) Praseodymium, 135) Neodymium, 136) Promethium, 137) Samarium, 138) Europium, 139) Gadolinium, 140) Terbium, 141) Dysprosium, 142) Holmium, 143) Erbium, 144) Thulium, 145) Ytterbium, 146) Actinium, 147) Thorium, 148) Protactinium, 149) Uranium, 150) Neptunium, 151) Plutonium, 152) Americium, 153) Curium, 154) Berkelium, 155) Californium, 156) Einsteinium, 157) Fermium, 158) Nobelium, 159) Radium, 160) Lawrencium, 161) Rutherfordium, 162) Dubnium, 163) Seaborgium, 164) Bohrium, 165) Hassium, 166) Meitnerium, 167) Darmstadtium, 168) Roentgenium, 169) Copernicium, 170) Elements 113-118, 171) Helium, 172) Lithium, 173) Beryllium, 174) Bore, 175) Fluor, 176) Aluminum, 177) Silicon, 178) Argon, 179) Scandium, 180) Chrome, 181) Nickel, 182) Copper, 183) Selenium, 184) Brome, 185) Krypton, 186) Rubidium, 187) Yttrium, 188) Sn, 189) Antimony, 190) Iodine, 191) Xenon, 192) Cesium, 193) Barium, 194) Lutecium, 195) Astate, 196) Radon, 197) Francium, 198) Mendelevium, 199) Mount, 200) Ununbium, 201) Ununtrium, 202) Ununquadium, 203) Ununpentium, 204) Ununhexium, 205) Ununseptium, 206) Ununoctium, 207) salts of these compounds 1) to 206), and 208) derivatives thereof.

The invention also relates to the method according to the invention, wherein the concentration of the compound that affects the growth of nanoparticle-producing cells and/or nanoparticle production, is a concentration in the pre-growth, growth and/or fed-batch medium/media that is: i) larger than 1 pico-Molar, 1 micro-Molar, 1 mili-Molar, 10⁻⁵⁰ M, 10⁻¹⁰ or 10⁻⁵ M, or ii) larger than 10⁻⁵⁰, 10⁻¹⁰, 10⁻⁵ or 10⁻³ gram of compound per liter of pre-growth, growth and/or fed-batch medium/media.

In one embodiment of the invention, the concentration of the compound that affects the growth of nanoparticle-producing cells and/or nanoparticle production, is a concentration in the pre-growth, growth and/or fed-batch medium/media that is: i) smaller than 10⁵⁰, 1, 10⁻⁵, 10⁻⁶ or 10⁻⁹ M, or ii) smaller than 10¹⁰, 1, 10⁻¹⁰ or 10⁻²⁰ gram of compound per liter of pre-growth, growth and/or fed-batch medium/media.

The invention relates to the method according to the invention, wherein the growth medium and/or fed-batch medium/media is/are supplemented by a fed-batch medium and:
i) the fed-batch medium has a pH that is lower, preferentially by at least 10⁻⁵, 0.1, 0.5, 1, 2, 3 or 5 pH unit(s), than a pH of the pre-growth and/or growth medium/media, and/or
ii) the concentration of at least one of the chemical elements, preferentially selected from the group consisting of: a) the phosphorous or phosphate source, b) the potassium source, c) the magnesium source, d) the iron source, e) the vitamin source, f) the calcium source, g) KH₂PO₄, h) MgSO₄, i) FeCl₃, j) thiamine, k) CaCl₂, and 1) derivatives thereof, is larger in the fed-batch medium than in the pre-growth and/or growth medium/media, preferentially by a factor of at least 10⁻⁵⁰, 10⁻¹⁰, 0, 1.1, 5, 10 or 10³.

In one embodiment, the above conditions i) and ii) are verified by considering the pH and concentration of at least one compound:
i) before the fed-batch medium has been inserted in the pre-growth and/or growth medium/media for the fed-batch medium, and/or
ii) before, during or after the fed-batch medium has been inserted in the pre-growth and/or growth medium/media for the pre-growth and/or growth medium/media.

The invention also relates to the method according to the invention, wherein:
the nanoparticle-producing cells are magnetotactic bacteria, and/or
the nanoparticles are magnetosomes.

In another embodiment of the invention, the nanoparticles are the nanoparticle-producing cells of high purity.

In another embodiment of the invention, the nanoparticles are high purity nanoparticles, preferentially high purity iron oxide based nanoparticles, where high purity nanoparticles are preferentially nanoparticles that comprise less than 100, 99, 90, 50, 20, 10, 1 or 0.1% in mass or number of atoms selected in the group consisting of: cadmium, lead, arsenic, mercury, cobalt, vanadium, nickel, lithium, antimony, copper and derivatives, where high purity iron oxide based nanoparticles are preferentially high purity nanoparticles that comprise more than 1, 50, 90, 93 or 99% in number of atoms or mass of iron and/or oxygen and/or iron oxide.

The invention also relates to the method according to the invention, wherein the pre-growth and/or growth medium/media comprise(s) a source of calcium, a source of carbon, a source of nitrogen, a source of phosphate or phosphorous, a source of sulfur, a source of iron, a source of vitamin, and a source of calcium, and:
the concentration of the source of carbon in the pre-growth and/or growth medium/media is larger, preferentially by a factor of at least 0, 0.5, 1.1, 2, 4, 10 or 100, than the concentrations of at least one compound selected from the group consisting of: the source of phosphate or phosphorous, the source of sulfur, the source of vitamin, and the source of calcium in the pre-growth and/or growth medium/media, and/or
the concentration of the source of nitrogen in the pre-growth and/or growth medium/media is larger, preferentially by a factor of at least 0, 0.5, 1.1, 2, 4, 10 or 100, than the concentrations of at least one compound selected from the group consisting of: the source of phosphate or phosphorous, the source of sulfur, the source of vitamin, and the source of calcium in the pre-growth and/or growth medium/media.

The invention also relates to the method according to the invention, further comprising a step of storing, amplifying, preparing, or inserting in the pre-growth and/or growth and/or fed-batch medium/media a bank of nanoparticle-producing cells, where such bank is preferentially stored, amplified, prepared in a bank medium that preferentially comprises at least 10⁻⁵⁰, 10⁻¹⁰, 10⁻¹, 0, 1, 5, 10, 50, 70, 90 or 99%, preferentially in number of compounds, of the same compounds as those of the pre-growth and/or growth and/or fed-batch medium/media, and preferentially at least one compound that is different from that of the pre-growth and/or growth and/or fed-batch medium/media, such as a vitamin, a mineral, a chelating agent, sucrose, and/or a cryo-protectant that is/are: i) not in the pre-growth and/or growth and/or fed-batch medium/media or ii) at a lower concentration in the pre-growth and/or growth and/or fed-batch medium/media than in the bank medium.

In one embodiment of the invention, the bank of nanoparticle-producing cells is a master, working or research cell bank of the nanoparticle-producing cells. In some cases, such bank comprises more than 1, 10, 10³, 10¹⁰ or 10²⁰ nanoparticle-producing cell(s), preferentially per milliliter or liter of pre-growth and/or growth and/or fed-batch medium. In some other cases, such bank comprises less than 10¹⁰⁰, 10⁵⁰, 10²⁰, 10¹⁰ or 10³ nanoparticle-producing cell(s).

In another embodiment of the invention, the cell bank is an assembly of at least one cell that is used to start the amplification of the nanoparticle-producing cells in the pre-growth and/or growth and/or fed-batch medium.

In one embodiment of the invention, the cell bank is prepared in the same or similar conditions as those of the pre-growth and/or growth step(s).

In another embodiment of the invention, the cell bank is prepared by bubbling or inserting a gas in the bank medium that comprises a low oxygen concentration, preferentially less than 50, 10 or 1% of oxygen, preferentially relatively to the volume of the bank medium.

The invention relates to the method according to the invention, further comprising a purifying step for obtaining high purity iron oxide based nanoparticle(s), preferentially starting from nanoparticles that are isolated from the nanoparticle-producing cells obtained at the end of the growth step, the purifying step preferentially comprising removal at least one impurity(ies) from the nanoparticle(s) produced in the growth step using at least one heating step in which the temperature of the nanoparticles according to the invention, which are preferentially produced in the growth step or essentially in the growth step, have a temperature that is increased to a temperature Tᵢ, and is then maintained at Tᵢ during a heating time tₕᵢ, which is preferentially comprised between 1 second and 20 years, where Tᵢ is preferentially comprised between 50° C. and 700° C.

In one embodiment of the invention, Tᵢ is larger than −273, −100, −50, 0, 1, 10, 20, 50, 100, 200, 500, 700 or 103° C.

In one embodiment of the invention, Tᵢ is smaller than 10²⁰, 10¹⁰, 10⁵, 10³, 100, 50, 10, 0, −10 or −50° C.

In still another embodiment of the invention, Tᵢ is between 10 and 10⁵, 50 and 10⁴, 100 and 10³, 150 and 700, or between 200 and 500° C.

In one embodiment of the invention, tₕᵢ is larger than 10⁻⁵⁰, 10⁻¹⁰, 10⁻⁵, 10⁻¹, 1, 10, 10², 10³, 10⁵ or 10¹⁰ second(s).

In another embodiment of the invention, tₕᵢ is lower than 10¹⁰⁰, 10⁵⁰, 10¹⁰, 10⁵, 10³, 10, 5, 2 or 1 second.

In still another embodiment of the invention, tₕᵢ is comprised between 10⁻⁵ seconds and 1 year, 1 second and 20 years, 1 second and 1 year, 1 second and 1 month, 1 second and 1 week, 1 second and day, or between 1 second and 1 hour.

In one embodiment of the invention, the time to increase the temperature to Tᵢ, preferentially from an initial temperature that preferentially corresponds to the temperature of the nanoparticles when they are inserted in the equipment used to heat them, is lower, preferentially by a factor of at least 1.1, 5, 10 or 10³, than tₕᵢ.

In another embodiment of the invention, the time to increase the temperature to Tᵢ, is larger, preferentially by a factor of at least 1.1, 5, 10 or 10³, than tₕᵢ.

In one embodiment of the invention, iron oxide based nanoparticles are nanoparticles that comprise more than 1%, 50%, 70%, 90% or 99%, preferentially in mass of iron oxide, preferentially without considering in this percentage coating or excipient material.

The invention also relates to nanoparticle-producing cells of high purity preferentially obtained by the method according to the invention, the nanoparticle-producing cells of high purity comprising more than 10⁻¹⁰, 1, 5, 10, 50, 75, 80, 90, 95, 99 or 99.9% of:
i) iron based on a ratio M_{Fe}C/M_{MC}, where M_{Fe}C is the mass of iron in the nanoparticle-producing cells of high purity and M_{MC} is the mass of iron and metals or metalloids other than iron in the nanoparticle-producing cells of high purity,
ii) iron and at least one other metal than iron selected in the group consisting of: Sodium, Magnesium, Aluminum, Potassium, Calcium, Scandium, Titanium, Chromium, Manganese, Zinc, Gallium, Strontium, Yttrium, Zirconium, Niobium, Molybdenum, Technetium, Indium, Cesium, Barium, Lanthanum, Cerium, Praseodymium, Neodymium, Samarium, Europium, Gadolinium, Terbium, Dysprosium, Holmium, Erbium, Thulium, Lutetium, Hafnium, Rhenium, and Tungstate, based on a ratio of M₁/M₂, where M₁ is the mass in the high purity iron oxide nanoparticle(s) of iron and of at least one other metal selected in the above group, and M₂ is the mass of all metals comprised in the high purity iron oxide nanoparticle(s), and/or
iii) iron and at least one other non-metal selected in the group consisting of: Hydrogen, Carbon, Nitrogen, Phosphorus, Sulfur, Fluorine, Chlorine, Bromine, Iodine, Helium, Neon, Argon, Krypton, Xenon, Radon, and Oxygen, based on a ratio M₃/M₄, where M₃ is the mass in the high purity iron oxide nanoparticle(s) of iron and of at least one other metal selected in the above group and M₄ is the mass of all chemical elements comprised in the high purity iron oxide nanoparticle(s).

The invention also relates to nanoparticle-producing cells of high purity or high purity iron oxide based nanoparticle(s) obtained by amplifying or growing nanoparticle-producing cells in a pure medium that comprises less than 1, 10⁻³, 10⁻⁶ or 10⁻⁹% of at least one heavy metal selected in the group consisting of: Cobalt, Manganese, Zinc, Nickel, Silver, Aluminum, Arsenic, Barium, Cadmium, Chrome, Copper, Molybdate, lead, Antimony, Selenium, Silica, Titan, Thallium, mercury, vanadium, gold, iridium, osmium, rhodium, ruthenium, platinum, lithium, antimony, Tin, Tungsten, and derivatives thereof, where this percentage is based on the ratio C_{FeM}/C_{MM}, where C_{FeM} is the concentration of iron in the pure medium and C_{MM} is the concentration of iron and metals or metalloids other than iron in the pure medium.

The invention also relates to high purity iron oxide based nanoparticle(s) preferentially obtained by the method according to the invention, the high purity iron oxide based nanoparticle(s) comprising more than 10⁻¹⁰, 1, 5, 10, 50, 75, 80, 90, 93, 95, 99 or 99.9% of:
i) iron based on a ratio of M_{FeN}/M_{MN}, where M_{FeN} is the mass of iron in the high purity iron oxide nanoparticle(s) and M_{MN} is the mass of iron and metals or metalloids other than iron in the high purity iron oxide nanoparticle(s),
ii) iron and at least one other metal than iron selected in the group consisting of: Sodium, Magnesium, Aluminum, Potassium, Calcium, Scandium, Titanium, Chromium, Manganese, Zinc, Gallium, Strontium, Yttrium, Zirconium, Niobium, Molybdenum, Technetium, Indium, Cesium, Barium, Lanthanum, Cerium, Praseodymium, Neodymium, Samarium, Europium, Gadolinium, Terbium, Dysprosium, Holmium, Erbium, Thulium, Lutetium, Hafnium, Rhenium, and Tungstate, based on a ratio of M₁/M₂, where M₁ is the mass in the high purity iron oxide nanoparticle(s) of iron and of at least one other metal than iron selected in the above group, and M₂ is the mass of all metals comprised in the high purity iron oxide nanoparticle(s), and/or
iii) iron and at least one other non-metal selected in the group consisting of: Hydrogen, Carbon, Nitrogen, Phosphorus, Sulfur, Fluorine, Chlorine, Bromine, Iodine, Helium, Neon, Argon, Krypton, Xenon, Radon, and Oxygen, based on a ratio M₃/M₄, where M₃ is the mass in the high purity iron oxide nanoparticle(s) of iron and of at least one other metal selected in the above group and M₄ is the mass of all non-metals comprised in the high purity iron oxide nanoparticle(s).

The invention also relates to the nanoparticle-producing cells of high purity according to the invention and/or the high purity iron oxide based nanoparticle(s) according to the invention, wherein the metals or metalloids other than iron in the high purity iron oxide nanoparticle(s) and/or nanoparticle-producing cells of high purity are selected in the group consisting of: Sodium, Magnesium, Aluminum, Potassium, Calcium, Scandium, Titanium, Chromium, Manganese, Zinc, Gallium, Strontium, Yttrium, Zirconium, Niobium, Molybdenum, Technetium, Indium, Cesium, Barium, Lanthanum, Cerium, Praseodymium, Neodymium, Samarium, Europium, Gadolinium, Terbium, Dysprosium, Holmium, Erbium, Thulium, Lutetium, Hafnium, Rhenium, and Tungstate, at least 5 different metals or metalloids, and derivatives thereof.

The invention also relates to the high purity iron oxide based nanoparticle(s) according to the invention, wherein the high purity iron oxide based nanoparticles are magnetosomes.

The invention also relates to a composition comprising the high purity iron oxide based nanoparticles according to the invention.

The invention also relates to the nanoparticle-producing cells of high purity, also designated as highly pure nanoparticle-producing cells, according to the invention, wherein highly pure nanoparticle-producing cells are magnetotactic bacteria.

The invention also relates to a composition comprising the highly pure nanoparticle-producing cells according to the invention.

The invention also relates to a composition comprising highly pure nanoparticle-producing cells and high purity iron oxide nanoparticles preferentially obtained by the method according to the invention, wherein:
the highly pure nanoparticle-producing cells comprise more than 0, 1, 10, 50, 70, 90, 95 or 99% of:
i) iron based on a ratio M_{Fe}C/M_{MFe}, where M_{Fe}C is the mass of iron in the highly pure nanoparticle-producing cells and MMC is the mass of iron and metals or metalloids other than iron in the highly pure nanoparticle-producing cells,
ii) iron and at least one other metal than iron selected in the group consisting of: Sodium, Magnesium, Aluminum, Potassium, Calcium, Scandium, Titanium, Chromium, Manganese, Zinc, Gallium, Strontium, Yttrium, Zirconium, Niobium, Molybdenum, Technetium, Indium, Cesium, Barium, Lanthanum, Cerium, Praseodymium, Neodymium, Samarium, Europium, Gadolinium, Terbium, Dysprosium, Holmium, Erbium, Thulium, Lutetium, Hafnium, Rhenium, and Tungstate, based on a ratio of M₁/M₂, where M₁ is the mass in the high purity iron oxide nanoparticle(s) of iron and of at least one other metal selected in the above group, and M₂ is the mass of all metals comprised in the high purity iron oxide nanoparticle(s), and/or
iii) iron and at least one other non-metal selected in the group consisting of: Hydrogen, Carbon, Nitrogen, Phosphorus, Sulfur, Fluorine, Chlorine, Bromine, Iodine, Helium, Neon, Argon, Krypton, Xenon, Radon, and Oxygen, based on a ratio M₃/M₄, where M₃ is the mass in the high purity iron oxide nanoparticle(s) of iron and of at least one other non-metal selected in the above group and M₄ is the mass of all non-metallic elements comprised in the high purity iron oxide nanoparticle(s), and the high purity iron oxide based nanoparticle(s) comprise more than 0, 1, 10, 50, 75, 93, 99 or 99.9% of:
i) iron based on a ratio of M_{FeN}/M_{MN}, where M_{FeN} is the mass of iron in the high purity iron oxide nanoparticle(s) and M_{MN} is the mass of iron and metals or metalloids other than iron in the high purity iron oxide nanoparticle(s),
ii) iron and at least one other metal than iron selected in the group consisting of: Sodium, Magnesium, Aluminum, Potassium, Calcium, Scandium, Titanium, Chromium, Manganese, Zinc, Gallium, Strontium, Yttrium, Zirconium, Niobium, Molybdenum, Technetium, Indium, Cesium, Barium, Lanthanum, Cerium, Praseodymium, Neodymium, Samarium, Europium, Gadolinium, Terbium, Dysprosium, Holmium, Erbium, Thulium, Lutetium, Hafnium, Rhenium, and Tungstate, based on a ratio of M₁/M₂, where M₁ is the mass in the high purity iron oxide nanoparticle(s) of iron and of at least one other metal selected in the above group, and M₂ is the mass of all metals comprised in the high purity iron oxide nanoparticle(s), and/or
iii) iron and at least one other non-metal selected in the group consisting of: Hydrogen, Carbon, Nitrogen, Phosphorus, Sulfur, Fluorine, Chlorine, Bromine, Iodine, Helium, Neon, Argon, Krypton, Xenon, Radon, and Oxygen, based on a ratio M₃/M₄, where M₃ is the mass in the high purity iron oxide nanoparticle(s) of iron and of at least one other non-metal selected in the above group and M₄ is the mass of all non-metallic elements comprised in the high purity iron oxide nanoparticle(s).

The invention also relates to a composition comprising highly pure nanoparticle-producing cells and/or high purity iron oxide nanoparticles, wherein:
the nanoparticle-producing cells of high purity comprise more than 0, 10⁻⁵⁰, 10⁻¹⁰, 10⁻⁵, 10⁻², 1, 5, 10, 25, 50, 75, 90, 95, 99 or 99% of:
i) iron based on a ratio M_{Fe}C/M_{MC}, where M_{Fe}C is the mass of iron in the nanoparticle-producing cells of high purity and M_{MC} is the mass of iron and metals or metalloids other than iron in the nanoparticle-producing cells of high purity,
ii) iron and at least one other metal than iron selected in the group consisting of: Sodium, Magnesium, Aluminum, Potassium, Calcium, Scandium, Titanium, Chromium, Manganese, Zinc, Gallium, Strontium, Yttrium, Zirconium, Niobium, Molybdenum, Technetium, Indium, Cesium, Barium, Lanthanum, Cerium, Praseodymium, Neodymium, Samarium, Europium, Gadolinium, Terbium, Dysprosium, Holmium, Erbium, Thulium, Lutetium, Hafnium, Rhenium, and Tungstate, based on a ratio of M₁/M₂, where M₁ is the mass in the high purity iron oxide nanoparticle(s) of iron and of at least one other metal selected in the above group, and M₂ is the mass of all metals comprised in the high purity iron oxide nanoparticle(s), and/or
iii) iron and at least one other non-metal selected in the group consisting of: Hydrogen, Carbon, Nitrogen, Phosphorus, Sulfur, Fluorine, Chlorine, Bromine, Iodine, Helium, Neon, Argon, Krypton, Xenon, Radon, and Oxygen, based on a ratio M₃/M₄, where M₃ is the mass in the high purity iron oxide nanoparticle(s) of iron and of at least one other non-metal selected in the above group and M₄ is the mass of all non-metallic elements comprised in the high purity iron oxide nanoparticle(s),
and
the high purity iron oxide based nanoparticle(s) comprise more than 0, 10⁻⁵, 10⁻¹⁰, 10⁻⁵, 10⁻³, 10⁻¹, 0, 1, 5, 10, 25, 50, 75, 93, 95, 99 or 99.9% of:
i) iron based on a ratio of M_{FeN}/M_{MN}, where M_{FeN} is the mass of iron in the high purity iron oxide nanoparticle(s) and M_{MN} is the mass of iron and metals or metalloids other than iron in the high purity iron oxide nanoparticle(s),
ii) iron and at least one other metal than iron selected in the group consisting of: Sodium, Magnesium, Aluminum, Potassium, Calcium, Scandium, Titanium, Chromium, Manganese, Zinc, Gallium, Strontium, Yttrium, Zirconium, Niobium, Molybdenum, Technetium, Indium, Cesium, Barium, Lanthanum, Cerium, Praseodymium, Neodymium, Samarium, Europium, Gadolinium, Terbium, Dysprosium, Holmium, Erbium, Thulium, Lutetium, Hafnium, Rhenium, and Tungstate, based on a ratio of M₁/M₂, where M₁ is the mass in the high purity iron oxide nanoparticle(s) of iron and of at least one other metal selected in the above group, and M₂ is the mass of all metals comprised in the high purity iron oxide nanoparticle(s), and/or
iii) iron and at least one other non-metal selected in the group consisting of: Hydrogen, Carbon, Nitrogen, Phosphorus, Sulfur, Fluorine, Chlorine, Bromine, Iodine, Helium, Neon, Argon, Krypton, Xenon, Radon, and Oxygen, based on a ratio M₃/M₄, where M₃ is the mass in the high purity iron oxide nanoparticle(s) of iron and of at least one other non-metal selected in the above group and M₄ is the mass of all non-metallic elements comprised in the high purity iron oxide nanoparticle(s),
wherein the nanoparticle-producing cells of high purity and/or the high purity iron oxide based nanoparticle(s) are preferentially obtained by cultivating the nanoparticle-producing cells in a growth medium, which is substantially free of at least one metal or non-metal or comprises less than 10 M, or 1 M, or 10⁻¹ M, or 10⁻³ M, or 10⁻⁶ M, or 1 nano-Molar of at least one metal or non-metal, wherein the at least one metal or non-metal is selected in the group consisting of: 1) cadmium, 2) lead, 3) arsenic, 4) mercury, 5) cobalt, 6) vanadium, 7) nickel, 8) lithium, 9) antimony, 10) copper, 11) Valadium, 12) Molybdate, 13) Selenium, 14) Baryum, 15) Chrome, 16) Strontium, 17) a radioactive chemical element, 18) Beryllium, 19) Rubidium, 20) Ruthenium, 21) Rhodium, 22) Palladium, 23) Promethium, 24) Ytterbium, 25) Tantalum, 26) Osmium, 27) Iridium, 28) Bismuth, 29) Polonium, 30) Francium, 31) Radium, 32) Actinium, 33) Thorium, 34) Protactinium, 35) Uranium, 36) Neptunium, 37) Plutonium, 38) Americium, 39) Curium, 40) Berkelium, 41) Californium, Einsteinium, 42) Fermium, 43) Mendelevium, 44) Nobelium, 45) Lawrencium, 46) Rutherfordium, 47) Dubnium, 48) Seaborgium, 49) Bohrium, 50) Hassium, 51) Meitnerium, 52) Darmstadtium, 53) Roentgenium, 54) Copernicium, 55) Nihonium, 56) Flerovium, 57) Moscovium, 58) Livermorium, 59) Astatine, 60) Tennessine, 61) Oganesson, and 62) derivatives thereof.

In one embodiment of the invention, the nanoparticle-producing cells of high purity and/or the high purity iron oxide based nanoparticle(s) are obtained by cultivating and/or amplifying the nanoparticle-producing cells in:
a) the pre-growth growth and/or growth medium/media,
and/or
b) a medium that does not comprise or does not comprise at a concentration that affects the growth of the nanoparticle-producing cells, at least one metal or metalloid other than iron preferentially selected in the group consisting of: Cobalt, Manganese, Zinc, Nickel, Silver, Aluminum, Arsenic, Barium, Cadmium, Chrome, Copper, Molybdate, lead, Antimony, Selenium, Silica, Titan, Thallium, mercury, vanadium, gold, iridium, osmium, rhodium, ruthenium, platinum, lithium, antimony, Tin, Tungsten, and derivatives thereof.

The invention also relates to the nanoparticle-producing cells of high purity according to the invention, and/or the high purity iron oxide based nanoparticle(s) preferentially obtained from the nanoparticle-producing cells of high purity according to the invention, wherein:
the nanoparticle-producing cell(s) of high purity is/are magnetotactic bacterium/bacteria, and/or
the high purity iron oxide nanoparticles is/are magnetosome(s).

The invention also relates to a composition, a medical device, a drug, a preparation, a suspension, a cosmetic composition, a plant composition, a biological composition, a mineral composition, and/or a nanoparticle composition, comprising the nanoparticle-producing cells of high purity according to the invention and/or the high purity iron oxide nanoparticle(s) according to the invention.

The invention also relates to the nanoparticle-producing cells of high purity preferentially comprising more than 1, 25, 50, 75, 90 or 99% of iron based on a ratio M_{FeC}/M_{MC}, where M_{FeC} is the mass of iron in the nanoparticle-producing cells of high purity and M_{M}C is the mass of iron and metals or metalloids other than iron in the nanoparticle-producing cells of high purity and/or the high purity iron oxide based nanoparticle(s) preferentially comprising more than 1, 10, 50, 75, 93 or 99% of iron based on a ratio of M_{FeN}/M_{MN}, where M_{FeN} is the mass of iron in the high purity iron oxide nanoparticle(s) and M_{MN} is the mass of iron and metals or metalloids other than iron in the high purity iron oxide nanoparticle(s), wherein the nanoparticle-producing cells of high purity and/or the high purity iron oxide based nanoparticle(s) are preferentially obtained by cultivating the nanoparticle-producing cells in a growth medium, which is substantially free of metals selected in the group consisting of: cadmium, lead, arsenic, mercury, cobalt, vanadium, nickel, lithium, antimony, and copper.

The term “substantially free” herein refers to substances that are not intentionally added to the medium but could be present as impurities.

In some cases, M_{FeN}/M_{MN} and/or M_{FeN}/M_{MN} can be smaller than 100, 99, 95, 90, 50, 25, 10, 5 or 1%.

The invention also relates to a method, preferentially the method according to the invention, for producing high purity iron oxide nanoparticles using nanoparticle-producing cells amplified in a pre-growth step followed by a growth step, wherein:
a) the pre-growth step is characterized by at least one of the following properties:
   a1) it comprises at least one sub-step i, during which nanoparticle-producing cells are amplified in a volume V_{PGSi} comprising a pre-growth medium,
   a2) V_{PGSi} does not vary by more than 1, 10, 50, 80, 90 or 99% between the beginning and the end of the sub-step i;
   a3) i is preferentially comprised between 1 and 5, 1 and 10, or between 1 and 1000;
   a4) V_{PGSi+1}/V_{PGSi} is comprised between 1.001 and 1000 or between 1.1 and 50;
   a5) each sub-step i lasts for a length of time comprised between 1 second and 1 year, 1 minute and 1 month, 10 minutes and 1 week, or between 60 minutes and 3 days;
   a6) at least one sub-step i lasts for more than 1 second or 1 hour;
   a7) the temperature of the pre-growth medium of at least one sub-step is comprised between 10 and 60° C., 20 and 50° C., or between 30 and 40° C.;
   a8) The concentration of oxygen or air or compressed air in the pre-growth medium decreases:
      from: a8i) an oxygen, air, or compressed air partial pressure that is preferentially larger than 50 mbar, most preferentially equal to 210 mbar, at the beginning of at the least one sub-step i, or a8ii) an oxygen or air or compressed air percentage in volume relative to the maximum volume occupied by oxygen, air, or compressed air, in saturated pre-growth medium, which is preferentially equal to a percentage comprised between 25 and 100% at the beginning of the at least one sub-step i, or a8iii) an oxygen or air or compressed air volume relative to the volume of the pre-growth medium, which is preferentially comprised between 5 to 25% at the beginning of the at least one sub-step i,
      down to: a8iv) an oxygen, air, or compressed air partial pressure that is preferentially lower than 100 mbar, most preferentially equal to 0 mbar, at the end of the at least one sub-step i, or a8v) an oxygen or air or compressed air percentage in volume relative to the maximum volume occupied by oxygen, air, or compressed air, in saturated pre-growth medium, which is preferentially equal to a percentage comprised between 0 and 50%, at the end of the at least one sub-step i, or a8vi) an oxygen or air or compressed air volume relative to the volume of the pre-growth medium, which is preferentially equal to 0 to 10% at the end of the at least one sub-step i.
   a9) The quantity of oxygen or air or compressed air brought to the Volume V_{PGSi} during at least one sub-step i of the pre-growth step is lower than 100 liters, 1000 mL or 100 mL of oxygen or air or compressed air per minute, preferentially during at least 0, 1, 5, 10 or 50% in time of the whole duration of the at least one sub-step i;
   a10) The pre-growth medium is agitated at a speed comprised between 0 and 100 rotations per minute or between 0 and 10³ meter per minute, where this speed is preferentially that of at least one compound, whether in liquid, gaseous, or solid state, of the pre-growth medium, during the whole or part of sub-step i;
   a11) the pH of the pre-growth medium is not maintained at a fixed pH, preferentially by not adding to the pre-growth medium a fed-batch medium preferentially comprising a source of iron or another medium than the pre-growth medium preferentially comprising a source of iron;
   a12) the pH of the pre-growth medium varies by more than 10⁻⁵, 10⁻¹, 0.5 or 1 pH unit between the beginning and the end of the at least one sub-step i, preferentially from a minimum value that is preferentially lower than 7 to a maximum value that is preferentially larger than 7.
   a13) the pH of the pre-growth medium is maintained at a pH comprised between 3 and 11 or between 6 and 8 by adding to the pre-growth medium a fed-batch medium comprising less than 10⁵, 10³, 10², 10 or 2 μM of iron or source of iron;
   a14) the pre-growth medium comprises a total concentration in at least one of iron, iron source, carbon, carbon source, nitrogen, and nitrogen source, which vary(ies) by less than 100, 50, 20, 10, 5 or 1% between the beginning and the end of the at least one sub-step i;
   a15) the pre-growth medium comprises a total concentration in iron or iron source that is lower than 10⁵ mM, 10³ mM, 10 mM, 2 mM of iron or iron source or 10⁵, 10³, 10², 50, 10, 5, 2, 1, 0.5 g of iron or iron source per liter of pre-growth medium; a16) the pre-growth medium comprises a total concentration in iron or iron source that is larger than 10⁻⁵⁰ M or 1 μM of iron or iron source or 0.4 ng of iron or iron source per liter of pre-growth medium;
   a17) the pre-growth medium comprises a total concentration in carbon or carbon source that is lower than 2 M or 260 g of carbon or carbon source per liter of pre-growth medium;
   a18) the pre-growth medium comprises a total concentration in carbon or carbon source that is larger than 0.1 nM or 0.1 ng of carbon or carbon source per liter of pre-growth medium;
   a19) the pre-growth medium comprises a total concentration in nitrogen or nitrogen source that is lower than 740 mM or 40 g of nitrogen or nitrogen source per liter of pre-growth medium;
   a20) the pre-growth medium comprises a total concentration in nitrogen or nitrogen source that is larger than 0.1 nM or 0.1 ng of nitrogen or nitrogen source per liter of pre-growth medium;
   a21) the quantity or concentration or percentage of carbon or carbon source consumed by the nanoparticle-producing cells between the beginning and the end of the at least one sub-step i is larger than:
      10⁻⁵⁰, 0.01, 1 or 10 g of carbon or carbon source per liter of pre-growth medium or 1 mM of carbon or carbon source, and/or
      10⁻¹⁰, 1, 50 or 75%, where this percentage is preferentially based on the ratio (Q_{Cf}−Q_{Ci})/Q_{Ci}, where Q_{Cf} and Q_{Ci} are the quantities of carbon contained in the pre-growth medium at the end and beginning of the at least one sub-step i, respectively,
   a22) the quantity or concentration or percentage of nitrogen or nitrogen source consumed by the nanoparticle-producing cells between the beginning and the end of the at least one sub-step i is larger than:
      10⁻⁵⁰, 0.001, 1 or 10 g of nitrogen or nitrogen source per liter of pre-growth medium or 0.1 mM of nitrogen or nitrogen source, and/or
      10⁻¹⁰, 1, 50 or 75%, where this percentage is preferentially based on the ratio (Q_{Nf}−Q_{Ni})/Q_{Ni}, where Q_{Nf} and Q_{Ni} are the quantities of nitrogen contained in the pre-growth medium at the end and beginning of the at least one sub-step i, respectively,
   a23) the quantity or concentration or percentage of iron or iron source consumed by the nanoparticle-producing cells between the beginning and the end of the at least one sub-step i is larger than:
      10⁻¹⁰, 0.0001, 1, 10 or 10¹⁰ mg of iron or iron source per liter of pre-growth medium or 0.5 μM of iron or iron source, and/or
      10⁻¹⁰, 1, 20, 50 or 75%, where this percentage is preferentially based on the ratio (Q_{Fef}−Q_{Fei})/Q_{Fei}, where Q_{Fef} and Q_{Fei} are the quantities of iron contained in the pre-growth medium at the end and beginning of the at least one sub-step i, respectively.
   a24) the quantity of carbon, carbon source, nitrogen, nitrogen source, iron, and/or iron source consumed by the nanoparticle-producing cells between the beginning and the end of the at least one sub-step i is smaller than the total concentration in carbon, carbon source, nitrogen, nitrogen source, iron, and/or iron source of the pre-growth medium;
   a25) the total concentration in carbon, carbon source, nitrogen, nitrogen source, iron, and/or iron source of the pre-growth medium does(do) not vary by more than 1, 10, 20, 50, 80 or 99% between the beginning and the end of the sub-step i;
   a26) the concentration in carbon, carbon source, nitrogen, nitrogen source, iron, and/or iron source consumed by the nanoparticle-producing cells of increases by more than 1, 10, 20, 50, 80, 90 or 99% between the beginning and the end of the sub-step i; wherein such conditions preferentially result in nanoparticle-producing cell(s) having at least one of the following properties:
   a27) the nanoparticle-producing cell(s) produce essentially no nanoparticles or the nanoparticle-producing cell(s) produce less than 10¹⁰, 10⁵, 1, 10⁻³ or 10⁻⁶ mg of nanoparticles per liter of pre-growth medium, where this quantity is preferentially the quantity of nanoparticles produced at the end of the at least one sub-step i or the difference between the quantity of nanoparticles produced at the end of the at least one sub-step i and the quantity of nanoparticles produced at the beginning of at least one sub-step i;
   a28) the nanoparticle-producing cells, preferentially without being concentrated at the end of the at least one sub-step i, lead to an optical density, preferentially measured between 0 and 10⁵ nm, most preferentially at 565 nm, which is characterized by at least one of the following properties:
      a28i) it varies within a range of optical densities comprised between 10⁻²⁰ and 10²⁰ or between 0.0001 and 40 during the at least one sub-step i of the pre-growth step;
      a28ii) it increases by a factor of more than 0, 0.5, 1, 1.1, 5, 10, 10³ or 10⁵ between the beginning and the end of the at least one sub-step i, where this factor is preferentially the ratio between the optical density measured at the end of sub-step i and the optical density measured at the beginning of sub-step i;
      a28iii) it increases by a factor of less than 10¹⁰, 10⁵, 2000, 10³, 10², 10, 5, 2 or 1 between the beginning and the end of the at least one sub-step i;
      a28iv) it has a maximum value at the end of the at least one sub-step i that is lower than 10⁵, 10³ or 100;
      a28v) it has a minimum value at the beginning of the at least one sub-step i that is larger than 0, 10⁻⁵⁰, 10⁻¹⁰, 0.0001, 10⁻³ or 10⁻¹;
   a29) the nanoparticle producing-cells are characterized by a doubling time or a duration for being multiplied in number by a factor of 2, preferentially during at least one sub-step i of the pre-growth step, which is:
      a29i) larger than 10⁻⁵⁰, 10⁻⁵, 1, 10, 10² or 10³ minute(s);
      a29ii) smaller than 10³, 10, 1 or 0.1 month; and/or
      a29iii) comprised between 1 second and 1 month or between 1 minute and 1 month;
b) a growth step comprising amplifying the nanoparticle-producing cell(s) originating from the pre-growth step and/or produced during the pre-growth step in at least one growth step, preferentially only one growth step, most preferentially a number of growth steps smaller than the number of sub-steps i of the pre-growth step, wherein the at least one growth step is characterized by at least one property selected from the group consisting of:
   b1) The nanoparticle-producing cells are amplified in a volume V_{GS} comprising a growth medium;
   b2) V_{GS} is larger, preferentially by a factor of at least 0, 1, 1.1, 5, 10 or 10³ than the volume of at least one sub-step i of the pre-growth step;
   b3) V_{GS} is equal to: V_{GS0}+V_{FB}, where V_{GS0} is the volume comprising the growth medium at the beginning of the growth step and V_{FB} is the volume of fed-batch medium that is added to the growth medium during the growth step;
   b4) V_{GS} varies by more than 10⁻³, 10⁻¹, 1, 5, 10, 25, 50 or 75% between the beginning and the end of the at least one growth step, preferentially by adding a fed-batch medium to the growth medium during the growth step;
   b5) The volume comprising the growth medium at the beginning of the at least one growth step, V_{GS0}, is larger than the volume of fed-batch medium added to the growth medium during the at least one growth step, V_{FB}, preferentially by a factor of at least 0, 1, 1.1, 1.5, 2, 5, 10 or 10³;
   b6) The duration of the at least one growth step is comprised between 1 minute and 1 month, preferentially between 40 hours and 15 days,
   b7) The duration of the at least one growth step is larger than the duration of the at least one sub-step i of the pre-growth step, preferentially by a factor of at least 0, 1, 1.1, 2, 5, 10 or 10³;
   b8) The temperature of the growth medium is comprised between 10 and 60° C., 20 and 50° C., or between 30 and 40° C.;
   b9) The concentration of oxygen or air or compressed air in the growth medium decreases:
      from b9i) an oxygen, air, or compressed air partial pressure that is preferentially larger than 1 or 10 mbar, most preferentially equal to 210 mbar, at the beginning of at the least one growth step, or b9ii) an oxygen or air or compressed air percentage in volume relative to the maximum volume occupied by oxygen, air, or compressed air, in saturated growth medium, which is preferentially equal to a percentage comprised between 10 and 100%, at the beginning of the at least one growth step, or 9iii) an oxygen or air or compressed air volume relative to the volume of the growth medium, which is preferentially comprised between 1 to 25% at the beginning of the at least one growth step,
      down to b9iv) an oxygen, air, or compressed air partial pressure that is preferentially lower than 50 or 500 mbar, most preferentially equal to 0 mbar, at the end of the at least one growth step, or b9v) an oxygen or air or compressed air percentage in volume relative to the maximum volume occupied by oxygen, air, or compressed air, in saturated growth medium, which is preferentially equal to a percentage comprised between 0 and 25%, at the end of the at least one growth step, or b9vi) an oxygen or air or compressed air volume relative to the volume of the pre-growth medium, which is preferentially comprised between 0 and 5% at the end of the at least one growth step.
   b10) The quantity of oxygen or air or compressed air brought to the volume V_{GS} during the whole growth step or part of the whole growth step is larger than 1, 10 or 200 mL of oxygen or air or compressed air per minute, preferentially during more that 1% in time of the whole duration of the growth step;
   b11) The quantity of oxygen or air or compressed air brought to the volume V_{GS} during the growth step is larger, preferentially by a factor of at least 0, 1, 1.1, 5, 10 or 10³ at the end than at the beginning of the growth step;
   b12) The quantity of oxygen or air or compressed air brought to the volume V_{GS} increases during the whole growth step or part of the whole growth step by an amount that is larger than 10⁻¹⁰, 10⁻⁵, 1, 10 or 10⁵ mL of oxygen or air or compressed air per minute;
   b13) The growth medium is agitated at a higher speed than the growth medium, preferentially at a speed larger than 1, 10 or 100 rotations per minute or between 0 and 10¹ meter per minute, where this speed is preferentially that of at least one compound, whether in liquid, gaseous, or solid state, of the growth medium, during the whole or part of the growth step;
   b14) The pH of the growth medium varies less than the pH of the pre-growth medium or is maintained at a fixed pH comprised between 1 and 14 or between 6.5 and 7.5 or is prevented from varying by more than 0.1, 0.5 or 10 pH unit, preferentially by adding to the growth medium a fed-batch medium or another medium than the growth medium;
   b15) The pH of the growth medium varies by less than 10, 0.5 or 0.1 pH unit between the beginning and the end of the growth step, preferentially from a minimum value that is preferentially lower than 7.5 to a maximum value that is preferentially larger than 6.5.
   b16) The growth medium comprises a total concentration in carbon, carbon source, nitrogen, nitrogen source, iron, and/or iron source that vary(ies) by more than 0, 10⁻⁵, 1 or 50% between beginning and the end of the at least one growth step,
   b17) The growth medium comprises a total concentration in carbon or carbon source that is larger than 10⁻⁵ mM or 0.1 mM or 10⁻⁵ g or 0.01 g of carbon or carbon source per liter of growth medium;
   b18) The growth medium comprises a total concentration in carbon or carbon source that is lower than 10³ M or 2 M or 10³ g or 180 g of carbon or carbon source per liter of growth medium;
   b19) The growth medium comprises a total concentration in nitrogen or nitrogen source that is larger than 10⁻⁵⁰ mM or 0.01 mM or 5·10⁻¹⁰ g or 0.00005 g of nitrogen or nitrogen source per liter of growth medium;
   b20) The growth medium comprises a total concentration in nitrogen or nitrogen source that is lower than 10¹⁰ mM or 111 mM or 10⁵ g or 6 g of nitrogen or nitrogen source per liter of growth medium;
   b21) The growth medium comprises a total concentration in iron or iron source that is larger than 10⁻⁵ nM or 1 nM or 10⁻¹⁰ g or 3·10⁻⁷ g of iron or iron source per liter of growth medium;
   b22) The growth medium comprises a total concentration in iron or iron source that is lower than 10⁵ or 1 mM or 10⁵ g or 0.3 g of iron or iron source per liter of growth medium;
   b23) The quantity or concentration or percentage of carbon or carbon source consumed by the nanoparticle-producing cells between the beginning and the end of the at least one growth step is larger than:
      10⁻¹⁰, 10⁻⁵, 0.1, 1, 10 or 10³ g of carbon or carbon source per liter of growth medium or 1 mM of carbon or carbon or carbon source, and/or
      10⁻⁵⁰, 10⁻¹⁰, 10⁻⁵, 10⁻¹, 1, 5, 10, 50 or 75%, where this percentage is based on the ratio (Q_{Cf}−Q_{Ci})/Q_{Ci}, where Q_{Cf} and Q_{Ci} are the quantities of carbon contained in the growth medium at the end and beginning of the at least one growth step, respectively,
   b24) the quantity or concentration or percentage of nitrogen or nitrogen source consumed by the nanoparticle-producing cells between the beginning and the end of the at least one growth step is larger than:
      10⁻¹⁰, 10⁻⁵, 0.01, 1 or 10 g of nitrogen or nitrogen source per liter of growth medium or 0.6 mM of nitrogen or nitrogen source, and/or
      10⁻⁵⁰, 10⁻¹⁰, 10⁻⁵, 10⁻¹, 1, 5, 10, 50 or 75%, where this percentage is preferentially based on the ratio (Q_{Nf}−Q_{Ni})/Q_{Ni}, where Q_{Nf} and Q_{Ni} are the quantities of nitrogen contained in the growth medium at the end and beginning of the at least one growth step, respectively,
   b25) the quantity or concentration or percentage of iron or iron source consumed by the nanoparticle-producing cells between the beginning and the end of the at least one growth step is larger than:
      10⁻¹⁰, 10⁻⁵, 0.01 or 1 mg of iron or iron source per liter of growth medium or 0.04 μM of iron or iron source, and/or
      10⁻⁵⁰, 10⁻¹⁰, 10⁻⁵, 10⁻¹, 1, 5, 10, 50 or 75%, where this percentage is preferentially based on the ratio (Q_{Fef}−Q_{Fei})/Q_{Fei}, where Q_{Fef} and Q_{Fei} are the quantities of iron contained in the growth medium at the end and beginning of the at least one growth step, respectively;
   b26) The quantity of iron or iron source brought to the growth medium between the beginning and the end of the at least one growth step, preferentially by the fed-batch medium, is larger than 10⁻¹⁰ mg or 0.3 mg of iron or iron source per liter of growth medium or 10⁻³ μM or 1 μM of iron or iron source;
   b27) The quantity of carbon or carbon source brought to the growth medium between the beginning and the end of the at least one growth step, preferentially by the fed-batch medium, is larger than 10⁻⁵⁰, 10⁻¹⁰, 10⁻⁵ or 0.07 g of carbon or carbon source per liter of growth medium or larger than 10⁻¹⁰, 10⁻⁵, 10⁻³, 0.8, 1 or 10³ mM of carbon or carbon source;
   b28) The quantity of nitrogen or nitrogen source brought to the growth medium between the beginning and the end of the at least one growth step, preferentially by the fed-batch medium, is larger than 10⁻¹⁰, 0.006 or 1 g of nitrogen or nitrogen source per liter of growth medium or 0.4 mM of nitrogen or nitrogen source;
   b29) the quantity of carbon, carbon source, nitrogen, nitrogen source, iron, and/or iron source consumed by the nanoparticle-producing cells between the beginning and the end of the at least one growth step is smaller than the total concentration in carbon, carbon source, nitrogen, nitrogen source, iron, and/or iron source of the growth medium; wherein such conditions preferentially result in nanoparticle-producing cell(s) having at least one of the following properties:
   b30) the nanoparticle-producing cell(s) produce(s) nanoparticles or the nanoparticle-producing cell(s) produce more than 10⁻⁵⁰, 10⁻¹⁰, 0.01 or 1 mg of nanoparticles per liter of growth medium, where this quantity is preferentially the quantity of nanoparticles produced at the end of the growth step or the difference between the quantity of nanoparticles produced at the end of the growth step and the quantity of nanoparticles produced at the beginning of the growth step;
   b31) the nanoparticle-producing cells, preferentially without being concentrated at the end of the at least one growth step, lead to an optical density, preferentially measured between 0 and 10⁴ nm, most preferentially at 565 nm, which is characterized by at least one of the following properties:
      b31i) the optical density at the end of the growth step is larger, preferentially by a factor of at least 0, 0.5, 1, 1.1, 1.5, 2, 5, 10 or 10¹ than the optical density at the end of at least one sub-step i of the pre-growth step;
      b31ii) the optical density of the growth step varies within a range of optical density comprised between 0.001 and 300;
      b31iii) the optical density increases by a factor of more than 0, 0.5, 1, 1.1, 5, 10 or 10³ between the beginning and the end of the at least one growth step, where this factor is preferentially the ratio between the optical density measured at the end of the at least one growth step and the optical density measured at the beginning of the at least one growth step;
      b31iv) the optical density increases by a factor of less than 10¹⁰, 10⁴ or 10 between the beginning and the end of the at least one growth step;
      b31v) the optical density has a maximum value at the end of the at least one growth step that is smaller than 10¹⁰, 10⁵, 300 or 10;
      b31vi) the optical density has a minimum value at the beginning of at least one growth step that is larger than 10⁻⁵⁰, 10⁻¹⁰, 0.001 or 0.01; and/or
      b32) the nanoparticle-producing cells are characterized by a doubling time or a duration for being multiplied in number by a factor of 2, preferentially during the whole growth step or part of the whole growth step, which is
      b32i) larger than 1 minute;
      b32ii) smaller than 1 month;
      b32iii) comprised between 1 minute and 1 month; and/or
      b32iv) lower, preferentially by a factor of at least 1.1, than the doubling time of at least one sub-step i of the pre-growth step;
         wherein preferentially the pre-growth, growth, and/or fed-batch medium/media does/do not comprise, preferentially as measured per liter of pre-growth, growth and/or fed-batch medium/media, more than: i) 1, 2, 3 or 6 different vitamins, ii) 10⁻⁹ mol of at least one vitamin, iii) 10⁻⁴ g of yeast extract, iv) 10⁻⁹ mol of at least one component of yeast extract, v) 1, 2, 5 or 10 components of yeast extract, vi) 10⁻⁵ g of peptone, vii) 1 or 2 different CMR agents, viii) 0.05 mg of at least one CMR agent, ix) 1, 2, or 5 different chelating agents, x) 10⁻⁸ mol of at least one chelating agent, xi) 1, 2 or 5 different amino acids, xii) 1 mg of at least one amino acid, xiii) 1, 2 or 5 different toxic or cytotoxic compounds, xiv) 1 mg of at least one toxic or cytotoxic compound, xv) 1, 3 or 7 different heavy metals different from iron, xvi) 1 mg of at least one heavy metal different from iron, xvii) more than 1, 2, 3, 4, 5, 6, 7, 8, 9, 10 metal(s) or chemical element(s) chosen among cadmium, lead, arsenic, mercury, cobalt, vanadium, nickel, lithium, antimony, and copper, xviii) 1 mg of cadmium, lead, arsenic, mercury, cobalt, vanadium, nickel, lithium, antimony, and/or copper, xix) 0.5 mL or 10⁻⁸ mol of Wolf's vitamin, xx) 10⁻⁹ mol of at least one component of Wolf's vitamin, xxi) 1, 2, 5 or 10 different components of Wolf's vitamin, xxii) 5 mL or 10⁻⁸ mol of Wolf's mineral, xxiii) 10⁻⁸ mol of at least one component of Wolf's mineral, xxiv) 1, 2, 5, 7, 10 or 15 different components of Wolf's mineral, xxv) 10⁻⁹ mol of mineral elixir, xxvi) 10⁻⁹ mol of at least one component of mineral elixir, and/or xxvii) 1, 2, 5, 10 or 14 different components of mineral elixir.

The invention also relates to the method according to the invention, wherein the end of the pre-growth and/or growth step(s) is/are characterized by at least one of the following properties:
i) the optical density of the pre-growth and/or growth medium/media saturates or increases by less than 1 O.D. unit per hour of pre-growth and/or growth step(s);
ii) the bio-mass of the pre-growth and/or growth medium/media saturates or increases by less than 1 g of nanoparticle-producing cells per hour of pre-growth and/or growth step(s);
iii) the nanoparticle-producing cells stop producing nanoparticles or produce less than 0.01 mg of nanoparticles per hour of pre-growth and/or growth step(s);
iv) the fed-batch medium is not anymore added to the pre-growth and/or growth medium/media; and
v) oxygen is not anymore bubbled to the pre-growth and/or growth medium/media.

The invention also relates to the method according to the invention, wherein the beginning of the pre-growth and/or growth step(s) is/are characterized by at least one of the following properties:
i) the optical density of the pre-growth and/or growth medium/media starts to increase, preferentially by more than 0.1 O.D. unit per day of pre-growth and/or growth step(s);
ii) the bio-mass of the pre-growth and/or growth medium/media starts to increase, preferentially by more than 0.1 g of nanoparticle-producing cells per day of pre-growth and/or growth step(s);
iii) the nanoparticle-producing cells start producing nanoparticles, preferentially more than 0.01 mg of nanoparticles per day of pre-growth and/or growth step(s); iv) the fed-batch medium is starts to be added to the pre-growth and/or growth medium/media;
v) oxygen starts to be bubbled to the pre-growth and/or growth medium/media.

The invention also relates to the method according to the invention, wherein the pre-growth, growth, and/or fed-batch medium composition(s) is/are defined hereafter.

Preferentially, the pre-growth medium comprises at least 1, 2, 3, 4, 5, 6, or 7 of the following chemical element(s) or derivative(s), preferentially derivative(s) being or comprising a source of carbon, nitrogen, phosphate, manganese, potassium, calcium, vitamin, and/or chlorine: i) Na lactate or a source of carbon, preferentially at a concentration comprised between 10⁻²⁰ or 0.0023 mol or gram of Na lactate or source of carbon per liter of pre-growth medium and 0.23 or 10⁵ mol or gram of Na lactate or source of carbon per liter of pre-growth medium, most preferentially at a concentration of 0.023±0.01 mol of Na lactate or source of carbon per liter of pre-growth medium, ii) ammonium chloride or source of nitrogen, preferentially at a concentration comprised between 10⁻²⁰ or 7.4 10⁻⁴ mol or gram of ammonium chloride or source of nitrogen per liter of pre-growth medium and 7.4 10⁻² or 10⁵ mol or gram of ammonium chloride or source of nitrogen per liter of pre-growth medium, most preferentially at a concentration of (7.5±1)·10⁻³ mol of ammonium chloride or source of nitrogen per liter of pre-growth medium, iii) KH₂PO₄ or source of potassium or source of phosphorous, preferentially at a concentration comprised between 10⁻²⁰ or 1.55 10⁻⁵ mol or gram of KH₂PO₄ or source of potassium or source of phosphorous per liter of pre-growth medium and 1.55 10⁻³ or 10⁵ mol or gram of KH₂PO₄ or source of potassium or source of phosphorous per liter of pre-growth medium, most preferentially at a concentration of (1.5±1)·10⁻⁴ mol of KH₂PO₄ or source of potassium or source of phosphorous per liter of pre-growth medium, iv) MgSO₄ or source of magnesium, preferentially at a concentration comprised between 10⁻²⁰ or 4.1·10⁻⁵ mol or gram of MgSO₄ or source of magnesium per liter of pre-growth medium and 4.1·10⁻³ or 10⁵ mol or gram of MgSO₄ or source of magnesium per liter of pre-growth medium, most preferentially at a concentration of (4±1)·10⁻⁴ mol of MgSO₄ or source of magnesium per liter of pre-growth medium, v) a source of iron or FeCl₃, preferentially at a concentration comprised between 10⁻²⁰ or 2·10⁻⁷ mol or gram of FeCl₃ or source of iron per liter of pre-growth medium and 10⁻⁵ or 10⁵ mol or gram of FeCl₃ or source of iron per liter of pre-growth medium, most preferentially at a concentration of (2±1) 10⁻⁶ mol of FeCl₃ or source of iron per liter of pre-growth medium, vi) thiamine or vitamin, preferentially at a concentration between 10⁻²⁰ or 8·10⁻⁹ mol or gram of vitamin or thiamine per liter of pre-growth medium and 8·10⁻⁷ or 10⁵ mol or gram of vitamin or thiamine per liter of pre-growth medium, most preferentially at a concentration of (8±2) 10⁻⁸ mol of vitamin or thiamine per liter of pre-growth medium, vii) CaCl₂ or source of calcium or chlorine, preferentially at a concentration between 10⁻²⁰ or 10⁻⁵ mol or or gram of CaCl₂ or source of calcium or chlorine per liter of pre-growth medium and 10⁻³ or 10⁵ mol or gram of CaCl₂ or source of calcium or chlorine per liter of pre-growth medium, most preferentially at a concentration of (1±0.8)·10⁻⁴ mol of CaCl₂ or source of calcium or chlorine per liter of pre-growth medium.

Preferentially, the growth medium comprises, preferentially before adding the fed-batch medium to the growth medium, at least 1, 2, 3, 4, 5, 6, or 7 of the following chemical element(s) or derivative(s), preferentially derivative(s) being or comprising a source of carbon, nitrogen, phosphate, manganese, potassium, calcium, vitamin, and/or chlorine: i) Na lactate or a source of carbon, preferentially at a concentration comprised between 10⁻²⁰ or 0.0014 mol or gram of Na lactate or source of carbon per liter of growth medium and 0.14 or 10⁵ mol or gram of Na lactate or source of carbon per liter of growth medium, most preferentially at a concentration of (0.014±0.01) mol of Na lactate or source of carbon per liter of growth medium, ii) ammonium chloride or source of nitrogen, preferentially at a concentration comprised between 10⁻²⁰ or 4.1 10⁻⁴ mol or gram of ammonium chloride or source of nitrogen per liter of growth medium and 4.1 10⁻² or 10⁵ mol or gram of ammonium chloride or source of nitrogen per liter of growth medium, most preferentially at a concentration of (4.1±1)·10⁻³ mol of ammonium chloride or source of nitrogen per liter of growth medium, iii) KH₂PO₄ or source of potassium or source of phosphorous, preferentially at a concentration comprised between 10⁻²⁰ or 1.55 10⁻⁵ mol or gram of KH₂PO₄ or source of potassium or source of phosphorous per liter of growth medium and 1.55 10⁻³ or 10⁵ mol or gram of KH₂PO₄ or source of potassium or source of phosphorous per liter of growth medium, most preferentially at a concentration of (1.5±1)·10⁻⁴ mol of KH₂PO₄ per liter of growth medium, iv) MgSO₄ or source of magnesium, preferentially at a concentration comprised between 10⁻²⁰ or 4.1·10⁻⁵ mol of MgSO₄ or source of magnesium per liter of growth medium and 4.1·10⁻³ or 10⁵ mol or gram of MgSO₄ or source of magnesium per liter of growth medium, most preferentially at a concentration of (4±1)·10⁻⁴ mol of MgSO₄ per liter of growth medium, v) a source of iron or FeCl₃, preferentially at a concentration comprised between 10⁻²⁰ or 10⁻⁷ of FeCl₃ or source of iron per liter of growth medium and 10⁻⁵ or 10⁵ mol of FeCl₃ or source of iron per liter of growth medium, most preferentially at a concentration of (2±1) 10⁻⁶ mol of FeCl₃ or source of iron per liter of growth medium, vi) thiamine or vitamin, preferentially at a concentration between 10⁻²⁰ or 8·10⁻⁹ mol or gram of vitamin or thiamine per liter of growth medium and 8·10⁻⁷ or 10⁵ mol or gram of vitamin or thiamine per liter of growth medium, most preferentially at a concentration of (8±2) 10⁻⁸ mol of vitamin or thiamine per liter of growth medium, vii) CaCl₂ or source of calcium or chlorine, preferentially at a concentration between 10⁻²⁰ or 10⁻⁵ mol or gram of CaCl₂ or source of calcium or chlorine per liter of growth medium and 10⁻³ or 10⁵ mol or gram of CaCl₂ or source of calcium or chlorine per liter of growth medium, most preferentially at a concentration of (1±0.8)·10⁻⁴ mol of CaCl₂ or source of calcium or chlorine per liter of growth medium.

Preferentially, the fed-batch medium comprises, preferentially before being added to the growth medium, at least 1, 2, 3, 4, 5, 6, or 7 of the following chemical element(s) or derivative(s), preferentially derivative(s) being or comprising a source of carbon, nitrogen, phosphate, manganese, potassium, calcium, vitamin, and/or chlorine: i) lactic acid or a source of carbon, preferentially at a concentration comprised between 10⁻²⁰ or 10⁻¹ mol or gram of lactic acid or source of carbon per liter of fed-batch medium and 10 or 10⁵ mol or gram of lactic acid or source of carbon per liter of fed-batch medium, most preferentially at a concentration of (1±0.5) mol of lactic acid or source of carbon per liter of fed-batch medium, ii) ammonia or source of nitrogen, preferentially at a concentration comprised between 10⁻²⁰ or 2.8 10⁻² mol or gram of ammonia or source of nitrogen per liter of fed-batch medium and 2.8 or 10⁵ mol or gram of ammonia or source of nitrogen per liter of fed-batch medium, most preferentially at a concentration of (2.8±1)·10⁻¹ mol of ammonia or source of nitrogen per liter of fed-batch medium, iii) KH₂PO₄ or a source of potassium or phosphorous, preferentially at a concentration comprised between 10⁻²⁰ or 1.7 10⁻³ mol of KH₂PO₄ or a source of potassium or phosphorous per liter of fed-batch medium and 1.7 10⁻¹ or 10⁵ mol or gram of KH₂PO₄ or a source of potassium or phosphorous per liter of fed-batch medium, most preferentially at a concentration of (1.7±1)·10⁻² mol of KH₂PO₄ per liter of fed-batch medium, iv) MgSO₄ or a source of magnesium, preferentially at a concentration comprised between 10⁻²⁰ or 2·10⁻⁴ mol or gram of MgSO₄ or a source of magnesium per liter of fed-batch medium and 2·10⁻² or 10⁵ mol or gram of MgSO₄ or a source of magnesium per liter of fed-batch medium, most preferentially at a concentration of (2±1)·10⁻³ mol of MgSO₄ per liter of fed-batch medium, v) a source of iron or FeCl₃, preferentially at a concentration comprised between 10⁻²⁰ or 10⁻⁴ mol or gram of FeCl₃ or source of iron per liter of fed-batch medium and 10⁻¹ or 10⁵ mol or gram of FeCl₃ or source of iron per liter of fed-batch medium, most preferentially at a concentration of (7±4) 10⁻³ mol of FeCl₃ or source of iron per liter of fed-batch medium, vi) thiamine or vitamin, preferentially at a concentration between 10⁻²⁰ or 10⁻⁸ mol or gram of vitamin or thiamine per liter of fed-batch medium and 10⁻⁴ or 10⁵ mol or gram of vitamin or thiamine per liter of fed-batch medium, most preferentially at a concentration of (2±1.5) 10⁻⁶ mol of vitamin or thiamine per liter of fed-batch medium, vii) CaCl₂ or source of calcium or chlorine, preferentially at a concentration between 10⁻²⁰ or 10⁻⁵ of CaCl₂ mol or gram or source of calcium or chlorine per liter of fed-batch medium and 10⁻² or 10⁵ mol or gram of CaCl₂ or source of calcium or chlorine per liter of fed-batch medium, most preferentially at a concentration of (1±0.8)·10⁻³ mol of CaCl₂ or source of calcium or chlorine per liter of fed-batch medium.

The invention also relates to the method according to the invention, wherein the pre-growth, growth, and/or fed-batch medium/media comprise(s) at least one of the sources selected from the group consisting of:
a source of carbon selected from the group consisting of: at least one compound comprising at least one atom of carbon, lactic acid, Na lactate, lactic acid, acetate, glycolate, glucose, pyruvate, succinate, carbon dioxide, glycerol and combinations thereof, at a concentration preferentially comprised between 1 nM and 2 Mol/L;
a source of iron selected from the group consisting of: at least one compound comprising at least one atom of iron, iron citrate, iron quinate, iron chloride, iron sulfate, FeCl3, and combinations thereof, at a concentration preferentially comprised between 1 nM and 2·10⁻³ Mol/L;
a source of nitrogen selected from the group consisting of: at least one compound comprising at least one atom of nitrogen, nitrate salt, nitrogen gas, ammonium, ammonia, ammonium salt, urea, an amino acid, ammonia gas, and combinations thereof, at a concentration preferentially comprised between 1 nM and 4 Mol/L;
a source of oxygen selected from the group consisting of: at least one compound comprising at least one atom of oxygen, oxygen or air or compressed air, preferentially in the form of a gas, the source of oxygen being in some cases bubbled or introduced to the growth medium, at a gas rate that is preferentially comprised between 5 mL of gas per minute and 50000 mL of gas per minute;
a source of phosphate consisting of at least one compound comprising at least one atom of phosphate, at a concentration preferentially comprised between 1 nM and 2·10⁻¹ Mol/L;
a source of potassium consisting of at least one compound comprising at least one atom of potassium, at a concentration preferentially comprised between 1 nM and 2·10⁻¹ Mol/L;
a source of sulfur or sulfate consisting of at least one compound comprising at least one atom of sulfur or sulfate, at a concentration preferentially comprised between 1 nM and 4·10⁻¹ Mol/L;
a source of manganese consisting of at least one compound comprising at least one atom of manganese, at a concentration preferentially comprised between 1 nM and 4·10⁻¹ Mol/L;
a source of vitamin selected from the group consisting of: at least one compound comprising at least one vitamin, Biotin, Calcium, pantothenate, Folic acid, Inositol, Nicotinic acid, p-Aminobenzoic acid, Pyridoxine HCl, Riboflavin, Thiamine, Thiamine HCL and derivatives thereof and combinations thereof, at a concentration preferentially comprised between 1 nM and 10⁻⁴ Mol/L, and
a source of calcium consisting of at least one compound comprising at least one atom of calcium, at a concentration preferentially comprised between 1 nM and 10⁻¹ Mol/L.

In one embodiment of the invention, the source(s) of carbon, nitrogen, potassium, phosphorous, magnesium, calcium, vitamin, iron, oxygen, and/or chlorine comprise at least 1, 2, 5, 10 or 10³ atoms of carbon, nitrogen, potassium, phosphorous, magnesium, calcium, vitamin, iron, oxygen, and/or chlorine, preferentially within the chemical formula or molecule(s) or component(s) of these sources.

In still another embodiment of the invention, when a compound or quantity or element or property P1 is higher, longer, or larger than a compound or quantity or element or property P₂, it means that that P1=α·P₂, where α is preferentially a number or integer larger than 1, or P1=α+P₂, where α is preferentially a number or integer larger than 0.

In still another embodiment of the invention, when a compound or a quantity or element or property P1 is lower, shorter, or smaller than a compound or a quantity or element or property P₂, it means that that P1=P₂/α, where α is preferentially a number or integer larger than 1, or P1=P₂−α, where α is preferentially a number or integer larger than 0.

In one embodiment of the invention, a CMR compound is a carcinogenic, mutagenic, and/or repro-toxic compound. In some cases, a carcinogenic compound is a compound that produces induces or is suspected to produce or induce a cancer, preferentially in a living organism or human. In some cases, a mutagenic compound is a compound that produces or induces or is suspected to produce or induce a mutation, modification, change in number or size of at least one gene, DNA, RNA, DNA strand, RNA strand, and/or nucleic acid, preferentially in a living organism or human. In some cases, a repro-toxic compound is a compound that produces or induces or is suspected to produce or induce a toxicity, mutation, modification, change of the reproductive organs, embryo, fetus, preferentially in a living organism or human.

In one embodiment of the invention, a toxic or cytotoxic compound is a compound that produces or induces or is suspected to produce or induce toxicity, death, loss of weight, damage towards organs, change in behavior, change in consumed food or water, necrosis, apoptosis, cellular internalization, change in number, shape and/or geometry of cell(s), preferentially in an individual or living organism.

In some cases, a compound is CMR or cytotoxic or toxic at a concentration that is larger than 10⁻⁶, 10⁻³, 10⁻¹, 1, 10, 10³ or 10⁶ μM.

In some other cases, a compound is CMR or cytotoxic or toxic at a concentration that is lower than 10²⁰, 10⁶, 10³, 10, 1, 10⁻¹, 10⁻³ or 10⁻⁶ μM.

The invention relates to a method for producing high purity iron oxide nanoparticles using nanoparticle-producing cell(s), comprising:
i) a pre-growth step consisting in amplifying the nanoparticle-producing cell(s) in a pre-growth medium such that nanoparticle-producing cell(s) produce(s) essentially no nanoparticles, and
ii) a growth step consisting in amplifying the nanoparticle-producing cell(s) originating from the pre-growth step in a growth medium such that nanoparticle-producing cell(s) produce(s) nanoparticles.

The invention relates to a method for producing high purity iron oxide nanoparticles using nanoparticle-producing cell(s), comprising:
i) a pre-growth step consisting in amplifying the nanoparticle-producing cell(s) in a pre-growth medium such that nanoparticle-producing cell(s) produce(s) essentially no nanoparticles, and/or
ii) a growth step consisting in amplifying the nanoparticle-producing cell(s) originating from the pre-growth step in a growth medium such that nanoparticle-producing cell(s) produce(s) nanoparticles.

In one embodiment of the invention, the growth medium is supplemented by a fed-batch medium.

In one embodiment of the invention, the growth medium supplemented by the fed-batch medium is the growth medium.

In one embodiment of the invention, the growth medium has at least one property in common with the pre-growth and/or fed-batch medium.

In one embodiment of the invention, the growth medium and/or pre-growth medium comprise(s) at least one source selected in the group consisting of: i), a source of carbon or sodium or lactate, preferentially sodium lactate, ii) a source of ammonium, preferentially ammonium chloride, iii) a source of magnesium, preferentially magnesium sulfate, iv) a source of potassium, preferentially potassium phosphate, v) a source of vitamin vitamin, preferentially thiamin, vi) a source of calcium, preferentially calcium chloride, and vii) a source of iron, preferentially iron chloride. Each source is preferentially comprised in the growth and/or pre-growth medium at a concentration comprised between 10⁻⁶ and 10³, 10⁻³ and 100 mM, 0.01 and 10 mM, or between 0.1 and 10 mM.

In one embodiment of the invention, in the pre-growth medium and/or growth medium, the concentration of the vitamin and/or source of calcium and/or source of iron is smaller, by a factor of at least 0, 0.5, 1, 1.1, 1.2, 1.5, 5, 10, 10³ or 10⁵ than the concentration of the source of sodium and/or the source of ammonium and/or the source of magnesium and/or the source of potassium.

In one embodiment of the invention, the fed-batch medium comprises at least one source selected in the group consisting of: i), a source of carbon, preferentially lactic acid, ii), ammonia, iii) a source of potassium, preferentially potassium phosphate, iv) a source of magnesium, preferentially magnesium sulfate, v) a source of iron, preferentially iron chloride, vi) a source of vitamin, preferentially thiamine, vii) a source of calcium, preferentially calcium chloride, and viii) a source of iron, preferentially iron chloride. Each source is preferentially comprised in the growth and/or pre-growth medium at a concentration comprised between 0.001 and 100 mM, 0.01 and 10 mM, or between 0.1 and 10 mM.

In one embodiment of the invention, in the fed-batch medium, the concentration of the vitamin and/or source of calcium is smaller, by a factor of at least 0, 0.5, 1, 1.1, 1.2, 1.5, 5, 10, 10³ or 10⁵, than the concentration of the source of sodium and/or the source of ammonium and/or the source of magnesium and/or the source of potassium and/or source of iron.

In one embodiment of the invention, the nanoparticle(s) according to the invention is/are or comprise(s) assemblies of more than 1, 2, 5, 10, 10³, 10⁵, 10¹⁰, 10²⁰, 10⁵⁰ or 10¹⁰⁰ nanoparticle(s), nanoparticles per liter of growth medium or nanoparticles per nanoparticle-producing cell. In some cases, iron oxide represents or is an assembly of more than 1, 10, 10³, 10⁵, 10¹⁰, 10²⁰, 10⁵⁰ or 10¹⁰⁰ atom(s) of iron and/or more than 1, 10, 10³, 10⁵, 10¹⁰, 10²⁰, 10⁵⁰ or 10¹⁰⁰ atom(s) of oxygen. In some other cases, the chemical element(s), and/or impurity(ies) comprised in the nanoparticles, are/is or represent(s) more than 1, 10, 10³, 10⁵, 10¹⁰, 10²⁰, 10⁵⁰ or 10¹⁰⁰ chemical element(s), and/or impurity(ies) comprised in the nanoparticles.

In another embodiment of the invention, the nanoparticle(s) according to the invention is/are or comprise(s) assemblies of less than 10¹⁰⁰, 10⁵⁰, 10²⁰, 10¹⁰, 10⁵, 10³, 100, 50, 10, 5 or 2 nanoparticle(s), nanoparticles per liter of growth medium or nanoparticles per nanoparticle-producing cell. In some cases, iron oxide represents or is an assembly of less than 1, 10, 10³, 10⁵, 10¹⁰, 10²⁰, 10⁵⁰ or 10¹⁰⁰ atom(s) of iron and/or less than 1, 10, 10³, 10⁵, 10¹⁰, 10²⁰, 10⁵⁰ or 10¹⁰⁰ atom(s) of oxygen. In still some other cases, the chemical element(s) and/or impurity(ies) comprised in the nanoparticles is/are or represent(s) less than 10¹⁰⁰, 10⁵⁰, 10²⁰, 10¹⁰, 10⁵, 10³, 100, 50, 10, 5 or 2 chemical elements, and/or impurity(ies) comprised in the nanoparticles.

In one embodiment of the invention, at least one impurity is comprised in the nanoparticle.

In one embodiment of the invention, the high purity iron oxide nanoparticles comprise a low quantity of impurity(ies), for example when the method enables to obtain nanoparticles with a low quantity of impurity. In some cases, the nanoparticle(s) does/do not comprise at least one impurity or comprise(s) or comprise(s) less than 10⁵⁰, 10²⁰, 10¹⁰, 10⁵, 10², 10, 5, 2, 5, 1, 10⁻², 10⁻¹⁰, 10⁻²⁰ or 10⁻⁵⁰ impurity(ies) or impurity(ies) per gram of nanoparticles or gram of impurity(ies) per gram of nanoparticles. In some other cases, the percentage, preferentially in mass, of impurity(ies) comprised inside or at the surface of the nanoparticle(s) is lower than 100, 90, 80, 70, 60, 50, 30, 20, 10, 5, 1, 0.1 or 0.001%. According to the invention, this percentage of impurity(ies) can in some cases be defined as the ratio between the number of atoms, quantity, mass, or volume of impurity(ies) comprised in the nanoparticle(s) divided by the total number of atoms, quantity, mass, or volume of all chemical element(s) comprised in the nanoparticle(s). In some cases, all chemical element(s) comprised in the nanoparticle(s) can be the sum of the iron oxide, doping material, and impurity(ies), comprised in the nanoparticle(s). In still some other cases, the concentration of the impurity(ies) comprised inside or at the surface of the nanoparticle(s) is lower than 10⁵⁰, 10³⁰, 10¹⁰, 10⁵, 10³, 500, 100, 50, 10, 1, 10⁻¹, 10⁻³, 10⁻⁵, 10⁻¹⁰ or 10⁻⁵⁰ μg of impurity(ies) per gram of nanoparticle(s).

In another embodiment of the invention, the high purity iron oxide nanoparticles comprise a large quantity of impurity(ies), for example when the impurity(ies) is/are added or incorporated to the nanoparticles after the production of the nanoparticles by the method. In some cases, the nanoparticles comprise more than 10⁻⁵⁰, 10⁻²⁰, 10⁻¹⁰, 10⁻⁵, 10⁻², 1, 2, 5, 10, 10³, 10⁵, 10¹⁰, 10²⁰ or 10⁵⁰ impurity(ies) or impurities per gram of nanoparticles or gram of impurity per gram of nanoparticles. In some cases, the nanoparticles comprise a large quantity of impurity(ies). In some cases, the percentage, preferentially in mass, of the impurity(ies) comprised inside or at the surface of the nanoparticle(s) is larger than 10⁻⁴⁰, 10⁻²⁰, 10⁻¹⁰, 10⁻⁵, 10⁻², 10⁻¹, 1, 5, 10, 25, 50, 75, 80 or 90%. In still some other cases, the concentration of impurity(ies) comprised inside or at the surface of the nanoparticle(s) is larger than 10⁻¹⁰⁰, 10⁻⁵⁰, 10⁻²⁰, 10⁻¹⁰, 10⁻⁵, 10⁻³, 10⁻², 10⁻¹, 1, 10, 50, 100, 10³, 10⁵ or 10¹⁰ μg of impurity(ies) per gram of nanoparticle(s).

In some cases, the impurities can be the same impurities, i.e. preferentially impurities comprising the same chemical elements.

In some other cases, the impurities can be different impurities, i.e. preferentially impurities comprising at least one different chemical element.

In one embodiment of the invention, the chemical element(s) is selected from the group consisting of: actinide, actinium, aluminium, americium, antimony, argon, arsenic, astatine, barium, berkelium, beryllium, bismuth, bohrium, boron, bromine, caesium, calcium, californium, carbon, cerium, chlorine, chromium, cobalt, copernicum, cadmium, copper, curium, darmstadtium, dubnium, dysprosium, einsteinium, erbium, europium, fermium, fleovium, fluorine, francium, gadolinium, gallium, germanium, gold, hafnium, helium, hessium, holmium, hydrogen, indium, iodine, iridium, iron, krypton, lanthanide, lanthanum, lawrencium, lead, lithium, livermorium, lutetium, magnesium, manganese, meitherium, mendelevium, mercury, molybdenum, neodymium, neon, neptunium, nickel, niobium, nitrogen, nobelium, osmium, oxygen, palladium, phosphorus, platinum, plutonium, polonium, potassium, praseodymium, proctactinium, promethium, radium, radon, rhenium, rhodium, roentgenium, rubidium, ruthenium, rutherfordium, samarium, selenium, silicon, silver, sodium, strontium, sulphur, scandium, seaborgium, tellurium, terbium, thorium, thulium, tin, tantalum, technetium, thallium, titanium, tungsten, ununoctium, ununpentium, ununseptium, ununtrium, uranium, vanadium, xenon, ytterbium, yttrium, zinc, zirconium, and a combination of several of these chemical element(s).

The invention also relates to the method according to the invention, wherein the impurity(ies) is/are at least one chemical element different from iron, oxygen, and/or iron oxide.

The invention also relates to the method according to the invention, wherein the impurity is preferentially carbon or carbonaceous material.

In one embodiment of the invention, the carbonaceous material comprises at least one carbon atom, preferentially but not necessarily mixed or assembled with other chemical element(s) than carbon.

In still another embodiment of the invention, the carbon or carbonaceous material originates from, is produced by, or comes from nanoparticle-producing cell(s).

The invention also relates to the method according to the invention, wherein the nanoparticle(s) obtained by the method comprise(s) iron oxide, where the iron oxide has at least one of the following properties: i), it comprise at least one atom of iron and one atom of oxygen, ii), it forms a crystallized or mineral structure, iii), it can have the chemical formula FeO, FeO₂, Fe₃O₄, Fe₄O₅, Fe₅O₆, Fe₅O₇, Fe₂₅O₃₂, Fe₁₃O₁₉, α-Fe₂O₃, β-Fe₂O₃, γ-Fe₂O₃, ε-Fe₂O₃, iv), it can be composed of wustite, iron dioxide, magnetite, hematite, maghemite, v), it can be in the epsilon phase, alpha phase, beta phase, gamma phase, vi), it can be in various levels of oxidations, vii), it has the formula Fe_{α}O_{β}D_{γ}, where α, β and/or γ is/are coefficients, preferentially stoichiometric coefficients. In some cases, α, β, and/or γ is/are equal to 0, 1, 2, 3, 4, 5, 6, 7, 8, 9, 10, 11, 12, 13, 14, 15, 16, 18, 19 or 20. In some other cases, α, β, and/or γ is/are larger than 0, 1, 2, 3, 4, 5, 6, 7, 8, 9, 10, 11, 12, 13, 14, 15, 16, 18, 19 or 20. In still some other cases, α, β, and/or γ is/are lower than 0, 1, 2, 3, 4, 5, 6, 7, 8, 9, 10, 11, 12, 13, 14, 15, 16, 18, 19 or 20. In some other cases, D is the doping material of the nanoparticles. In some cases, the doping material can be selected from the group consisting of: Aluminum, antimonite, barium, chrome, copper, gold, manganese, silver, tin, titanium, and zinc.

In one embodiment of the invention, the iron oxide comprised in the nanoparticles is the predominant chemical element of the nanoparticle. In some cases, the high purity iron oxide nanoparticles can comprise a large quantity of iron oxide. In some cases, the percentage, preferentially in mass, of iron oxide comprised in the nanoparticle(s), is larger than 10⁻⁴⁰, 10⁻²⁰, 10⁻¹⁰, 10⁻⁵, 10⁻², 10⁻¹, 1, 5, 10, 25, 50, 75, 80, 90, 99 or 99.9%. According to the invention, this percentage of iron oxide can in some cases be defined as the ratio between the number of atoms, quantity, mass, or volume of iron oxide in the nanoparticle(s) divided by the total number of atoms, quantity, mass, or volume of all chemical element(s) comprised in the nanoparticle(s).

In still some other cases, the concentration of iron oxide, comprised in the nanoparticle(s) is larger than 10⁻¹⁰⁰, 10⁻⁵⁰, 10⁻²⁰, 10⁻¹⁰, 10⁻⁵, 10⁻³, 10⁻², 10⁻¹, 1, 10, 50, 100, 10³, 10⁵ or 10¹⁰ μg of iron oxide, per gram of nanoparticle(s).

In one embodiment of the invention, the high purity iron oxide nanoparticles comprise a low quantity of iron oxide, for example when the nanoparticles are treated and/or partly or fully destroyed and/or administered to an organism, or when the method does not enable to incorporate a large quantity of iron oxide in the nanoparticles. In some cases, the percentage, preferentially in mass, of iron oxide, comprised inside or at the surface of the nanoparticle(s), is lower than 100, 90, 80, 70, 50, 30, 10, 5, 1, 0.1 or 0.001%. In some other cases, the concentration of iron oxide, comprised in the nanoparticle(s) can be lower than 10⁵⁰, 10³⁰, 10¹⁰, 10⁵, 10³, 500, 100, 50, 10, 1, 10⁻¹, 10⁻³, 10⁻⁵, 10⁻¹⁰, or 10⁻⁵⁰ μg of iron oxide per gram of nanoparticle(s).

In another embodiment of the invention, the percentage, concentration, number of atoms, quantity, mass, or volume of iron oxide comprised in the nanoparticle(s) is larger, preferentially by a factor of 1.00001, 1.001, 1.1, 2, 5, 10, 50, 10², 10³, 10⁵, 10¹⁰, 10²⁰ or 10⁵⁰, than the percentage, concentration, number of atoms, quantity, mass, or volume of impurity(ies) comprised in the nanoparticle(s).

In one embodiment of the invention, the iron oxide and/or impurity(ies) is/are comprised or inserted: i) inside the nanoparticle(s), ii) at the surface of the nanoparticle(s), iii) outside of the nanoparticle(s), iv) in the crystalline or amorphous structure of the nanoparticle(s), v) in a defect of the nanoparticle(s), and/or vi) in a vacancy of the nanoparticle(s).

In one embodiment of the invention, the iron oxide and/or impurity(ies) is/are in interaction, such as electrostatic, strong, weak, nuclear, metallic, Van der Waals, Debye, London, or hydrogen interactions with the nanoparticle(s).

In one embodiment of the invention, the iron oxide and/or impurity(ies) is/are located at a distance from the nanoparticle(s), preferentially from the center or surface of the nanoparticle(s), which is lower than 10⁵⁰, 10²⁰, 10¹⁰, 10⁵, 10³, 100, 10, 5 or 1 nm. In some cases, the center of the nanoparticles is the region or volume or location or assembly of chemical elements that is at the middle of the largest, lowest, and/or average dimension of the nanoparticle such as half of the diameter of a spherical nanoparticle or half of the largest, lowest, and/or average length of a nanoparticle. In some other cases, the surface of the nanoparticles is the region or location or assembly of chemical elements that is at the largest distance from the center of the nanoparticle while remaining in the nanoparticle.

In still another embodiment of the invention, the iron oxide and/or impurity(ies) is/are located at a distance from nanoparticle(s), preferentially from the center or surface of the nanoparticle(s), which is larger than 0.001, 0.01, 0.1, 1, 10, 100, 10³, 10⁵, 10¹⁰, 10²⁰ or 10⁵⁰ nm.

In another embodiment of the invention, the nanoparticle(s) according the invention comprise(s) a core and/or a coating, which preferentially surrounds the core of the nanoparticle(s).

In one embodiment of the invention, the core and/or coating of the nanoparticles possess at least one property in common with the nanoparticles such as the concentration in iron oxide and/or impurity(ies).

In one embodiment of the invention, the nanoparticle(s), the core and/or coating of the nanoparticle(s), has/have at least one of the following properties:
(a) magnetic, diamagnetic, superparamagnetic, ferromagnetic, ferrimagnetic, and/or paramagnetic behavior(s) or property(ies), preferentially observed under the application of magnetic field of strength preferentially larger than 10⁻⁵, 10⁻⁴⁰, 10⁻²⁰, 10⁻¹⁰, 10⁻⁵, 10⁻² or 10⁻¹ T, preferentially observed at temperatures lower than 10¹⁰, 10⁵, 10³, 10², 10 or 1 K. In some cases, the core can have different magnetic property(ies) from the coating. For example, the core can be ferromagnetic or superparamagnetic while the coating can be diamagnetic or paramagnetic.
(b) a crystalline part or structure comprising at least 1, 2, 5, 10, 50, 100, 10³, 10⁵, 10⁷, 10⁹, 10²⁰ or 10⁵⁰ crystalline plane(s) or crystalline ordered structures, which can preferentially be observed or measured under electron microscopy. In some cases, the core can have a different crystalline structure from the coating. For example, the core can comprise more than 1, 5, 10, 10³ or 10⁵ crystalline plane(s) or crystalline ordered structure(s) while the coating can have less than 10⁵, 10³, 10, 5 or 2 crystalline planes or crystalline ordered structures.
(c) a composition made of metal(s) or metal oxide(s), preferentially iron oxide, most preferentially maghemite and/or magnetite. In some cases, the core comprises a different composition from the coating. For example, the core comprises more than 1, 5, 10, 25, 50, 75, 90, 95 or 99 percent or percent in mass of iron oxide while the coating comprises less than 99, 95, 90, 75, 50, 10, 5 or 1 percent or percent in mass of iron oxide. This percentage can be the ratio between the quantity, volume, number of atoms, mass of iron oxide comprised in the core and/or coating divided by the total quantity, total volume, total number of atoms, total mass, of all chemical element(s) comprised in the core and/or coating.
(d) single domain, or be magnetically mono-domain,
(e) a magnetic microstructure, which can be characterized by the presence of magnetic field lines, which can be oriented in a preferential direction such as an axis of easy magnetization or a crystallographic direction of the core of the nanoparticle(s) such as [111], where such a magnetic microstructure can under certain conditions be observable, in particular by electronic holography,
(f) a size comprised between 1 nm and 10⁵ μm, 1 nm and 10³ μm, 1 nm and 100 μm, 1 nm and 10 μm, 1 nm and 1 μm, 5 nm and 1 μm, 5 and 500 nm, 5 and 250 nm, 5 and 100 nm, 5 and 80 nm, 5 and 60 nm, 10 nm and 1 μm, 10 and 500 nm, 10 and 250 nm, 10 and 100 nm, 10 and 80 nm, 10 and 60 nm, 15 nm and 1 μm, 15 and 500 nm, 15 and 250 nm, 15 and 100 nm, 15 and 80 nm, 15 and 60 nm, 20 nm and 1 μm, 20 and 500 nm, 20 and 250 nm, 20 and 100 nm, 20 and 80 nm, or between 20 et 60 nm,
(g) a size in some cases larger than 0.1, 1, 2, 5, 10, 15, 20, 25, 30, 35 or 40 nm,
(h) a size in some other cases lower than 10¹⁰, 10⁵, 10⁴, 2000, 1000, 500, 400, 300, 200, 150, 120, 100, 95, 90, 80, 75, 70, 65, 60, 55, 50, 45, 40, 35, 30, 25, 20, 15, 10 or 5 nm,
(i) a zeta potential, charge, or surface charge comprised between −10¹⁰ mV and 10¹⁰ mV, −10⁵ mV and 10⁵ mV, −10⁴ mV and 10⁴ mV, −10³ mV, −10² mV and 10² mV, −10 and 10 mV, preferentially at pH comprised between 0 and 14, 1 and 13, 2 and 12, 3 and 11, 4 and 10, 5 and 9, or between 6 and 8.
(j) a zeta potential, charge, or surface charge, which is in some cases larger than −10⁵⁰, −10²⁰, −10¹⁰, −10⁵, −10³, −10, −5, −1, 0, 5, 10, 20, 50, or 100 mV, preferentially at pH larger than 0, 1, 2, 3, 4, 5, 6, 7, 8, 9, 10, 11, 12 or 13.
(k) a zeta potential, charge, or surface charge, which is in some other cases larger than −10⁵⁰, −10²⁰, −10¹⁰, −10⁵, −10³, −10, −5, −1, 0, 5, 10, 20, 50, or 100 mV, preferentially at pH lower than 14, 13, 12, 11, 10, 9, 8, 7, 6, 5, 4, 3, 2, 1 or 0.
(l) a zeta potential, charge, or surface charge, which is in some other cases lower than 10⁵⁰, 10²⁰, 10¹⁰, 10⁵, 10³, 10, 5, 1, 0, −5, −10, −20, −50, or −100 mV, preferentially at pH larger than 0, 1, 2, 3, 4, 5, 6, 7, 8, 9, 10, 11, 12 or 13.
(m) a zeta potential, charge, or surface charge, which is in some other cases lower than 10⁵⁰, 10²⁰, 10¹⁰, 10⁵, 10³, 10, 5, 1, 0, −5, −10, −20, −50, or −100 mV, preferentially at pH lower than 14, 13, 12, 11, 10, 9, 8, 7, 6, 5, 4, 3, 2, 1 or 0.
(n) an isoelectric point comprised between 0 and 14, 1 and 13, 2 and 12, 3 and 11, 4 and 10, 5 and 9, or between 6 and 8,
(o) in some cases, an isoelectric point in some cases larger than 0, 1, 2, 3, 4, 5, 6, 7, 8, 9, 10, 11, 12 or 13, and/or
(p) in some other cases, an isoelectric point in some other cases lower than 14, 13, 12, 11, 10, 9, 8, 7, 6, 5, 4, 3, 2, 1 or 0.

In one embodiment of the invention, the core and/or coating is/are synthesized by the nanoparticle-producing cell(s).

In another embodiment of the invention, the core and/or coating is/are not synthesized by the nanoparticle-producing cell(s).

In one embodiment of the invention, the cell(s) producing the nanoparticle(s), also designated as nanoparticle-producing cell(s) or (the) cell(s), are eukaryotic or prokaryotic cell(s). In some cases, they are the cell(s) produced by or comprised in or amplified in the pre-growth and/or growth medium/media.

In one embodiment of the invention, less than 100, 80, 70, 50, 10, 20, 10, 5, 2, 1, 0.1 or 10⁻¹⁰% of nanoparticle-producing cells comprise or produce at least one nanoparticle. In some cases, this percentage can be the ratio between the number of cells comprised in the pre-growth and/or growth medium/media that comprise or produce at least one nanoparticle divided by the total number of cells in the pre-growth and/or growth medium/media.

In another embodiment of the invention, more than 100, 80, 70, 50, 10, 20, 10, 5, 2, 1, 0.1 or 10⁻¹⁰% of nanoparticle-producing cells comprise or produce at least one nanoparticle.

In one embodiment of the invention, the nanoparticle-producing cell(s) are whole cell(s).

In still another embodiment of the invention, the nanoparticle-producing cell(s) are parts of the cell(s) such as cell membrane, vesicle, enzyme, protein, lipid, DNA, RNA, organelle, compartment, cytoplasm, viruses, comprised in, originating from, replicating in, or produced by the synthetizing cell(s).

In one embodiment of the invention, the synthetizing cells are the cells synthetizing the nanoparticles, preferentially when they grow or divide or are comprised in the growth and/or fed-batch medium, preferentially not when they grow or divide or are comprised in the pre-growth medium.

In one embodiment of the invention, the nanoparticle(s) synthesized by the cell(s) is/are designated as cell-synthesized nanoparticle(s).

In one embodiment of the invention, the nanoparticle-producing cell(s) synthesize(s) the nanoparticle(s) inside the cell(s). Preferentially nanoparticle(s) is/are synthesized inside cell(s) when they are synthesized, assembled, crystallized, partly or fully: i), by or in or near or inside part of the cell such as an organelle, Golgi vesicle or apparatus, endosome, exosome, ribosome, endoplasmic reticulum, actin filament, nucleus, peroxisome, microtubule, lysosome, mitochondrion, filament, centrosome, flagellum, or the cell membrane, ii) in a region that is located inside the cell(s), or iii) in a region located at a distance from part of the cell(s) that is lower than 10⁵, 10³, 100, 10 or 1 nm.

In another embodiment of the invention, the nanoparticle-producing cell(s) synthesize(s) the nanoparticle(s) outside the cell(s). Preferentially nanoparticle(s) is/are synthesized outside the cell(s) when it/they is/are synthesized, assembled, crystallized, partly or fully: i) in a region that is located outside the cell(s), or ii) in a region located at a distance from part of the cell(s) that is larger 1, 10, 100, 10¹ or 10¹ nm.

In some cases, the cell(s) is/are assemblies of more than 1, 10, 10³, 10⁵, 10¹⁰, 10²⁰, 10⁵⁰ or 10¹⁰⁰ cell(s), preferentially per liter of growth medium. In some other cases, the cell(s) is/are assemblies of less than 10¹⁰⁰, 10⁵⁰, 10²⁰, 10¹⁰, 10⁵, 10³, 100, 50, 10, 5 or 2 cell(s), preferentially per liter of growth medium.

In one embodiment of the invention, the nanoparticle-producing cell(s) is/are eukaryotic cell(s), preferentially belonging to humans, animals, plants, trees, flours, branches, mushrooms, fungi, archae, birds, fishes, pigeons, trout, mammals, ants, bees, or insects.

In one embodiment of the invention, the nanoparticle-producing cell(s) is/are prokaryotic cell(s) or bacteria.

In some cases, the nanoparticle-producing cells can be Mycobacterium, preferentially Mycobacterium paratuberculosis, Shewanella, preferentially Shewanella oneidensi, Geothrix, preferentially Geothrix fermentans. These bacteria preferentially synthesize nanoparticle(s) outside the cells.

In some other cases, the nanoparticle-producing cells can be magnetotactic bacteria, such as Magnetospirillum magneticum strain AMB-1, magnetotactic coccus strain MC-1, three facultative anaerobic vibrios strains MV-1, MV-2 and MV-4, the Magnetospirillum magnetotacticum strain MS-1, the Magnetospirillum gryphiswaldense strain MSR-1, a facultative anerobic magnetotactic spirillum, Magnetospirillum magneticum strain MGT-1, and an obligate anaerobe, Desulfovibrio magneticus RS-1. These bacteria preferentially synthetize nanoparticle(s) inside the cell(s).

In one embodiment of the invention, the nanoparticle-producing cell(s) is/are cultivated in or using a pre-growth medium for/during the pre-growth step, and/or in or using a growth medium for/during the growth step, and/or in or using a fed-batch medium during the growth step. In some cases, the pre-growth and/or growth medium/media is/are the medium/media in which nanoparticle-producing cell(s) is/are amplified. In some cases, the fed-batch medium is the medium that is added to the growth medium, preferentially during the growth step.

In one embodiment of the invention, the total pre-growth and/or growth medium/media can comprise at least one source of chemical element, water, and nanoparticle-producing cells. In some other cases, the partial pre-growth and/or growth medium/media comprises at least one source of chemical element, water, without nanoparticle-producing cells. In still some other cases, the pre-growth and/or growth medium/media comprises only nanoparticle-producing cells.

In one embodiment of the invention, the pre-growth and/or growth and/or fed-batch medium/media comprise at least one source, preferentially one source of a chemical element, or comprise at least one chemical element, preferentially in a liquid, gaseous, and/or solid state.

In some cases, the pre-growth and/or growth and/or fed-batch medium/media is/are in a liquid, gaseous, and/or solid state.

In one embodiment of the invention, the concentration of a chemical element such as iron in the pre-growth and/or growth medium/media is the concentration of this chemical element in: i) the total pre-growth and/or growth medium/media, ii) the partial pre-growth and/or growth medium/media, or iii) the nanoparticle-producing cells.

In one embodiment of the invention, a certain quantity or volume of cell(s) can be a certain quantity or volume of growth medium comprising these cell(s). In some other cases, a certain quantity or volume of cell(s) can be a certain quantity or volume of cell(s) without water or without the aqueous environment of the cell(s) or after water or the aqueous environment of the cell(s) has been removed, for example by lyophilization.

In one embodiment of the invention, the conditions of amplifications of cells during the pre-growth and/or growth steps enable preventing genetic modifications of the nanoparticle-producing cells. In some cases, the genetic modifications of the nanoparticle-producing cell are modifications of at least 10⁻⁵⁰, 10⁻²⁰, 10⁻¹⁰, 10⁻⁵, 10⁻³, 10⁻¹, 1, 5, 10, 50, 75, 90 or 95% of gene(s), part(s) of genes, DNA portion(s), or nucleotide(s) This percentage can be the ratio between the number or quantity of gene(s), part(s) of genes, DNA portion(s), or nucleotide(s) that have been modified in the nanoparticle-producing cell(s) and the total number or quantity of all gene(s), part(s) of genes, DNA portion(s), nucleotide(s) that belong to the nanoparticle-producing cell(s).

In still another embodiment of the invention, the pre-growth and/or growth medium/media comprise a majority of water, preferentially of purified, deionized or ultrapure water, preferentially more than 10⁻⁵⁰, 10⁻²⁰, 10⁻¹⁰, 10⁻⁵, 10⁻³, 1, 10, 50, 75, 80, 90, 99, 99.99 or 99.99999 percent or percent in mass of water. This percentage can be the ratio between the quantity, mass, volume, or number of atoms of water comprised in the pre-growth and/or growth medium/media divided by the total quantity, mass, volume, or number of atoms of all chemical element(s) comprised in the pre-growth and/or growth medium/media.

In one embodiment of the invention, the pre-growth and/or growth medium/media comprise(s) at least one chemical element or one source of chemical element. In some cases, the concentration of a chemical element, such as iron, in the pre-growth and/or growth medium/media, is the concentration of this chemical element at any time of the pre-growth and/or growth step. In some cases, this concentration can be measured by estimating the number of moles, the mass, or the volume of/occupied by this chemical element divided by the total number of moles, total mass, or total volume of/occupied by all chemical elements in the pre-growth and/or growth medium/media

In one embodiment of the invention, the pre-growth and/or growth medium/media comprise(s) at least one source of carbon. In some cases, the source of carbon comprises the chemical element of the periodic table C. In some cases, the source of carbon can be selected in the list consisting of: acetate, glycolate, glucose, lactate, pyruvate, succinate, carbon dioxide, glycerol, and a derivative or combination of these compounds.

In one embodiment of the invention, the growth and/or pre-growth medium/media comprise(s) at least one source of nitrogen. In some cases, the source of nitrogen comprises the chemical element of the periodic table N. In some cases, the source of nitrogen can be selected from the group consisting of: ammonium salts, nitrate salts, urea, amino acids, ammonium salts, ammonia, nitrogen gas, and a derivative or combination of these compounds.

In one embodiment of the invention, the pre-growth and/or growth medium/media comprise(s) at least one source of sulfur or sulfate. In some cases, the source of sulfur or sulfate comprises the chemical element of the periodic table S. In some cases, the source of sulfur or sulfate can be sulfate salts or hydrogen sulfide.

In one embodiment of the invention, the pre-growth and/or growth medium/media comprise(s) at least one source of phosphorous or phosphate. In some cases, the source of phosphorous or phosphate comprises the chemical element of the periodic table P. In some cases, the source of phosphorous or phosphate can be phosphate salts.

In one embodiment of the invention, the pre-growth and/or growth medium/media comprise(s) at least one source of calcium. In some cases, the source of calcium comprises the chemical element of the periodic table Ca. In some cases, the source of calcium can be a calcium salt.

In one embodiment of the invention, the pre-growth and/or growth medium/media comprise(s) at least one source of Potassium. In some cases, the source of Potassium comprises the chemical element of the periodic table K. In some cases, the source of Potassium is a Potassium salt.

In one embodiment of the invention, the pre-growth and/or growth medium/media comprise(s) at least one source of Magnesium. In some cases, the source of Magnesium comprises the chemical element of the periodic table Mg. In some cases, the source of Magnesium is a Magnesium salt.

In one embodiment of the invention, the pre-growth and/or growth medium/media comprise(s) at least one source of oxygen. In some cases, the source of oxygen comprises the chemical element of the periodic table O. In some cases, the source of oxygen is an organic compound, carbon dioxide, or di-oxygen.

In one embodiment of the invention, the pre-growth and/or growth medium/media comprise(s) at least one source of hydrogen. In some cases, the source of hydrogen comprises the chemical element of the periodic table H. In some cases, the source of hydrogen is an organic compound, or di-hydrogen.

In one embodiment of the invention, the pre-growth and/or growth medium/media comprise(s) at least one source of iron. In some cases, the source of iron comprises the chemical element of the periodic table Fe. In some cases, the source of iron is or consists of or comprises iron. In some cases, the source of iron is iron citrate, iron quinate, iron chloride, or iron sulfate.

In one embodiment of the invention, the pre-growth and/or growth medium/media comprise(s) at least one source of sulfur. In some cases, the source of sulfur comprises the chemical element of the periodic table S. In some cases, the source of sulfur is comprised in at least one vitamin.

In one embodiment of the invention, the sources of carbon, nitrogen, sulfur, sulfate phosphorous, phosphate, calcium, Potassium, Magnesium, Oxygen, hydrogen, or iron, comprise(s) more than 10⁻¹⁰⁰, 10⁻⁵⁰, 10⁻²⁰, 10⁻¹⁰, 10⁻⁵, 10⁻³, 10⁻¹, 1, 5, 10, 25, 50, 75, 80, 90 or 95 percent in mass of carbon, nitrogen, sulfur, sulfate, phosphorous, phosphate, calcium, Potassium, Magnesium, Oxygen, hydrogen, or iron, respectively. In some cases, they are in a gaseous, liquid, or solid state. In some other cases, they can be used to prepare the pre-growth and/or growth medium/media. In some cases, the pre-growth and/or growth medium/media comprise(s) more than 2, 3, 4, 5, 10, 50, 100, 10³, 10⁵, 10¹⁰ or 10⁵⁰ different sources of carbon, nitrogen, sulfur, sulfate, phosphorous, phosphate, calcium, potassium, magnesium, oxygen, hydrogen, and/or iron. In some other cases, the pre-growth and/or growth medium/media comprise(s) less than 2, 3, 4, 5, 10, 50, 100, 10³, 10⁵, 10¹⁰ or 10⁵⁰ different sources carbon, nitrogen, sulfur, sulfate, phosphorous, phosphate, calcium, potassium, magnesium, oxygen, hydrogen, and/or iron.

In one embodiment of the invention, at least one source of carbon, nitrogen, sulfur, sulfate, phosphorous, phosphate, calcium, potassium, magnesium, oxygen, hydrogen, and/or iron of the pre-growth medium is the same as that of the growth medium.

In another embodiment of the invention, at least one source of carbon, nitrogen, sulfur, sulfate, phosphorous, phosphate, calcium, potassium, magnesium, oxygen, hydrogen, and/or iron of the pre-growth medium is different from that of the growth medium.

In another embodiment of the invention, the pre-growth and/or growth medium/media comprise(s) the source(s) of carbon, nitrogen, sulfur, sulfate, phosphorous, phosphate, calcium, potassium, magnesium, oxygen, hydrogen, and/or iron at a concentration that is larger than 10⁻¹⁰⁰, 10⁻⁵⁰, 10⁻²⁰, 10⁻¹⁰, 10⁻⁵, 10⁻³, 10⁻¹, 1, 10, 10², 10³, 10⁵ or 10¹⁰ mM.

In another embodiment of the invention, the pre-growth and/or growth medium/media comprise(s) the source(s) of carbon, nitrogen, sulfur, sulfate, phosphorous, phosphate, calcium, potassium, magnesium, oxygen, hydrogen, and/or iron, at a concentration that is lower than 10¹⁰⁰, 10⁵⁰, 10²⁰, 10¹⁰, 10⁵, 10³, 10, 1, 10⁻¹, 10⁻³, 10⁻⁶, 10⁻⁹, 10⁻²⁰, 10⁻⁵⁰ or 10⁻¹⁰⁰ mM.

In an another embodiment of the invention, the pre-growth and/or growth medium/media is/are prepared using pharmaceutical grade or ultrapure chemicals or chemical element(s).

In still another embodiment of the invention, medium impurity(ies) is/are impurity(ies) comprised in the pre-growth and/or growth and/or fed-batch medium/media.

In one embodiment of the invention, the pre-growth and/or growth medium/media comprise(s) a low quantity of medium impurity(ies). In some cases, the percentage of medium impurity(ies) is lower than 100, 10²⁰, 10¹⁰, 10⁵, 10², 10, 5, 1, 0.1, or 0.001%. Preferentially, the pre-growth and/or growth medium/media comprise(s) a quantity or concentration of medium impurity(ies), which is lower, preferentially by a factor of at least 1.00001, 1.1, 1.5, 2, 5, 10, 10³, 10¹⁰ or 10²⁰, than the quantity or concentration of at least one source of carbon, nitrogen, sulfur, sulfate, phosphorous, phosphate, calcium, potassium, magnesium, oxygen, hydrogen, and/or iron.

According to the invention, the percentage in medium impurity(ies) can in some cases be defined as the ratio between the number of atoms, quantity, mass, or volume of medium impurity(ies) comprised in the pre-growth and/or growth medium/media divided by the total number of atoms, quantity, mass, or volume of all chemical element(s) comprised in the pre-growth and/or growth medium/media. In some other cases, the concentration of medium impurity(ies) comprised in the pre-growth and/or growth medium/media is lower than 10⁵⁰, 10³⁰, 10¹⁰, 10⁵, 10³, 500, 100, 50, 10, 1, 10⁻¹, 10⁻³, 10⁻⁵, 10⁻¹⁰ or 10⁻⁵⁰ μg of medium impurity(ies) per mL of pre-growth and/or growth medium/media.

In still another embodiment of the invention, the pre-growth and/or growth medium/media does/do not comprise at least one medium impurity.

In one embodiment of the invention, the pre-growth and/or growth medium/media comprise(s) a significant quantity of medium impurity(ies). In some cases, the percentage, preferentially in mass, of medium impurity(ies), is larger than 0, 10⁻⁴⁰, 10⁻²⁰, 10⁻¹⁰, 10⁻⁵, 10⁻², 10⁻¹, 1, 5, 10, 25, 50, 75, 80, or 90%. In some other cases, the concentration of medium impurity(ies) comprised in the pre-growth and/or growth medium/media is larger than 10⁻¹⁰⁰, 10⁻⁵⁰, 10⁻²⁰, 10⁻¹⁰, 10⁻⁵, 10⁻³, 10⁻², 10⁻¹, 1, 10, 50, 100, 10³, 10⁵, or 10¹⁰ μg of medium impurity(ies) per mL of pre-growth and/or growth medium/media.

In some cases, the nanoparticles produced or obtained or resulting from the pre-growth and/or growth medium/media can be magnetosomes.

In one embodiment of the invention, the method according to the invention comprises a pre-growth step, which consists in amplifying the nanoparticle-producing cell(s) in a pre-growth medium so that nanoparticle-producing cell(s) produce(s) essentially no nanoparticles.

In one embodiment of the invention, the nanoparticle-producing cell(s) used to start the pre-growth step are cell(s) with at least one of the following properties: i) they are the cells before the pre-growth step, preferentially more than 0.001, 0.1, 1, 5, 10, 10³, 10⁵ or 10¹⁰ hour(s) before the beginning of the pre-growth step, ii) they are comprised in a cellular bank such as a master cell bank, a working cell bank, or a research cell bank, iii) they comprise more than 1, 5, 10, 10³, 10⁵ or 10¹⁰ nanoparticle(s) per cell, iv) they are comprised in a liquid or medium, preferentially identical or similar in composition to the pre-growth and/or growth medium/media, preferentially comprising a majority of water, v) they are comprised in a medium with a concentration in medium impurity(ies) lower than 100, 10, 1, 10⁻¹, 10⁻², 10⁻³, 10⁻⁵ or 10⁻¹⁰ μM, vi) they are comprised in a medium or kept in conditions that enables maintaining or having less than 100, 10, 1, 0.1 or 0.01 gram of impurity(ies) per gram of nanoparticle(s), v) they are comprised in a volume comprised between 10⁻¹⁰⁰ and 10¹⁰⁰, 10⁻⁵⁰ and 10⁵⁰, 10⁻³⁰ and 10³⁰, 10⁻²⁰ and 10²⁰, 10⁻¹⁰ and 10¹⁰, 10⁻⁶ and 10⁵, 10⁻⁶ and 10⁴, 10⁻⁶ and 10², or between 10⁶ and 1 liter, vi) they are comprised in a volume at least 10 times lower than the volume of the first pre-growth step, vii) they are or represent a number of cell(s), preferentially per liter of pre-growth and/or growth medium/media, comprised between 1 and 10¹⁰⁰, 2, and 10⁵⁰, 3 and 10²⁰, or between 10 and 10¹⁰ cells, vii) they have an optical density comprised between 10⁻⁵⁰ and 10⁵⁰, 10⁻²⁰ and 10²⁰, 10⁻¹⁰ and 10, 10⁻⁵ and 10⁵, 10⁻⁵ and 10³, 10⁻⁵ and 10², 10⁻⁵ and 1, 10⁻⁵ and 10⁻¹, 10⁻⁵ and 10⁻², or between 10⁻⁵ and 10⁻³, viii) they have a number of cell division, preferentially per hour or per hour per liter of pre-growth and/or growth medium/media, which is lower than 1, 10, 10³, 10⁵, 10¹⁰, 10²⁰, 10⁵⁰ or 10¹⁰⁰, ix) they are stored or kept at a temperature below 100, 50, 25 or 0° C., preferentially at 77 K or −20° C.

In one embodiment of the invention, the nanoparticle-producing cells used to start the pre-growth step have at least one of the following properties: i) they are comprised in a medium with a concentration in medium impurity larger than 10⁻⁵, 10⁻²⁰, 10⁻¹⁰, 10⁻⁵, 10⁻², 10⁻¹, 1 or 10 μM, ii) they are comprised in a medium or kept in conditions that enable maintaining or having more than 10⁻⁴⁰, 10⁻²⁰ or 10⁻¹⁰ gram of impurity(ies) per gram of nanoparticle(s), iii) they have a number of cell division, preferentially per hour or per hour per liter of pre-growth and/or growth medium/media, which is larger than 1, 10, 10³, 10⁵, 10¹⁰, 10²⁰, 10⁵⁰ or 10¹⁰⁰, iv) they comprise less than 1, 5, 10, 10³, 10⁵ or 10¹⁰ nanoparticle(s) per cell. This can be the case when/if they are maintained or originate from a medium that comprises a sufficiently low iron concentration to prevent the production of nanoparticles.

In one embodiment of the invention, at least one the property(ies) of the nanoparticle-producing cells used to start the pre-growth step enables preventing the death or destruction or disappearance or denaturation or inactivation of the nanoparticle-producing cell(s).

In an embodiment of the invention, the optical density of the cell(s) is measured when the cells are comprised in the pre-growth and/or growth medium/media, in a solution, or in water, preferentially after the growth medium has been removed and the cells have been re-suspended in water. In some cases, the optical density of the cell(s) is measured at a wavelength larger than 1, 2, 5, 10, 50, 100, 200, 300, 400, 450, 500, 550, 600, 900, 10³, 10⁵ or 10⁷ nm. In some other cases, the optical density of the cell(s) is measured at a wavelength lower than 10⁷, 10⁵, 10³, 900, 600, 550, 500, 450, 400, 300, 200, 100, 50, 10, 5, 2 or 1 nm. In sill some other cases, the optical density of the cells is measured at a wavelength comprised between 1 and 10⁷ nm, 50 and 10⁵ nm, 100 and 10³ nm, 200 and 900 nm, or between 400 and 800 nm.

In one embodiment of the invention, the number of cell amplification, between two time points t₀ and t₁ of the pre-growth and/or growth step(s), is equal to or is proportional to: i), the ratio between the optical density measured at t₁ and the optical density measured at to and/or ii), the ratio between the number of cells at t₁, n(t₁), and the number of cells at to, n(t₀).

In one embodiment of the invention, the speed or rate of cell division is [n(t₁)−n(t₀)]/(t₁−t₀).

In another embodiment, the speed or rate of cell division is: n(t₁)−n(t₀)/[(t₁−t₀)·V], where V is the volume of the pre-growth and/or growth medium in which the cells are cultured or amplified.

In one embodiment of the invention, the pre-growth step begins by thawing or heating, preferentially from a temperature below 100, 50, 25, 10 or 0° C. to a temperature above 0, 10, 25, 50 or 100° C., the nanoparticle-producing cell(s) used to start the pre-growth step. After that, the nanoparticle-producing cells are preferentially inserted or added the pre-growth medium. In some cases, this initial phase of the pre-growth step takes place during a lapse of time comprised between 10⁻⁵⁰ and 10⁵⁰, 10⁻⁵⁰ and 10¹⁰, 10³⁰ and 10⁵, 10⁻²⁰ and 10³, 10⁻¹⁰ and 10², or between 10⁻⁵ and 10 hour(s).

In another embodiment of the invention, the pre-growth step is divided in sub-steps 0, 1, . . . , i, . . . , j, corresponding to the amplifications in the different, preferentially increasing, volumes V₀, V₁, . . . , Vᵢ, . . . , Vⱼ, where i is an integer designating the number i of amplifications in different volumes (0<i<j), j is an integer designating the total number of amplifications in different volumes, V₀, Vᵢ, and Vⱼ are the initial, i^{th}, and final volume of amplification, respectively. In some cases, cell amplification or the number of cell amplification in different volumes can be important during the pre-growth step, for example when the pre-growth step begins from a low quantity of cells, preferentially lower than 10¹⁰⁰, 10⁵⁰, 10²⁰, 10¹⁰, 10⁵, 10³, 10², 10, 5, 3 or 2 cells, preferentially comprised in one liter or one milliliter or one microliter of pre-growth medium or aqueous solution. In these cases, i and/or j can be larger than 1, 2, 3, 4, 5, 6, 7, 8, 9, 10, 15, 20, 10, 10³, 10⁵ or 10¹⁰. In some other cases, cell amplification or the number of cell amplification in different volumes can be low, for example when the pre-growth step begins from a large quantity of cells, preferentially larger than 2, 3, 5, 10, 10², 10³, 10⁵, 10¹⁰, 10²⁰, 10⁵⁰ or 10¹⁰⁰ cells, preferentially comprised in one liter or one milliliter or one microliter of pre-growth medium or aqueous solution. In these cases, i and/or j is/are lower than 10¹⁰, 10⁵, 10³, 10², 10, 5, 4, 3, 2 or 1.

In one embodiment of the invention, the ratio Vᵢ/Vᵢ₋₁ is large, preferentially larger than 10⁻⁵⁰ 10⁻³⁰, 10⁻²⁰, 10⁻¹⁰, 10⁻⁵, 10⁻³, 10⁻², 10⁻¹, 1, 1.00001, 1.0001, 1.001, 1.01, 1.1, 1.2, 1.5, 2, 3, 4, 5, 7, 10, 10², 10³, 10⁵ or 10¹⁰. In some cases, Vᵢ/Vᵢ₋₁ is large when between the sub-step i−1 and the sub-step i of the pre-growth step: the number of cell division, preferentially per hour or per hour per liter of pre-growth medium, is larger than 1, 5, 10, 10³, 10¹⁰ or 10²⁰, or when the cell optical density increases by a factor of more than 1.00001, 1.1, 2, 5, 10, 10³, 10⁵ or 10⁷ per hour.

In another embodiment of the invention, the ratio Vᵢ/Vᵢ₋₁ is low, preferentially lower than 10¹⁰⁰, 10⁵⁰, 10¹⁰, 10⁵, 10³, 10², 10, 5, 3, 2, 1.01, 1.001, 1.000001, 1, 10⁻⁵, 10⁻¹⁰ or 10⁻⁵⁰. In some cases, Vᵢ/Vᵢ₋₁ is low when the number of cell division, preferentially per hour or per hour per liter of pre-growth medium, is lower than 10⁵⁰, 10¹⁰, 10³, 10², 10, 5 or 1, or when the cell optical density increases by a factor of less than 1.00001, 1.1, 2, 5, 10, 10³, 10⁵ or 10⁷ per hour.

In some cases, the number of pre-growth steps in different volumes can be increased by decreasing Vᵢ/Vᵢ₋₁. In some other cases, the number of pre-growth steps in different volumes can be decreased by increasing Vᵢ/Vᵢ₋₁.

In one embodiment of the invention, the pre-growth step and/or at least one of its sub-step(s) last(s) and/or take(s) place until the optical density of the bacterial suspension in volume Vᵢ (1<i<j) reaches a value that is: i), above 10⁻⁵⁰, 10⁻³⁰, 10⁻²⁰, 10⁻¹⁰, 10⁻⁵, 10⁻³, 10⁻¹, 1, 5, 10, 15, 50, 10², 10³ or 10⁵, and/or ii), larger, preferentially by a factor of more than 1.000001, 1.0001, 1.01, 1.1, 1.5, 2, 5, 10, 10², 10³, 10⁵, 10¹⁰ or 10²⁰, at the end than at the beginning of the pre-growth step and/or at the end than at the beginning of at least one sub-step of the pre-growth step.

In one embodiment of the invention, the pre-growth step and/or at least one of its sub-step(s) last(s) and/or take(s) place until the optical density of the bacterial suspension in volume Vᵢ (1<i<j) reaches a value that is: i), lower than 10⁻⁵⁰, 10⁻³⁰, 10⁻²⁰, 10⁻¹⁰, 10⁻⁵, 10⁻³, 10⁻¹, 1, 5, 10, 15, 50, 10², 10³ or 10⁵, and/or ii), lower, preferentially by a factor of more than 1.000001, 1.0001, 1.01, 1.1, 1.5, 2, 5, 10, 10², 10³, 10⁵, 10¹⁰ or 10²⁰, at the end than at the beginning of the pre-growth step and/or at the end than at the beginning of at least one sub-step of the pre-growth step.

In one embodiment of the invention, the beginning of the amplification in volume V₀ takes place at time t_{PG0b}, the end of the amplification in volume V₀ takes place at time t_{PG0e}, the beginning of the amplification in volume Vᵢ occurs at time t_{PGib}, the end of amplification in volume Vᵢ takes place at time t_{PGie}, the beginning of the amplification in volume Vⱼ takes place at time t_{jb}, and/or the end of the amplification in volume Vⱼ takes place at time t_{PGje}.

In one embodiment of the invention, the length of time that separates: i), the beginning of the pre-growth step, at time t_{PG0b}, and the end of the pre-growth step, at time t_{PGje}, which is equal to t_{PGje}−t_{PG0b}, and/or ii), the beginning of sub-step i, t_{PGib}, and the end of sub-step i, t_{PGie}, which is equal to t_{PGie}−t_{PGib}, is/are larger than or equal to 10⁻²⁰, 10⁻¹⁰, 10⁻⁵, 10⁻³, 10⁻², 10⁻¹, 1, 2, 5, 10, 24, 100, 10³, 10⁵ or 10⁷ hour(s). In some cases, t_{PGje}−t_{PG0b} and/or t_{PGie}−t_{PGib} is/are large when the cells divide with difficulty or slowly, preferably at a speed or rate lower than 10⁻⁵⁰, 10⁻²⁰, 10⁻¹⁰, 10⁻⁵, 10⁻³, 10⁻¹, 1, 10, 10³ or 10⁵ cellular division(s) per hour or cellular division(s) per hour per liter of pre-growth medium.

In still another embodiment of the invention, t_{PGje}−t_{PG0b} and/or t_{PGie}−t_{PGib} is/are lower than or equal to 10⁴⁰, 10³⁰, 10²⁰, 10¹⁰, 10⁵, 10³, 10², 10, 5, 2, 1, 10⁻¹, 10⁻³, 10⁻⁵ or 10⁻¹⁰ hour(s). In some cases, t_{PGje}−t_{PG0b} and/or t_{PGie}−t_{PGib} is/are low when the cells divide easily or rapidly, preferably at a speed or rate larger than 10⁻⁵⁰, 10⁻²⁰, 10⁻¹⁰, 10⁻⁵, 10⁻³, 10⁻¹, 1, 10, 10³ or 10⁵ cellular division(s) per hour(s) or cellular division(s) per hour per liter of pre-growth medium.

In one embodiment of the invention, the nanoparticle-producing cells are amplified during the pre-growth step by introducing at t_{PGib} or t_{PG0b} or by maintaining during the pre-growth step or at least one of its sub-step an iron concentration in the pre-growth medium that is lower than 10¹⁰⁰, 10²⁰, 10⁵, 10³, 10, 5, 1, 10⁻¹, 10⁻³ or 10⁻⁵ μM, preferentially to avoid nanoparticle synthesis that could prevent cellular amplification.

In another embodiment of the invention, the nanoparticle-producing cells are amplified during the pre-growth step by introducing at t_{PGib} or t_{PG0b} or by maintaining during the pre-growth step or at least one of its sub-step an iron concentration in the pre-growth medium that is larger than 10⁻⁵⁰, 10⁻²⁰, 10⁻¹⁰, 10⁻⁵, 10⁻³, 10⁻² or 10⁻¹ μM, preferentially to enable efficient cellular metabolism.

In another embodiment of the invention, the nanoparticle-producing cells are amplified during the pre-growth step by introducing at t_{PGib} or t_{PG0b} or by maintaining during the pre-growth step or at least one of its sub-step an iron concentration in the pre-growth medium between 10⁻¹⁰ and 10¹⁰, 10⁻⁵ and 10⁵, 10⁻³ and 10³, 10⁻¹ and 1 μM, 10⁻¹ and 10 μM, or between 10⁻² and 100 μM.

In another embodiment of the invention, the nanoparticle-producing cells are amplified during the pre-growth step or at least one of its sub-steps by consuming oxygen. In some cases, the percentage of oxygen in the pre-growth medium decreases from: i), a value above 10⁻⁵⁰, 10⁻¹⁰, 10⁻⁵, 10⁻³, 1, 5, 10, 20, 50, 75, 90, 95, 99 or 99.9%, preferentially from 21% or a value comprised between 10 and 30%, at t_{PGib} or t_{PG0b}, down to a value below 99.9, 95, 90, 80, 75, 50, 20, 5, 2, 1, 10⁻³, 10⁻⁵, 10⁻¹⁰ or 10⁻⁵⁰%, preferentially 0% or a value comprised between 0 and 10%, at t_{PGie} or t_{PGje}, and/or ii), the percentage of oxygen in the pre-growth medium decreases by a factor of more than 1.0001, 1.001, 1.1, 1.2, 1.5, 2, 5, 10, 50, 10², 10³, 10⁵ or 10¹⁰, preferentially between t_{PG0b} and t_{PGje} and/or between t_{PGib} and t_{PGie}. In some cases, oxygen is not added to the pre-growth medium during the pre-growth step or at least one of its sub-steps, resulting in a decrease in the percentage of oxygen in the pre-growth medium due to the consumption of oxygen by the bacteria. In some other cases, oxygen is added to the pre-growth medium during the pre-growth step or at least one of its sub-steps, resulting in a variation of oxygen percentage in the pre-growth medium that is due both to the consumption of oxygen by the bacteria and to the addition of oxygen to the pre-growth medium.

In one embodiment of the invention, the percentage of oxygen, preferentially O₂, in the pre-growth and/or growth medium/media is the percentage of dissolved oxygen, preferentially O₂, in the pre-growth and/or growth medium/media. In some cases, a percentage of 100% can correspond to the maximum quantity of O₂ solubilized in the pre-growth and/or growth medium/media, comprised between 10⁻¹ and 10²⁰ mg, preferentially comprised between 1 and 10 mg of dissolved O₂ per liter of pre-growth and/or growth medium/media.

In one embodiment of the invention, the pre-growth step consists in amplifying the cells, where such amplification is associated with, corresponds to, or is: i) a speed or rate or number of cell division, preferentially per unit volume such as one liter of pre-growth medium, which is larger, preferentially by a factor of more than 1.000001, 1.0001, 1.01, 1.1, 1.2, 1.5, 2, 5, 10, 10², 10³, 10⁵, 10¹⁰, 10²⁰ or 10⁵⁰, at the beginning than end of the pre-growth step or one of its sub-step, or at t_{PGie} or t_{PGje} than at t_{PGib} or t_{PG0b}, ii) a speed or rate or number of cell division, preferentially per unit volume such as one liter of pre-growth medium, which increases from a number of cells or cells per hour lower than or equal to 10¹⁰⁰, 10⁵⁰, 10²⁰, 10¹⁰, 10⁵, 10³, 10², 10, 5 or 2, at the beginning of the pre-growth step or one of its sub-step or at t_{PGib} or t_{PG0b}, up to a number of cells or cells per hour larger than or equal to 2, 5, 10, 10², 10³, 10⁵, 10¹⁰, 10²⁰, 10⁵⁰ or 10¹⁰⁰, at the end of the pre-growth step or one of its sub-step or at t_{PGie} or t_{PGje}, iii) an optical density, preferentially measured for cells comprised in a fixed pre-growth volume such as one liter, which is larger, preferentially by a factor of more than 1.00001, 1.1, 1.2, 1.5, 2, 5, 10, 10³, 10⁵, 10¹⁰, 10²⁰ or 10⁵⁰, at the end of the pre-growth step or one of its sub-step or at t_{PGie} or t_{PGje} than at the beginning of the pre-growth step or one of its sub-step or at t_{PGib} or t_{PG0b}, or which increases from an optical density lower than or equal to 10, 1, 10⁻¹, 10⁻² or 10⁻³ at the beginning of the pre-growth step or one of its sub-step or at t_{PGib} or t_{PG0b} to an optical density larger than or equal to 10⁻¹⁰, 10⁻², 10⁻¹, 1 or 10 at the end of the pre-growth step or one of its sub-step or at t_{PGie} or t_{PGje}.

In one embodiment of the invention, preferentially during, at the beginning, or at the end of the pre-growth step or of at least one of its sub-steps, cells that produce essentially no nanoparticles have or are characterized by at least one of the following properties: i) a number of nanoparticles comprised in the cells that is lower than 10³; 10², 50, 20, 10, 5, 2 or 1, preferentially lower than 10 or 5, or between 0 and 10³, preferentially between 0 and 10 or between 0 and 5, ii) a percentage of cells with at least one nanoparticle that is lower than 100, 99, 90, 80, 50, 20, 10, 1, 0.1%, preferentially lower than 10 or 1% or comprised between 0 and 99%, 0 and 50, 0 and 10, preferentially between 0 and 5%, where this percentage is preferentially the ratio between the number of cells with at least one nanoparticle and the total number of cells, preferentially comprised in the pre-growth medium, iii) an optical density larger than 10⁻⁵⁰, 10⁻²⁰, 10⁻¹⁰, 10⁻⁵, 10⁻³, 10⁻², 0.1, 0.2, 0.5, 1, 5, 10 or 100, iv) a number of cells larger than 1, 5, 10, 10², 10³, 10⁵, 10¹⁰, 10²⁰, 10⁵⁰ or 10¹⁰⁰, v) they are comprised in a volume larger than 0.0001, 0.001, 0.1, 1, 10, 50, 10², 10³, 10⁵ or 10¹⁰ liter(s), vi) a number of cell generation comprised between 1 and 10¹⁰, 1 and 10³, preferentially between 50 and 300, vii) a ratio between the optical density measured at the end of the pre-growth step, OD_{PG}E, and the beginning of the pre-growth step, OD_{PGB}, OD_{PG}E/OD_{PGB}, larger than 1, 2, 5, 10, 15, 25, 50, 100, 10³, 10⁵, 10¹⁰, 10⁵⁰ or 10¹⁰⁰, and/or viii) the ratio between the optical density measured at the end of sub-step i of the pre-growth step, OD_{PGiE}, and the optical density measured at the beginning of sub-step i of the pre-growth step, OD_{PGiB}, OD_{PGiE}/OD_{PGiB}, larger than or equal to 1, 2, 5, 10, 15, 25, 50, 100, 10³, 10⁵, 10¹⁰, 10⁵⁰ or 10¹⁰⁰.

In one embodiment of the invention, the cells that produce essentially no nanoparticles are non-magnetic cells.

In one embodiment of the invention, preferentially during, at the beginning or at the end of the pre-growth step or at least one of its sub-steps, a percentage of non-magnetic cells larger than 10⁻⁵⁰, 10⁻²⁰, 10⁻¹⁰, 10⁻⁵, 10⁻³, 10⁻¹, 1, 5, 10, 50 or 75%, is obtained. In some cases, the percentage of non-magnetic cells is based on the ratio n_{NMC}/(n_{MC}+n_{NMC}), where n_{MC} and n_{NMC} are the numbers of magnetic cells and non-magnetic cells, respectively.

In another embodiment of the invention, non-magnetic cells don't display a magnetic response, where the magnetic response can be the orientation of at least one cell parallel to a magnetic field or a movement of the cell at a speed that is proportional to the strength of the magnetic field, where the strength of the magnetic field can be larger than 10⁻⁹, 10⁻³, 10⁻¹, 1, 10³ or 10⁶ mT and/or the magnetic field is preferentially applied on the cell(s).

In one embodiment of the invention, the method comprises a growth step consisting in amplifying the nanoparticle-producing cell(s) originating from the pre-growth step in a growth medium so that nanoparticle-producing cell(s) produce(s) nanoparticles. In some cases, the growth step is carried out in a fermenter or apparatus, which enables to control the temperature, pH, iron concentration, and/or concentration of oxygen of the growth medium.

In another embodiment of the invention, the growth step begins by inserting the cells obtained from the pre-growth step in the growth medium. In some cases, the growth step or at least one of its sub-steps takes place during a lapse of time comprised between 10⁻⁵⁰ and 10⁵⁰, 10⁻⁵⁰ and 10¹⁰, 10³⁰ and 10⁵, 10⁻²⁰ and 10³, 10⁻¹⁰ and 10², or between 10⁻⁵ and 24 hour(s). In some other cases, the growth step or at least one of its sub-steps takes place during a lapse of time lower than 10²⁰, 10¹⁰, 10⁵, 10³, 10², 10, 5, 2, 1, 10⁻¹, 10⁻², 10⁻¹, 10⁻⁵, 10⁻¹⁰ or 10⁻²⁰ hour(s). In still some other cases, the growth step or at least one of its sub-step takes place during a lapse of time larger than 10⁻²⁰, 10⁻¹⁰, 10⁻⁵, 10⁻¹, 10⁻², 10⁻¹, 1, 2, 5, 10, 10², 10³, 10⁵, 10¹⁰ or 10²⁰ hour(s).

In one embodiment of the invention, the growth step consists in amplifying the cells during successive sub-steps GS_{O} . . . GSᵢ . . . GSⱼ, where t_{GS0}b, t_{GSib}, t_{GSjb}, are the beginning of steps 0, i, and j, and t_{GS0e}, t_{GSie}, t_{GSje}, are the ends of steps 0, i, and j, where 0<i<j. In some cases, i and/or j is/are larger than 1, 2, 3, 4, 5, 6, 7, 8, 9, 10, 15, 20, 10, 10³, 10⁵ or 10¹⁰. In some other cases, i and/or j is/are lower than 1, 2, 3, 4, 5, 6, 7, 8, 9, 10, 15, 20, 10, 10³, 10⁵ or 10¹⁰.

In one embodiment of the invention, each subset i consists in bubbling or bringing a different amount of oxygen to/in the growth medium and/or bringing a different quantity of iron to/in the growth medium, preferentially with the help of a fed-batch medium.

In one embodiment of the invention, the growth step comprises at least one of the following sub-steps during which a gas, such as compressed air or a gas comprising more than 1% of O₂, is introduced to the growth medium, preferentially under stirring conditions at 1 to 10¹⁰, 5 to 10⁵, 10 to 10⁴, 100 to 10³, or 100 to 300 rotations per minute, and wherein:
During the first sub-step that lasts for 10⁻³ to 10³ hours or preferentially for 2 to 16 hours, the debit of the gas is comprised between 0 and 10¹⁰, preferentially between 0.001 and 40 mL/min per liter of growth medium, resulting in an increase of the optical density of the cells, from a value comprised between 10⁻¹⁰ and 10³, preferentially between 0.08 and 0.12 at the beginning of the first sub-step to a value at the end of the first sub-step larger by a factor of more than 1.0000001, 1.1, 1.5, 2, 5, 10 or 10³ than the value at the beginning of the first sub-step or to a value comprised between 10⁻⁹ and 10⁴, preferentially between 0.2 and 1.
During the second sub-step that lasts for 10⁻³ to 10³ hours or preferentially for 2 to 120 hours, the debit of the gas is increased by a factor larger than 1.0000001, 1.1, 1.5, 2, 5, 10 or 10¹ compared with the first sub-step or is comprised between 0 and 10¹⁰, preferentially between 1 and 50 mL/min per liter of growth medium, resulting in an increase of the optical density of the cells from a value at the beginning of the second sub-step that is equal to that obtained at the end of the first stub-step or that is comprised between 10⁻⁹ and 10⁴, preferentially between 0.2 and 1, to a value at the end of the second sub-step larger by a factor of more than 1.0000001, 1.1, 1.5, 2, 5, 10 or 10³ than the value at the beginning of the second sub-step or to a value comprised between 10⁻⁹ and 10⁴, preferentially comprised between 0.5 and 4.
During the third sub-step that lasts for 10⁻³ to 10¹ hours or preferentially for 2 to 120 hours, the debit of the gas is increased by a factor larger than 1.0000001, 1.1, 1.5, 2, 5, 10 or 10³ compared with the second sub-step or is comprised between 0 and 10¹⁰, preferentially between 50 and 120 mL/min per liter of growth medium, resulting in an increase of the optical density of the cells from a value at the beginning of the third sub-step that is equal to that obtained at the end of the second sub-step or that is comprised between 10⁻⁹ and 10⁴, preferentially between 0.5 and 4 to a value at the end of the third sub-step larger by a factor of more than 1.0000001, 1.1, 1.5, 2, 5, 10 or 10³ than the value at the beginning of the third sub-step or to a value comprised between 10⁻⁹ and 10⁴, preferentially comprised between 1 and 8 at the end of the third sub-step.
During the fourth sub-step that lasts for 10⁻³ to 10³ hours, preferentially for 2 to 120 hours, the debit of the gas is increased by a factor larger than 1.0000001, 1.1, 1.5, 2, 5, 10 or 10³ compared with the third sub-step or is comprised between 0 and 10¹⁰, preferentially between 200 and 300 mL/min per liter of growth medium, resulting in an increase of the optical density of the cells from a value at the beginning of the fourth sub-step that is equal to that obtained at the end of the third sub-step or that is comprised between 10⁻⁹ and 10⁴, preferentially between 1 and 8 to a value preferentially at the end of the fourth sub-step larger by a factor of more than 1.0000001, 1.1, 1.5, 2, 5, 10 or 10¹ than the value at the beginning of the fourth sub-step or to a value comprised between 10⁻⁹ and 10⁴, preferentially between 2 and 16 at the end of the fourth sub-step.
During the fifth sub-step that lasts for 10⁻³ to 10¹ hours, preferentially for 2 to 120 hours, the debit of the gas is increased by a factor larger than 1.0000001, 1.1, 1.5, 2, 5, 10 or 10³ compared with the fourth sub-step or is comprised between 0 and 10¹⁰, preferentially between 300 and 500 mL/min per liter of growth medium, resulting in an increase of the optical density of the cells from a value at the beginning of the fifth sub-step that is equal to that obtained at the end of the fourth sub-step or that is comprised between 10⁻⁹ and 10⁴, preferentially between 2 and 16 to a value at the end of the fifth sub-step larger by a factor of more than 1.0000001, 1.1, 1.5, 2, 5, 10 or 10³ than the value at the beginning of the fifth sub-step or to a value comprised between 10⁻⁹ and 10⁴, preferentially comprised between 4 and 32 at the end of the fifth sub-step.

In one embodiment of the invention, during sub-step i, preferentially sub-steps 2 to 5: i) the percentage of oxygen is maintained above 0.01% or 0.1 mBar by the debit of air and below 0.9% or 9 mBar due to the consumption of oxygen by the cells, ii) the debit of the gas is comprised between 0 and 10¹⁰, 1 and 10⁵, 5 and 10⁴, 10 and 10¹ mL/min per liter of growth medium, iii) the growth medium is stirred at a speed comprised between 1 and 10⁵, 10 and 10⁴, 50 and 10³, or between 100 and 500 rotations per minute, iv) the debit of the gas can be decreased by increasing the stirring rate of the media, v) the debit of the gas can be increased by decreasing the stirring rate of the media, and/or vi) the optical density of the cells increases from a value preferentially comprised between 10⁻⁵⁰ and 10³ at the beginning of sub-step i to a value preferentially comprised between 10⁻²⁰ and 10⁵ at the end of sub-step i.

In one embodiment of the invention, the growth step comprises at least one sub-step during which: i) the pH of the growth medium is maintained at a fixed or determined or chosen pH, comprised between 0 and 14, 2 and 13, 4 and 11, 5 and 10, preferentially comprised between 5 and 8, most preferentially equal to 6.9, preferentially by adding an acidic source of iron comprised in a fed-batch medium, preferentially under stirring conditions at 1 to 10¹⁰, preferentially 100 to 300 rotations per minute. In some cases, the growth medium comprises an iron concentration at the beginning of the growth step or one of its sub-step that is: i) lower than 10¹⁰, 10⁵ or 10² μM, preferentially lower than 10 or 2 μM, and/or ii) comprised between 10⁻¹⁰ and 10¹⁰, 10⁻⁵ and 10⁵, 10⁻³ and 10³ μM, preferentially comprised between 0.2 and 20 μM. In some other cases, during the growth step or one of its sub-step, the iron concentration of the growth medium increases, preferentially with the addition of the fed-batch medium to the growth medium, to reach a value at the end of the growth step or one of its sub-step that is: i) larger than 10⁻¹⁰, 10⁻⁵, 10⁻¹ or 1 μM, preferentially larger than 2 μM and/or ii) comprised between 10⁻¹⁰ and 10¹⁰ μM, preferentially between 2 μM and 5 mM or between 2 μM and 0.5 mM.

In one embodiment of the invention, the growth step comprises at least one of the following sub-step(s), wherein:
During the first sub-step, which lasts for 10⁻³ to 10³, preferentially 2 to 16 hours, the insertion of the fed-batch medium to the growth medium yields an iron concentration in the growth medium comprised between 10⁻¹⁰ and 10¹⁰ μM, preferentially between 2 and 20 μM, in some cases without taking into account the consumption of iron by the bacteria, in some other cases by taking into account the consumption of iron by the bacteria. This preferentially results in a production of nanoparticles that increases by a factor larger than 1.0000001, 1.1, 1.5, 2, 5, 10 or 10¹ from the beginning to the end of the first sub-step, or from a value comprised between 10⁻¹⁰ and 10¹⁰, preferentially between 0.001 and 0.1 mg of nanoparticles per liter of growth medium at the beginning of the first sub-step to a value comprised between 10⁻¹⁰ and 10¹⁰, preferentially between 1 and 10 mg of nanoparticles per liter of growth medium at the end of the first sub-step.
During the second sub-step, which lasts for 10⁻³ to 10³, preferentially 2 to 120 hours, the insertion of the fed-batch medium to the growth medium yields an iron concentration in the growth medium comprised between 10⁻¹⁰ and 10¹⁰ μM, preferentially between 20 and 40 μM, in some cases without taking into account the consumption of iron by the bacteria, in some other cases by taking into account the consumption of iron by the bacteria. This preferentially results in a production of nanoparticles that increases by a factor larger than 1.0000001, 1.1, 1.5, 2, 5, 10 or 10¹ from the beginning to the end of the second sub-step, or from a value comprised between 10⁻¹⁰ and 10¹⁰, preferentially 1 and 10 mg of nanoparticles per liter of growth medium at the beginning of the second sub-step to a value comprised between 10⁻¹⁰ and 10¹⁰, preferentially between 2 and 20 mg of nanoparticles per liter of growth medium at the end of the second sub-step.
During the third sub-step, which lasts for 10⁻³ to 10³, preferentially 2 to 120 hours, the insertion of fed-batch medium to the growth medium yields an iron concentration in the growth medium comprised between 10⁻¹⁰ and 10¹⁰ μM, preferentially between 40 and 150 μM, in some cases without taking into account the consumption of iron by the bacteria, in some other cases by taking into account the consumption of iron by the bacteria. This preferentially results in a production of nanoparticles that increases by a factor larger than 1.0000001, 1.1, 1.5, 2, 5, 10 or 10¹ from the beginning to the end of the third sub-step, or from a value comprised between 10⁻¹⁰ and 10¹⁰, preferentially between 2 and 20 mg of nanoparticles per liter of growth medium at the beginning of the third sub-step to a value comprised between 10⁻¹⁰ and 10¹⁰, preferentially 4 and 40 mg of nanoparticles per liter of growth medium at the end of the third sub-step.
During the fourth sub-step of the growth step, which lasts for 10⁻³ to 10³, preferentially for 2 to 120 hours, the insertion of the fed batch medium to the growth medium yields an iron concentration in the growth medium comprised between 10⁻¹⁰ and 10¹⁰ μM, preferentially between 150 and 500 μM, in some cases without taking into account the consumption of iron by the bacteria, in some other cases by taking into account the consumption of iron by the bacteria. This preferentially results in a production of nanoparticles that increases by a factor larger than 1.0000001, 1.1, 1.5, 2, 5, 10 or 10¹ from the beginning to the end of the fourth sub-step, or from a value comprised between 10⁻¹⁰ and 10¹⁰, preferentially from a value comprised between 4 and 40 mg of nanoparticles per liter of growth medium at the beginning of the fourth sub-step to a value comprised between 10⁻¹⁰ and 10¹⁰, preferentially between 8 and 80 mg of nanoparticles per liter of growth medium at the end of the fourth sub-step.
During the fifth sub-step of the growth step, which lasts for 10⁻³ to 10³, preferentially 2 to 120 hours, the insertion of the fed batch medium to the growth medium yields an iron concentration in the growth medium comprised between 10⁻¹⁰ to 10¹⁰ μM, preferentially between 500 and 1000 μM, in some cases without taking into account the consumption of iron by the bacteria, in some other cases by taking into account the consumption of iron by the bacteria. This preferentially results in a production of nanoparticles that increases by a factor larger than 1.0000001, 1.1, 1.5, 2, 5, 10 or 10¹ from the beginning to the end of the second sub-step, or from a value comprised between 10⁻¹⁰ and 10¹⁰, preferentially between 8 and 80 mg of nanoparticles per liter of growth medium at the beginning of the fifth sub-step to a value comprised between 10⁻¹⁰ and 10¹⁰, preferentially between 16 and 160 mg of nanoparticles per liter of growth medium at the end of the fifth sub-step.

In one embodiment of the invention, during the growth step or sub-step i of the growth step, preferentially sub-steps 1 to 5: i), the iron concentration of the growth medium is increased above 10⁻¹⁰ μM, preferentially 2 μM, preferentially by adding a fed-batch medium comprising iron to the growth medium and below 10¹⁰ mM, preferentially 5 mM, due to the consumption of iron by the cells, ii), the total quantity of iron per liter of growth medium brought to the growth medium is comprised between 10⁻⁶ and 15, preferentially between 2·10⁻⁴ and 1.5 g of iron per liter of growth medium, and/or iii) the quantity of nanoparticles increases from a value between 0 and 500 mg, preferentially between 0 and 80 mg of nanoparticles per liter of growth medium at the beginning of the growth step or one of its sub-step to a value comprised between 1 and 10⁵ mg, preferentially between 10 and 200 mg of nanoparticles per liter of growth medium at the end of the growth step or one of its sub-step.

In one embodiment of the invention, the grow step consists in amplifying the cells, were such amplification is associated with, or corresponds to: a speed or rate or number of cell division or an optical density, which is larger, preferentially by a factor of more than 1.000001, 1.0001, 1.01, 1.1, 1.2, 1.5, 2, 5, 10, 10², 10³, 10⁵, 10¹⁰, 10²⁰ or 10⁵⁰ during the growth step or at least one of its sub-step than during the pre-growth step or at least one of its sub-step.

In one embodiment of the invention, preferentially during, at the beginning, or at the end of the growth step or of at least one of its sub-steps, nanoparticle-producing cells have or are characterized by at least one of the following properties: i) a number of nanoparticles comprised in the cells that is larger than 1, 2, 5, 10, 50, 10² or 10³, preferentially larger than 0, 1 or 2, or between 0 and 10³, preferentially between 0 and 100 or between 0 and 10, ii) a percentage of cells with at least one nanoparticle that is larger than 10⁻⁴, 10⁻², 10⁻¹, 1, 5, 10, 50, 75 or 95, preferentially larger than 10 or 50% or comprised between 0 and 99%, 10 and 75, 5 and 90, preferentially between 20 and 100%, iii) an optical density larger than 10⁻⁵⁰, 10⁻²⁰, 10⁻¹⁰, 10⁻⁵, 10⁻³, 10⁻², 0.1, 0.2, 0.5, 1, 5, 10 or 100, iv) a number of cells larger than 1, 5, 10, 10², 10³, 10⁵, 10¹⁰, 10²⁰, 10⁵⁰ or 10¹⁰⁰, v) cells comprised in a volume larger than 0.0001, 0.001, 0.1, 1, 10, 50, 10², 10³, 10⁵ or 10¹⁰ liter(s), vi) a number of cell generation comprised between 1 and 10¹⁰, 1 and 10³, preferentially between 50 and 300, vii) a ratio between the optical density measured at the end of the growth step, OD_{GE}, and the beginning of the growth step, OD_{GB}, OD_{G}E/OD_{GB}, which is larger than 1, 2, 5, 10, 15, 25, 50, 100, 10³, 10⁵, 10¹⁰, 10⁵⁰ or 10¹⁰⁰, or viii) a ratio between the optical density measured at the end of sub-step i of the growth step, OD_{GiE}, and the beginning of sub-step i of the growth step, OD_{GiB}, OD_{GiE}/OD_{GiB}, which is larger than 1, 2, 5, 10, 15, 25, 50, 100, 10³, 10⁵, 10¹⁰, 10⁵⁰ or 10¹⁰⁰.

In one embodiment of the invention, the cells that produce essentially no nanoparticles are non-magnetic cells.

In one embodiment of the invention, at the beginning, preferentially during, at the beginning or at the end of the growth step or at least one of its sub-steps, a percentage of magnetic cells larger than 10⁻⁵⁰, 10⁻²⁰, 10⁻¹⁰, 10⁻⁵, 10⁻³, 10⁻¹, 1, 5, 10, 50 or 75%, is obtained. In some cases, the percentage of magnetic cells is equal to n_{MC}/(n_{MC}+n_{NMC}), where n_{MC} and n_{NMC} are the numbers of magnetic cells and non-magnetic cells, respectively.

In another embodiment of the invention, magnetic cells are cells that display a magnetic response.

In some cases, the pre-growth step, the growth step, or at least one of their sub-step, is carried out at a temperature larger than −250, −200, −150, −100, −50, −20, −10, −5, −2, −1, 0, 1, 2, 5, 10, 20, 50, 75, 100, 10³, 10⁵ or 107° C. or with a temperature variation larger than 10⁻⁵, 10⁻³, 10⁻², 0.1, 1, 5, 10, 50, 100 or 150° C. In some other cases, the pre-growth step, growth step, or at least one of their sub-steps, is carried out at a temperature lower than 10⁷, 10⁵, 10³, 100, 75, 50, 40, 30, 20, 10, 5, 2, 1, 0, −1, −2, −5, −10, −20, −50, −100, −150, −200 or −250° C. or with a temperature variation lower than 10⁵, 10³, 10², 50, 20, 10, 5, 2, 1 or 0.1° C.

In some cases, the pre-growth step, growth step, or at least one of their sub-steps, is carried out at a pH larger than 1, 2, 3, 4, 5, 6, 7, 8, 9, 10, 11, 12, 13 or 14. pH or with a pH variation larger than 10⁻¹⁰, 10⁻⁷, 10⁻⁵, 10⁻⁴, 10⁻³, 10⁻¹, 1, 2, 3, 4, 5, 6, 7, 8, 9, 10, 11, 12, 13 or 14 pH unit(s). In some other cases, the pre-growth step, the growth step, or at least one of their sub-steps, is carried out at a pH lower than 1, 2, 3, 4, 5, 6, 7, 8, 9, 10, 11, 12, 13 or 14 or with a pH variation lower than 10⁻¹⁰, 10⁻⁷, 10⁻⁵, 10⁻⁴, 10⁻³, 10⁻¹, 1, 2, 3, 4, 5, 6, 7, 8, 9, 10, 11, 12, 13 or 14 pH unit(s).

In some cases, the temperature, temperature variation, pH, or pH variation, is sufficiently large to enable at least 1, 5, 10, 10³, 10⁵, 10¹⁰ or 10⁵⁰ cell division(s) or cell division(s) per hour. In some other cases, the temperature, temperature variation, pH, or pH variation, is sufficiently low to prevent the destruction, disappearance, or denaturation of more than 1, 5, 10, 10³, 10⁵, 10¹⁰ or 10⁵⁰ cell(s) or cell(s) per hour.

The invention also relates to the method according to the invention, wherein the pre-growth medium does not comprise iron or at least one source of iron.

The invention relates to the method according to the invention, wherein the pre-growth medium comprises iron or at least one source of iron, wherein the nature and/or the quantity of iron or source of iron, preferentially essentially, does not allow the production of the nanoparticles by the cells while allowing cell growth.

In one embodiment of the invention, the nature of the source of iron is the composition, chemical formula, type of iron source, or is the iron source itself. In some cases, the iron source is a ferric or ferrous iron source. In some cases, it can be or comprise or be made of or have the chemical formula Cl₃Fe, C₁₀H₁₂FeN₂NaO₈, Fe₂O₁₂S₃, C₆H₈FeNO₇, C₆H₅FeO₇, FeH₁₈N₃O₁₈, C₃₀H₂₁FeN₃O₁₅⁻³, FeO₄P, C₆H₇FeO₈, Fe₂H₂O₁₃S₃, Fe₂H₁₂O₁₈S₃, C₁₀H₁₂FeN₂NaO₈, C₁₀H₁₃FeN₂O₈, FeH₂₈NO₂₀S₂, C₁₀H₁₅FeN₂NaO₈, C₁₀H₁₄FeN₂NaO₈⁺⁴, C₁₄H₂₁FeN₃O₁₀, C₁₈Fe₇N₁₈, Fe₄H₂O₂₂S₅, Fe₄O₂₁P₆, F₃Fe, C₆H₁₁FeNO₇⁺³, C₆H₁₁FeNO₇, C₁₈H₁₅FeO₉, C₁₂H₂₉Fe₅Na₂O₂₃, C₁₂H₂₂Fe₂O₁₄, C₁₈H₂₁FeO₆, C₁₈H₂₄FeO₆, C₆H₅FeO₇, C₁₀H₁₆FeN₃O₈, C₄H₁₀FeO₅, Cs₄H₁₀₅FeO₆, AsFeH₁₃O₉⁺, AsFeO₄, Fe⁺³, C₆H₁₂FeN₃O₁₂, C₆H₁₈As₃FeO₆, FeH₂O₅P, C₂₁H₂₁FeO₉S₃, C₆H₁₁FeNaO₇⁺³, C₁₄H₂₂FeN₃NaO₁₀, FeNaO₇P₂, C₃H₉As₃Fe₃O₉, C₁₈H₂₄Fe₄O₄₂P₆, C₆H₁₁FeO₁₀, C₉H₁₈FeN₃S₆, Cl₃FeO₁₂, C₆H₉FeNO₇⁺, Cr₃Fe₂O₁₂, C₆H₁₀FeNO₈, FeH₃O₃, C₁₅H₃₀FeN₃S₆, C₃₀H₂₇FeN₃O₁₅, C₃FeN₃S₃, C₆H₁₂FeKO₆⁺⁴, FeH₃O₃, FeN₃O₉, C₃H₃FeO₆, C₆H₈FeO₇, C₂₄H₄₅FeO₆, FeO₆P₃, Fe₂H₁₄O₁₉S₃, C₁₈H₃₃FeO₂₁, C₆H₉FeO₉, C₁₈H₂₇FeO₂₄, C₆FeN₆⁻³, C₁₀H₁₂FeN₂O₈⁻, C₂₂H₃₆N₄O₁₃, C₃FeN₃, C₆H₁₂FeN₃O₁₂, C₆H₉FeO₆, C₁₈H₂₇FeO₆, FeH₄O₆P, C₂₁H₁₅FeO₉, FeH₈O₈P, C₆H₆FeNO₆, C₄FeKO₈, C₁₂H₁₂Fe₂O₁₈, C₃₃H₃₅FeN₄O₄, Cl₃FeH₄O₂, C₂₄H₄₅FeO₆, C₁₀H₁₅FeN₂O₇, FeH₄NO₈S₂, C₃₂H₃₁FeN₄O₅, Fe₂H₆O₃, AlF₆Fe, C₄H₄FeNO₈, C₈₁H₈₄FeN₃O₃₃, Fe₂S₃, Cl₃FeH₁₄O₇, C₁₈H₆FeN₉O₂₁, Cl₃FeO₉, FeI₃, C₆H₁₄FeO₁₀, C₆H₁₀FeO₈, C₅₅H₈₀FeN₁₇O₂₁S₃, C₁₀H₁₆FeN₅O₁₃P₃, C₁₈H₃₄FeO₁₆⁺³, C₁₂H₁₂Fe₂O₁₅, C₆FeNa₃O₁₂, C₁₀H₁₂FeKN₂O₈, C₂₁H₂₄FeN₃O₉, C₆H₆Fe₂O₁₂, C₆Fe₂O₁₂, AsFe, C₃₅H₃₃FeN₅O₁₁⁻³, Cl₃FeH₂O, C₁₈H₃₀Fe₂N₆O₁₂, FeI₃O₉, C₁₀H₁₈FeN₂NaO₁₁, Cl₃FeH₁₈O₉, Cr₂FeH₄NO₈, C₉H₂₁Fe₂O₁₈P₃, C₁₈H₃₄FeO₂, C₃₀H₂₇FeO₆, C₃₀H₂₄FeN₃O₁₅, CS₄H₁₀₂FeO₆, Fe₄H₁₈O₃₀P₆, Fe₂Se₃, C₅₄H₉₉FeO₆, C₁₈H₂₁FeO₆, C₁₀H₁₈FeN₂O₇⁺², C₁₀H₁₈FeN₂O₇⁺², C₁₀H₁₉FeN₃O₈, C₂₂H₁₄FeO₄, C₃₉H₆₃FeN₆O₁₅⁺³, C₁₀H₁₉FeN₃O₈, C₄FeNaO₈, FeO₄V, C₆H₁₅FeN₃O₁₂, C₆Fe₂O₁₂, C₁₈H₂₄Fe₂O₂₄⁻⁶, C₁₈H₁₉FeN₂NaO₆, C₁₈H₁₉FeN₂NaO₆, C₁₂H₁₈Fe₂O₁₂, C₆FeK₃N₆, C₂₄H₄₇FeO₂₅⁻, C₁₈H₃₈FeO₁₉, C₁₅H₂₁FeO₆, C₁₈H₃₉FeO₂₄, C₆H₁₁FeNO₇, C₆H₁₂FeO₆, C₁₂H₂₈FeO₁₄, FeHO₂, C₄₅H₃₆FeN₃O₆, Fe₃H₂O₄, Fe₂O₃, C₃₆H₇₂FeO₆, C₁₂H₁₈Fe₂O₁₅, C₉H₁₈FeO₉, FeH₆O₃, C₅₄H₁₀₂FeO₆, C₄₂H₈₄FeO₆, C₁₆H₃₁FeO₂⁺², C₃₆H₆₉FeO₆, Fe₃H₈O₄, C₈H₁₅Fe₂O₂⁺⁴, C₁₂H₄₈Fe₂N₁₂O₁₂S₃, C₄₈H₉₆FeO₆, C₉H₁₅FeO₉, C₃₅H₃₉N₅O₁₁, C₄₂H₈₁FeO₆, C₄₈H₉₃FeO₆, C₁₀H₂₄O₂, Fe₂H₁₈O₂₁S₃, FeH₁₂N₃O₁₅, C₂₄H₂₃FeN₁₀O₆S₂, C₁₈H₁₄Cl₃FeN₁₀S₂, C₂₁H₁₅FeO₆, Fe₂H₁₀O₁₇S₃, C₁₀H₁₉FeN₃O₈, C₁₈H₂₀FeN₂NaO₆, C₃F₉FeO₉S₃, C₅H₁₄FeO₄, C₆H₁₉FeNO₁₁, C₁₈H₁₆FeN₂NaO₆, C₃₂H₃₆N₄O₉, C₁₅H₃₀FeO₆, C₁₅H₂₄FeO₆, C₁₅H₁₅F₉FeO₆, C₂₁H₂₁Cl₃FeIS, C₆H₁₂Fe₂O₁₈, C₆H₁₈FeO₁₂, C₆H₁₅FeO₁₂, C₆H₁₈Fe₂O₁₈, C₆H₈FeO₇, C₆H₁₃FeO₁₁, C₆H₄Fe₂N₇, FeH₂O₄S, C₄₂H₆₀N₁₂O₁₆, C₆Fe₂N₆, C₃Fe₂O₉, C₁₆₂H₂₉₇FeO₂₇⁻⁶, C₂₁H₂₇Cl₄FeN₂₀, C₆H₄FeNaO₇, C₂₇H₅₀FeN₆O₁₀, C₂₅H₄₈FeN₆O₈, C₂₇H₄₈FeN₆O₉, C₆H₇FeO₆⁺², Fe₂H₂O₄, C₁₄H₂₆FeN₅O₁₀, C₁₄FeH₄N, C₁₃FeH₁₂O₁₈, C₆H₁₇FeN₂NaO₇⁺³, C₁₀H₁₁FeNO₆, C₁₅H₁₅F₉FeO₆, C₆Fe₂N₆Na, C₉H₂₁Fe₂O₁₈P₃, C₂₁H₂₇ClN₂O, C₂H₃FeO, C₁₀H₁₂Fe₂N₂₀₈, FeH₃O₃P, C₇H₅FeO₂, C₇H₅FeO₂, FeI₃₀₁₂, C₃H₄FeNO₂S, C₂H₂FeNO₂, C₁₂H₁₂Fe₂O₁₂, C₈H₇FeNO₃, C₂HFe, C₆H₇FeO₂S₄, C₆H₁₁FeO₆, C₁₄H₁₉FeO₁₂, BFeH₃O₃, C₂₁H₁₈FeO₁₅, C₃₅H₅₆FeN₆O₁₃, C₁₂H₃₀FeO₃, CHFe, C₄₇H₄₈FeNO₁₄, Fe₂H₆O₃, Fe₂O₉Sn₃, C₁₈H₁₈FeO₃, Fe₂O₉Se₃, Fe₂O₉Si₃, Fe₂O₉S₃, Br₃FeO₉, FeN₃O₆, C₂₄H₅₄FeO₃, C₆₆H₁₂₉FeO₆, FeP, C₆H₁₈FeO₂₄P₆⁺³, C₃₃H₇₂FeO₃, C₄₀H₇₅FeO₄, C₂H₃FeS, C₃FeN₃, C₂₁H₃₉FeO₆, FeSi, C₃₀H₂₉FeN₃O₁₆, C₂₂H₃₆FeN₄O₁₃, C₃₀H₅₇FeO₆, C₆₀Hu₁₇FeO₆, C₁₈H₁₂FeN₃O₆, C₁₈H₃₁FeO₂⁺², FeS₂, C₆H₁₁FeN₄O₂, C₆H₅FeO₇, C₆H₅FeS, C₁₀H₁₃FeN₂O₁₀⁻, C₈H₁₃FeOS₂, C₂₇H₅₁FeO₆, C₂₄H₄₄FeO₂₅⁻, C₆H₁₅FeN₃O₆, C₆H₁₂FeO₉, C₁₃FeO₉S₃, CFeNS, Fe₄H₁₂O₁₂Si₃, C₃H₆FeO₁₂, C₄H₃FeO₄S₂, C₄H₄FeO₆, C₆H₃FeN₃O₆, C₅H₅FeO₂, C₁₀H₂₄FeN₄O₉, C₁₄H₁₉FeN₃NaO₁₀, C₁₀H₁₄FeN₂Na₂O₈, C₃₆H₄₄FeN₄, C₆FeNa₃O₁₂, Fe₂H₃OS₃, C₁₆H₂₇FeO₄, C₆H₈Fe₂O₁₃, C₆H₇FeO₃, C₄H₄FeO₆S₂, C₂H₅FeN₂, C₅H₇FeOS₂, C₁₈H₁₈FeNa₆O₂₁, C₃H₉FeO₉S₃, C₂₄H₅₄FeO₁₂P₃, C₃₆H₅₅FeN₆O₁₁, Fe₂H₂O₁₀Si₃, C₂H₄FeNO₂, C₄H₁₁FeN₂O₄, AsFeH₂O₅, C₁₂H₁₃FeO₁₃, C₃₆H₆₇FeO₆, C₁₂H₁₃FeO₁₃, C₃H₆FeN₃O₆, C₁₈H₁₅FeO₉S₃, C₃₆H₇₅FeO₁₂S₃, Fe₂H₄O₅, C₂₈H₂₄FeN₄⁺³, F₃Fe, C₃₀H₃₀FeO₆, BFe, C₂H₈N₂O₄, C₈H₅FeN₂O₅, Fe₂H₄O₁₁Se₃, C₆H₇FeO₆S₄, C₄H₁₀FeN₃, C₆H₁₂Fe₂O₁₅, C₁₅H₂₃FeO₅, C₈H₁₂FeNO₁₂, C₄₉H₅₆ClFeN₄O₆, FeH₄NO₈S₂, C₃₆H₇₅FeO₉S₃, B₃F₁₂Fe, FeP, Fe₂H₂₀O₂₂S₃, C₁₃FeH₁₂O₁₅, C₁₈H₉FeN₆, Fe₂H₁₂O₁₅Se₃, C₅₆H₅₁FeN₄, Fe₂H₈O₁₃Se₃, C₄₄H₂₇FeN₄, C₃₃H₃₀FeN₄O₆⁻², CrFeO₃, C₁₈H₁₂FeN₃O₁₅S₃, C₁₃FeH₁₈O₂₁, C₆H₅FeNa₃O₁₃, C₁₈H₁₄FeNi₃O₉S₂, C₁₅H₂₄FeO₆, C₂₄H₂₇FeO₉S₃, C₂₇H₅₄FeN₃S₆, C₁₃FeH₁₂₀₆, C₁₆H₃₆Pb, C₈H₁₈Fe₂O₁₂P₂, C₁₃FeH₂₄O₁₂, C₂₄H₃₀FeO₉S₃, C₂₁H₂₄FeO₉S₃, C₁₈H₁₅FeO₁₂S₃, Cl₃FeH₂₀O₁₀, C₂₈H₂₄FeN₆O₆⁺, C₆₆H₁₂₁Fe₂NaO₆₅, Cr₃FeH₃O₁₂, C₁₂H₂₈Fe₂O₁₄, C₃H₈FeNS₂Zn⁻, F₃FeH₆O₃, C₃₀H₅₁FeO₆, C₃₀H₄₈Fe₄N₆O₂₄, C₃₀H₁₈FeN₃O₆, C₂₀H₃₆FeO₄, C₆H₆FeK₃O₁₅, C₁₅H₆F₁₈FeO₆, C₁₀H₁₃FeN₂O₈, C₆FeN₆, C₁₅H₃F₁₈FeO₆, C₁₅H₁₂FeN₃O₃S₃, C₂₁H₂₃FeO₁₀S₃, FeH₂O⁺³, C₂₄H₄₄FeNaO₂₈, Cr₃FeO₆, Fe₂H₂O⁺⁶, C₆H₁₂FeN₉, FeH₅NO₄S, C₂K₂O₄, C₁₈H₁₃FeN₆, C₃₀H₂₇FeO₆, C₃₄H₃₈N₄O₄, C₁₃FeH₁₅O₁₈, C₆H₁₈FeO₆P₃S₆, C₆H₁₁C₁FeNO₁₀S₂, C₅H₄F₃FeO₂⁺², C₆H₆Cr₂O₁₂, C₄H₃CrKO₈, C₂MgO₄, C₁₂H₂₅FeO₁₄, C₂H₂MgO₄⁺², C₂CrO₄⁺, C₂HNaO₄, C₂HKO₄, C₆Cr₂O₁₂, C₂H₂FeO₄, C₂H₄MgO₆, C₆AlO₁₂³, C₆Al₂O₁₂, C₂Li₂O₄, C₂MgO₄, C₄₄H₃₀N₄O₁₂S₄, C₁₀H₁₉FeN₂NaO₁₀, C₅H₄CuFeN₆O₃, C₁₀H₁₄FeN₂NaO₉, C₃₀H₁₅FeN₃Na₃O₁₅S₃, C₂₇H₁₅FeN₁₂O₆, C₉H₁₈FeN₃S₆, C₃₀H₃₀FeN₃O₁₅⁺³, C₉H₁₈FeN₃S₆, C₆FeN₆, C₁₈Fe₇N₁₈, C₁₈H₁₈FeN₂NaO₆, C₃₀H₂₁FeN₁₂₀₆, C₄₄H₃₀FeN₄⁺³, C₁₄H₁₈FeK₂N₃₀₁₀, C₁₀H₁₆FeN₂NaO₈, C₃₃H₂₉FeNO₁₁⁺, C₂₅H₁₈FeN₄O₆S⁺, C₃₅H₂₄FeN₆O₂S⁺, C₃₂H₃₂C₁FeN₄O₆, C₃₀H₁₂F₉FeN₁₂O₆, C₃₀H₁₈C₁₃FeN₁₂O₉, C₆₀H₇₂FeN₉O₉⁺³, C₆₀H₆₆FeN₉O₉⁺³, C₁₅H₂₄FeO₆, C₂₂H₂₅C₁₂FeN₃O₉⁺, C₁₈H₂₃C₁₃FeN₃O₁₂, C₁₁H₂₄FeNO₁, C₄₉H₅₄FeN₄O₉⁺, C₄₂H₅₄C₁₈Fe₂N₄O₂, C₄₄H₂₆Cl₄FeN₄⁺³, C₃₄H₃₂FeN₄O₄⁺, C₄₄H₃₈FeN₈⁺⁷, C₉H₁₁Cl₂FeN₄O₂S, C₁₈H₃₂FeN₄O₈⁺³, C₃₄H₃₂ClFeN₄O₆, or C₁₉H₂₅FeN₄O₆. In some other cases, the iron source can be or comprise or be made of or have the chemical formula: Fe⁺², FeH₁₄O₁₁S, FeH₈N₂O₈S₂, FeO₄S, Cl₂Fe, FeS, C₄H₂FeO₄, C₁₂H₂₆FeO₁₆, C₄H₅FeNO₄, C₁₂H₁₀Fe₃O₁₄, C₁₆H₃₀FeO₄, FeH₂O₅S, C₁₀H₁₂FeN₂Na₂O₈, As₂Fe₃O₈, CFeO₃, C₆H₁₂FeO₆, FeH₁₂N₂O₁₂, C₁₂H₁₀Fe₃O₁₄, C₆H₅FeNaO₇, C₃₄H₃₂FeN₄O₄, C₁₂H₂₂FeO₁₄, C₁₂H₁₄FeO₁₂, C₆H₁₀FeO₆, C₄H₈FeN₂O₄, C₁₂H₂₈FeO₁₆, FeI₂, FeH₄N₂₀₆S₂, C₃₄H₃₂FeN₄₀₄⁻², C₃₄H₃₂FeN₄O₄, F₂Fe, C₆H₁₈FeO₉, C₆H₅FeO₇⁻, C₂FeO₄, C₄H₄FeO₄, Cl₂FeO₈, Fe₃O₈P₂, FeO, B₂F₈Fe, FeH₈O₈S, C₄H₆FeO₄, C₄H₄FeO₄, C₁₂H₁₀FeNa₄O₁₄, C₂₂H₁₄FeO₄, C₂H₄FeO₆, C₁₂H₂₄FeO₁₄, C₁₄H₂₀FeN₃O₁₀⁻, Cl₂FeH₈O₄, C₁₂H₈FeN₂O₄, C₄H₈FeO₄, C₅H₇FeNO₄, C₈H₁₂FeN₂O₈, C₁₂H₁₀Fe₃O₁₄, C₆H₁₆FeO₉, C₁₉H₁₉FeN₇O₁₀S, C₁₀H₁₆FeN₂O₈, C₁₂H₁₀Ca₂FeO₁₄, C₂H₆FeO₆, C₃₆H₇₀FeO₄, C₆H₆FeO₇, C₄H₂FeO₄, C₃₆H₂₁Cl₂FeN₉O₁₄, C₃₂H₆₂FeO₄, FeH₂O₂, C₄H₆FeO₆, C₆H₈CaFeO₇⁺⁴, C₄H₁₀Cl₂FeN₂O₄, C₃₆H₂₄Cl₂FeN₆O₈, C₆H₁₄FeO₇, C₁₂H₁₆FeO₁₂, BFe, C₃₂H₁₆FeN₈, C₁₂H₂₆FeO₁₅, C₁₂H₁₀Fe₃O₁₄, FeH₈I₂O₄, C₄H₁₀FeN₂O₈S, C₃₀H₂₄Cl₂FeN₆O₈, C₃₉H₃₀Cl₂FeN₆O₈, C₁₂H₁₄FeO₁₂, C₃₀H₂₄FeN₆⁺², C₄H₂FeO₄₂, C₄H₄FeO₄, C₁₀H₁₆FeO₄, C₃₆H₂₄FeN₆O₄S, C₂H₄FeO₆, C₂H₂FeO₆, C₈H₁₅Fe₂O₂⁺⁴, C₃₂H₁₆FeN₈, C₁₂H₁₆Fe₃O₁₄, C₁₂H₂₄FeO₁₄, C₂FeN₂S₂, C₁₂H₁₆FeN₆O₄, C₁₄H₂₀FeN₃O₁₀, C₁₂H₇FeN₃O₆S, C₂₀H₁₂FeN₄, C₁₂H₁₆Ca₂FeO₁₄, C₄₆H₅₄FeO₉, C₆H₅FeO₇, FeH₄O₆S, C₁₀H₁₅FeN₂NaO₇, C₁₀H₆FeN₄O₈, Fe₂P, C₄H₄FeO₆, C₁₄H₂₆FeO₁₆, Cl₂FeH₁₂O₁₄, C₄H₈Cl₂FeN₂O₄, C₆Fe₃N₆, C₄H₁₂As₂FeO₈, C₁₀H₁₆FeO₄, FeH₂₀N₂O₁₄S₂, C₁₆H₃₀FeO₄, C₄₀H₄₀FeN₈O₄⁺, Fe₂Na₈O₂₁P₆, C₁₄H₈FeO₁₀, C₁₄H₈FeO₄, C₁₂H₂₀FeO₄, C₈H₈FeS, C₅H₄FeO, C₂H₃FeNO₂, C₁₀H₁₄FeN₂O₈, C₆H₂FeN₃O₇⁺, C₂H₂Fe, C₁₀H₆FeN₂, C₆H₅FeN₃O₇, C₇₂H₁₂₄FeO₈⁻², FeH₂₂N₂O₁₅S₂, C₄₀H₇₈FeO₄, FeH₂N₂O₆⁺², C₄₄H₈₆FeO₄, C₁₀H₂₀FeN₂O₈S₂, C₂₀H₃₈FeO₄, C₃₆H₆₆FeO₄, C₂₄H₄₆FeO₄, C₂₉H₂₆FeP⁺, C₃₆H₆₄FeO₆, C₁₄H₂₆FeO₄, C₂₆H₂₈FeNP, C₂₈H₅₄FeO₄, C₃₆H₃₂FeN₄O₄, C₃₆H₃₆FeN₄O₈, C₆H₉FeNO₇⁺, C₅H₆FeO₂, C₄H₁₁BFeO₄, C₈H₁₉BFeO₄, C₄H₄FeO₄S₂, C₆H₆FeO₇, C₁₈H₃₄FeO₄, C₁₂H₂₀FeO₁₃, C₄H₄FeO₆, C₅H₇FeNO₃, Fe₃H₈O₄, C₂FeN₂S₂, FeH₂O₂, Fe₃H₂O₄, C₄₄H₂₈FeN₄, C₂H₆FeO₅, Fe₂H₆O₁₁S₂, C₃H₄FeN₂O₃, Fe₃H₂O₉P₂, C₆H₁₄Fe₃N₃₀₇⁻³, C₄H₁₀FeN₂O₆, C₁₂FeH₂O, FeO₄W, C₆H₅FeO₃P, C₆H₈FeO₇, FeTe, C₄H₂FeO₄, C₂₀H₂₀C₁₂FeN₈, C₁₄H₁₂FeO₆, C₃H₃FeO₇P, C₄H₇FeNO₄, FeO₃Si, C₁₂FeH₁₂₀₆, C₁₂FeH₂O₉, FeH₁₀O₉S, FeH₁₂₀₁₀S, C₈H₁₇FeO₃P, C₄H₁₄FeO₈, Fe₃H₁₆O₁₆P₂, F₆FeSi, C₇₂H₄₂FeN₆Na₆O₂₂S₇, FeH₄O₅S, C₃₉H₃₀FeN₆O₄S, C₄₀H₅₀O₄, C₄H₁₀FeN₂O₄, C₂H₄FeN₂O₄S, Br₂FeH₂O, C₉₈H₂₀₀FeN₁₀, C₃₆H₂₁FeN₉O₁₀S, C₁₀H₁₀Fe, C₂H₆FeN₂, F₆FeH₁₂O₆Si, C₄₈H₄₈FeN₆O₄S, FeO₄S, C₂H₁₀FeN₂O₈S₂, C₄₄H₂₇FeN₅O, C₃₀H₂₄FeN₆O₄S, C₆H₈O₆, C₆H₇NaO₆, FeH₄O₂⁺², FeH₂O⁺², C₃H₇FeNO₇S, C₃₀H₁₈FeN₃NaO₆, C₂H₁₈FeN₂O₁₂S₂, C₄H₄FeO₄, C₇H₇FeN₄O⁺, Br₂Fe, C₁₈H₂₂C₁₂FeN₂, C₃₂H₂₈FeN₆O₆S₂, C₁₂H₁₄MgO₁₂, C₂H₅FeNO₆S, C₄₅H₆₀FeN₂O₈, C₃₀H₂₂C₁₂FeN₂, C₃₈H₂₆FeN₈O₂S₂, C₃₀H₂₈FeN₂O₆, C₁₄H₁₂C₁₆FeO₄, C₁₂H₁₄Fe, C₃₆H₃₆C₁₂FeN₆O₈, C₁₇H₁₄FeN₄O₄S, C₂₄H₃₀FeN₄O₄, C₃₄H₃₂C₁FeN₄O₆, C₁₂H₁₂Fe, Fe₃H₁₄O₁₂P₂⁺⁶, C₃₂H₁₆FeN₈, FeS₂, C₁₆H₁₅FeNO₂⁺², C₂₉H₂₀FeO₆, C₂₃H₂₈FeO₂, C₁₁H₁₀FeO₂, C₁₃H₁₄FeO₂, C₁₂H₁₂FeO₂, C₄₆H₄₈FeN₄O₆⁺², C₄₇H₅₉FeN₁₃₀₈⁺², C₄₆H₅₉FeN₁₃O₈⁺², C₄₈H₆₂FeNi₂O₈S⁺², C₅₀H₆₅FeN₁₃O₈⁺², C₄₈H₆₃FeN₁₃O₈⁺², C₄₈H₆₂FeNi₂O₈S⁺², C₅₅H₇₆FeN₁₄O₉⁺², C₂₅H₁₉FeN₃, C₁₅H₁₇FeN₃OS⁺², C₂₂H₂₃FeN₃OS⁺², C₂₆H₂₈C₁FeN₃, C₂₈H₃₃C₁FeN₄, C₂₇H₃₁C₁FeN₄, C₂₉H₃₅C₁FeN₄, C₃₀H₃₇C₁FeN₄, C₂₈H₃₃C₁FeN₄, C₂₇H₃₀C₁FeN₃, C₂₆H₂₈C₁FeN₃, C₂₉H₃₅C₁FeN₄, C₂₇H₃₀C₁FeN₅O⁺², C₄₁H₃₈C₁FeN₅O₃₊₂, C₄₂H₄₁FeN₅O₃⁺², C₄₁H₃₈FFeN₅O₃⁺², C₄₂H₄₇FeN₅O₃⁺², C₄₃H₄₉FeN₅O₃⁺², C₄₂H₄₁FeN₅O₃⁺², C₄₂H₄₀ClFeN₅O₃⁺², C₄₂H₄₀ClFeN₅O₃⁺², C₄₂H₄₀FFeN₅O₃⁺², C₄₁H₄₅FeN₅O₃⁺², C₄₂H₄₇FeN₅O₃⁺², C₄₁H₃₉FeN₅O₃⁺², C₂₂H₂₅FeN₅O₅⁺², C₂₄H₂₃C₁FeN₄O₂⁺², C₂₄H₂₃FFeN₄O₂⁺², C₂₄H₂₄FeN₄O₂⁺², C₁₅H₂₁FeN₃S⁺², C₂₉H₃₄FeN₄O₂⁺², C₂₈H₃₁C₁FeN₄O₂⁺², C₂₈H₃₁FFeN₄O₂⁺², C₃₀H₃₅C₁FeN₄O₃⁺², C₃₀H₃₅FFeN₄O₃⁺², C₂₈H₃₂FeN₄O₂⁺², C₂₇H₃₀FeN₄O₂⁺², C₂₆H₂₇C₁FeN₄O₂⁺², C₃₀H₃₆FeN₄O₃⁺², C₂₈H₃₁C₁FeN₄O₃⁺², C₂₈H₃₁FFeN₄O₃⁺², C₂₈H₃₂FeN₄O₃⁺², C₂₇H₂₉C₁FeN₄O₃⁺², C₂₆H₂₇FFeN₄O₂⁺², C₂₆H₂₈FeN₄O₂⁺², C₂₆H₂₈FeN₄O₂⁺², C₂₇H₂₉FFeN₄O₃⁺², C₂₇H₃₀FeN₄O₃⁺², C₂₆H₂₇C₁FeN₄O₃⁺², C₂₆H₂₇FFeN₄O₃⁺², C₂₆H₂₈FeN₄O₃⁺², C₂₅H₂₅C₁FeN₄O₃⁺², C₂₅H₂₅FFeN₄O₃⁺², C₂₅H₂₆FeN₄O₃⁺², C₂₄H₂₃C₁FeN₄O₃⁺², C₂₄H₂₃FFeN₄O₃⁺², C₂₄H₂₄FeN₄O₃⁺², C₂₅H₂₅C₁FeN₄O₂⁺², C₂₅H₂₅FFeN₄O₂⁺², C₂₅H₂₆FeN₄O₂⁺², C₂₅H₂₆FeN₄O₂⁺², C₂₉H₃₂C₁FeN₇⁺², C₃₃H₃₂C₁FeN₇⁺², C₂₂H₂₇C₁FeN₃RuS⁺, C₁₈H₁₉C₁FeN₃RuS⁺, C₁₉H₁₉BFeO₃⁺², C₂₈H₂₅C₁FeN₄O⁺², C₃₁H₃₈FeN₄O₃, C₂₉H₃₄FeN₄O₃, C₃₁H₄₁FeN₃O, C₂₈H₃₂FeN₄O₃, C₂₆H₂₉FeN₃O₂, C₂₆H₃₀FeN₂O, C₃₁H₃₆FeN₄O₃, C₃₀H₃₅FeN₅O₄, C₂₉H₃₅FeN₅O₃, C₃₂H₄₁FeN₅O₃, C₃₅H₃₈FeN₄O₃, C₃₂H₄₀FeN₄O₃, C₁₉H₁₃BBr₂F₂FeO₂, C₁₉H₁₄BClF₂FeO₂, C₁₉H₁₄BBrF₂FeO₂, C₁₉H₁₅BF₂FeO₂, C₂₁H₂₀FeO₄, C₂₀H₁₈FeO₃, C₂₀H₁₈FeO₃, C₂₀H₁₈FeO₃, C₁₉H₁₄F₂FeO₂, C₁₉H₁₄Br₂FeO₂, C₁₉H₁₅BrFeO₂, C₁₄H₁₂FeO₃, C₂₁H₁₉BF₂FeO₄, C₂₀H₁₇BF₂FeO₃, C₂₀H₁₇BF₂FeO₃, C₂₀H₁₇BF₂FeO₃, C₁₉H₁₃BF₄FeO₂, C₁₉H₁₃BC₁₂F₂FeO₂, C₂₁H₂₉AuCl₂FeN₄S⁺, C₃₀H₂₄Cl₂FeN₆⁺², C₂₂H₂₁Cl₂FeN₃⁺², C₂₃H₂₂FeN₆⁺², C₂₁H₁₉FeN₇⁺², C₂₃H₂₄FeN₆O⁺², C₄₇H₆₄FeN₁₄O₉, C₄₆H₆₀FeN₁₂₀₁₀, C₄₁H₅₃FeN₁₁₀₇, C₄₇H₆₅FeNi₅O₈, C₄₅H₅₉FeN₁₃O₉, C₄₂H₅₄FeN₁₂₀₇, C₄₃H₆₇FeNi₅O₈, C₄₈H₆₅FeN₁₃O₈, C₄₇H₆₄FeN₁₂₀₈, C₅₄H₇₇FeN₁₇O₉, C₅₁H₇₁FeN₁₅O₁₀, C₁₉H₁₆FeO₂, C₄₄H₄₈FeN₉O₁₇P₃, C₁₃H₉Cl₂FeN₃O₆S, C₁₉H₁₅FeNO₃, C₂₀H₁₈FeO₂, C₂₀H₁₈FeO₃, C₂₁H₂₀FeO₃, C₁₇H₂₀FeN₂O₂, C₁₈H₁₅FeNO, C₁₇H₁₄FeOS, C₁₇H₁₄FeOS, C₁₇H₁₄FeO₂, C₂₂H₂₂FeO₄, C₂₀H₁₈FeO₂, C₂₀H₁₈FeO₂, C₁₉H₁₄Cl₂FeO, C₂₁H₂₀FeO₃, C₄₈H₂₈FeN₄O₈, C₁₇H₁₅FeNS, C₃₄H₃₀FeN₄O₄⁻², C₃₀H₂₆Br₂FeN₄O₄, C₁₀H₁₈FeN₂O₇⁺², C₁₄H₁₂FeO₄, C₄₄H₂ClFeN₄, C₆₄H₆₄FeN₈O₁₂S₄, C₅₆H₅₆FeN₈O₈S₄, C₅₆H₄₄Br₈FeN₄, C₅₆H₅₂FeN₄, C₅₂H₄₀FeN₈O₁₂S₄, C₄₄H₃₂FeN₈O₈S₄, or C₄₄H₂₈FeN₄. In some other cases, the source of iron has the chemical formula CₐH_{b}Fe_{c}O_{d}NₑS_{f}Br_{g}ClₕPᵢNaⱼAsₖKₗAlₘCrₙVₒIₚB_{q}FᵣTeₛWₜ, where a, b, c, d, e, f, g, h, I, j, k, l, m, n, o, p, q, r, s, t are coefficients that can be equal to 0, 1, 2, 3, 4, 5, 6, 7, 8, 9, 10, 11, 12, 13, 14, 15, 16, 17, 18, 19, 20, or any other integer between 21 and 1000000000000, where the C, H, Fe, O, N, S, Br, Cl, P, Na, As, K, Al, Cᵣ, V, I, B, F, Te, and W atoms preferentially occupy the first, second, third, fourth, fifth, sixth, seventh, eighth, ninth, tenth, eleventh, twelve, thirteen's, fourteenth, fifteenth, sixteen's, seventeenth, eighteens, nineteenth, twentieth position, respectively. In some cases, the at least one atom of the chemical formula can occupy any one of the 20 positions of the formula. In some other cases, the source of iron can comprise the chemical functional groups selected in the group consisting of: Hydrocarbons, Alkane (R(CH₂)nH), Alkene (R₂C═CR₂), Alkyne (RC≡CR′), Benzene derivative (RC₆H₅); Groups containing halogen, Haloalkane (RX), Groups containing oxygen, Alcohol (ROH), Carbonyl (RCOR′), Aldehyde (RCHO), Acyl halide (RCOX), Carbonate (ROCOOR′), Carboxylate (RCOO—), Carboxylic acid (RCOOH), Ester (RCOOR′), Methoxy (ROCH₃), Hydroperoxide (ROOH), Peroxide (ROOR′), Ether (ROR′), Hemiacetal (RCH(OR′)(OH)), Hemiketal (RC(OR″)(OH)R′), Acetal (RCH(OR′)(OR″)), Ketal (RC(OR″)(OR′″)R′), Orthoester (RC(OR′)(OR″)(OR′″)), Heterocycle (PhOCOPh), Orthocarbonate ester (C(OR)(OR′)(OR″)(OR″)), Groups containing nitrogen, Amide (RCONR₂), Amines (RNH₂, R₂NH, R₃N, R₄N⁺), Imine (RC(═NH)R′, RC(═NR″)R′, RC(═NH)H, RC(═NR′)H, Imide ((RCO)₂NR′), Azide (RN3), Azo compound (RN₂R′), Cyanates (ROCN, RNCO), Nitrate (RONO₂), Nitrile (RCN, RNC), Nitrite (RONO), Nitro compound (RNO₂), Nitroso compound (RNO), Oxime (RCH═NOH), Pyridine derivative (RC₅H₄N), Groups containing sulfur, Thiol (RSH), Sulfide (RSR′), Disulfide (RSSR′), Sulfoxide (RSOR′), Sulfone (RSO₂R′), Sulfinic acid (RSO₂H), Sulfonic acid (RSO₃H), Thiocyanate (RSCN, RNCS), Thioketone (RCSR′), Thial (RCSH), Groups containing phosphorus, Phosphine (R₃P), Phosphonic acid (RP(═O)(OH)₂), Phosphate (ROP(═O)(OH)₂), Phosphodiester (HOPO(OR)₂), Groups containing boron, Boronic acid (RB(OH)₂), Boronic ester (RB(OR)₂), Borinic acid (R₂BOH), Borinic ester (R₂BOR), and a combination of several of these groups. In some other cases, the iron source can be an iron chelating agent.

In some cases, the quantity of the iron source is the quantity or concentration of the iron source or of iron, preferentially originating from the iron source, preferentially in the pre-growth and/or growth medium/media.

The invention also relates to the method according to the invention, wherein iron or the source of iron in the pre-growth medium consists of or comprises Fe²⁺ and/or Fe³⁺.

In some cases, a source of iron comprises Fe²⁺ when it comprises Fe₂ in its chemical formula.

In some other cases, a source of iron comprises Fe³⁺ when it comprises Fe₃ in its chemical formula.

The invention relates to the method according to the invention, wherein the concentration of iron or of the iron source in the pre-growth medium is lower than 20 μM. In some cases, the concentration of iron or of the iron source in the pre-growth medium is lower than 10¹⁰⁰, 10⁵⁰, 10²⁰, 10¹⁰, 10⁵, 10³, 10², or 20 μM. In some other cases, the concentration of iron or of the iron source in the pre-growth medium is larger than 0, 10⁻⁵⁰, 10⁻²⁰, 10⁻⁵, 10⁻¹, 1, 5, 10 or 20 μM. In still some other cases, the concentration of iron or of the iron source in the pre-growth medium is between 10⁻⁵⁰ and 10⁵⁰, 10⁻¹⁰ and 10¹⁰, 10⁻¹⁰ and 10⁵, 10⁻¹⁰ and 10³, or between 10⁻¹⁰ and 1 μM.

The invention relates to the method according to the invention, wherein the growth medium comprises iron or at least one source of iron, wherein the nature and/or quantity of iron or source of iron allows the production of the nanoparticles by the nanoparticle-producing cells and/or cell growth.

The invention relates to the method according to the invention, wherein the source of iron of the growth medium is the same as the source of iron of the pre-growth medium.

The invention relates to the method according to the invention, wherein the concentration of iron or source of iron in the growth medium is larger or equal than the concentration of iron or source of iron in the pre-growth medium.

In some cases, the concentration of iron or of the iron source in the growth medium is lower than 10¹⁰⁰, 10⁵⁰, 10²⁰, 10¹⁰, 10⁵, 10³, 10², or 20 μM. In some other cases, the concentration of iron or of the iron source in the growth medium is larger than 0, 10⁻⁵⁰, 10⁻²⁰, 10⁻⁵, 10⁻¹, 1, 5, 10 or 20 μM. In still some other cases, the concentration of iron or of the iron source in the growth medium is between 10⁻⁵⁰ and 10⁵⁰, 10⁻¹⁰ and 10¹⁰, 10⁻¹⁰ and 10⁵, 10⁻¹⁰ and 10³, or between 10⁻¹⁰ and 1 μM.

The invention relates to the method according to the invention, wherein the growth medium is supplemented by a fed-batch medium.

In one embodiment of the invention, the fed-batch medium comprises at least one source in common with the pre-growth and/or growth medium/media. In some cases, the concentration of this source is equal or at least 1.00001, 1.1, 2, 5, 10, 10³ or 10⁵ larger in the fed-batch medium than in the pre-growth and/or growth medium/media. In some other cases, the concentration of this source is at least 10⁵, 10³, 10, 1, 1.1 or 1.00000001 lower in the fed-batch medium than in the pre-growth and/or growth medium/media.

The invention relates to the method according to the invention, wherein the fed-batch medium comprises iron or a source of iron with at a concentration larger than 10⁻⁵⁰, 10⁻²⁰, 10⁻¹⁰, 10⁻⁵, 10⁻¹, 1, 5, 10, 20, 50, 100, 10³ or 10⁵ μM. In some other cases, the fed-batch medium comprises iron or a source of iron at a concentration lower than 10⁵⁰, 10²⁰, 10¹⁰, 10⁵, 10², 10⁻², 10⁻¹⁰ or 10⁻²⁰ μM. In still some other cases, the fed-batch medium comprises iron or a source of iron at a concentration comprised between 10⁻⁵⁰ and 10⁵⁰, 10⁻¹⁵ and 10¹⁵, 10⁻¹⁰ and 10⁵, 10⁻⁵ and 10⁵, between 10⁻³ and 10³ μM, or between 0.5 nM and 50 M, preferentially before adding the fed-batch medium to the growth medium.

In one embodiment of the invention, the fed-batch medium is acidic or has a pH lower than 7, 6, 5, 4 or 3, preferentially lower than 2. In some cases, the fed-batch medium has a pH larger than 0 or 1.

The invention relates to the method according to the invention, wherein the fed-batch medium is introduced in the growth medium at a rate comprised between 10⁻¹⁵ liter per hour and 10¹⁵ liter(s) per hour or between 10⁻¹⁵ μM of iron per hour and 10¹⁵ μM of iron per hour. In some cases, the fed-batch is added to the growth medium at a low rate, preferentially at a rate that is lower than 10¹⁰⁰, 10⁵⁰, 10²⁰, 10¹⁰, 10⁵, 10², 10, 5, 1, 10⁻², 10⁻³, 10⁻⁵ or 10⁻¹⁰ liter(s) of fed-batch medium per minute or μM of iron per minute, preferentially when the number of cell division in the growth medium is low, preferentially lower than 10²⁰, 10¹⁰, 10⁵, 10³, 10, 1 cell division(s) per second or hour or day or month. In some other cases, the fed-batch medium is added to the growth medium a high rate, preferentially at a rate larger than 10⁻¹⁰⁰, 10⁻⁵⁰, 10⁻²⁰, 10⁻¹⁰, 10⁻⁵, 10⁻², 1, 5, 10, 10², 10³, 10⁵ or 10¹⁰ liter(s) of fed-batch medium per minute or μM of iron per minute, preferentially when the number of cell division in the growth medium is large, preferentially larger than 1, 2, 5, 10, 10³, 10⁵, 10¹⁰ or 10²⁰ cell division(s) per second or hour or day or month In some cases, between two sub-steps of the growth step, the debit of the fed-batch medium decreases, preferentially by a factor of more than 1.0000001, 1.00001, 1.0001, 1.01, 1.1, 1.2, 1.5, 2, 5, 10, 10³, 10⁵, 10⁷ or 10⁹. In some other cases, between two sub-steps of the growth step, the debit of the fed-batch medium increases, preferentially by a factor of more than 1.0000001, 1.00001, 1.0001, 1.01, 1.1, 1.2, 1.5, 2, 5, 10, 10³, 10⁵, 10⁷ or 10⁹.

In one embodiment of the invention, before being added to the growth medium, the fed-batch medium has an iron concentration that is larger by a factor of at least 1.000001, 1.001, 1, 1.5, 2, 5, 10, 10², 10³ or 10⁵ than the iron concentration of the growth medium.

In another embodiment of the invention, preferentially after being added to the growth medium, the fed-batch medium becomes part of the growth medium.

The invention relates to the method according to the invention, wherein the pre-growth and/or growth medium/media according to the invention comprise(s) only one vitamin selected from the group consisting of Biotin, Calcium pantothenate, Folic acid, Inositol, Nicotinic acid, p-Aminobenzoic acid, Pyridoxine HCl, Riboflavin, Thiamine HCL, and any derivative of these vitamins.

In some cases, the pre-growth and/or growth medium/media can comprise less than 10⁵⁰, 10²⁰, 10¹⁰, 10⁵, 10³, 100, 75, 50, 25, 10, 5, 3, 2 or 1 vitamin(s) or different vitamins. In some cases, different vitamins can be vitamins that comprise at least 1, 2, 5, 10, 10², 10³, 10⁵, 10¹⁰, 10²⁰, or 10⁵⁰ different chemical element(s). In some other cases, the pre-growth and/or growth medium/media comprise(s) more than 1, 2, 5, 10, 10², 10³, 10⁵ or 10¹⁰ vitamin(s) or different vitamins.

In some cases, the vitamin(s) can be water soluble vitamin(s). In some other cases, the vitamin(s) can be fat soluble vitamin(s). In still some other cases, the vitamin(s) can be belong to A, D, E, K, B₁, B₂, B₃, B₅, B₆, B₇, B₉, B₁₂, or C vitamin(s). In still some other cases, the vitamin is not produced by the nanoparticle-producing cells. In still some other cases, the vitamin is a vitamin used to treat a disease such as a disease caused by or associated with vitamin deficiency.

In still another embodiment of the invention, the vitamin(s) is/are selected from the group consisting of: Adenosylcobalamin, Aminobenzoic acid, Ascorbic acid, Biotin, Calcium D-(+)-pantothenate, Carotene Thiamine, Carotenoids beta, Cholecalciferol (D3), Cyanocobalamin, Cyanacobalamine, Ergocalciferol (D₂), Folates, Folic acid, Folinic acid, hydroxocobalamin, Inositol, Menaquinones (K₂), Methylcobalamin, Niacin, Niacinalide, Niacinamide, Nicotinamide riboside, Nicotinic acid, Pantothenic acid, Phylloquinone (K1), Pyridoxal, Pyridoxamine, Pyridoxine, p-Thioctic acid, Pyridoxal, Pyridoxamine, Pyridoxine, Pyridoxine hydrochloride, Retinal, Retinoic acid, Retinol, Riboflavin, Thiamine, Timaine, Tocopherol, or Tocotrienols, and a derivative or combination of one or several of this/these vitamin(s).

In still another embodiment of the invention, the at least one vitamin comprised in the growth medium is biotin, folic acid, riboflavin, nicotinic acid or thiamin HCl.

In some cases, the vitamin(s) comprised in the pre-growth medium is/are the same as the vitamin(s) comprised in the growth medium. In some other cases, the vitamin(s) comprised in the pre-growth medium is/are different from the vitamin(s) comprised in the growth medium.

The invention also relates to the method according to the invention, wherein the concentration of at least one vitamin comprised in the pre-growth and/or growth medium/media is lower than 10¹⁰⁰, 10⁵⁰, 10²⁰, 10¹⁰, 10⁵, 10³, 10, 1, 10⁻¹, 10⁻³, 10⁻⁴, 10⁻⁶, 10⁻⁹, 10⁻²⁰, 10⁻⁵⁰ or 10⁻¹⁰⁰ M, or preferentially lower than 0.002 mol/L.

In another embodiment of the invention, the pre-growth and/or growth medium/media comprise(s) at least one vitamin or one chemical element comprised in at least one vitamin at a concentration that is larger than 10⁻¹⁰⁰, 10⁻⁵⁰, 10⁻²⁰, 10⁻¹⁰, 10⁻⁵, 10⁻⁴, 10⁻³, or 10⁻¹, 1, 10, 10², 10³, 10⁵ or 10¹⁰M.

The invention also relates to the method according to the invention, wherein the pre-growth and/or growth medium/media comprise at least one vitamin at a concentration, which is lower, preferentially by a factor of more than 1.0001, 1.2, 1.5, 2, 5, 10, 10³ or 10⁵, than the concentration of the source of carbon, nitrogen, sulfur, sulfate, phosphorous, phosphate, calcium, potassium, magnesium, oxygen, hydrogen, and/or iron. In some cases, the cells(s) do not require a large concentration of vitamins to grow, divide, and/or to synthetize nanoparticle(s).

The invention also relates to the method according to the invention, wherein the pre-growth and/or growth medium/media comprise(s), per gram or mL of growth or pre-growth medium, less than: i) 1 mg of yeast extract, ii) 1 mg of at least one component of yeast extract, iii) 1 mg of peptone, iv) 1 mg of at least one component of peptone, v) 1 mg of CMR agent, vi) 1 mg of at least one chelating agent, vii) 1 mg of at least one amino acid, viii) 1 mg of a toxic or cytotoxic compound, and/or ix) 1 mg of at least one heavy metal.

In one embodiment of the invention, the pre-growth and/or growth medium/media comprise(es) a concentration in yeast extract, peptone, CMR agent, chelating agent, amino acid, toxic or cytotoxic compound, and/or heavy metal, which is/are lower than 10¹⁰⁰, 10⁵⁰, 10²⁰, 10¹⁰, 10⁵, 10², 10, 1, 10⁻³ or 10⁻⁵ μg of yeast extract, peptone, CMR agent, chelating agent, amino acid, and/or heavy metal per liter or milliliter of pre-growth and/or growth medium/media. In some cases, this situation can occur when yeast extract, peptone, CMR agent, chelating agent, amino acid, toxic or cytotoxic compound, and/or heavy metal, has/have been removed from or are not comprised in the pre-growth and/or growth medium/media.

In one embodiment of the invention, the first and/or second medium/media comprise(s) a concentration in yeast extract, peptone, amino acid(s), and/or heavy metal(s), which is/are larger than 10⁻¹⁰⁰, 10⁻⁵⁰, 10⁻²⁰, 10⁻¹⁰, 10⁻⁵, 10⁻², 10⁻¹, 1, 10, 10³ or 10⁵ μg of yeast extract, peptone, amino acid(s), and/or heavy metal(s) per liter or milliliter of pre-growth and/or growth medium/media. In some cases, this situation can occur when yeast extract, peptone, CMR agent, chelating agent, amino acid, and/or heavy metal, has/have been added, preferentially unwillingly, to the pre-growth and/or growth medium/media.

In one embodiment of the invention, the yeast extract is or comprise peptides, amino acids, purine bases, pyrimidine bases, and/or hydro-soluble vitamins of B group.

In one embodiment of the invention, the amino acid(s) is/are alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and/or valine.

In one embodiment of the invention, the heavy metal(s) is/are arsenic (As), cadmium (Cd), chrome (Cr), copper (Cu), mercury (Hg), nickel (Ni), lead (Pb), selenium (Se), and/or zinc (Zn).

In one embodiment of the invention, the agent(s) that is/are carcinogenic, mutagenic, or toxic for reproduction, also designated as CMR agent(s), is/are preferentially Nitriloacetic acid, Trisodium salt, and/or Boric acid.

In one embodiment of the invention, the toxic or cytoxic compound is a compound that produces the death of a cell or organism, preferentially of the nanoparticle-producing cells, preferentially when it is introduced in the pre-growth and/or growth medium/media, preferentially at a concentration larger than 10⁻¹⁰, 10⁻⁵, 10⁻², 1, 5, 10, 10³, or 10 μM, preferentially at a concentration comprised between 10⁻¹⁰ and 10¹⁰ μM.

In one embodiment of the invention, the pre-growth and/or growth medium/media does/do not comprise the minerals selected in the group consisting of: selected in the group consisting of: C₆H₆NO₆Na₃, Nitriloacetic acid trisodium salt, MnO₄S H₂O, Manganese (II) sulfate monohydrate, NaCl, sodium chloride, CoN₂O₆ 6H₂O, Cobalt (II) nitrate hexahydrate, O₄SZn 7 H₂O, Zinc sulfate heptahydrate, CuO₄S 5H₂O, Copper(II) sulfate pentahydrate, AlKO₈S₂ 12H₂O, Aluminium potassium sulfate dodecahydrate, H₃BO₃, Boric acid, Na₂MoO₄ 2H₂O, Sodium molybdate dihydrate, Cl₂Ni 6H₂O, Nickel(II) chloride hexahydrate, Na₂SeO₃, Sodium selenite, and a derivative or combination one or several of these compounds.

In one embodiment of the invention, the pre-growth and/or growth medium/media comprise(s) a concentration in mineral(s), which is lower than 10¹⁰⁰, 10⁵⁰, 10²⁰, 10¹⁰, 10⁵, 10², 10, 1, 10⁻¹, 10⁻³ or 10⁻⁵ μg of mineral(s) per liter or milliliter of growth medium. In some cases, this situation can occur when the minerals have been removed from the pre-growth and/or growth medium/media.

In one embodiment of the invention, the first (pre-growth) and/or second growth medium/media comprise(s) a concentration in mineral(s), which is larger than 10⁻¹⁰⁰, 10⁻⁵⁰, 10⁻²⁰, 10⁻¹⁰, 10⁻⁵, 10⁻², 10⁻¹, 1, 10, 10³ or 10⁵ μg of mineral(s) per liter or milliliter of growth medium. In some cases, this situation can occur when the minerals have been removed, preferentially unwillingly, to the pre-growth and/or growth medium/media.

The invention also relates to the method accord to the invention, which comprises an additional step of purifying high purity iron oxide nanoparticle(s) by removing at least one impurity(ies) from the nanoparticle(s).

In one embodiment of the invention, the additional step of purifying high purity iron oxide nanoparticles consists in removing impurity(ies) from nanoparticle(s) and/or denaturing and/or destroying impurity(ies) comprised in the nanoparticles.

In one embodiment of the invention, the additional step of purifying the nanoparticles is preceded by a preceding step of isolating or extracting the nanoparticles from the cells. In some cases, the preceding step is a step of recovering the nanoparticles. In some cases, the preceding step is carried out by: i) mixing the cells, preferentially obtained from the growth step, with a detergent such as KOH or NaOH, ii) heating the cells at a temperature larger than −270, −250, −200, −150, −100, −50, −30, −10, −5, 0, 5, 10, 20, 30, 50, 75, 100, 150, 200, 500, 10³, 10⁵ or 10¹⁰° C. or comprised between −270 and 10¹⁰, −100 and 10⁵, or between 0 and 100° C., iii) inducing a temperature gradient larger than 10⁻⁵⁰, 10⁻²⁰, 10⁻¹⁰, 10⁻⁵, 10⁻³, 10⁻¹, 1, 2, 5, 10, 10³, 10⁵ or 10¹⁰° C. per hour, minute or second, or comprised between 10⁻⁵⁰ and 10¹⁰° C. per hour, minute or second, iii) applying a pressure on the cells, preferentially larger than 1, 10, 100, 500, 10³, 10⁴, 10⁵, 10⁶, 10⁷, 10⁸ or 10⁹ atmosphere(s), or comprised between 1 and 10⁹ atmosphere(s), using for example a French press, and/or iv) sonicating the cells, preferentially at a power larger than 10⁻⁵⁰, 10⁻²⁰, 10⁻⁵, 10⁻¹, 1, 10, 10², 10³, 10⁵ or 10¹⁰ W.

In another embodiment of the invention, the additional step of purifying the nanoparticles enables removing: i) a percentage in mass of impurity(ies) that is larger than 10⁻²⁰, 10⁻¹⁰, 10⁻⁵, 10⁻², 10⁻¹, 1, 5, 10, 25, 50, 75, 80 or 90%, or ii) more than 10⁻⁵⁰, 10⁻²⁰, 10⁻¹⁰, 10⁻⁵, 10⁻¹, 1, 5, 10, 10³, 10⁵ or 10¹⁰ μg of impurity(ies) per gram of nanoparticle(s).

In one embodiment of the invention, impurity(ies), preferentially removed by the additional step of purifying the nanoparticles, is/are carbon or carbonaceous material and/or is/are not iron oxide. Preferentially, this/these impurity(ies) are located in the coating of the nanoparticles.

In one embodiment of the invention, the impuritity(ies) is/are removed from the core and/or coating of the nanoparticles, preferentially from the coating of the nanoparticles. Preferentially, the impuritity(ies) that is/are removed is/are shallow impurity(ies). In some other cases, the impurity(ies) that is/are removed is/are deep impurity(ies).

The invention also relates to the additional step of the method of purifying the high purity iron oxide nanoparticles by removing at least one impurity(ies) from the nanoparticle(s), comprising at least one heating steps in which the temperature of the nanoparticles is increased to a temperature T₀, and is then maintained at T₀ during a heating time that is preferentially comprised between 1 second and 1 minute, 1 second and 1 hour, 1 second and 12 hours, 1 second and 1 day, 1 second and 1 week, 1 second and 1 month, or between 1 second and 1 year, where T₀ is preferentially comprised between −200 and 10⁵, −100 and 10⁵, −50 and 10⁵, −10 and 10⁵, 0 and 105° C., 10 and 10⁵, 20 and 10⁵, 30 and 10⁵, 100 and 10⁵, 200 and 105° C., 100 and 10⁴, 100 and 10³, or between 100 and 500° C.

The invention also relates to the additional step of the method of purifying the high purity iron oxide nanoparticles by removing at least one impurity(ies) from the nanoparticle(s), comprising at least two heating steps in which:
During step 1, the temperature of the nanoparticles is increased to a temperature T₁, and is then maintained at T₁ during a heating time that is comprised between 1 second and 20 years, where T₁ is comprised between 150° C. and 250° C.
During step 2, the temperature of the nanoparticles is increased to a temperature T₂, and is then maintained at T₂ during a heating time that is comprised between 1 second and 20 years, where T₂ is comprised between 350° C. and 450° C.

In some cases, the additional step of the method of purifying the high purity iron oxide nanoparticles or the heating step can be designated as purifying method.

The invention relates to the method for removing at least one impurity from high purity iron oxide nanoparticles, comprising an additional step between steps 1 and 2, in which the temperature of the nanoparticles is increased to a temperature T₃ and is then maintained at T₃ during a heating time that is comprised between 1 second and 20 years, where T₃ is comprised between 250° C. and 350° C.

In some cases, the temperature of the nanoparticles is maintained at the temperature T₁, T₂, and/or T₃, during a heating time that is smaller than 100 years, 50 years, 20 years, 10 years, 5 years, 2 years, 1 year, 11 months, 6 months, 3 months, 2 month, 1 month, 3 weeks, 2 weeks, 1 week, 6 days, 5 days, 3 days, 1 day, 23 hours, 12 hours, 6 hours, 1 hour, 50 minutes, 30 minutes, 20 minutes, 10 minutes, 5 minutes, 2 minutes, 1 minute, 50 seconds, 30 seconds, 10 seconds, 1 seconds, 1 millisecond or 1 microsecond. In some other cases, the temperature of the nanoparticles is maintained at the temperature T₁, T₂, and/or T₃, during a heating time that is larger than 1 microsecond, 1 millisecond, 1 second, 10 seconds, 30 seconds, 50 seconds, 1 minute, 2 minutes, 5 minutes, 10 minutes, 1 hour, 6 hours, 12 hours, 23 hours, 1 day, 3 days, 5 days, 6 days, 1 week, 2 weeks, 3 weeks, 1 month, 2 months, 3 months, 6 months, 11 months, 1 year, 2 years, 5 years, 10 years, 20 years, 50 years or 100 years. In still some other cases, the temperature of the nanoparticles is maintained at temperature T₁, T₂, and/or T₃, during a heating time that is comprised between 1 microsecond and 100 years, 1 second and 20 years, 1 second and 1 year, 1 second and 1 month, 1 second and 1 day, 1 minute and 1 day, 5 minutes and 1 day, 10 minutes and 12 hours, 30 minutes and 6 hours, or between 30 minutes and 3 hours.

In an embodiment of the invention, the heating time is larger than the time during which the temperature is increased to T₁, T₂, and/or T₃, preferentially by a factor of more than 1.001, 1.1, 1.5, 2, 5, 10, 10³, 10⁵ or 10¹⁰.

In some cases, T₁ is comprised between −273° C. and 250° C., −200° C. and 250° C., −100 and 250° C., 0 and 250° C., 50 and 250° C., 150 and 250° C., or between 180 and 220° C. In some other cases, T₂ is comprised between 200 and 10⁵, 250 and 10⁵, 300 and 10⁵, 350 and 10⁵, 350 and 10³, 350 and 500, 350 and 450, or between 360 and 400° C. In still some cases, T₃ is comprised between −273 and 10⁵, −200 and 10³, −100 and 500, −50 and 200, 0 and 500, 100 and 500, 200 and 500, 200 and 400, or between 250 and 350° C.

In still another embodiment of the invention, T₃ is comprised between T₁ and T₂. In some cases, T₃ is lower than T₂, preferentially by a factor of more than 1.0001, 1.1, 1.2, 1.5, 2, 5, 10, 10³ or 10⁵. In some other cases, T₃ is larger than T₁, preferentially by a factor of more than 1.0001, 1.1, 1.2, 1.5, 2, 5, 10, 10³ or 10⁵.

In one embodiment of the invention, the temperature of the nanoparticles is the temperature of the heating apparatus or furnace used to heat the nanoparticles and/or comprising the nanoparticles, preferentially before, during, or after the treatment of the nanoparticles by the purifying method.

In one embodiment of the invention, the interval of temperatures that separates T₁ and T₂, designated as [T₁, T₂], is such that: i), the nanoparticles display the largest variation or loss of weight or mass as a function of temperature and/or ii), the derivative of the variation or loss of weight or mass of the nanoparticles as a function of temperature is the largest.

In one embodiment of the invention, the ratio [% W(T₂)−% W(T₁)]/(T₂−T₁), where % W(T₂) and % W(T₁) are the percentages in weight or mass of the nanoparticles at T₂ and T₁, respectively, is larger than 10⁻⁵⁰, 10⁻³⁰, 10⁻²⁰, 10⁻¹⁰, 10-s, 0.01, 0.02, 0.03, 0.05, 0.1, 0.2, 0.3, 0.5, 1, 10 or 10⁵%/° C. In some cases, this ratio is large when the percentage in mass of carbon in the nanoparticles, preferentially before treating the nanoparticles by or with the purifying method, is large, preferentially larger than 10⁻²⁰, 10⁻¹⁰, 10⁻⁵, 10⁻², 10⁻¹, 1, 5, 10, 20, 50, 75, 85, 95 or 100%/° C.

In another embodiment of the invention, the ratio [% W(T₂)−% W(T₁)]/(T₂−T₁) is lower than 10⁵⁰, 10³⁰, 10²⁰, 10¹⁰, 10⁵, 10, 5, 2, 1, 0.5, 0.05, 10⁻³, 10⁻⁵, 10⁻¹⁰ or 10⁻²⁰%/° C. In some cases, this ratio is low when the percentage in mass of carbon in the nanoparticles, preferentially before treating the nanoparticles by or with the purifying method, is low, preferentially lower than 100, 95, 80, 70, 50, 30, 20, 10, 5, 2, 1, 10⁻¹, 10⁻³, 10⁻⁵, 10⁻¹⁰ or 10⁻²⁰%/° C.

In one embodiment of the invention, the temperature of the nanoparticles is maintained at T₁, T₂, and/or T₃, when T₁, T₂, and/or T₃ vary(ies) by less than 10⁵, 10³, 90, 80, 70, 60, 50, 40, 30, 20, 10, 5, 3, 2, 1, 10⁻⁵, 10⁻¹⁰ or 10⁻²⁰%. In some cases, for each temperature T₁, T₂, and/or T₃, this percentage is equal to Tₘₐₓᵢ−Tₘᵢₙᵢ/Tₐᵥᵢ, where Tₘₐₓᵢ, Tₘᵢₙᵢ, and Tₐᵥᵢ (i=1, 2, 3) are the maximum, minimum, and average temperatures reached during the heating time or during the heating step, preferentially after or when the temperature is maintained at a temperature T₁, T₂, and/or T₃. In some cases, this percentage is low when the furnace or heating apparatus enables to maintain the temperature stable without large fluctuations and/or when the nanoparticles are not prone to endothermic and/or exothermic reactions. In some cases, the endothermic reaction is a reaction in which heat or energy is transferred from the medium surrounding the nanoparticles to the nanoparticles. In some other cases, the exothermic reaction is a reaction in which heat or energy is transferred from the nanoparticles to the medium surrounding the nanoparticles.

In one embodiment of the invention, the temperature of the nanoparticles is not maintained at T₁, T₂, and/or T₃, when the temperature of the nanoparticles varies by more than 10⁵, 10³, 90, 80, 70, 60, 50, 40, 30, 20, 10, 5, 3, 2, 1, 10⁻⁵, 10⁻¹⁰ or 10⁻²⁰%. In some cases, this percentage is large when the furnace or heating apparatus does not enable to maintain the temperature stable without large fluctuations and/or when the nanoparticles are prone to endothermic and/or exothermic reactions.

In one embodiment of the invention, the temperatures T₁ and/or T₂ is/are determined by:
i) measuring the variation of the percentage in weight or mass of the nanoparticles as a function of temperature when the nanoparticles are heated between two temperatures T_{T<T1} and T_{T>T2}, where T_{T<T1} is lower than T₁ and T_{T>T2} is larger than T₂,
ii) measuring or representing or considering or examining or using at least one peak of the derivative of the variation of this percentage as a function of temperature,
iii) estimating or deducing from the variation of the percentage in weight or mass of the nanoparticles as a function of temperature, the interval of temperature in which this variation is maximum, where the minimum and maximum temperatures of this interval are T₁ and T₂, respectively,
iv) estimating or deducing from the position of at least one peak in the plot of the derivative of the variation of the percentage in weight or mass of the nanoparticles the two temperatures T₁ and T₂, resulting in the minimum values of the derivative at the beginning and end of the peak, respectively, and T₃ resulting in the maximum value of the derivative at the middle of the peak, and
v) estimating or deducing from the position of at least one peak in the plot of the variation of the heat flux of the nanoparticles as a function of temperatures, the temperatures T₁, T₂, T₃, preferentially located at the beginning of the at least one peak.

In some cases, the temperature where or for which or at which the peak starts to display a decrease of the derivative of the variation of the percentage in weight or mass of the nanoparticles as a function of temperature is at the beginning of the peak. In some other cases, the temperature where or for which or at which the peak stops to display an increase of the derivative of the variation of the percentage in weight or mass of the nanoparticles as a function of temperature is the end of the peak.

In another embodiment of the invention, the heat flux of the nanoparticles is the heat flux produced by the nanoparticles or released from the nanoparticles or originating from the nanoparticles, preferentially when the nanoparticles are heated with a heating apparatus such a furnace. Preferentially, the heat flux can be measured with an apparatus or using a thermo-analytical method, or using Differential thermo-analysis (DTA) or using differential scanning calorimetry (DSC).

In one embodiment of the invention, the heating step i, where i is preferentially an integer larger or equal to/than 0, of the purifying method according to the invention comprises at least one of the following phase(s), wherein:
during the first phase, the temperature of the nanoparticles is increased from a temperature Tᵢ up to a temperature Tᵢₐᵥ, during a lapse of time t_{i1P},
during the second phase, the temperature of the nanoparticles is maintained at the temperature Tᵢₐᵥ, during a lapse of time t_{i2P}, and
during the third phase, the temperature of the nanoparticles is decreased from Tᵢₐᵥ to T_{f}, during a lapse of time t_{i3P}.

The invention also relates to a method of purification according to the invention comprising at least one heating step i, where the heating step comprises at least one of the following first, second and/or third phase(s), wherein:
during the first phase, the temperature of the nanoparticles is increased from a temperature Tᵢ up to a temperature Tᵢₐᵥ, during a lapse of time t_{i1P},
during the second phase, the temperature of the nanoparticles is maintained at the temperature Tᵢₐᵥ, during a lapse of time t_{i2P}, and
during the third phase, the temperature of the nanoparticles is decreased from Tᵢₐᵥ to T_{f}, during a lapse of time t_{i3P}.

In some cases, Tᵢ and/or t_{i1P}, is/are at least 1.0001, 1.1, 1.5, 2, 5, 10 or 100 times lower than Tᵢₐᵥ and/or t_{i2P}. In some cases, Tᵢₐᵥ is equal to T₁, T₂, or T₃, and/or t_{i1P} is equal to the time during which the temperature is increased to reach T₁. In still some other cases, T_{f} and/or t_{i3P} does/do not differ by a factor of more than 1.0001, 1.1, 1.5, 2, 5, 10, 10² or 10⁵ from Tᵢ and/or t_{i1P}.

The invention also relates to the method, preferentially purifying method, according to the invention, wherein more than 10% in mass of carbon or carbonaceous material is removed from the nanoparticles, where this percentage is based on the ratio (% C_{AT}−% C_{BT})/% C_{BT}, where % C_{AT} and % C_{BT} are the percentages of carbon or carbonaceous material after and before treating the nanoparticles with the method, respectively.

In some cases, (% C_{AT}−% C_{BT})/% C_{BT} is larger than 10⁻⁵⁰, 10⁻²⁰, 10⁻⁵, 10⁻², 10⁻¹, 1, 5, 10, 50, 75, 90, 95 or 99%. This can be the case when the purifying method is efficient or when the quantity of carbon or carbonaceous material comprised in the nanoparticle before treating the nanoparticles with the purifying method is lower than a certain threshold, preferentially lower than 99, 90, 70, 60, 50, 40, 30, 20, 10 or 1%.

In some other cases, (% C_{AT}−% C_{BT})/% C_{BT} is lower than 99, 90, 70, 60, 50, 40, 30, 20, 10 or 1%.

This can be the case when the purifying method is not efficient or when the quantity of carbon or carbonaceous material comprised in the nanoparticle before treating the nanoparticles with the purifying method is larger than a certain threshold, preferentially larger than 99, 90, 70, 60, 50, 40, 30, 20, 10 or 1%.

In still some other cases, (% C_{AT}−% C_{BT})/% C_{BT} is between 0.1 and 100, 1 and 99, 10 and 99, 50 and 99, or between 80 and 99%.

The invention relates to high purity iron oxide based nanoparticle(s) obtained by the method according to the invention.

The invention also relates to high purity nanoparticle(s) or high purity nanoparticles that are not obtained by the method.

In one embodiment of the invention, the high purity nanoparticles, preferentially the coating of these nanoparticles, comprise: i), between 0.8 and 0.999999999 g of iron oxide per gram of nanoparticle, and/or ii) between 10⁻⁴⁰ and 10⁵ μg of impurity(ies) per gram of nanoparticle.

In one embodiment of the invention, the high purity nanoparticles comprise a percentage in mass of carbon or carbonaceous material that is lower than 90, 10, 5, 2, preferentially 1, 0.5, 0.4 or 0.3%. In some cases, such a low percentage in mass of carbon enables to coat the nanoparticles with a coating that does not originates from the nanoparticle-producing cells.

In one embodiment of the invention, the SAR (specific absorption rate) of the high purity iron oxide nanoparticles is larger than 10⁻¹⁰⁰, 10⁻⁵⁰, 10⁻²⁰, 10⁻¹⁰, 10⁻⁵, 10⁻³, 10⁻¹, 1, 10, 10³ or 10⁵ Watt per gram of nanoparticles. In some cases, the SAR of the nanoparticles is the largest when the quantity of impurities in the nanoparticles is the lowest. In some cases, the SAR of the high purity iron oxide nanoparticles is comprised between 10⁻¹⁰⁰ and 10¹⁰⁰, 10⁻¹ and 10⁵, or between 0.1 and 10³ Watt per gram of nanoparticles. In some cases, the SAR of the nanoparticles is proportional to the slope, preferentially initial slope, of the temperature variation with time of the nanoparticles, (ΔT/δt), preferentially surrounded by a medium such as water, biological material, body part, or tissue, where (ΔT/δt) is preferentially estimated in ° C./sec., where SAR=α(ΔT/δt). In some cases, α=Cᵥ/Cₙₐₙₒ, where Cᵥ is the specific heat capacity, preferentially of water, biological material, body part, or tissue, comprising the nanoparticles, and Cₙₐₙₒ is the nanoparticle concentration or quantity or number of nanoparticles, preferentially comprised in water, biological material, body part, or tissue. In some cases, the SAR is measured by exposing the high purity iron oxide nanoparticles to a radiation, preferentially a radiation that produces heat, preferentially a laser, magnetic field, alternating magnetic field, acoustic wave, ultrasound, radiofrequency.

In one embodiment of the invention, the high purity iron oxide nanoparticles have a size distribution that is lower than 10⁵⁰, 10²⁰, 10¹⁰, 10⁵, 10³, 10², 10, 1, 10⁻¹, 10⁻² or 10⁻⁵ nm. In some cases, the nanoparticle size distribution is low when the method according to the invention enables the fabrication of nanoparticles with a low size distribution.

In another embodiment of the invention, the high purity iron oxide nanoparticles, preferentially at a concentration larger than 10⁻⁶, 10⁻³, 10⁻¹, 1 or 10 mg of nanoparticles per ml or per mm³ or per cell, destroy more than 1, 10, 10³, 10⁶ or 10⁹ cell(s).

The invention also relates to high purity iron oxide nanoparticle(s) that are produced at a yield larger than 10⁻⁵⁰, 10⁻³⁰, 10⁻¹⁰, 10⁻⁵, 10⁻², 10⁻¹, 1, 5, 10, 50, 10², 10³ or 10⁵ mg of nanoparticle(s) or mg of iron comprised in nanoparticle(s), preferentially per cell, preferentially per liter of pre-growth and/or growth medium/media.

The invention also relates to the nanoparticle(s) obtained by the method according to the invention, wherein the yield of nanoparticle production is lower than 10⁵⁰, 10³⁰, 10¹⁰, 10⁵, 10², 10, 5, 1, 10⁻¹, 10⁻², 10⁻³ or 10⁻⁵ mg of nanoparticle(s) or mg of iron comprised in nanoparticle(s), preferentially per cell, preferentially per liter of pre-growth and/or growth medium/media.

The invention also relates to high purity iron oxide based nanoparticle(s) according to the invention, wherein the high purity iron oxide nanoparticles is/are magnetosome(s).

In one embodiment of the invention, the magnetosomes are nanoparticles produced by magnetotactic bacteria that are preferentially treated following at least one of the following steps: i) nanoparticles are extracted and/or isolated from the bacteria, preferentially to obtain magnetosomes comprising crystallized minerals surrounded by a biological membrane, ii) the biological membrane is removed, preferentially using the purifying step, iii) the magnetosomes are coated with a coating that does not come from the nanoparticle-producing cell for stabilization, preferentially to avoid that the magnetosomes aggregate and/or sediment.

The invention also relates to a composition, drug, medical device, diagnostic composition, therapeutic composition, or cosmetic composition, comprising the high purity iron oxide nanoparticle(s) according to the invention.

In another embodiment of the invention, the high purity iron oxide nanoparticles yield: i) medical or therapeutic activity, for example by enabling the destruction of pathological cells, viruses, bacteria, cancer cells, or by being less toxic towards healthy tissues than pathological cells, viruses, bacteria, cancer cells, ii) diagnostic activity, for example by enabling the detection of pathological cells, viruses, bacteria, cancer cells, or by being less toxic towards healthy tissues, and/or, iii) cosmetic activity, for example by improving the appearance of a human.

In another embodiment of the invention, the high purity iron oxide nanoparticles are non-immunogenic or non-pyrogenic. In this case, they preferentially: i) attract or result in the appearance of a low number of immune cells, preferentially less than 1, 5, 10, 10³, 10¹⁰, 10⁵⁰ or 10¹⁰⁰ immune cells and/or ii) produce a temperature increase of a living organism lower than 10⁵, 10³, 10², 50, 20, 10, 5, 2, 1 or 0.1° C.

The invention also relates to the nanoparticles according to the invention, preferentially the high purity iron oxide based nanoparticles, for use in the treatment of a disease, which is preferentially selected in the group consisting of: i) a disease associated with a proliferation of cells that is different from the cellular proliferation in a healthy individual, ii) a disease associated with the presence of pathological cells such as tumor or cancer cells in the body part or in the individual, iii) a disease associated with the presence of a pathological site, i.e a site comprising pathological cells, in an individual or body part, iv) a disease or disorder or malfunction of the body part, v) a disease associated with the presence of radio-resistant or acoustic-resistant or laser-resistant or magnetic field resistant cells, vi) an infectious disease, vii) an auto-immune disease, viii) a neuropathology, ix) a cancer, x) a tumor, xi) a disease comprising or due to at least one cancer or tumor cell, xii) a cutaneous condition, xiii) an endocrine disease, xiv) an eye disease or disorder, xv) an intestinal disease, xvi) a communication disorder, xvii) a genetic disorder, xviii) a neurological disorder, xix) a voice disorder, xx) a vulvovaginal disorder, xxi) a liver disorder, xxii) a heart disorder, xxiii) a heating disorder, xxiv) a mood disorder, xxv) anemia, preferentially iron anemia, xxvi) a personality disorder, xxvii) aids, notably neuro-aids, xxviii) Parkinson, xxix) Alzheimer, xxx) bacterial and/or fungi infection or contamination, xxxi) blood disease due for example to an absence or lack of efficient coagulation, and xxxii) a disease due to a deficiency in immune function or an immune disease.

In one embodiment of the invention, the cancer or tumor selected from the group consisting of: the cancer of an organ, cancer of blood, cancer of a system of a living organism, adrenal cancer, anal cancer, bile duct cancer, bladder cancer, bone cancer, brain cancer, breast cancer, cervical cancer, colon/rectum cancer, endometrial cancer, esophagus cancer, eye cancer, gallbladder cancer, heart cancer, kidney cancer, laryngeal and hypopharyngeal cancer, leukemia, liver cancer, lung cancer, nasal cavity and paranasal sinus cancer, nasopharyngeal cancer, neuroblastoma, non-Hodgkin lymphoma, oral cavity and oropharyngeal cancer, osteosarcoma cancer, ovarian cancer, pancreatic cancer, pancreatic penile cancer, prostate cancer, retinoblastoma, rhabdomyosarcoma, salivary gland cancer, sarcoma, skin cancer, small intestine cancer, stomach cancer, testicular cancer, thymus cancer, thyroid cancer, uterine cancer, uterine sarcoma cancer, vaginal cancer, vulvar cancer, waldenstrom macroglobulinemia wilms tumor, castleman disease ewing family of tumor, gastrointestinal carcinoid tumor, gastrointestinal stromal tumor, myelodysplastic syndrome pituitary tumor, and a cancerous disease such as gestational trophoblastic disease, Hodgkin disease, kaposi sarcoma, malignant mesothelioma, and multiple myeloma.

In still another embodiment of the invention, the treatment of the disease by the nanoparticles according to the invention occurs or is activated when the nanoparticles are exposed to a radiation and preferentially does not occur or is not activated when the nanoparticles are not exposed to a radiation.

In still another embodiment, the radiation is a laser, an acoustic wave such as an ultrasound, X-ray, gamma ray, and/or a magnetic field, preferentially an alternative magnetic field.

In still some other embodiment, the power or intensity of the radiation is between 10⁻⁵⁰, 10⁻²⁰, 10⁻¹⁰, 10⁻⁵, 10⁻¹, 0, 1 mT, T, W, W/cm, W/cm² or W/cm³ and 1, 5, 10, 10³, 10⁵, 10¹⁰, 10²⁰ or 10⁵⁰ mT, T, W, W/cm, W/cm² or W/cm³.

In still another embodiment of the invention, the property(ies) or features, preferentially of the nanoparticle(s) or method, described in each individual embodiment or section or sentence of this patent application can be combined to result in a combination of property(ies) or features, preferentially of the nanoparticle(s) or method.

In still another embodiment of the invention, when a compound such as the nanoparticle or chemical element has a property in a condition 1 (P₁) that is higher, longer, or larger by a factor α than a property in a condition 2 (P₂), it means that P₁=α·P₂ or P₁=α+P₂, where α is preferentially a number or integer larger than 1 or 0.

In still another embodiment of the invention, when a compound such as the nanoparticle or chemical element has a property in a condition 1 (P₁) that is smaller, or shorter by a factor α than a property in a condition 2 (P₂), it means that P₁=P₂/α or P₁=P₂−α, where α is preferentially a number or integer larger than 1 or 0.

The invention also relates to the high purity magnetosomes according to the invention, wherein each magnetosome has a structure that comprises at least (i) a magnetosome mineral or magnetosome central part or magnetosome core and optionally (ii) a magnetosome coating surrounding the magnetosome mineral or magnetosome central part or magnetosome core. Each magnetosome comprises a metallic composition, which may be specified relative to the magnetosome/magnetosome composition (i.e., the entire magnetosome composition) or magnetosome mineral or magnetosome core or magnetosome central part or magnetosome coating.

The invention also relates to high purity magnetosomes according to the invention preferentially produced or synthesized by magnetotactic bacteria, preferentially using a pre-growth and/or growth and/or fed-batch medium(a), wherein the metallic composition of the magnetosome mineral or magnetosome core or magnetosome central part or magnetosome coating or magnetosome composition, is characterized by at least one property selected in the group consisting of:
i) the mass of Cd preferentially comprised in 1 gram of or magnetosome mineral or magnetosome core or magnetosome central part or magnetosome coating or magnetosome composition, is smaller than 350000, 35000, 3500, 350, 35, 3.5, 0.35, 0.035, or 0.0035 μg,
ii) the percentage in mass of Cd preferentially comprised in the magnetosome mineral or magnetosome core or magnetosome central part or magnetosome coating or magnetosome composition is smaller than 100, 99, 95, 30, 50, 3, 3·10⁻¹, 3·10⁻², 3·10⁻³, 3·10⁻⁴ or 0%,
iii) the mass of Pb preferentially comprised in 1 gram of or magnetosome mineral or magnetosome core or magnetosome central part or magnetosome coating or magnetosome composition, is smaller than 500000, 50000, 5000, 500, 50, 5 or 0.5 μg,
iv) the percentage in mass of Pb preferentially comprised in the magnetosome mineral or magnetosome core or magnetosome central part or magnetosome coating or magnetosome composition is smaller than 100, 50, 5, 5·10⁻¹, 5·10⁻², 5·10⁻³ or 0%,
v) the mass of Ni preferentially comprised in 1 gram of magnetosome mineral or magnetosome core or magnetosome central part or magnetosome coating or magnetosome composition, is smaller than 2000000, 200000, 20000, 2000, 200, 20 or 2 μg,
vi) the percentage in mass of Ni preferentially comprised in the magnetosome mineral or magnetosome core or magnetosome central part or magnetosome coating or magnetosome composition is smaller than 10², 99, 95, 50, 10, 1, 10⁻¹, 10⁻², 10⁻³ or 0%,
vii) the mass of Co preferentially comprised in 1 gram of or magnetosome mineral or magnetosome core or magnetosome central part or magnetosome coating or magnetosome composition is smaller than 1000000, 10000, 1000, 100, 10 or 1 μg,
viii) the percentage in mass of Co preferentially comprised in the magnetosome mineral or magnetosome core or magnetosome central part or magnetosome coating or magnetosome composition is smaller than 10², 99, 95, 50, 10, 1, 10⁻¹, 10⁻², 10⁻³ or 0%,
ix) the mass of Al preferentially comprised in 1 gram of or magnetosome mineral or magnetosome core or magnetosome central part or magnetosome coating or magnetosome composition is smaller than 5000 000, 50 000, 5000, 500, 50, 5, or 0.5 μg,
x) the percentage in mass of Al comprised in the magnetosome mineral or magnetosome core or magnetosome central part or magnetosome coating or magnetosome composition is smaller than 100, 99, 95, 50, 30, 10, 5·10⁻¹, 5·10⁻², 5·10⁻³ or 0%,
xi) the mass of Zn preferentially comprised in 1 gram of or magnetosome mineral or magnetosome core or magnetosome central part or magnetosome coating or magnetosome composition, is smaller than 5000 000, 50 000, 5000, 500, 50, 5, or 0.5 μg,
xii) the percentage in mass of Zn preferentially comprised in the magnetosome mineral or magnetosome core or magnetosome central part or magnetosome coating or magnetosome composition is smaller than 100, 99, 95, 50, 30, 10, 5·10⁻¹, 5·10⁻², 5·10⁻³ or 0%,
xiii) the mass of Mn preferentially comprised in 1 gram of magnetosome mineral or magnetosome core or magnetosome central part or magnetosome coating or magnetosome composition is smaller than 1000 000, 100 000, 10 000, 1000, 100, 10, 1 or 0.1 μg,
xiv) the percentage in mass of Mn comprised in the magnetosome mineral or magnetosome core or magnetosome central part or magnetosome coating or magnetosome composition is smaller than 100, 99, 95, 50, 25, 10, 1, 0.1, 0.01 or 0%,
xv) the mass of Li preferentially comprised in 1 gram of magnetosome mineral or magnetosome core or magnetosome central part or magnetosome coating or magnetosome composition is smaller than 500 000, 50 000, 5 000, 500, 50, 5, 0.5 or 0.05 μg,
xvi) the percentage in mass of Li preferentially comprised in the magnetosome mineral or magnetosome core or magnetosome central part or magnetosome coating or magnetosome composition is smaller than 100, 50, 25, 10, 1, 10⁻¹, 10⁻², 5·10⁻³, 10⁻³, 10⁻⁴ or 0%.
xvii) the mass of Cu preferentially comprised in 1 gram of or magnetosome mineral or magnetosome core or magnetosome central part or magnetosome coating or magnetosome composition is smaller than 100 000, 10 000, 1000, 100, 10, 1 or 0.1 μg,
xviii) the percentage in mass of Cu preferentially comprised in the magnetosome mineral or magnetosome core or magnetosome central part or magnetosome coating or magnetosome composition is smaller than 100, 50, 10, 1, 0.1, 10⁻² or 0%,
xix) in or for a given or fixed quantity or mass of Fe preferentially comprised in the magnetosome mineral or magnetosome core or magnetosome central part or magnetosome coating or magnetosome composition, the quantity or mass of at least one metal preferentially comprised in the magnetosome or magnetosome mineral or magnetosome core or magnetosome central part or magnetosome coating or magnetosome composition preferentially selected in the group consisting of: Cd, Pb, Ni, Co, Al, Mn, Zn, Li, and Cu, is smaller, preferentially at least 10 000, 1000, 100, 10, 5 or 2 times smaller than the quantity or mass of Mg preferentially comprised in the or magnetosome mineral or magnetosome core or magnetosome central part or magnetosome coating or magnetosome composition,
xx) the percentage in mass of iron in the magnetosome mineral or magnetosome core or magnetosome central part is larger than 10, 50, 99 or 99.9%, and
xxi) the percentage in mass of at least one metal different from iron in the magnetosome mineral or magnetosome core or magnetosome central part is smaller than 10, 5, 1 or 0.1%,
wherein the mass of magnetosome mineral or magnetosome core or magnetosome central part or magnetosome coating or magnetosome composition is preferentially estimated as or corresponds to the mass of Fe comprised in the magnetosome mineral or magnetosome core or magnetosome central part or magnetosome coating or magnetosome composition.
wherein the percentage in mass of at least one compound comprised in the magnetosome or magnetosome mineral or magnetosome core or magnetosome central part or magnetosome coating or magnetosome composition is preferentially estimated relatively to the mass of all metals or of Fe and/or Mg comprised in the magnetosome mineral or magnetosome core or magnetosome central part or magnetosome coating or magnetosome composition,
wherein the at least one metal preferentially originates from the pre-growth and/or growth and/or fed-batch medium(a), most preferentially from the fed-batch medium.

In some cases, the at least one metal is preferentially selected among at least one metal different from Fe and/or Mg.

The invention also relates to high purity magnetosomes according to the invention preferentially produced or amplified by magnetotactic bacteria, wherein the metallic composition of the magnetosome composition or magnetosome mineral or magnetosome core or magnetosome central part is characterized by at least one property selected in the group consisting of:
i) the mass of Cd preferentially comprised in 1 gram of magnetosome mineral or magnetosome core or magnetosome central part or magnetosome coating or magnetosome composition, preferentially estimated in mass of Fe, is larger than 0, 10⁻¹⁰, 10⁻⁵, 10⁻³, 10⁻¹, 1, 10³ or 10⁵ μg,
ii) the percentage in mass of Cd preferentially relatively to the mass of all metals comprised in the magnetosome mineral or magnetosome core or magnetosome central part or magnetosome coating or magnetosome composition is larger than 0, 10⁻¹⁰, 10⁻⁵, 10⁻³, 10⁻¹, 1, 10, 25, 50 or 75%,
iii) the mass of Pb preferentially comprised in 1 gram of magnetosome mineral or magnetosome core or magnetosome central part or magnetosome coating or magnetosome composition, preferentially estimated in mass of Fe, is larger than 0, 10⁻¹⁰, 10⁻⁵, 10⁻³, 10⁻¹, 1, 10³ or 10⁵ μg,
iv) the percentage in mass of Pb preferentially relatively to the mass of all metals comprised in the magnetosome or magnetosome mineral or magnetosome core or magnetosome central part or magnetosome coating or magnetosome composition is larger than 0, 10⁻¹⁰, 10⁻⁵, 10⁻³, 10⁻¹, 1, 10, 25, 50 or 75%.
v) the mass of Ni preferentially comprised in 1 gram of magnetosome mineral or magnetosome core or magnetosome central part or magnetosome coating or magnetosome composition, preferentially estimated in mass of Fe, is larger than 0, 10⁻¹⁰, 10⁻⁵, 10⁻³, 10⁻¹, 1, 10³ or 10⁵ μg,
vi) the percentage in mass of Ni preferentially relatively to the mass of all metals comprised in the magnetosome mineral or magnetosome core or magnetosome central part or magnetosome coating or magnetosome composition is larger than 0, 10⁻¹⁰, 10⁻⁵, 10⁻³, 10⁻¹, 1, 10, 25, 50 or 75%,
vii) the mass of Co preferentially comprised in 1 gram of magnetosome mineral or magnetosome core or magnetosome central part or magnetosome coating or magnetosome composition, preferentially estimated in mass of Fe, is larger than 0, 10⁻¹⁰, 10⁻⁵, 10⁻³, 10⁻¹, 1, 10³ or 10⁵ μg,
viii) the percentage in mass of Co preferentially relatively to the mass of all metals comprised in the magnetosome mineral or magnetosome core or magnetosome central part or magnetosome coating or magnetosome composition is larger than 0, 10⁻¹⁰, 10⁻⁵, 10⁻³, 10⁻¹, 1, 10, 25, 50 or 75%,
ix) the mass of Al preferentially comprised in 1 gram of magnetosome mineral or magnetosome core or magnetosome central part or magnetosome coating or magnetosome composition, preferentially estimated in mass of Fe, is larger than 0, 10⁻¹⁰, 10⁻⁵, 10⁻³, 10⁻¹, 1, 10³ or 10⁵ μg,
x) the percentage in mass of Al preferentially relatively to the mass of all metals comprised in the magnetosome mineral or magnetosome core or magnetosome central part or magnetosome coating or magnetosome composition is larger than 0, 10⁻¹⁰, 10⁻⁵, 10⁻³, 10⁻¹, 1, 10, 25, 50 or 75%,
xi) the mass of Zn preferentially comprised in 1 gram of magnetosome mineral or magnetosome core or magnetosome central part or magnetosome coating or magnetosome composition, preferentially estimated in mass of Fe, is larger than 0, 10⁻¹⁰, 10⁻⁵, 10⁻³, 10⁻¹, 1, 10³ or 10⁵ μg,
xii) the percentage in mass of Zn preferentially relatively to the mass of all metals comprised in the magnetosome or magnetosome mineral or magnetosome core or magnetosome central part or magnetosome coating or magnetosome composition is larger than 0, 10⁻¹⁰, 10⁻⁵, 10⁻³, 10⁻¹, 1, 10, 25, 50 or 75%,
xiii) the mass of Mn preferentially comprised in 1 gram of magnetosome mineral or magnetosome core or magnetosome central part or magnetosome coating or magnetosome composition, preferentially estimated in mass of Fe, is larger than 0, 10⁻¹⁰, 10⁻⁵, 10⁻³, 10⁻¹, 1, 10³ or 10⁵ μg,
xiv) the percentage in mass of Mn preferentially relatively to the mass of all metals comprised in the magnetosome mineral or magnetosome core or magnetosome central part or magnetosome coating or magnetosome composition is larger than 0, 10⁻¹⁰, 10⁻⁵, 10⁻³, 10⁻¹, 1, 10, 25, 50 or 75%,
xv) the mass of Li preferentially comprised in 1 gram of magnetosome mineral or magnetosome core or magnetosome central part or magnetosome coating or magnetosome composition, preferentially estimated in mass of Fe, is larger than 0, 10⁻¹⁰, 10⁻⁵, 10⁻³, 10⁻¹, 1, 10³ or 10⁵ μg,
xvi) the percentage in mass of Li preferentially relatively to the mass of all metals comprised in the magnetosome mineral or magnetosome core or magnetosome central part or magnetosome coating or magnetosome composition is larger than 0, 10⁻¹⁰, 10⁻⁵, 10⁻³, 10⁻¹, 1, 10, 25, 50 or 75%,
xvii) the mass of Cu preferentially comprised in 1 gram of magnetosome mineral or magnetosome core or magnetosome central part or magnetosome coating or magnetosome composition preferentially estimated in mass of Fe, is larger than 0, 10⁻¹⁰, 10⁻⁵, 10⁻³, 10⁻¹, 1, 10¹ or 10⁵ μg,
xviii) the percentage in mass of Cu preferentially relatively to the mass of all metals comprised in the magnetosome minerals or magnetosome core or magnetosome central part or magnetosome coating or magnetosome composition is larger than 0, 10⁻¹⁰, 10⁻⁵, 10⁻³, 10⁻¹, 1, 10, 25, 50 or 75%,
xix) in or for a given or fixed quantity or mass of Fe preferentially comprised in the magnetosome mineral or magnetosome core or magnetosome central part magnetosome composition, the quantity or mass of at least one metal preferentially comprised in the magnetosome mineral or magnetosome core or magnetosome central part or magnetosome composition preferentially selected in the group consisting of: Cd, Pb, Ni, Co, Al, Mn, Zn, Li, and Cu, is at least 0, 1, 2, 5 or 10 times larger than the quantity or mass of Mg preferentially comprised in the magnetosome mineral or magnetosome core or magnetosome central part or magnetosome composition,
xx) the percentage in mass of iron in the magnetosome mineral or magnetosome core or magnetosome central part is smaller than 100, 99.9, 90 or 50%, and
xxi) the percentage in mass of at least one metal different from iron in the magnetosome mineral or magnetosome core or magnetosome central part is larger than 0, 1, 5, 10, 30 or 50%, The invention relates to high purity magnetosomes according to the invention, wherein the metallic composition of the magnetosome is characterized by at least one property selected in the group consisting of:
i) the magnetosome central part, magnetosome core or magnetosome mineral part comprises more than 95, 99 or 99.9% of iron, preferentially relatively to the mass of iron and at least one other metal comprised in the magnetosome central part, magnetosome core or magnetosome mineral part, and
ii) the magnetosome composition comprises more than 90, 95 or 99% of iron and Magnesium, wherein Magnesium is preferentially comprised outside of the magnetosome central part, magnetosome core or magnetosome mineral part.

The invention also relates to the high purity magnetosomes according to the invention, wherein the magnetosome composition is such that:
i) it comprises the magnetosome central part, magnetosome core or magnetosome mineral part,
ii) it does not comprise at least one compound preferentially synthesized or produced by at least one magnetotactic bacterium, which is selected in the group consisting of: i) a magnetosome coating, preferentially magnetosome biological coating, ii) at least one protein, preferentially a non-denatured or an activated protein, iii) at least one lipid, preferentially a non-denatured or an activated lipid, iv) at least one lipopolysaccharide, preferentially a non-denatured or an activated lipopolysaccharide, v) at least one endotoxin, preferentially a non-denatured or an activated endotoxin, vi) at least one piece of DNA, preferentially a non-denatured or non-modified or an activated DNA, vi) at least one piece of RNA, preferentially a non-denatured or non-modified or an activated RNA, v) at least one biological compound, preferentially a non-denatured or non-modified or an activated biological compound, and/or
iii) it comprises a magnetosome coating surrounding the magnetosome central part, magnetosome core or magnetosome mineral part, wherein the magnetosome coating does not comprise at least one compound mentioned in ii).

The invention also relates to the high purity magnetosomes according to the invention, wherein the magnetosome, or magnetosome composition, is such that it comprises three parts:
i) A first part, which is the magnetosome central part or magnetosome mineral or magnetosome central part or magnetosome core;
ii) A second part, which is the magnetosome coating surrounding the magnetosome mineral or magnetosome central part or magnetosome core;
iii) A third part, which is the environment or material or liquid or excipient or water surrounding the magnetosome coating;
wherein the first part preferentially serves to enhance the effect of at least one radiation or thermal perturbation applied on the composition preferentially to more efficiently destroy or deactivate at least one pathological cell;
wherein the second part preferentially serves to stabilize the second part or to yield magnetosome chain arrangement;
wherein the third part preferentially serves to enable the administration of the composition to the body art, i.e. without the third part it is preferentially not feasible or not easy or not convenient to administer the composition to the body part, wherein the third part preferentially serves to preserve partly or fully the first and/or second part(s) preferentially before during or after administration of the composition in or to the body part.

The invention also relates to the high purity magnetosomes according to the invention, wherein the magnetosome composition is such that:
i) it comprises the magnetosome central part, magnetosome core or magnetosome mineral part,
ii) it does not comprise at least one compound preferentially synthesized or produced by at least one magnetotactic bacterium, which is selected in the group consisting of: i) a magnetosome coating, preferentially magnetosome biological coating, ii) at least one protein, preferentially a non-denatured or an activated protein, iii) at least one lipid, preferentially a non-denatured or an activated lipid, iv) at least one lipopolysaccharide, preferentially a non-denatured or an activated lipopolysaccharide, v) at least one endotoxin, preferentially a non-denatured or an activated endotoxin, vi) at least one piece of DNA, preferentially a non-denatured or non-modified or an activated DNA, vi) at least one piece of RNA, preferentially a non-denatured or non-modified or an activated RNA, v) at least one biological compound, preferentially a non-denatured or non-modified or an activated biological compound.

The invention also relates to high purity magnetosomes according to the invention, wherein the magnetosome composition is such that:
i) the magnetosome comprises at least one metal of group A selected in the group of: Aluminum, Barium, Calcium, Cerium, Cesium, Chromium, Dysprosium, Erbium, Europium, Gadolinium, Gallium, Hafnium, Holmium, Indium, Iron, Lanthanum, Lutetium, Magnesium, Manganese, Molybdenum, Niobium, Neodymium, Potassium, Praseodymium, Rhenium, Samarium, Scandium, Sodium, Strontium, Technetium, Terbium, Thulium, Titanium, Tungsten, Yttrium, Zinc, Zirconium, and
ii) the magnetosome does not comprise at least one metal of group B or comprises at least one metal of group B in smaller quantity than at least one metal of group A, where the at least one metal of group B is selected in the group consisting of: Antimony, Arsenic, Cadmium, Cobalt, Copper, Gold, Iridium, Lead, Lithium, Mercury, Nickel, Osmium, Palladium, Platinum, Rhodium, Ruthenium, Selenium, Silver, Thallium, Tin, and Vanadium.

In some cases, the magnetosome comprises more, preferentially at least 1.0001, 1.1, 1.5, 2, 5 or 10 time(s) more at least one metal of group A than at least one metal of group B.

The invention also relates to the high purity magnetosomes according to the invention comprising more than 50, 95 or 99% of iron and Mg, based on a ratio of (M_{FeN}+M_{MgN})/M_{MN}, where M_{FeN} is the mass of iron in the high purity magnetosomes, M_{MgN} is the mass of magnesium in the high purity magnetosomes, and M_{MN} is the mass of iron and metals or metalloids other than iron in the high purity iron oxide nanoparticle(s), wherein iron is preferentially comprised partly or fully in the magnetosome mineral or magnetosome core or magnetosome central part, wherein magnesium is preferentially comprised partly or fully outside of the magnetosome mineral or magnetosome core or magnetosome central part or magnetosome coating, wherein magnesium is preferentially comprised in the magnetosome composition preferentially in the form of a magnesium salt.

The invention also relates to the high purity magnetosomes according to the invention comprising more than 50, 95 or 99% of iron and Mg, preferentially based on a ratio of (M_{FeN}+M_{MgN})/M_{MN}, where M_{FeN} is the mass of iron in the high purity magnetosomes (i.e., in the magnetosome composition), M_{MgN} is the mass of magnesium in the high purity magnetosomes (i.e., in the magnetosome composition,) and M_{MN} is the mass of iron and metals or metalloids other than iron in the high purity iron oxide nanoparticle(s) or composition,
wherein iron is preferentially comprised partly or fully in the magnetosome mineral or magnetosome core or magnetosome central part,
wherein magnesium is preferentially comprised partly or fully outside of the magnetosome mineral or magnetosome core or magnetosome central part or magnetosome coating,
wherein magnesium is preferentially comprised partly or fully in the environment or exterior or external medium of the magnetosome mineral or magnetosome core or magnetosome central part or magnetosome coating,
wherein magnesium is preferentially comprised in the magnetosome composition preferentially in the form of a magnesium salt.

In some cases, the high purity magnetosome or nanoparticle can designate the core or mineral part or central part or coating of the high purity magnetosome or nanoparticle.

In some cases, the high purity magnetosome or nanoparticle can designate a composition comprising the high purity magnetosome or nanoparticle.

The invention also relates to the high purity magnetosomes according to the invention, wherein the magnetosome comprises a percentage in mass of magnesium that is larger than 0, 0.1, 0.5, 1, 2, 2.5, 5, 10, 25, 50, 75, 80, 90 or 99%, preferentially relative to the mass of at least one or of all metals in the magnetosome, preferentially except iron, alternatively preferentially relative to the mass of magnetosome.

The invention also relates to the high purity magnetosomes according to the invention, wherein the magnetosome (i.e., the magnetosome composition) comprises magnesium at:
i) a percentage in mass that is larger 0.1, 0.5, 1, 2 or 2.5%;
and/or
ii) a percentage in mass or concentration that is larger than the percentage in mass or concentration of Manganese, preferentially by a factor of at least 1, 1.1, 2, 5, 10 or 10³;
wherein the percentage in mass is preferentially estimated relatively to the mass of iron and at least one other metal comprised in the magnetosome composition,
wherein the large quantity of magnesium in the magnetosome composition is preferentially obtained by reducing the concentration of manganese in at least one medium used for magnetosome production and/or by reducing the quantity of manganese in the magnetosome composition.

In some cases, the high purity magnetosomes according to the invention comprises a percentage in mass of magnesium that is lower than 100, 99, 90, 75, 50, 40, 20, 10, 5, 2, 1 or 0%, preferentially relative to the mass of at least one or of all metals in the magnetosome, preferentially except iron, alternatively preferentially relative to the mass of magnetosome.

The invention also relates to the high purity magnetosomes according to the invention, wherein the magnetosome comprises a percentage in mass of magnesium or of magnesium and iron that is larger than the percentage in mass of at least 1, 2, 3, 4, 5 or 10 different metals preferentially selected in the group selected from: As, Cd, Hg, Pb, Co, Ni, V, Ag, Au, Ir, Os, Pd, Pt, Rh, Ru, Se, Tl, Ba, Cr, Cu, Li, Mo, Sb, Sn, Al, B, Ca, Fe, K, Mg, Mn, Na, W, and Zn.

The invention also relates to the high purity magnetosomes according to the invention, wherein the magnetosome metallic composition (i.e., the metallic composition of the magnetosomes or of the magnetosome composition) is the metallic composition of:
i) the magnetosome core or magnetosome central part or magnetosome mineral,
ii) the region of the magnetosome composition that comprises crystallized iron or iron oxide, preferentially with a nanometric size or a size smaller than 500 nm for individual crystallized iron or iron oxide entities or particles, preferentially observable by a method such as electron microscopy,
iii) the magnetosome synthesized by magnetotactic bacteria without at least one compound selected in the group consisting of: i) coating, ii) at least one compound that does not belong to the magnetosome core or magnetosome central part or magnetosome mineral, iii) at least one excipient or water or liquid.

The invention also relates to the high purity magnetosomes according to the invention, wherein the metallic composition is the metallic composition of the magnetosome core, central part, mineral part or of the magnetosome synthesized by magnetotactic bacteria preferentially without the addition of a coating that is chemical, synthetic or not synthesized by magnetotactic bacteria.

The invention also relates to the high purity magnetosomes according to the invention, wherein the magnetosome metallic composition is the metallic composition of:
i) the magnetosome core or magnetosome central part or magnetosome mineral,
ii) the region of the magnetosome composition that comprises crystallized iron or iron oxide, preferentially with a nanometric size or a size smaller than 100, or 500 nm for individual crystallized iron or iron oxide entities or particles, preferentially observable by a method such as electron microscopy,
iii) the magnetosome synthesized by magnetotactic bacteria without at least one compound selected in the group consisting of: i) coating, ii) at least one compound that does not belong to the magnetosome core or magnetosome central part or magnetosome mineral, iii) at least one excipient or water or liquid.

In some cases, the metallic composition can be the metallic composition of the magnetosome core, central part, mineral part, coating or of the magnetosome core, central, mineral part, and coating.

In some cases, the magnetosome core, magnetosome central part, and magnetosome mineral part can be equivalent.

The invention also relates to the high purity magnetosomes according to the invention, wherein the metallic composition of the magnetosomes consists of:
preferentially predominantly or more than 0, 1, 10, 25, 50, 90, 92, 95 or 99% of iron or magnesium or iron and magnesium, preferentially comprised in the magnetosome core;
preferentially predominantly or more than 0, 1, 10, 25, 50, 90, 92, 95 or 99% of another metal than iron and/or magnesium in the magnetosome coating preferentially surrounding the magnetosome core partly or fully;

The invention also relates to the high purity magnetosomes according to the invention, wherein the magnetosomes composition consists of:
predominantly or more than 50 or 90 or 99% of iron in the magnetosome core or magnetosome central part or magnetosome mineral;
predominantly or more than 50 or 90 or 99% of another metal than iron or of a carbonaceous material preferentially not originating partly or fully from magnetotactic bacteria in the magnetosome coating surrounding the magnetosome core or magnetosome mineral or magnetosome central part;
   and/or
predominantly or more than 50 or 90 or 99% of another metal than iron or of a carbonaceous material preferentially not originating partly or fully from magnetotactic bacteria, preferentially comprised in the magnetosome coating surrounding the magnetosome core or magnetosome mineral or magnetosome central part;

The invention also relates to the high purity magnetosomes according to the invention, wherein the metallic composition of the magnetosomes consists of:
preferentially less than 100, 50, 25, 10, 5, 2 or 10, or 1% of iron or magnesium or iron and magnesium, preferentially comprised in the magnetosome core;
preferentially less than 100, 50, 25, 10, 5, 2 or 10, or 1% of another metal than iron and/or magnesium in the magnetosome coating preferentially surrounding the magnetosome core partly or fully;

The invention also relates to a composition according to the invention comprising the high purity magnetosomes according to the invention.

The invention also relates to the composition according to the invention comprising the high purity magnetosomes according to the invention, wherein the magnetosomes are preferentially obtained by cultivating magnetotactic bacteria in a growth and/or pre-growth and/or fed-batch medium, which is preferentially substantially free of at least one metal or non-metal or preferentially comprises less than 1 nano-Molar of at least one metal or non-metal, wherein the at least one metal or non-metal is preferentially selected in the group consisting of: 1) cadmium, 2) lead, 3) arsenic, 4) mercury, 5) cobalt, 6) vanadium, 7) nickel, 8) lithium, 9) antimony, 10) copper, 11) Vanadium, 12) Molybdate, 13) Selenium, 14) Barium, 15) Chrome, 16) Strontium, 17) a radioactive chemical element, 18) Beryllium, 19) Rubidium, 20) Ruthenium, 21) Rhodium, 22) Palladium, 23) Promethium, 24) Ytterbium, 25) Tantalum, 26) Osmium, 27) Iridium, 28) Bismuth, 29) Polonium, 30) Francium, 31) Radium, 32) Actinium, 33) Thorium, 34) Protactinium, 35) Uranium, 36) Neptunium, 37) Plutonium, 38) Americium, 39) Curium, 40) Berkelium, 41) Californium, Einsteinium, 42) Fermium, 43) Mendelevium, 44) Nobelium, 45) Lawrencium, 46) Rutherfordium, 47) Dubnium, 48) Seaborgium, 49) Bohrium, 50) Hassium, 51) Meitnerium, 52) Darmstadtium, 53) Roentgenium, 54) Copernicium, 55) Nihonium, 56) Flerovium, 57) Moscovium, 58) Livermorium, 59) Astatine, 60) Tennessine, 61) Oganesson, and 62) derivatives thereof.

The invention also relates to the high purity magnetosomes according to the invention preferentially produced by a method comprising the following step(s):
a) preferentially a pre-growth step comprising amplifying the magnetotactic bacteria in a pre-growth and/or fed-batch medium/media,
and
b) preferentially a growth step comprising amplifying the magnetotactic bacteria originating from the pre-growth step in a growth and/or fed-batch medium/media, wherein the fed-batch medium preferentially comprises an iron source, preferentially to enable
magnetosome production,
   wherein preferentially the fed-batch medium in the pre-growth step and the growth step, when it is present, is a medium that supplements the pre-growth and/or growth medium/media,
wherein the fed-batch medium preferentially comprises an iron source or a higher concentration of iron than the pre-growth and/or growth medium(a), preferentially to enable magnetosome production,
wherein preferentially more, preferentially at least 1, 2, 5, 10, 100, 10³, 10⁵ time(s) more magnetosomes are produced in the growth step than in the pre-growth step.

The invention also relates to the high purity magnetosomes according to the invention preferentially produced by magnetotactic bacteria amplified in a pre-growth and/or growth and/or fed-batch medium(a) comprising at least one metal or at least one compound selected among Co, Ni, Se, Cu, Mo, Al, Ca, Mg, and Mn at concentration(s) estimated in gram of compound or metal per liter of pre-growth and/or growth and/or fed-batch medium(a) that is or are lower than 10⁻⁶, 10⁻⁵, 10⁻⁴, 10⁻³, 10⁻² or 10⁻¹ g of Co, 10⁻⁶, 10⁻⁵, 10⁻⁴, 10⁻³, 10⁻² or 10⁻¹ g of Ni, 10⁻¹⁰, 10⁻⁹, 10⁻⁸, 10⁻⁷, 10⁻⁶, 10⁻⁵, 10⁻⁴, 10⁻³, 10⁻² or 10⁻¹ g of Se, 10⁻⁹, 10⁻⁸, 10⁻⁷, 10⁻⁶, 10⁻⁵, 10⁻⁴, 10⁻³, 10⁻² or 10⁻¹ g of Cu, 10⁻¹⁰, 10⁻⁹, 10⁻⁸, 10⁻⁷, 10⁻⁶, 10⁻⁵, 10⁻⁴ or 10⁻³ g of Mo, 10⁻¹⁰, 10⁻⁹, 10⁻⁸, 10⁻⁷, 10⁻⁶, 10⁻⁵, 10⁻⁴, 10⁻³, 10⁻² or 10¹ g of Al, 10⁻¹⁰, 10⁻⁹, 10⁻⁸, 10⁻⁷, 10⁻⁶, 10⁻⁵, 10⁻⁴, 10⁻³, 10⁻² or 10⁻¹ g of Mg, 10⁻¹⁰, 10⁻⁹, 10⁻⁸, 10⁻⁷, 10⁻⁶, 10⁻⁵, 10⁻⁴, 10⁻³, 10⁻² or 10⁻¹ g of Mn, 10⁻¹⁰, 10⁻⁹, 10⁻⁸, 10⁻⁷, 10⁻⁶, 10⁻⁵, 10⁻⁴, 10⁻³, 10⁻² or 10⁻¹ g of Zn, 10⁻¹⁰, 10⁻⁹, 10⁻⁸, 10⁻⁷, 10⁻⁶, 10⁻⁵, 10⁻⁴, 10⁻³, 10⁻² or 10⁻¹ g of at least one metal.

The invention also relates to the high purity magnetosomes according to the invention produced by magnetotactic bacteria amplified in or using a pre-growth and/or growth and/or fed-batch medium(a) comprising at least one compound selected among As, Cd, Hg, Pb, Co, Ni, V, Ag, Au, Ir, Os, Pd, Pt, Rh, Ru, Se, Tl, Ba, Cr, Cu, Li, Mo, Sb, Sn, Al, B, Ca, Fe, K, Mg, Mn, Na, W, and Zn at concentration(s) estimated in gram of compound per liter of pre-growth and/or growth and/or fed-batch medium(a) that is or are lower than: 2·10⁻⁷ for As; 10⁻¹² for Cd; 10⁻¹² for Hg; 10⁻¹² for Pb; 2·10⁻⁴ for Co; 10⁻¹² for V; 10⁻¹² for Ag; 10⁻¹² for Au; 10⁻¹² for Ag; 10⁻¹² for Au; 10⁻¹² for Ir; 10⁻¹² for Os; 10⁻¹² for Pd; 10⁻¹² for Pt; 10⁻¹² for Rh; 5·10⁻⁹ for Ru; 10⁻¹² for Ir; 8·10⁻⁸ for Se; 8·10⁻¹⁰ for Tl; 10⁻¹² for Ba; 10⁻¹² for Cr; 10⁻¹² for Cu; 2·10⁻⁵ for Mo; 10⁻¹² for Sb; 10⁻¹² for Sn; 10⁻¹⁰ for Al; 4·10⁻³ for Ca; 2·10⁻¹ for K; 9·10⁻⁴ for Mn; 6·10⁻¹ for W; 10⁻⁴ for Zn.

In some cases, the high purity magnetosomes according to the invention preferentially produced by magnetotactic bacteria are amplified in a pre-growth and/or growth and/or fed-batch medium(a) comprising at least one metal or at least one compound selected among Co, Ni, Se, Cu, Mo, Al, Ca, Mg, and Mn at concentration(s) estimated in gram of compound or metal per liter of pre-growth and/or growth and/or fed-batch medium(a) that is or are larger than 0, 10⁻⁶, 10⁻⁵, 10⁻⁴, 10⁻³, 10⁻² or 10⁻¹ g of Co, 0, 10⁻⁶, 10⁻⁵, 10⁻⁴, 10⁻³, 10⁻² or 10⁻¹ g of Ni, 0, 10⁻¹⁰, 10⁻⁹, 10⁻⁸, 10⁻⁷, 10⁻⁶, 10⁻⁵, 10⁻⁴, 10⁻³, 10⁻² or 10⁻¹ g of Se, 0, 10⁻⁹, 10⁻⁸, 10⁻⁷, 10⁻⁶, 10⁻⁵, 10⁻⁴, 10⁻³, 10⁻² or 10⁻¹ g of Cu, 0, 10⁻¹⁰, 10⁻⁹, 10⁻⁸, 10⁻⁷, 10⁻⁶, 10⁻⁵, 10⁻⁴ or 10⁻³ g of Mo, 0, 10⁻¹⁰, 10⁻⁹, 10⁻⁸, 10⁻⁷, 10⁻⁶, 10⁻⁵, 10⁻⁴, 10⁻³, 10⁻² or 10⁻¹ g of Al, 0, 10⁻¹⁰, 10⁻⁹, 10⁻⁸, 10⁻⁷, 10⁻⁶, 10⁻⁵, 10⁻⁴, 10⁻³, 10⁻² or 10⁻¹ g of Mg, 0, 10⁻¹⁰, 10⁻⁹, 10⁻⁸, 10⁻⁷, 10⁻⁶, 10⁻⁵, 10⁻⁴, 10⁻³, 10⁻² or 10⁻¹ g of Mn, 0, 10⁻¹⁰, 10⁻⁹, 10⁻⁸, 10⁻⁷, 10⁻⁶, 10⁻⁵, 10⁻⁴, 10⁻³, 10⁻² or 10⁻¹ g of Zn, 0, 10⁻¹⁰, 10⁻⁹, 10⁻⁸, 10⁻⁷, 10⁻⁶, 10⁻⁵, 10⁻⁴, 10⁻³, 10⁻² or 10⁻¹ g of at least one metal.

The invention also relates to the high purity magnetosomes according to the invention produced by magnetotactic bacteria amplified in or using a fed-batch medium(a) comprising at least one compound selected among As, Cd, Hg, Pb, Co, Ni, V, Ag, Au, Ir, Os, Pd, Pt, Rh, Ru, Se, Tl, Ba, Cr, Cu, Li, Mo, Sb, Sn, Al, B, Ca, Fe, K, Mg, Mn, Na, W, and Zn at concentration(s) estimated in gram of compound per liter of fed-batch medium that is or are lower than: 7·10⁻⁶ for As; 10⁻¹² for Cd; 10⁻¹² for Hg; 10⁻¹² for Pb; 2·10⁻³ for Co; 4·10⁻⁴ for Ni; 10⁻¹² for V; 10⁻⁶ for Ag; 10⁻¹² for Au; 10⁻¹² for Ir; 10⁻¹² for Os; 10⁻⁵ for Pd; 10⁻¹² for Pt; 10⁻⁸ for Ru; 5·10⁻⁶ for Se; 10⁻⁷ for Ti; 10⁻⁶ for Ba; 10⁻⁵ for Cr; 5·10⁻⁶ for Cu; 2·10⁻⁶ for Li; 2·10⁻⁴ for Mo; 10⁻¹² for Sb; 10⁻¹² for Sn; 10⁻⁴ for Al; 3·10⁻⁴ for Ca; 3 for K; 10⁻² for Mn; 10⁻³ for Zn;

The invention also relates to the high purity magnetosomes according to the invention produced by magnetotactic bacteria amplified in or using a fed-batch medium(a) comprising at least one compound selected among: Cobalt, Molybdenum, and Manganese, at concentration(s) in the fed-batch medium that is/are:
at least 200, 500 or 1000 times smaller than the concentration of iron for Cobalt;
at least 2000, 5000 or 10000 times smaller than the concentration of iron for Molybdenum;
at least 50, 100 or 1000 times smaller than the concentration of iron for Manganese;

The invention also relates to high purity magnetosomes according to the invention preferentially produced by magnetotactic bacteria amplified in a pre-growth and/or growth and/or fed-batch medium(a) comprising at least one metal or compound preferentially selected among Co, Ni, Se, Cu, Mo, Al, Ca, Mg, and Mn at a concentration that is or are:
preferentially at least 0, 1, 1.01, 1.1, 2, 5, 10, 100 or 1000 time(s) smaller than the concentration of iron or Mg in the pre-growth and/or growth and/or fed-batch medium(a) for at least one metal, preferentially Co, Ni, Cu, Mo and/or Mn,
   and/or
preferentially at least 0, 1, 1.01, 1.1, 2, 5, 10, 100 or 1000 time(s) smaller than the concentration of iron or Mg in the pre-growth and/or growth and/or fed-batch medium for at least one metal, preferentially Se.

The invention also relates to high purity magnetosomes according to the invention preferentially produced by magnetotactic bacteria amplified in a pre-growth and/or growth and/or fed-batch medium(a) comprising at least one metal or compound preferentially selected among Ca, Na, P, a concentration that is larger preferentially at least 0, 1, 1.01, 1.1, 2, 5, 10, 100 or 1000 time(s) larger than the concentration of iron or Mg in the pre-growth and/or growth and/or fed-batch medium(a).

The invention also relates to the high purity magnetosomes according to the invention preferentially produced by magnetotactic bacteria amplified in a pre-growth and/or growth and/or fed-batch medium(a) comprising at least one metal or compound preferentially selected among As, Cd, Hg, Pb, Co, Ni, V, Ag, Au, Ir, Os, Pd, Pt, Rh, Ru, Se, Tl, Ba, Cr, Cu, Li, Mo, Sb, Sn, Al, B, Ca, Fe, K, Mg, Mn, Na, W, and Zn, with at least one property selected in the group consisting of:
i) at least one metal or one of these compounds, preferentially Se, has a concentration that is lower than 1, 10⁻¹, 10⁻², 10⁻³, 10⁻⁴, 10⁻⁵, 10⁻⁶, 10⁻⁷, 10⁻⁸ or 10⁻⁹ gram per liter of pre-growth, growth and/or fed-batch medium(a);
ii) at least three metals or three of these compounds, preferentially Se, Cu, and Mo, have concentrations that are lower than 1, 10⁻¹, 10⁻², 10⁻³, 10⁻⁴, 10⁻⁵, 10⁻⁶, 10⁻⁷, 10⁻⁸ or 10⁻⁹ gram per liter of pre-growth, growth and/or fed-batch medium(a);
iii) at least four metals or four of these compounds, preferentially Se, Cu, Mo, and Al, have concentrations that are lower than 1, 10⁻¹, 10⁻², 10⁻³, 10⁻⁴, 10⁻⁵, 10⁻⁶, 10⁻⁷, 10⁻⁸ or 10⁻⁹ gram per liter of pre-growth, growth and/or fed-batch medium(a).

In some cases, the high purity magnetosomes according to the invention preferentially produced by magnetotactic bacteria amplified in a pre-growth and/or growth and/or fed-batch medium(a) can comprise at least one metal or compound preferentially selected among As, Cd, Hg, Pb, Co, Ni, V, Ag, Au, Ir, Os, Pd, Pt, Rh, Ru, Se, Tl, Ba, Cr, Cu, Li, Mo, Sb, Sn, Al, B, Ca, Fe, K, Mg, Mn, Na, W, and Zn, with a concentration that is larger than 0, 1, 10⁻¹, 10⁻², 10⁻³, 10⁻⁴, 10⁻⁵, 10⁻⁶, 10⁻⁷, 10⁻⁸ or 10⁻⁹ gram per liter of pre-growth, growth and/or fed-batch medium(a); The invention also relates to the high purity magnetosomes according to the invention preferentially produced by magnetotactic bacteria using a fed-batch medium comprising at least one metal or compound preferentially selected among As, Cd, Hg, Pb, Co, Ni, V, Ag, Au, Ir, Os, Pd, Pt, Rh, Ru, Se, Tl, Ba, Cr, Cu, Li, Mo, Sb, Sn, Al, B, Ca, Fe, K, Mg, Mn, Na, W, and Zn, wherein:
at least one metal or one of these compounds, preferentially Se, has a concentration that is 10⁹, 10⁶, 10⁵, 10³, 100, 10, 5, 2 or 1 times lower than the concentration of iron or magnesium (Mg);
at least three metals or three of these compounds, preferentially Se, Cu, and Mo, have concentrations that are 10⁹, 10⁶, 10⁵, 10⁴, 10³, 100, 10, 5, 2 or 1 times lower than the concentration of iron or Mg;
at least four metals or four of these compounds, preferentially Se, Cu, Mo, and Al, have concentrations that are 10⁹, 10⁶, 10⁵, 10⁴, 10³, 100, 10, 5, 2 or 1 times lower than the concentration of iron or Mg.

The invention also relates to the high purity magnetosomes according to the invention preferentially produced by magnetotactic bacteria using a fed-batch medium comprising at least one metal or compound preferentially selected among As, Cd, Hg, Pb, Co, Ni, V, Ag, Au, Ir, Os, Pd, Pt, Rh, Ru, Se, Tl, Ba, Cr, Cu, Li, Mo, Sb, Sn, Al, B, Ca, Fe, K, Mg, Mn, Na, W, and Zn, wherein at least one metal or one of these compounds, has a concentration that is 0, 10⁹, 10⁶, 10⁵, 10³, 100, 10, 5, 2 or 1 times larger than the concentration of iron or magnesium (Mg); The invention also relates to the high purity magnetosomes according to the invention preferentially produced by magnetotactic bacteria amplified in a pre-growth and/or growth and/or fed-batch medium(a) comprising at least one metal, preferentially As, Cd, Hg, and/or Pb at concentration(s) that is/are lower than 10⁻⁹, 10⁻⁶, 10⁻³, 10⁻¹ or 1 gram per liter of per liter of pre-growth, growth and/or fed-batch medium(a).

The invention also relates to the high purity magnetosomes according to the invention preferentially produced by magnetotactic bacteria amplified in a pre-growth and/or growth and/or fed-batch medium(a) comprising at least one metal, preferentially As, Cd, Hg, and/or Pb, at concentration(s) that is/are larger than 0, 10⁻⁹, 10⁻⁶, 10⁻³, 10⁻¹ or 1 gram per liter of per liter of pre-growth, growth and/or fed-batch medium(a).

The invention also relates to high purity magnetosomes according to the invention, preferentially produced by magnetotactic bacteria amplified in a pre-growth and/or growth and/or fed-batch medium(a) preferentially comprising at least one metal, preferentially As, Cd, Hg, and/or Pb, at concentration(s) that is/are at least 1, 2, 5, 10, 10³, 10⁶ or 10⁹ lower than the concentration of iron or Mg (magnesium).

The invention also relates to high purity magnetosomes according to the invention, preferentially produced by magnetotactic bacteria amplified in a pre-growth and/or growth and/or fed-batch medium(a) preferentially comprising at least one metal, preferentially As, Cd, Hg, and/or Pb, at concentration(s) that is/are at least 0, 1, 2, 5, 10, 10³, 10⁶ or 10⁹ larger than the concentration of iron or Mg (magnesium).

The invention also relates to high purity nanoparticles produced through a chemical synthesis having at least one property in common with the magnetosome of high purity according to the invention, where this property is preferentially selected in the group consisting of: i) a nanoparticle composition, ii) a nanoparticle chain arrangement, and iii) a ferrimagnetic or ferromagnetic behavior.

The invention also relates to high purity nanoparticles produced through a chemical synthesis, where the composition of the medium used for their production has at least one property or compound or compound composition in common with the property or compound or compound composition of the pre-growth and/or growth and/or fed-batch medium(a) used to amplify magnetotactic bacteria and/or to produce or synthesize the high purity magnetosomes.

The invention also relates to the high purity nanoparticles produced through a chemical synthesis having at least one property in common with the magnetosome of high purity of claim 1, where this property is preferentially selected in the group consisting of: i) a nanoparticle composition, ii) a nanoparticle chain arrangement, and iii) a ferrimagnetic or ferromagnetic behavior.

The invention will be further disclosed by the following non-limiting examples.

### EXAMPLES

### Material and Methods:

Optical density measurements of suspensions of whole magnetotactic bacteria to assess bacterial growth: The optical density of the different suspensions of magnetotactic bacteria was measured at 565 nm, designated as OD_{565 nm}, using a Secomam UviLine9400 spectrophotometer. The value of OD_{565 nm} is proportional to the concentrations of bacteria in the suspensions.

Measurement of the magnetic response of living magnetotactic bacteria using optical microscopic observations of these bacteria under the application of a magnetic field: 1 mL of a suspension of MSR-1 magnetotactic bacteria was centrifugated at 14500 rpm for 10 minutes. The growth medium was removed and replaced by a volume of PBS 0.1× to reach an OD₅₆₅ nm of 0.5. 1 μL of this suspension of MSR-1 magnetotactic bacteria was deposited on a parallelipedic microscope slide (Menzel-Glaser, 24 mm×60 mm, 0.13-16 mm thickness) for microscopic observation using a Zeiss Primo Vert optical microscope with X40 magnification. Four small cubic Neodinium magnets of strength 1.3 T (Supermagnet, N42 W-10-N 10×10×10 mm) were positioned on the microscope's platform at a distance from the suspension of bacteria of ˜2 cm in such a way that it either generated a magnetic field parallel to the position of the observer or to the line between the two binoculars (position 1) or perpendicular to this position (position 2). 20 seconds after positioning the magnet in position 1 or 2, the percentage of bacteria aligned in the direction of the magnetic field was estimated, by considering 200 magnetotactic bacteria. The bacteria that were not aligned in the same direction as the magnetic field generated by the magnet were considered as non-magnetic. Their number is designated as n_{BNM}. The bacteria that were aligned in the direction of the magnetic field generated by the magnet were considered as magnetic. Their number was designated as n_{BM}. The percentage of magnetic bacteria was then given by n_{BM}/(n_{BM}+n_{BNM}). A positive magnetic response of magnetotatic bacteria corresponded to n_{BM}/(n_{BM}+n_{BNM})>0.5. A negative magnetic response of magnetotatic bacteria corresponds to n_{BM}/(n_{BM}+n_{BNM})<0.5.

In some cases, the percentage of magnetic bacteria can be measured by optical observation under microscope in the presence of a magnetic field.

Measurement of intracellular iron concentration: The iron concentration inside magnetotactic bacteria was determined by a destructive iron assay. For that, 2 mL of MSR-1 magnetotactic bacteria were centrifuged at 14500 g for 10 min. The bacterial pellet was then washed twice with 1×PBS and MilliQ water. After the second washing, the bacterial pellet was collected and 1 mL of 12N hydrochloric acid (HCl) was added to the pellet under the chemical hood. The sample was heated at 75° C. for 2 hours under stirring at 300 rpm to transform intracellular iron to Fe³⁺ and Fe²⁺ ions. The Fe²⁺ ions were then oxidized to Fe³⁺ with hydrogen peroxide (H₂O₂) at 20%. The presence of Fe³⁺ ions was revealed by the addition of potassium thiocyanate (KCN, 2 mol/L) in an acidic medium, which led to the formation of a red-orange solution whose color depended on the concentration of Fe³⁺ in the sample. As soon as the KCN was added, the absorbance of the solution was measured at 476 nm. The iron concentration in the sample was then estimated using a determined relation between the value of the absorbance measured at 476 nm and the concentration in iron (III) chloride. This method makes it possible to estimate the total intracellular iron concentration.

Analysis of the elemental chemical composition of the magnetosomes by ICP-AES: Following fermentation, MSR-1 magnetotactic bacteria were concentrated by tangential filtration in a volume of 5 L to reach an optical density between 25 and 30. Bacteria were then lysed during 1 hour in a 1M KOH solution under stirring at 150 rpm at a temperature of 80° C. The bacterial lysate containing the magnetosomes was placed against a Neodynium magnet for 12 hours. The magnetosomes were then separated from the bacterial lysate and re-suspended in 10×PBS This washing procedure was repeated two times with 10×PBS and three times in MilliQ water. The magnetosomes were then lyophilized and heated in a muffle furnace in conditions described below to obtain a magnetosome powder comprising high purity iron oxide crystals with a low content of carbon. For the analysis of the elemental chemical composition, a solution of 500 μg of this powder was mixed with 200 μl of 12N HCl and 10 ml of 2% filtered HNO₃. The ICP-AES measurement of the powder gives the quantity of chemical elements comprised in the magnetosomes, in μg of these chemical elements (Ag, Al, As, Ba, Cd, Co, Cr, Cu, Mn, Mo, Ni, Pb, Sb, Se, Si, Sn, Ti, Tl, W, and Zn) per g of iron comprised in the magnetosomes.

Chemical products used for the preparation of the growth media: Aluminum potassium sulfate dodecahydrate (AlK(SO₄)₂12H₂O, ref. NFG A6435, Merck); Ammonia hydroxide (NH₄OH, ref. NFG 1336-21-6, Acros Organics; ref. FG 105422, Merck); Ammonium chloride (NH₄Cl, ref. NFG A9434, Merck; ref. FG 1011420001, Merck); Ammonium sulfate ((NH₄)₂SO₄, ref. NFG A4418); Biotin (C₁₀H₁₆N₂O₃S, ref. NFG B4639, Merck; ref. FG B301, Merck); Boric acid (H₃BO₃, ref. NFG B6768, Merck); Calcium chloride (CaCl₂, ref. NFG 223506, Merck; ref. FG 1.42002, Merck); Calcium pantothenate (HOCH₂C(CH₃)₂CH(OH)CONHCH₂CH₂CO₂ 0.1/2Ca, reference FG C0400000, Merck); Cobalt(II) nitrate hexahydrate (Cobalt(II) nitrate hexahydrate, ref. FG 239267, Merck); Copper(II) sulfate pentahydrate (CuO₄S·5H₂O) (ref NFG C8027, Merck), DL-methionine (CH₃SCH₂CH₂CH(NH₂)COOH, ref. NFG M2768, Merck); DL-tryptophan (C₁H₁₂N₂O₂, ref. NFG T3300, Merck); EDTA ((HO₂CCH₂)₂NCH₂CH₂N(CH₂CO₂H)₂, ref. NFG E6758, Merck); Ferric citrate (C₆H₅FeO₇, ref. NFG F3388, Merck; ref. FG B301, Merck); Folic acid (C₁₉H₁₉N₇O₆, ref. NFG F7876, Merck; ref. FG F0300000, Merck); Inositol (C₆H₁₂₀₆, ref. FG PHR1351, Merck); Iron(II) sulfate heptahydrate (FeO₄S·7H₂O, ref. NFG F8633, Merck; ref. FG 1.03963, Merck); Iron(III) oxalate hexahydrate (Fe₂(C₂₀₄)₃.6H₂O, ref. NFG 381446, Merck); L-histidine (C₆H₉N₃O₂, ref. FG PHR1108, Merck); Magnesium sulfate heptahydrate (MgSO₄.7H₂O, ref. NFG 63138, Merck; ref. FG 105882, Merck); Manganese(II) sulfate monohydrate (MnO₄S·FHO, ref. NFG M7899, Merck); Nickel(II) chloride hexahydrate (Cl₂Ni·6H₂O, ref. NFG N6136, Merck); Nicotinic acid (C₆H₅NO₂, ref. NFG N4126, Merck); Nitrilotriacetic acid trisodium salt (C₆H₆NO₆Na₃, ref. NFG N0253, Merck); p-Aminobenzoic acid (H₂NC₆H₄CO₂H, ref. NFG A9878, Merck); Potassium phosphate dibasic (K₂HPO₄, ref. NFG P₈₂₈₁, Merck; ref. FG 105101, Merck); Potassium phosphate monobasic (KH₂PO₄, refe. NFG P₉₇₉₁, Merck); Protoporphyrin IX (C₃₄H₃₄N₄O₄, ref. NFG P₈₂₉₃, Merck); Pyridoxine HCl (C₁₂H₁₇ClN₄OS·HCl, ref. NFG P₉₇₅₅, Merck); Riboflavin (C₁₇H₂₀N₄O₆, ref. NFG R9504, Merck; ref. FG PHR1054, Merck); Sodium chloride (NaCl, ref. NFG S₇₆₅₃, Merck); Sodium lactate (C₃H₅NaO₃, ref. NFG L1375, Merck; ref. FG 106522, Merck); Sodium molybdate dihydrate (Na₂Mo₄·2H₂O, ref. NFG M1003, Merck); Sodium selenite pentahydrate (Na₂SeO₃·5H₂O, ref. FG 89771, Merck); Thiamine HCL (C₁₂H₁₇ClN₄OS·HCl, ref. NFG 47858, Merck, ref. FG PHR1037, Merck); Yeast extract (ref. NFG Y1625, Merck); Zinc sulfate heptahydrate (O₄SZn·7H₂O, ref NFG Z0251, Merck). NFG designates Non-pharmaceutical grade chemicals used to prepare the growth media; FG designates Pharmaceutical grade chemicals used for the preparation of the growth media. We also used deionized water (H₂O), with a resistivity of 15 MΩ.

Composition of the different mineral elixirs: The composition of the different mineral elixirs (V0, CB1, V2, CB2, CB3, CB4, CB5, CB7, CB9, CB10, CB11, CB12, CB13) is given in table 6, where the quantity (in gram) of the different chemicals used to prepare 1 liter of these elixirs is indicated.

Composition of the different yeast extracts: The composition of the different yeast extracts (YE, YNBWAA, YNBWoAA, YNBWoAA.AS) is given in table 7, where the quantity (in gram) of the different chemicals used to prepare 1 liter of these yeast extracts is indicated. YNBWAA, YNBWoAA, YNBWoAA.AS designate reduced yeast extracts while YE designates non-reduced yeast extract (reference: Y0875, Sigma). YE comprises nitrogenous compounds, carbon, sulfur, trace nutrients, vitamin B complex and other important growth factors.

Composition of the different vitamin cocktails: The composition of the different vitamin cocktails (Vit1×, Vit5×, Vit10×, Vit0.5×, Vit0.1×) is given in table 8, where the quantity (in gram) of the different chemicals used to prepare 1 liter of these vitamin cocktails is indicated.

Composition of pre-growth media for condition 1 (table 1): One liter of pre-growth media comprises in one liter of deionized water 2.6 g of sodium lactate, 0.4 g of ammonium chloride, 0.1 g of magnesium sulfate heptahydrate, 0.5 g of potassium phosphate dibasic, 0.1 g of yeast extract YE (Table 7), and 0.5 mL of either one of the mineral elixirs V0, CB1, V2, CB2, CB3, CB4, CB5, CB7, CB9, CB10, CB11, CB12, or CB13 (Table 6).

Composition of growth media for condition 1 (table 1): One liter of growth media comprises in one liter of deionized water 2.6 g of sodium lactate, 0.4 g of ammonium chloride, 0.1 g of magnesium sulfate heptahydrate, 0.5 g of potassium phosphate dibasic, 0.1 g of yeast extract YE (Table 7), 0.5 mL of either one of the mineral elixirs V0, CB1, V2, CB2, CB3, CB4, CB5, CB7, CB9, CB10, CB11, CB12, or CB13 (Table 6), and 10 mL of ferric citrate (20 mM initial concentration).

Composition of pre-growth media for condition 2 (table 2): One liter of pre-growth media comprises in one liter of deionized water: 2.6 g of sodium lactate, 0.4 g of ammonium chloride, 0.1 g of magnesium sulfate heptahydrate, 0.5 g of potassium phosphate dibasic, 0.1 g of either one of yeast extract YE, YNBWAA, YNBWoAA, YNBWoAA.AS (Table 7), and 0.5 mL of mineral elixir CB3 (Table 6).

Composition of growth media for condition 2 (table 2): One liter of growth media comprises in one liter of deionized water 2.6 g of sodium lactate, 0.4 g of ammonium chloride, 0.1 g of magnesium sulfate heptahydrate, 0.5 g of potassium phosphate dibasic, 0.1 g of either one of yeast extracts YE, YNBWAA, YNBWoAA, or YNBWoAA.AS (Table 7,), 0.5 mL of mineral elixir CB3 (Table 6)., and 10 mL of ferric citrate (20 mM initial concentration).

Composition of pre-growth media for condition 3 (table 3): One liter of pre-growth media comprises in one liter of deionized water 2.6 g of sodium lactate, 0.4 g of ammonium chloride, 0.1 g of magnesium sulfate heptahydrate, 0.5 g of potassium phosphate dibasic, 0.1 mL of either one of vitamins Vit1×, Vit5×, Vit10×, Vit5×, Vit0.5×, or Vit0.1× (Table 8), and 0.5 mL of mineral elixir CB3 (Table 6).

Composition of growth media for condition 3 (table 3): One liter of growth media comprises in one liter of deionized water 2.6 g of sodium lactate, 0.4 g of ammonium chloride, 0.1 g of magnesium sulfate heptahydrate, 0.5 g of potassium phosphate dibasic, 0.1 mL of either one of vitamin cocktails Vit1×, Vit5×, Vit10×, Vit5×, Vit0.5×, or Vit0.1× (Table 8), 0.5 mL of mineral elixir CB3 (Table 6), and 10 mL of ferric citrate (20 mM initial concentration).

Composition of pre-growth media for condition 4 (table 4): One liter of pre-growth media comprises in one liter of deionized water 2.6 g of sodium lactate, 0.4 g of ammonium chloride, 0.1 g of magnesium sulfate heptahydrate, 0.5 g of potassium phosphate dibasic, 0.1 mL of either one of individual vitamins Bt, CP, FA, I, NA, AA, P, R, or T (Table 9), 0.5 mL of mineral elixir CB3 (Table 6).

Composition of growth media for condition 4 (table 4): One liter of growth media comprises in one liter of deionized water 2.6 g of sodium lactate, 0.4 g of ammonium chloride, 0.1 g of magnesium sulfate heptahydrate, 0.5 g of potassium phosphate dibasic, 0.1 mL of either one of the individual vitamins Bt, CP, FA, I, NA, AA, P, R, or T (Table 9), 0.5 mL of mineral elixir CB3 (Table 6), and 10 mL of ferric citrate (20 mM initial concentration).

Composition of pre-growth media for condition 5 (table 5): The composition of the different pre-growth media with various concentrations of the main components of the pre-growth media, i.e. sodium lactate, ammonium chloride, magnesium sulfate heptahydrate, potassium phosphate dibasic (N, SL0, SL0.5×, SL0.2×, SL0.1×, AC0, AC0.5×, AC0.2×, AC0.1×, MG0, MG0.5×, MG0, MG0.5×, MG0.2×, MG0.1×, P, P0.5×, P0.2×, P0.1×) is given in table 5, where the quantity (in gram) of the different chemicals used to prepare 1 liter of these pre-growth media is indicated.

Composition of growth media for condition 5 (table 5): The composition of the different growth media with various concentrations of the main components of the pre-growth media, i.e. sodium lactate, ammonium chloride, magnesium sulfate heptahydrate, potassium phosphate dibasic (N, SL0, SL0.5×, SL0.2×, SL0.1×, AC0, AC0.5×, AC0.2×, AC0.1×, MG0, MG0.5×, MG0, MG0.5×, MG0.2×, MG0.1×, P, P0.5×, P0.2×, P0.1×) is given in table 5, where the quantity (in gram) of the different chemicals used to prepare 1 liter of these growth media is indicated.

Composition of pre-growth media, growth media and fed-batch medium for condition 6, prepared using non-pharmaceutical grade chemicals (table 14(a)): The pre-growth media B1 and B4 comprise in one liter of deionized water 2.6 g of sodium lactate, 0.4 g of ammonium chloride, 0.1 g of magnesium sulfate heptahydrate, 0.5 g of potassium phosphate dibasic, 0.1 mL of vitamin cocktail Vit 0.1× (Table 8), and 0.5 mL of mineral elixir CB3 (Table 6). The growth media B1 and B4 comprise in one liter of deionized water 104 g of sodium lactate, 16 g of ammonium chloride, 1.2 g of magnesium sulfate heptahydrate, 2.8 g of potassium phosphate dibasic, 3.2 mL of vitamin cocktail Vit0.1× (Table 8), 2.8 mL of mineral elixir CB3 (Table 6). The fed-batch media B1 and B4 comprise in one liter of water 100 g of lactic acid, 4.8 g of ammoniac, 6 g of potassium phosphate dibasic, 2.4 of magnesium sulfate heptahydrate, 1 mL of vitamin cocktail Vit0.1× (Table 8), 7 mL of mineral elixir CB3 (Table 6), and either 1.8 g of ferric citrate (B1) or 2 g of iron III chloride (B4).

Composition of pre-growth media, growth media and fed-batch medium for condition 6, prepared using pharmaceutical grade chemicals (table 14(b)): The pre-growth media B2 and B3 comprise in one liter of deionized water 2.6 g of sodium lactate, 0.4 g of ammonium chloride, 0.1 g of magnesium sulfate heptahydrate, 0.5 g of potassium phosphate dibasic, 0.1 mL of vitamin cocktail Vit 0.1× (Table 8), and 0.5 mL of mineral elixir CB3 (Table 6). The growth media B1 and B4 comprise in one liter of deionized water 104 g of sodium lactate, 16 g of ammonium chloride, 1.2 g of magnesium sulfate heptahydrate, 2.8 g of potassium phosphate dibasic, 3.2 mL of vitamin cocktail Vit0.1× (Table 8), 2.8 mL of mineral elixir CB3 (Table 6). The fed-batch media B1 and B4 comprise in one liter of water 100 g of lactic acid, 4.8 g of ammoniac, 6 g of potassium phosphate dibasic, 2.4 of magnesium sulfate heptahydrate, 1 mL of vitamin cocktail Vit0.1× (Table 8), 7 mL of mineral elixir CB3 (Table 6), and either 1.8 g of ferric citrate (B2) or 2 g of iron III chloride (B3).

Stock of MSR-1 magnetotactic bacteria used for the different cultures: MSR-1 magnetotactic bacteria are commercialized by the company DSMZ under the reference DSM 6361. After reception, the MSR-1 bacterial suspensions were stored at an OD_{565 nm} (optical density measured at 565 nm) of 0.01, corresponding approximately to a bacterial concentration of 5·10⁷ bacteria per ml of culture medium (DSMZ Medium 380 used to grow the MSR1 Magnetospirillum Mediums strain DSMZ 6361) in a −80° C. freezer in 15 mL tubes (5 mL of bacterial suspension per tube) or in 1.5 mL Eppendorf tubes (600 μL of bacterial suspension per tube). Suspensions of MSR-1 bacteria stored in the freezer at −80° C. constitute the cellular stock. In some cases, the culture and/or pre-culture growth medium/media can be the same as the growth and/or pre-growth medium/media.

Notation: the number X after D in DX designates the number of day following the beginning of the pre-growth step, preferentially the day at which magnetotactic bacteria are inserted in the pre-growth medium for the first time or during the first sub-step of the pre-growth step.

### Example 1: Determination of the Minimum Mineral Elixir Enabling Bacterial Growth and Magnetosome Synthesis

This example describes the experimental protocol used to reduce as much as possible the mineral elixir composition while enabling the growth of MSR-1 magnetotactic bacteria and the synthesis of magnetosomes by these bacteria. In this example non-pharmaceutical grade chemicals were used for the preparation of the growth media. The composition of 1 liter of pre-growth and growth media used in this example (condition 1) are indicated in table 1. During the first day of the experiment (D1), a first step consisted in collecting the tubes of 15 ml containing 5 mL of MSR-1 cellular stock tube from the freezer at −80° C., in thawing the tubes by letting them at room temperature for 10 minutes. In a hood, we collected from these tubes 100 μl comprising 5·10⁶ MSR-1 magnetotactic bacteria that we have inserted in a 50 mL tube comprising 8 mL of filtered pre-growth medium. As a whole, 13 different culture conditions were tested corresponding to the 13 different mineral elixirs tested. The 50 ml tubes were incubated for 6 days between D1 and D6 in an incubator at 29.5° C. under shaking conditions at 150 rpm. A second step consisted in adding an iron source to the growth media to enable the synthesis of magnetosomes by MSR-1 bacteria. After 6 days of pre-growth, at D6, the 50 ml tubes were placed in a hood. 30 ml of filtered culture medium were then added to the 50 mL tubes and the bacteria grew between D6 and D13. A positive magnetic response at D13 and a ratio between the optical density at D13 and the optical density at D6 larger than 1 was observed for V0, V2, CB2, CB3, CB4, CB5, CB7, CB10, CB11, CB12, CB13 (condition 1). In contrast, no magnetic response for conditions CB1 and CB9, where the concentrations of chemical elements that comprise the mineral elixirs are lower than 10⁻⁵ g/l. In conclusion, the minimal mineral elixir that enables the growth of MSR-1 bacteria with a large increase in optical density (OD_{565 nmD13}/OD_{565 nmD6} larger than 4.8) and the synthesis of magnetosomes (positive magnetic response) is CB13, composed only of iron sulfate heptahydrate at a concentration of 1 g/l and of calcium chloride at a concentration of 20 g/l.

### Example 2: Determination of a Growth Medium without Yeast Extract, Enabling the Growth of Magnetotactic Bacteria and the Synthesis of Magnetosomes

This example describes the experimental protocol used to determine the reduced medium replacing the yeast extract that enables the growth of MSR-1 magnetotactic bacteria and the synthesis of magnetosomes by these bacteria. In this example we used non-pharmaceutical grade chemicals for the preparation of the growth media. The compositions of pre-growth and growth media, in one liter of de-ionized water, are indicated in table 2 (condition 2), table 3 (condition 3), and table 4 (condition 4). During the first day of the experiment (D1), a first step consists in collecting the tubes of 15 ml containing 5 mL of MSR-1 cellular stock tube from the freezer at −80° C., in thawing the tubes by letting them at room temperature for 10 minutes. In a hood, we collected from these tubes 100 μl comprising 5·10⁶ MSR-1 magnetotactic bacteria that we have inserted in a 50 mL tube comprising 8 mL of filtered pre-growth medium of either condition 2 (table 2), condition 3 (table 3), or condition 4 (table 4). The 50 ml tubes were incubated for 6 days between D1 and D6 in an incubator at 29.5° C. under shaking conditions at 150 rpm. A second step consisted in adding an iron source to the growth media to enable the synthesis of magnetosomes by MSR-1 bacteria. After 6 days of pre-growth, at D6, the 50 ml tubes were placed in a hood. 30 ml of filtered growth medium were then added to the 50 mL tubes (condition 2, table 2, condition 3, table 3, condition 4, table 4) at D6 and the bacteria grew between D6 and D13. Tables 11 and 12 indicate that a magnetic response larger than 90% at D13 and a ratio between the optical density measured at D13 and the optical density measured at D6 larger than 1, was observed for yeast extracts YE, YNBWAA, YNBWAA, YNBWoAA, YNBWoAA, YNBWoAA.AS (condition 2), for Vit1×, Vit0.5×, Vit0.1×, (condition 3), Biotin (Bt), folic acid (FA), nicotinic acid (NA), Riboflavin (R), Thiamine HCl (T) (condition 4). In contrast, the magnetic response is 0 for conditions Vit5×, Vit10× (condition 3) and very low for conditions CP, I, AA, P (condition 4). In conclusion, the yeast extract can be replaced by a single vitamin, which is biotin, folic acid, riboflavin, nicotinic acid or thiamin HCl. These vitamins yield values of OD_{565 nmD13}/OD_{565 nmD6} of 9.8 (biotin), 2.9 (folic acid), 4.8 (riboflavin), 2.4 (nicotinic acid), 5.8 (thiamin HCl) and 90% of magnetic response (table 11).

### Example 3: Determination of the Minimal Concentrations of the Main Components of the Growth Media (Sodium Lactate, Ammonium Chloride, Magnesium Sulfate, Potassium Phosphate), Enabling the Growth of Magnetotactic Bacteria and the Synthesis of Magnetosomes by these Bacteria

This example describes the experimental protocol used to determine the reduced growth medium, enabling the growth of MSR-1 magnetotactic bacteria and the synthesis of magnetosomes by these bacteria. In this example we used non-pharmaceutical grade chemicals for the preparation of the growth media. We varied the concentration of sodium lactate (conditions SL0, SL0.5×, SL0.2×, SL0.1×), ammonium chloride (AC0, AC0.5×, AC0.2×, AC0.1×), magnesium sulfate heptahydrate (MG0, MG0.5×, MG0.2×, MG0.1×), potassium phosphate dibasic (P0, P0.5×, P0.2×, P0.1×). The chemical compositions and concentrations of the pre-growth and growth media N, SL0, SL0.5×, SL0.2×, SL0.1×, AC0, AC0.5×, AC0.2×, AC0.1×, MG0, MG0.5×, MG0.2×, MG0.1×, P0, P0.5×, P0.2×, P0.1×, are summarized in table 5 for 1 liter of growth media. During the first day of the experiment (D1), a first step consists in collecting the tubes of 15 ml containing 5 mL of MSR-1 cellular stock tube from the freezer at −80° C., in thawing the tubes by letting them at room temperature for 10 minutes. Under a hood, we collected from these tubes 100 μl comprising 5·10⁶ MSR-1 magnetotactic bacteria that we have inserted in a 50 mL tube filled with 8 mL of pre-growth media (condition 5, table 5). The 50 ml tubes were incubated for 6 days between D1 and D6 in an incubator at 29.5° C. under shaking conditions at 150 rpm. A second step consisted in adding an iron source to the growth media to enable the synthesis of magnetosomes by MSR-1 bacteria. After 6 days of pre-growth, at D6, the 50 ml tubes were placed in a hood. 30 ml of filtered growth media were then added to the 50 mL tubes at D6 and the bacteria grew between D6 and D13. For conditions N, P0.5×, P0.2×, a growth ratio, or ratio between the optical density measured at D13 and the optical density measured at D6, was larger than 1, and a positive magnetic response (magnetic response >90%) was observed at D13. It indicates that these conditions enabled the bacteria to grow and produce magnetosomes. By contrast, for conditions SL0, SL0.5×, SL0.2×, SL0.1×, AC0, AC0.5×, AC0.2×, AC0.1×, MG0, MG0.5×, MG0.2×, MG0.1×, P0.1×, P0, the synthesis of the magnetosomes was very low (magnetic response <50%). In conclusion, the concentration of potassium phosphate can be reduced in the growth medium by a factor of 2 or 5 without affecting the growth and production of magnetosomes. Indeed, these conditions yield values of OD_{565 nmD13}/OD_{565 nmD6} of 1.5 (condition P0.5×), 2.1 (condition P0.2×), and a percentage of positive magnetic response among bacteria larger than 90% (table 13). By contrast, the concentrations of the other chemicals of the medium (ammonium chloride, sodium lactate, magnesium phosphate) could not be reduced without significantly affecting the growth and/or the magnetic response of MSR-1 magnetotactic bacteria.

### Example 4: Determination of the Iron Source in 1-Liter Fermenters Enabling the Growth of Magnetotactic Bacteria and the Synthesis of Magnetosomes by these Bacteria and of the Reduction of Impurities Obtained by Using High Pharmaceutical Grade Chemical Products for the Preparation of the Pre-Growth and/or Growth Medium/Media

This example describes the experimental protocol used to determine the iron source, which enables the growth of MSR-1 magnetotactic bacteria and the synthesis of magnetosomes by these bacteria as well as the reduction of impurity(ies) comprised in the magnetosomes obtained by using pharmaceutical grade chemicals (condition 6). In this example we used pharmaceutical grade chemicals to prepare B2 and B3 growth media (table 14(b)) and non-pharmaceutical grade chemicals to prepare B1 and B4 growth media (table 14(a)). The compositions of the pre-growth medium, growth medium, and fed-batch medium are indicated for 1 liter of medium in tables 14(a) and 14(b). During the first day (D1), a first step of pre-growth consists in collecting 1 tube of 1.5 mL Eppendorf containing 600 μL of MSR-1 cellular stock tube from the freezer at −80° C., in thawing the tube by letting them at room temperature for 10 minutes. Under a hood, we collected from these tubes 300 μl comprising 1.5·10⁷ MSR-1 magnetotactic bacteria that we have inserted in a 500 mL sterile bottle filled with 250 mL of filtered pre-growth medium. The 500 ml bottle was incubated for 7 days between D1 and D7 in an incubator at 29.5° C. A second step of pre-growth was then carried out in a larger 2 L bottle. After 7 days of pre-growth, at D8, the 500 mL bottle was placed in a hood. The pre-growth medium containing the MSR-1 bacteria was manually transferred in a 2 L sterile bottle filled with 1.5 L of filtered pre-growth medium for the second step of pre-growth. The 2 L bottle was incubated for 1 day between D8 and D9 in an incubator at 29.5° C. under shaking conditions at 150 rpm. During the ninth day (D9), the growth step started. For that, 4 fermenters (conditions, B1, B2, B3, B4) of 1.5 L were filled with 780 mL of deionized water and autoclaved. The fermenters were then filled with 20 mL of filtered growth medium. Each of the 4 fermenters (conditions, B1, B2, B3, B4) was then filled with 200 mL of pre-growth medium containing the MSR-1 bacteria originating from the second step of pre-growth. Between day D9 and D11, an acidic fed-batch medium comprising an iron source was added to the growth medium to enable the synthesis of the magnetosomes by MSR-1 bacteria, while maintaining the pH of the growth medium at 6.9. During the growth step, the temperature was maintained at 29.5° C., airflow at 0.05 mL/min and agitation at 200 rpm. The optical densities, measured at 565 nm, of the bacterial suspensions at different days of the pre-growth step (DO and D8) and growth step (D9, D10, D11) are indicated in table 15 for conditions B1, B2, B3, and B4. Following fermentation at D13, MSR-1 cells from fermenters B1, B2, B3, and B4 (conditions B1 to B4) were concentrated by centrifugation at 4000 rpm during 45 min. To lyse the bacteria, MSR-1 cells from fermenters B1, B2, B3, B4, were re-suspended in 15 mL of 1 M KOH solution and heated at 80° C. during 2 h in a sonicating bath at 25 kHz in 20 mL glass bottles. After bacterial lysis, magnetosomes from MSR-1 cells were separated from the organic material using a Neodymium magnet over-night. At D14, magnetosomes from conditions B1, B2, B3, B4 were washed two times using 15 mL of 10× Phosphate-buffered saline and two times using 15 mL of deionized water using a Neodymium magnet. During each wash, the magnetosome suspensions were positioned against a Neodymium magnet for 2 hours that attracts the magnetosomes. The supernate containing organic debris were discarded and replaced either by 15 mL of 10× Phosphate-buffered saline or 15 mL of deionized water. At D16, after the last wash the supernate were discarded and magnetosomes from conditions B1, B2, B3, B4 were transferred in ceramic cups and dried by positioning them against a Neodymium magnet over-day. At D17, the remaining liquid were discarded and magnetosomes were inserted in ceramic cups and positioned in a muffle furnace and heated at 200° C. for 30 min, 300° C. for 1 hour, and 380° C. for 1 hour. At D17, ˜1 mg of purified magnetosomes from conditions B1, B2, B3, B4 were inserted in 15 mL tubes filled with 200 μL of HCL 12N. The tubes of 15 mL containing the magnetosomes were vortexed and incubated at room temperature for 2 hours and then filled with 9.8 mL of HNO₃ 2%. After that, the concentration in μg of elemental impurities per gram of nanoparticles were measured by ICP-AES. The results of these measurements are indicated in table 16 for conditions B1, B2, B3, B4, where elemental impurities are Ag (silver), Al (aluminium), As (arsenic), Ba (baryum), Cd (cadmium), Co (cobalt), Cr (chrome), Cu (copper), Mn (manganese), Mo (molybdenum), Ni (nickel), Pb (lead), Sb (antimony), Se (selenium), Si (silica), Sn (tin), Ti (titanium), Tl (tallium), W (tungstate), Zn (zinc). In conclusion, the condition B3 yields the largest value of OD_{565 nmD11}/OD_{565 nmD9} of 26.8 and a percentage of positive magnetic response among bacteria (>90%) (table 15), indicating that Iron(III) chloride is the best iron source. Furthermore, except for Pb, elemental impurity concentrations are reduced in condition B3 where pharmaceutical grade chemicals were used compared with condition B4 where non-pharmaceutical grade chemicals were used (table 16).

### Examples 5 (Purifying Method)

### Materials and Methods:

Note: In this example, weight could be replaced by mass, preferentially leading to the same meaning.

### Equipment Used to Analyze and Heat the Various Samples:

TGA-DSC: “Thermogravimetric analysis” (TGA) coupled to “Differential Scanning Calorimetry” (DSC) is used to measure heat flow (in mW) or the percentage of mass loss of powders comprising lyophilized magnetosomes (treated or not) or lyophilized whole bacteria or lyophilized SIGMA nanoparticles as a function of the heating temperature of these powders. For the measurements, the powders are heated at a rate of 6° C. per minute between 20° C. and 600° C. The derivative of the percentage of mass variation of the powders is also plotted as a function of temperature. The TGA-DSC profiles make it possible to define the temperatures for which the material, preferentially organic material, located in or at the surface of the magnetosomes or nanoparticles will be degraded, removed from the nanoparticles, or transformed. ATG and DSC analyzes were performed with the SDT Q600 (TA Instrument). It consists of a sealed enclosure, a furnace with temperature control, a micro-balance, and a thermocouple to measure the temperature. A mass of 3 mg of lyophilized bacteria, lyophilized magnetosomes, SIGMA nanoparticle powder, is used for TGA-DSC analysis

CHNS: “Elemental Carbon, Hydrogen Nitrogen and Sulfur Analyzer” CHNS measurements are carried out using a CHNS analyzer (Flash Elemental Analyzer EA 1112 from Thermo Fischer scientific) using a mass of 3 mg per measurement of lyophilized magnetosomes (conditions of treatment no 1, 2, 3, 4, 5, 6, 7, 8, 9, 10, 11), lyophilized whole bacteria, lyophilized SIGMA nanoparticles (not treated). CHNS measurements enable to determine the percentages in mass of carbon and nitrogen in the different powders. A mass of 3 mg of lyophilized bacteria, lyophilized magnetosomes, and SIGMA nanoparticle powder, is used for CHNS measurements.

Furnace: A muffle furnace (Nabertherm L9/11/B410) is used to heat 30 mg or 500 mg of magnetosomes extracted from magnetotactic bacteria following condition no 1 or 2 without heat treatment above 200° C. or with heat treatment above 200° C. (following condition no 3, 4, 5, 6, 7, 8, 9, 10 or 11). For that, the 30 or 500 mg powder of each sample is deposited in an uncovered porcelain cup and placed at the center of the furnace. A program is used to carry out the different heating conditions. The furnace enables to maintain the temperature of the nanoparticles or the temperature inside the furnace at a given temperature plus or minus 2° C., or the furnace enables to obtain a stable temperature between 20° C. and 380° C. with a fluctuation of 2° C. maximum.

Sample comprising whole magnetotactic bacteria (Sample 0): Magnetotactic bacteria obtained from condition 1 (mineral elixir V2, table 1) were collected and concentrated using a tangential filtration system to an optical density, measured at 565 nm (OD_{565 nm}), between 100 and 200. Sample 0 comprises concentrated whole magnetotactic bacteria.

Samples Comprising Magnetosomes Extracted from Magnetotactic Bacteria without Heat Treatment Above 200° C. (Samples 1 and 2):

Condition 1 of lysis (Sample 1): 100 ml of sample 0, concentrated at an OD_{565 nm} of 120, were mixed with 400 ml of 5M NaOH and sonicated and heated at 60° C. for 1 hour using a sonic bath to lyse the bacteria. The treated magnetosomes were then isolated from the bacterial debris by placing a Neodinium magnet overnight against the wall of the container containing the lysed bacteria suspension and by replacing the supernatant containing the medium and bacterial debris by 1×PBS. The resulting suspension was then sonicated for 20 seconds at 10W in the presence of 1×PBS, placed against a Neodinium magnet for 15 minutes, the supernatant was removed and the treated magnetosomes were resuspended in 1×PBS. This sequence of sonication and magnetic separation was repeated four times. For an entire fermenter, this treatment was repeated 10 times in 10 different volumes. Pyrogenic magnetosome chains extracted from MSR-1 magnetotactic bacteria were thus obtained, i.e. approximately 500 mg in iron of magnetosomes comprised in 1.7 ml of water. Sample 1 comprises magnetosomes obtained from condition 1 of lysis.

Condition 2 of lysis (Sample 2): Concentrated magnetotactic bacteria were frozen at −80° C. for 48 hours. After thawing and dilution of the concentrate with MilliQ water to obtain an OD₅₆₅ nm of 30, an amount of KOH was added to obtain to the concentrated bacteria to obtain a final KOH concentration of 1M. This solution was transferred to a polypropylene (PP) bottle and placed in the water bath at 80° C. with stirring at 150 rpm with a mechanical stirring pad (Fisher Scientific), for 30 minutes. Then, the content of the bottle was transferred into 4 other glass bottles of 2 L. Each bottle was placed against a NdFeB magnet for 12 hours to magnetically separate the extracted magnetosomes from bacterial debris. The magnetosomes were then washed 6 times in 500 mL bottles by magnetic selection, until a clear supernatant was obtained. The first two washes were carried out with 10×PBS, which makes it possible to return to a neutral pH. Then the other four washes were carried out with water. After lysis, the basic pH of the lysate, caused by the KOH, was brought back to a neutral pH so as not to damage the magnetosomes. Pyrogenic magnetosome chains extracted from the strain MSR-1 were thus obtained, i.e. about 500 mg in iron of magnetosomes in 1.7 ml. Sample 2 comprises magnetosomes obtained after condition 2 of lysis.

Samples Comprising Magnetosome Extracted from Magnetotactic Bacteria and Treated with Phenol-Chloroform (Condition 3):

Condition 3 of treatment (Sample 3): 100 μl of the suspension containing 30 mg in iron of magnetosomes obtained following condition 1 of lysis were mixed with 200 ml of a solution containing 1% Triton X-100 and 1% SDS. The mixture was heated overnight at 50° C., was placed against a Neodinium magnet, the supernatant was removed and replaced with 80 mL of phenol at pH 8. The obtained suspension was heated for 2 hours under sonication at 60° C., held overnight at 60° C. without sonication, placed against a magnet, the supernatant of the suspension was removed and replaced with 80 mL of chloroform. The suspension containing the chloroform was placed against a magnet of Neodinium, the supernatant was removed and the residual chloroform adsorbed at the surface of the treated magnetosomes was removed by heating these magnetosomes for 2 hours under a hood. Finally, the cores of the magnetosomes thus obtained were desorbed from the glass wall from the tubes containing them by adding 80 ml of 1M NaOH heated for 1 hour at 60° C. in a sonic bath. The suspension containing the cores of the magnetosomes was placed against a Neodinium magnet. The supernatant was removed and replaced with sterile MilliQ water. The suspension was sonicated for 20 seconds at 10 W. This washing sequence was repeated four times. Purified pyrogen-free magnetosomes were obtained in a small volume of pyrogen-free water. Sample 3 comprises magnetosomes obtained after condition 3 of treatment.

Samples Comprising Magnetosome Extracted from Magnetotactic Bacteria and Heated at Temperatures Above 200° C. (Samples 4 to 11):

Condition 4 of heat treatment (Sample 4): 100 μl of the suspension containing approximately 30 mg in iron of magnetosomes extracted from MSR-1 magnetotactic bacteria following condition 2 of lysis were lyophilized, introduced into a porcelain crucible, and baked in the furnace Nabertherm L9/11/B410. The heating protocol was as follows. The temperature of the furnace was increased from 20° C. to 200° C. at a rate of 6° C./min until the temperature of the furnace reaches 200° C. and the temperature of 200° C. in the furnace was maintained during one hour. Then the temperature of the furnace was decreased from 200° C. to 25° C. in 12 hours. Sample 4 comprises magnetosomes obtained after condition 4 of treatment.

Condition 5 of heat treatment (Sample 5): 100 μl of the suspension containing approximately 30 mg in iron of magnetosomes extracted from MSR-1 magnetotactic bacteria following condition 2 of lysis were lyophilized, introduced into a porcelain crucible, and baked in the furnace Nabertherm L9/11/B410. The heating protocol was as follows. The temperature of the furnace was increased from 20° C. to 400° C. at a rate of 6° C./min until the temperature of the furnace reaches 400° C. The temperature in the furnace was maintained at 400° C. for one hour. Then the temperature of the furnace was decreased from 400° C. to 25° C. in 20 hours. Sample 5 comprises magnetosomes obtained after condition 5 of treatment.

Condition 6 of heat treatment (Sample 6): 100 μl of a suspension comprising 30 mg in iron of magnetosomes prepared according to condition 2 of lysis, were lyophilized and then introduced into a porcelain crucible and heated in the furnace Nabertherm L9/11/B410. The heat treatment was as follows. The temperature of the furnace was increased from 20° C. to 200° C. in 20 min at a rate of 9° C./min. The temperature of the furnace was then maintained at 200° C. for 30 minutes. The temperature of the furnace was then increased from 200° C. to 300° C. in 10 min at a rate of 10° C./min. The temperature of the furnace was then maintained at 300° C. for 1 hour. Then the temperature of the furnace was decreased from 300° C. to 25° C. in 12 hours. Sample 6 comprises magnetosomes obtained after condition 6 of treatment.

Condition 7 of heat treatment (Sample 7): 100 μl of a suspension comprising 30 mg in iron of magnetosomes prepared according to condition 2 of lysis, were lyophilized and then introduced into a porcelain crucible and heated in the furnace Nabertherm L9/11/B410. The heat treatment was as follows. The temperature of the furnace was increased from 20° C. to 200° C. in 20 min at a rate of 9° C./min. The temperature of the furnace was then maintained at 200° C. for 30 minutes. The temperature of the furnace was then increased from 200° C. to 300° C. in 10 min at a rate of 10° C./min. The temperature of the furnace was then maintained at 300° C. for 1 hour. The temperature of the furnace was then increased from 300 to 380° C. in 10 min at a rate of 8° C./min. The temperature of the furnace was then maintained at 380° C. for 1 hour. The temperature of the furnace was then increased from 380° C. to 550° C. in 20 min at a rate of 8.5° C./min. The temperature of the furnace was then maintained at 550° C. for 1 h. Then the temperature of the furnace was decreased from 550° C. to 25° C. in 20 hours. Sample 7 comprises magnetosomes obtained after condition of treatment

Condition 8 of heat treatment (Sample 8): 100 μl of the suspension containing 30 mg in iron of magnetosomes prepared according to condition 2 of lysis, were lyophilized and then introduced into a porcelain crucible and baked in the furnace Nabertherm L9/11/B410. The heat treatment was as follows. The temperature of the furnace was increased from 20° C. to 200° C. in 20 min at a rate of 9° C./min. The temperature of the furnace was then maintained at 200° C. for 30 minutes. The temperature of the furnace was then increased from 200° C. to 300° C. in 10 min at a rate of 10° C./min. The temperature of the furnace was then maintained at 300° C. for 1 hour. The temperature of the furnace was increased from 300° C. to 380° C. in 10 min at a rate of 8° C./min. The temperature of the furnace was then maintained at 380° C. for 1 h. Then the temperature of the furnace was decreased from 380° C. to 25° C. in 12 hours. Sample 8 comprises magnetosomes obtained after condition 8 of treatment.

Condition 9 of heat treatment (Sample 9): 100 μl of the suspension containing 30 mg in iron of magnetosomes prepared according to condition 1 of lysis, were lyophilized and then introduced into a porcelain crucible and heated in the furnace Nabertherm L9/11/B410. The heat treatment was as follows. The temperature of the furnace was increased from 20° C. to 200° C. in 20 min at a rate of 9° C./min. The temperature of the furnace was then maintained at 200° C. for 30 minutes. The temperature of the furnace was then increased from 200° C. to 300° C. in 10 min at a rate of 10° C./min. The temperature of the furnace was then maintained at 300° C. for 1 hour. The temperature of the furnace was increased from 300° C. to 380° C. in 10 min at a rate of 8° C./min. The temperature of the furnace was then maintained at 380° C. for 1 h. Then the temperature of the furnace was decreased from 380° C. to 25° C. in 12 hours. Sample 9 comprises magnetosomes obtained after condition 9 of treatment.

Condition 10 of heat treatment (Sample 10): 1.7 mL of a suspension containing 500 mg in iron of magnetosomes prepared according to condition no 2 of lysis, were lyophilized and then introduced into a porcelain crucible and heated in the furnace Nabertherm L9/11/B410. The heat protocol was as follows. The temperature of the furnace was increased from 20° C. to 200° C. in 2 hours 30 min at a rate of 1.2° C./min. The temperature of the furnace was then maintained at 200° C. for 1 hour. The temperature of the furnace was then increased from 200° C. to 300° C. in 1 h20 min at a rate of 1.25° C./min. The temperature of the furnace was then maintained at 300° C. for 2 hours. The temperature of the furnace was then increased from 300° C. to 380° C. in 1 hour 20 min at a rate of 1° C./min. The temperature of the furnace was then maintained at 380° C. for 2 h. Then the temperature of the furnace was decreased from 380° C. to 25° C. in 12 hours. Sample 10 comprises magnetosomes obtained after condition 10 of treatment.

Condition 11 of heat treatment (Sample 11): 1.7 mL of a suspension containing 500 mg in iron of magnetosomes prepared according to condition no 1 of lysis, were lyophilized and then introduced into a porcelain crucible and heated in the furnace Nabertherm L9/11/B410. The heat treatment was as follows. The temperature of the furnace was increased from 20° C. to 200° C. in 2 hours and 30 minutes at a rate of 1.2° C./min. The temperature of the furnace was then maintained at 200° C. for 1 hour. The temperature of the furnace was then increased from 200° C. to 300° C. in 1 h20 min at a rate of 1.25° C./min. The temperature of the furnace was then maintained at 300° C. for 2 hours. The temperature of the furnace was then increased from 300° C. to 380° C. in 1 hour 20 min at a rate of 1° C./min. The temperature of the furnace was then maintained at 380° C. for 2 h. Then the temperature of the furnace was decreased from 380° C. to 25° C. in 12 hours. Sample 11 comprises magnetosomes obtained after condition 11 of treatment.

Chemically synthesized nanoparticles (SIGMA, reference: 637106, batch no: MKBK2270V): Powder of chemically synthesized nanoparticles have been purchased from SIGMA. They have a size of 35±13 nm and they comprise in addition to iron oxide, 198 ppm of Aluminum (Al), 600 ppm of Calcium (Ca) 74 ppm of Chromium (Cr), 72 ppm of Magnesium (Mg), 642.5 ppm of Manganese (Mn), 30 ppm of Nickel (Ni), 128 ppm of Sodium (Na), 34 ppm of Titanium (Ti), 8.3 ppm of Vanadium (V), 56.5 ppm of Zinc (Zn).

### Results:

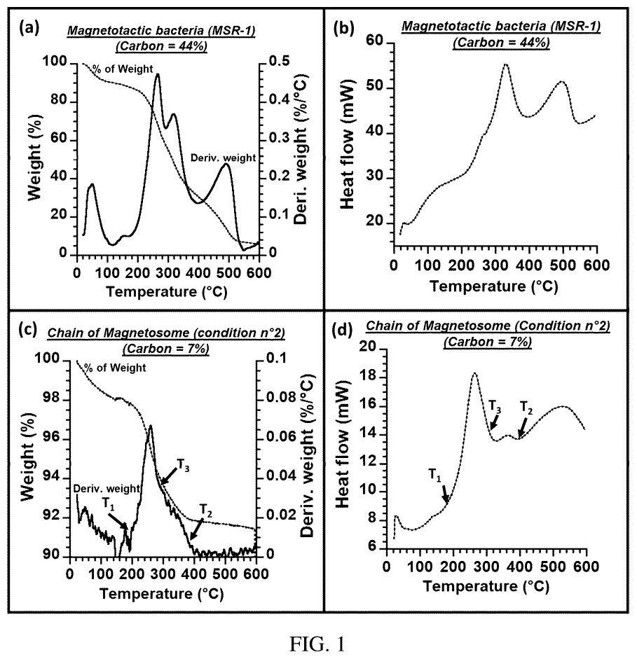

FIG. 1 (a) shows the percentage of weight loss of a sample comprising 3 mg of lyophilized whole MSR-1 magnetotactic bacteria (Sample 0) as a function of sample temperature, when the temperature of the sample is increased from 20° C. to 600° C. at a rate of 6° C. per minute, as well as the first derivative of this percentage. These measurements have been carried out with a combined TGA-DTA/DSC apparatus, measuring both heat flow using Differential Scanning Calorimetry and weight changes using Thermogravimetry in a material as a function of temperature. CHNS measurements of 3 mg of lyophilized whole MSR-1 magnetotactic bacteria (Sample 0) have shown that they contain a large percentage of carbon of 44%, before being heated (table 19). The percentage in weight of this sample decreases from 100% at 20° C. down to 5.5% at 600° C., indicating that the sample loses most of weight between 20 and 600° C. More specifically, it appears in FIG. 1(a) that the slope of the variation of the percentage of weight as a function of temperature is the largest within two temperature ranges: between 200 and 400° C. (interval 1) and between 400 and 540° C. (interval 2). Between 200 and 400° C., the variation as a function of temperature of the slope of the percentage of weight as a function of temperature displays a double peak whose maximum are at 260° C. and 315° C. This double peak could be due to the loss by the whole magnetotactic bacteria of organic material, preferentially of type 1, for the peak centered at 260° c. and of organic material, preferentially of a different type than type 1 such as type 2, for the peak centered at 315° C. Between 400 and 540° C., the variation as a function of temperature of the slope of the percentage of weight variation as a function of temperature displays a peak. This peak could be due to the loss by the nanoparticles of organic material, preferentially of a different type than type 1 or type 2 such as type 3.

FIG. 1(b) shows the heat flow in milliwatt of a sample comprising 3 mg of lyophilized whole magnetotactic bacteria as a function of sample temperature, when the temperature of the sample is increased from 20° C. to 600° C. at a rate of 6° C. per minute. These measurements have been carried out with a TGA-DSC apparatus. FIG. 1(b) shows two peaks with maximum heat flow observed at temperatures of 330° C. and 500° C. The peak centered at 330° C. could be attributed to the combustion of the mass of nanoparticles that has been lost or removed from the nanoparticles or transformed between 200 and 400° C. The peak at 500° C. could be attributed to the combustion of the mass of nanoparticles that has been lost between 500 and 540° C.

FIG. 1(c) shows the percentage of variation of the weight of a sample comprising 3 mg of lyophilized chains of magnetosomes prepared according to condition 2 of lysis as a function of sample temperature, when the temperature of the sample is increased from 20° C. to 600° C. at a rate of 6° C. per minute, as well as the first derivative of this percentage. These measurements have been carried out with a TGA-DSC apparatus. CHNS measurements of lyophilized chains of magnetosomes (condition no 2) have shown that they contain a percentage of carbon of 7%, before being heated, which is much lower than the percentage of carbon in whole magnetotactic bacteria (table 19). The percentage in weight of the sample comprising chains of magnetosomes decreases from 100% at 20° C. down to 91.4% at 600° C., indicating that the sample comprising chains of magnetosomes loses much less mass, i.e. 8.6%, than the sample comprising whole magnetotactic bacteria between 20° C. and 600° C. More specifically, it appears in FIG. 1(c) that the slope of the variation of the percentage in weight of the chains of magnetosomes as a function of temperature is the largest within the temperature range of 200 to 400° C. Between 200 and 400° C., the variation as a function of temperature of the slope of the percentage in weight of the chains of magnetosomes as a function of temperature displays a double peak whose maximum are at 260° C. and 315° C., similar positions than those of the double peak observed with whole bacteria. This double peak could be due to the loss by the chains of magnetosomes of organic material, preferentially organic material of type 1, for the peak centered at 260° c. and of organic material, preferentially organic material of type 2 for the peak centered at 315° C., where this organic material likely comes from the organic membrane layer surrounding the mineral iron oxide core of the magnetosomes. FIG. 1(d) shows the heat flow in milliwatt of a sample comprising 3 mg of lyophilized chains of magnetosomes as a function of the sample temperature, when the temperature of the sample is increased from 20° C. to 600° C. at a rate of 6° C. per minute. These measurements have been carried out with a TGA-DSC apparatus. FIG. 1(d) shows three peaks with maximum heat flow observed at temperatures of 250° C., 360° C., and 525° C. The peaks centered at 250° C. and 360° C. could be attributed to the combustion of the mass of nanoparticles that has been lost between 200 and 400° C. The peak at 525° C. could be attributed to the combustion of the mass of nanoparticles that has been lost above 500° C. and/or to the oxidation of the magnetosomes from an iron oxide composition of magnetite, maghemite, or an intermediate composition between magnetite and maghemite into hematite, which could result in heat flow possibly caused by an exothermic reaction.

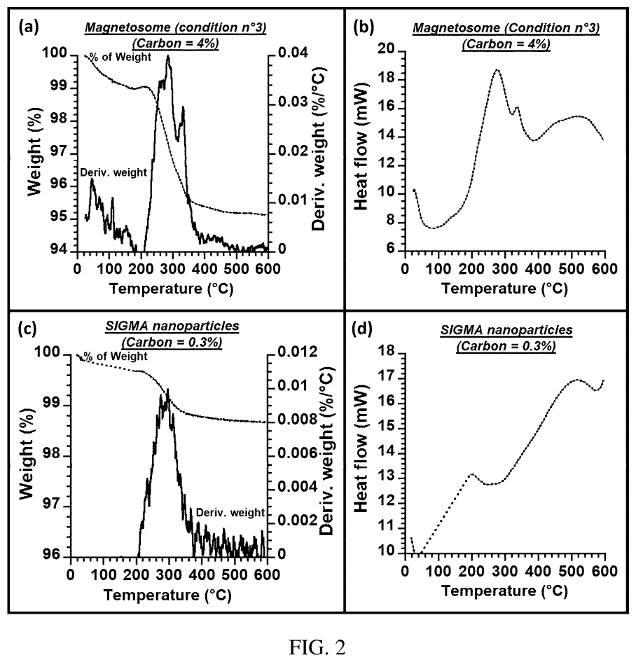

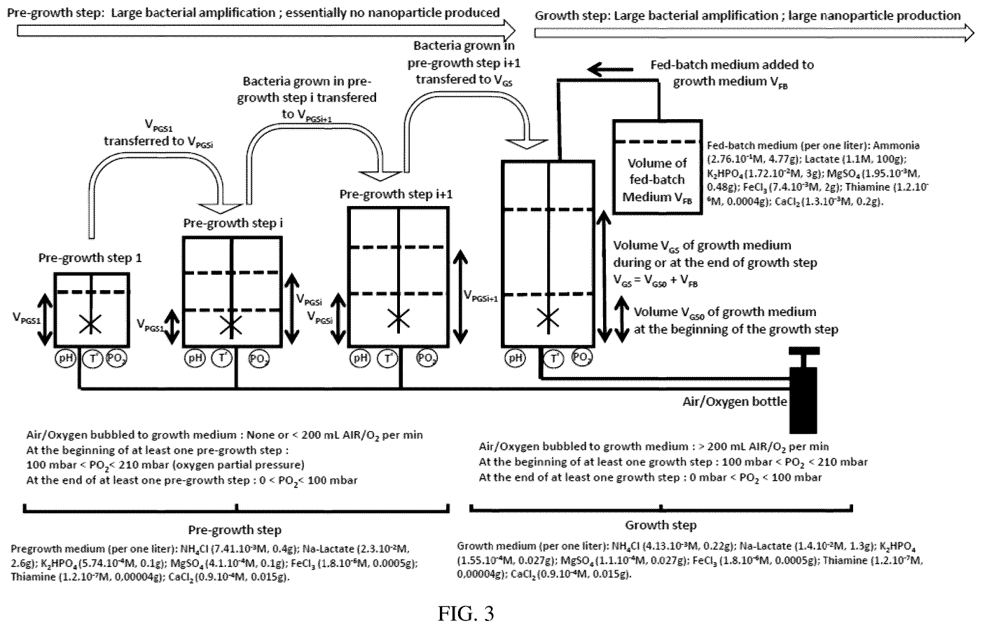

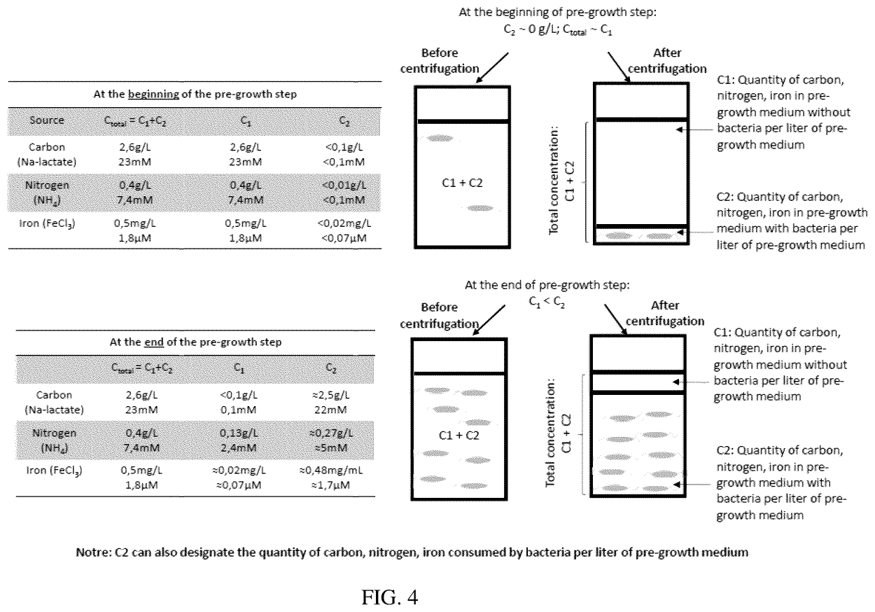

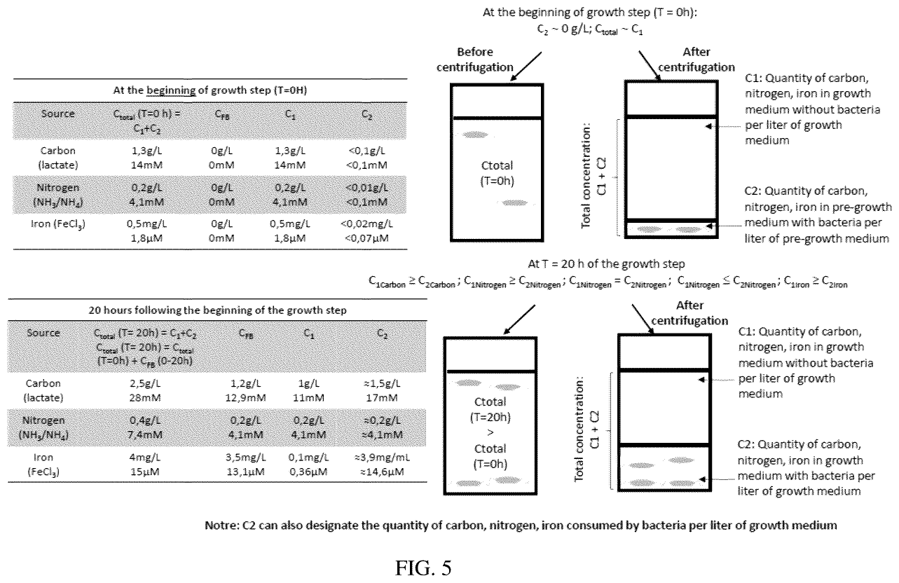

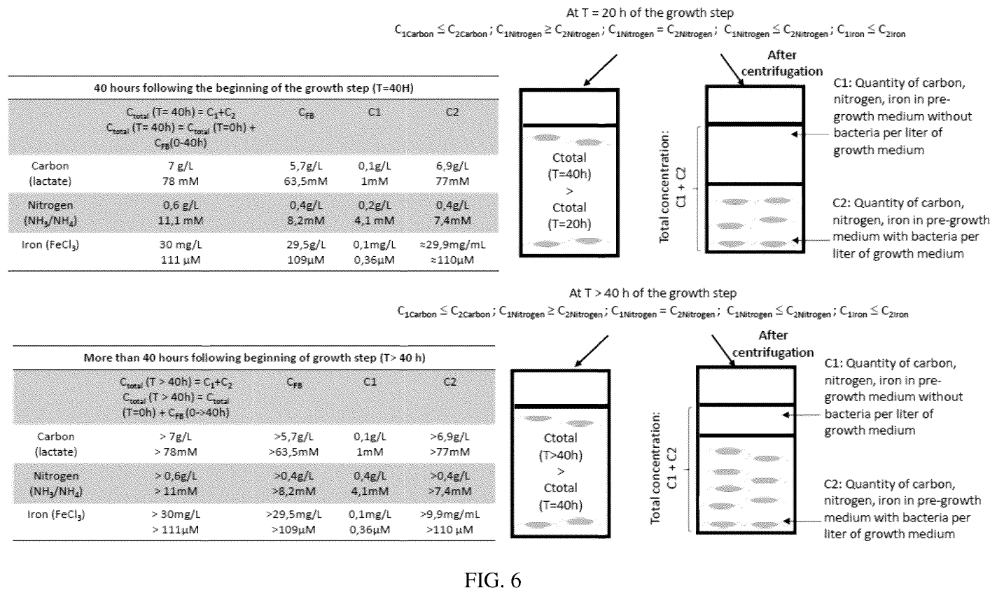

FIG. 2(a) shows the variation of the percentage in weight of a sample comprising 3 mg of lyophilized magnetosomes (Sample 3), prepared according to condition 3 as a function of the sample temperature, when the temperature of the sample is increased from 20° C. to 600° C. at a rate of 6° C. per minute, as well as the first derivative of this percentage. These measurements have been carried out with a TGA-DSC apparatus. CHNS measurements of lyophilized magnetosomes prepared according to condition 3 have shown that they contain a percentage of carbon of 4%, before being heated, which is lower than the percentage of carbon in magnetosomes prepared according to condition 2. The percentage in weight of the sample comprising magnetosomes (Sample 3) decreases from 100% at 20° C. down to 95.1% at 600° C., indicating that sample 3 comprising magnetosomes prepared according to condition 3 loses less weight, i.e. 4.9%, than sample 2 comprising magnetosomes prepared according to condition 2. More specifically, it appears in FIG. 2(a) that the slope of the variation of the percentage of weight as a function of temperature is the largest within the temperature range of 200 to 400° C. Between 200 and 400° C., the variation as a function of temperature of the slope of the percentage of weight as a function of temperature displays a quadruple peak whose maximum are at 264° C., 286° C., 325, and 331° C. The two peaks at 264° C. and 325° C. can be associated to peak shoulders. This quadruple peak could be due to the loss by the nanoparticles of organic material, preferentially of type 1, for the peak centered at 264° c., of organic material, preferentially of type 3, for the peak centered at 286° C., of organic material, preferentially of type 2 for the peak centered at 325° C., and of organic material, preferentially of type 4, for the peak centered at 331° C., where this organic material likely comes from some organic material surrounding or at the surface of the mineral iron oxide core of the magnetosomes. FIG. 2(b) shows the heat flow in milliwatt of a sample comprising 3 mg of lyophilized magnetosomes, prepared according to condition 3 (Sample3), as a function of the sample temperature, when the temperature of the sample is increased from 20° C. to 600° C. at a rate of 6° C. per minute. These measurements have been carried out with out with a TGA-DSC apparatus. FIG. 2(b) shows four peaks with maximum heat flow observed at temperatures of 277° C., 335° C., 455° C. and 522° C. The peaks centered at 277° C. and 335° C. could be attributed to the combustion of the mass of nanoparticles that has been lost between 200 and 400° C. The peaks at 455 and 522° C. could be attributed to the combustion of the mass of nanoparticles that has been lost above 500° C. and/or to the oxidation of the magnetosomes from an iron oxide composition of magnetite, maghemite, or an intermediate composition between magnetite and maghemite into hematite, that could result in heat flow possibly caused by an exothermic reaction.

FIG. 2(c) shows the variation of the percentage in weight of a sample comprising 3 mg of powder of SIGMA nanoparticles, purchased from Merck Sigma, as a function of the sample temperature, when the temperature of the sample is increased from 20° C. to 600° C. at a rate of 6° C. per minute, as well as the first derivative of this percentage. These measurements have been carried out with a TGA-DSC apparatus. CHNS measurements of lyophilized SIGMA nanoparticles have shown that they contain a percentage of carbon of 0.3%, before being heated, which is lower than the percentage of carbon in magnetosomes prepared according to condition 3 (Sample 3). The percentage in weight of the sample comprising SIGMA nanoparticles decreases from 100% at 20° C. down to 98.7% at 600° C., indicating that the sample comprising SIGMA nanoparticles loses less mass, i.e. 1.3%, than the magnetosomes prepared according to conditions 2 and 3. More specifically, it appears in FIG. 2(c) that the slope of the variation of the percentage in weight of SIGMA nanoparticles as a function of temperature is the largest within the temperature range of 200 to 400° C. Between 200 and 400° C., the variation as a function of temperature of the slope of the percentage of weight of SIGMA nanoparticles as a function of temperature displays a peak whose maximum is at 296° C. This peak could be due to the loss by the nanoparticles of organic material, preferentially of type 5, where this organic material could come from organic material adsorbed at the surface or comprised in or at the surface of the SIGMA nanoparticles. FIG. 2(d) shows the heat flow in milliwatt of a sample comprising 3 mg of powder of SIGMA nanoparticles as a function of the sample temperature, when the temperature of the sample is increased from 20° C. to 600° C. at a rate of 6° C. per minute. These measurements have been carried out with a TGA-DSC apparatus. FIG. 2(d) shows two peaks with maximum heat flow observed at temperatures of 200° C., and 515° C. The peak centered at 200° C. could be attributed to the combustion of the mass of nanoparticles that has been lost between 200 and 400° C. The peak centered at 515° C. could be attributed to the combustion of the mass of nanoparticles that has been lost above 500° C. and/or to the oxidation of the magnetosomes from an iron oxide composition of magnetite, maghemite, or an intermediate composition between magnetite and maghemite into hematite, that could result in heat flow possibly caused by an exothermic reaction resulting from the oxidation.

Determination of the different types of impurities, preferentially organic material, that can be removed, released, or dissociated from the nanoparticles: Each temperature corresponding or leading to the maximum value of the derivative of the variation of the percentage in weight of magnetosomes or Sigma nanoparticles as a function of temperature could be associated to a certain type of organic material that is removed from the nanoparticles. Hence, by knowing the values of these temperatures, it is possible to compare between different samples the type of organic material that can be removed from nanoparticles.

Determination of the temperatures at which the magnetosomes were heated in the furnace: In the different samples studied (whole magnetotactic bacteria, FIG. 1(a), chains of magnetosomes extracted from magnetotactic bacteria, FIG. 1(c), extracted and heated magnetosomes, FIG. 2(a)), the majority of organic material is removed or released from the magnetosomes extracted from magnetotactic bacteria between 200 and 400° C., i.e. the weight variation (%) and the derivative of the variation in weight (%/° C.) are the largest within this temperature range. We have therefore chosen to heat the magnetosomes extracted from magnetotactic bacteria at different temperatures comprised between 200 and 400° C.

Determination of the lysis method that leads to the lowest quantity of carbon in the magnetosomes: The magnetosomes extracted from magnetotactic bacteria with KOH have a lower quantity of carbon than the magnetosome extracted from magnetotactic bacteria with NaOH (7.1% with KaOH, Sample 2, compared with 14% with NaOH, Sample 1). The magnetosomes extracted from magnetotactic bacteria with KOH and heated at 200° C. for 30 min, 300° C. for 1 hour, and 380° C. for 1 hour have a lower percentage of carbon than the magnetosomes extracted from magnetotactic bacteria with NaOH and heated at 200° C. for 30 min, 300° C. for 1 hour, and 380° C. for 1 hour (0.3% with KOH, Sample 8, compared with 1% with NaOH, Sample 9). The magnetosomes extracted from magnetotactic bacteria with KOH and heated at 200° C. for 1 hour, 300° C. for 2 hours, and 380° C. for 2 hours have a lower percentage of carbon than the magnetosomes extracted from magnetotactic bacteria with NaOH and heated at 200° C. for 1 hour, 300° C. for 2 hours, and 380° C. for 2 hours (0.23% with KOH, Sample 10, compared with 0.8% with NaOH). This indicates that KOH is the best lysis method to yield a low percentage of carbon in the magnetosomes and therefore to have a high level of purification.

The magnetosomes extracted from magnetotactic bacteria with NaOH and then purified by a chemical method using phenol and chloroform possess a percentage in carbon, which is larger than that of the magnetosomes extracted from magnetoatctic bacteria with NaOH and heated at 200° C. during 30 minutes, 300° C. during 1 hour and 380° C. during 1 hour (1% of carbon with sample 9 compared with 5% of carbon with sample 3).

Determination of the heating temperature that leads to the lowest quantity of carbon in the magnetosomes: Considering the magnetosomes lysed with KOH, heating them at 400° C. during 1 hour leads to a lower quantity of carbon than heating them at 200° C. during 1 hour (3% of carbon at 400° C., Sample 5, compared with 5% of carbon at 200° C., Sample 4), indicating that increasing the heating temperature enables to remove more carbon.

Determination of the number of heating steps that leads to the lowest quantity of carbon in the magnetosomes: Considering the magnetosomes lysed with KOH, heating them at two different temperatures of 200 and 300° C. (Sample 6) or three different temperatures of 200° C., 300° C., and 380° C. (Sample 8), enables to remove more carbon than heating them at only one temperature (0.65% of carbon remains in the magnetosomes when they are heated at 200° C. and 300° C., Sample 6, and 0.3% of carbon remains in the magnetosomes when they are heated at 200, 300, and 380° C., Sample 8). This suggests that in order to reach a low level of carbon in the magnetosomes, magnetosomes can be heated at more than two different temperatures comprised between 200° C. and 380° C., such as 200° C., 300° C., and 380° C.

Determination of the heating time that leads to the lowest quantity of carbon in the magnetosomes: Considering the magnetosomes lysed with KOH and heated at 200° C., 300° C., and 380° C., increasing the heating time at 200° C. from 30 min to 1 hour and increasing the heating time at 300° C. and 380° C. from 1 hour to 2 hours slightly decreases the percentage of carbon that remains in the magnetosomes after heat treatment (0.23% for Sample 10 compared with 0.3% for Sample 8).

In conclusion, we have developed a method for heating nanoparticles, called magnetosomes, which are produced by specific cells called magnetotactic bacteria, which enables to reach a very low percentage in carbon, similar to that found in chemically synthesized nanoparticles, which are not synthesized by cells.

### TABLES

Table 1: Compositions in one liter of water of the pre-growth and growth media used to grow MSR-1 magnetotactic bacteria in 50 milliliter tubes following condition 1. In this condition, 13 different pre-growth media and 13 different growth media were prepared using 13 different mineral elixirs (VO, CB1, V2, CB2, CB3, CB4, CB5, CB7, CB9, CB10, CB11, CB12, CB13), whose composition in one liter of deionized water is given in table 6.

Table 2: Compositions in one liter of water of the pre-growth and growth media used to grow MSR-1 magnetotactic bacteria in 50 milliliter tubes following condition 2. In this condition, 4 different pre-growth media and 4 different growth media were prepared using 4 different yeast extracts (YE, YNBWAA, YNBWoAA, YNBWoAA.AS), whose composition in one liter of deionized water is given in table 7.

Table 3: Compositions in one liter of water of the pre-growth and growth media used to grow MSR-1 magnetotactic bacteria in 50 milliliter tubes following condition 3. In this condition, 5 different pre-growth media and 5 different growth media were prepared using 5 different vitamin cocktails (Vit1×, Vit5×, Vit10×, Vit0.5×, Vit0.1×), whose composition in one liter of deionized water is given in table 8.

Table 4: Compositions in one liter of water of the pre-growth and growth media used to grow MSR-1 magnetotactic bacteria in 50 milliliter tubes following condition 4. In this condition, 9 different pre-growth media and 9 different growth media were prepared using 9 different individual vitamins (Bt, CP, FA, I, NA, AA, P, R, T), whose composition in one liter of deionized water is given in table 9.

Table 5: Compositions in one liter of water of the pre-growth and growth media used to grow MSR-1 magnetotactic bacteria in 50 milliliter tubes following condition 5. In this condition, 4 different concentrations of sodium lactate (SL0, SL0.5×, SL0.2×, SL0.1×), ammonium chloride (A0, A0.5×, A.2×, A0.1×), Magnesium sulfate heptahydrate (MG0, MG0.5×, MG0.2×, MG0.1×), Potassium phosphate dibasic (P0, P0.5×, P0.2×, P0.1×) were tested.

Table 6: Compositions in one liter of water of the different mineral elixirs (VO, CB1, V2, CB2, CB3, CB4, CB5, CB7, CB9, CB10, CB11, CB12, CB13).

Table 7: Composition in one liter of water of the different yeast extracts (YE, YNBWAA, YNBWoAA, YNBWoAA.AS).

Table 8: Compositions in one liter of water of the different vitamin cocktails (Vit1×, Vit5×, Vit10×, Vit0.5×, Vit0.1×).

Table 9: Compositions in one liter of water of the different individual vitamins (Biotin Bt, Calcium pantothenate CP, Folic acid FA, Inositol I, Nicotinic acid NA, p-Aminobenzoic acid AA, Pyridoxine HCl P, Riboflavin R, Thiamine HCl T).

Table 10: For condition 1, optical density measured at the end of the pre-growth step, 6 days following the beginning of growth, OD_{D}6, or 13 days following the beginning of growth, OD_{D13}, ratio OD_{D13}/OD_{D6}, and percentage of magnetic response.

Table 11: For conditions 2 and 3, optical density measured at the end of the pre-growth step, 6 days following the beginning of growth, OD_{D}6, or 13 days following the beginning of growth, OD_{D13}, ratio OD_{D13}/OD_{D6}, and percentage of magnetic response.

Table 12: For condition 4, optical density measured at the end of the pre-growth step, 6 days following the beginning of growth, OD_{D}6, or 13 days following the beginning of growth, OD_{D13}, ratio OD_{D13}/OD_{D6}, and percentage of magnetic response.

Table 13: For condition 5, optical density measured at the end of the pre-growth step, 6 days following the beginning of growth, OD_{D}6, or 13 days following the beginning of growth, OD_{D13}, ratio OD_{D13}/OD_{D6}, and percentage of magnetic response.

Table 14(a): For condition 6 of growth of MSR-1 magnetotactic bacteria in one liter fermenter, compositions of pre-growth medium, growth medium, and fed-batch medium for B1 and B4, prepared using non-pharmaceutical grade chemicals.

Table 14(b): For condition 6 of growth of MSR-1 magnetotactic bacteria in one liter fermenter, compositions of pre-growth medium, growth medium, and fed-batch medium for B2 and B3, prepared using pharmaceutical grade chemicals.

Table 15: For condition 6, optical density and number of cells per mL measured at the beginning of the pre-growth step (DO) for bacteria inserted in a volume of 250 mL pre-growth media, at the end of the pre-growth step (D9) for bacteria grown in 1.5 liter pre-growth media, at the beginning of the growth step (D9) when bacteria are grown in 800 mL growth media, at D9 of the growth step, at D11 of the growth step. Ratio between the optical density of the bacteria measured at D11 and the optical density of the bacteria measured at D9. Percentage of magnetic response measured at D11.

Table 16: For magnetosomes produced under condition 6, extracted from magnetotactic bacteria and purified to remove more than 99% of carbonaceous material, concentration of impurities (Ag, Al, As, Ba, Cd, Co, Cr, Cu, Mn, Mo, Ni, Pb, Sb, Se, Si, Sn, Ti, W, Zn) in μg of impurities per gram of magnetosome, for media B1, B2, B3, and B4.

Table 17: For the fermenters B1, B2, B3, B4, the iron concentrations comprised in: i), the pre-growth medium at DO (day 0), D8, D9, ii), the growth medium 0 h after the start of the growth step at D9, 6 h after the start of the growth step at D9, 12 h after the start of the growth step at D9, 24 h after the start of the growth step at D10, 48 h after the start of the growth step at D11, iii) volume of fed-batch medium introduced in the growth medium 0 h after the start of the growth step at D9, 6 h after the start of the growth step at D9, 12 h after the start of the growth step at D9, 24 h after the start of the growth step at D9, 48 h after the start of the growth step at D11.

Table 18: Conditions of treatments for the different samples (Sample 0 to sample 11), including the condition of lysis (using NaOH, KOH, or NaOH+Phenol and chloroform), the initial temperature before heating the sample (Tᵢ), the temperature T₁, the rate rᵢ₁ at which the temperature is increased from Tᵢ to T₁, the time t₁ during which the temperature is maintained at T₁, the temperature T₂, the rate rᵢ₂ at which the temperature is increased from T₁ to T₂, the rate rᵢ₂ at which the temperature is increased from T₁ to T₂, the time t₂ during which the temperature is maintained at T₂, the temperature T₃, the rate rᵢ₃ at which the temperature is increased from T₁ to T₃, the rate r₃₂ at which the temperature is increased from T₃ to T₂, the time t₃ during which the temperature is maintained at t₃, the temperature T₄, the rate r₃₄ at which the temperature is increased from T₃ to T₄, the time t₄ during which the temperature is maintained at T₄, the final temperature T_{f}, the rate r_{1f} at which the temperature is decreased from T₁ to T_{f}, the rate r_{2f} at which the temperature is decreased from T₂ to T_{f}, the rate r₃f at which the temperature is decreased from T₃ to T_{f}, the rate r₄f at which the temperature is decreased from T₄ to T_{f}.

Table 19: For the different samples (Sample 3 to Sample 11), the percentages in mass of carbon (% C) and nitrogen (% N) after the treatment of the nanoparticles by conditions 3 to 11, the percentages in mass of carbon and (% Ci) and nitrogen (% Ni) for the magnetosomes extracted from magnetotactic bacteria following condition 1 or 2 before heat treatment (samples 4 to 11) or before treatment with phenol-chloroform (sample 3). Values of A % C=% Ci−% C, A % N=% Ni−% N, (100 Δ % C)/% Ci, (100·Δ % N)/Δ % Ni.

Table 20: A comparison of metallic composition of magnetosomes after biomineralization in a Non reduced medium (VO) or in a Reduced medium (CB3).

Table 21: Elemental compositions in gram per one liter of reduced and non-reduced growth and Fed-batch media. The N/A symbol corresponds to an element that has not been added to a medium.

Table 22: Elemental compositions in gram per one liter of reduce and non-reduce growth and Fedbatch media measured by ICP-MS. A concentration of “1·10⁻¹²” means “<1·10⁻¹²”.

TABLE 1
Condition 1: 13 different mineral elixers tested
(MSR-1 magnetotactic bacteria grown in 50 mL tubes)

| | | Chemical | | | |
|---|---|---|---|---|---|
| | Name of chemicals | formula | Quantity | | Concentration |
| Pregrowth | Sodium lactate | C₃H₅NaO₃ | 2.6 | g | 2.30.10⁻²M |
| medium | Ammonium chloride | NH₄Cl | 0.4 | g | 7.41.10⁻³M |
| | Magnesium sulfate heptahydrate | MgSO₄•7H₂O | 0.1 | g | 4.06.10⁻⁴M |
| | Potassium phosphate dibasic | K₂HPO₄ | 0.5 | g | 2.87.10⁻³M |
| | Yeast extract (Table 7, YE) | / | 0.1 | g | / |
| | Mineral elixir: V0, CB1, V2, CB2, CB3, CB4, | / | 0.5 | mL | / |
| | CB5, CB7, CB9, CB10, CB11, CB12, or CB13, Table 6 | | | | |
| | Deionized water | H₂O | 1 | L | / |
| Growth | Sodium lactate | C₃H₅NaO₄ | 2.6 | g | 2.30.10⁻²M |
| medium | Ammonium chloride | NH₄Cl | 0.4 | g | 7.41.10⁻³M |
| | Magnesium sulfate heptahydrate | MgSO₄•7H₂O | 0.1 | g | 4.06.10⁻³M |
| | Potassium phosphate dibasic | K₂HPO₅ | 0.5 | g | 2.87.10⁻³M |
| | Yeast extract (Table 7, YE) | / | 0.1 | g | / |
| | Mineral elixir: V0, CB1, V2, CB2, CB3, CB4, | / | 0.5 | mL | / |
| | CB5, CB7, CB9, CB10, CB11, CB12, or CB13, Table 6 | | | | |
| | Ferric citrate (20 mM) | C₆H₅FeO₇ | 10 | mL | 200 μM |
| | Deionized water | H₂O | 1 | L | / |

TABLE 2
Condition 2: 4 different yeast extracts tested:
(MSR1 megnetotactic bacteria grown in 50 mL tubes)

| | Name of chemicals | Chemical formula | Quantity | | Concentration |
|---|---|---|---|---|---|
| Pregrowth | Sodium lactate | C₃H₅NaO₃ | 2.6 | g | 2.30.10⁻²M |
| medium | Ammonium chloride | NH₄Cl | 0.4 | g | 7.41.10⁻³M |
| | Magnesium sulfate heptahydrate | MgSO₄•7H₂O | 0.1 | g | 4.06.10⁻⁴M |
| | Potassium phosphate dibasic | K₂HPO₄ | 0.5 | g | 2.87.10⁻³M |
| | Yeast extract: YE, YNBWAA, YNBWoAA, | / | 0.1 | g | / |
| | YNBWoAA.AS Table 7 | | | | |
| | Mineral elixir CB3 (Table 6) | / | 0.5 | mL | [FeO₄S•7H₂O] = 1.8.10⁻⁶M |
| | | | | | [CaCl₂] = 1.4.10⁻³M |
| | Deionized water | H₂O | 1 | L | / |
| Growth | Sodium lactate | C₃H₅NaO₄ | 2.6 | g | 2.30.10⁻²M |
| medium | Ammonium chloride | NH₄Cl | 0.4 | g | 7.41.10⁻³M |
| | Magnesium sulfate heptahydrate | MgSO₄•7H₂O | 0.1 | g | 4.06.10⁻⁴M |
| | Potassium phosphate dibasic | K₂HPO₅ | 0.5 | g | 2.87.10⁻³M |
| | Yeast extract: YE, YNBWAA, YNBWoAA, | / | 0.1 | g | / |
| | YNBWoAA.AS Table 7 | | | | |
| | Mineral elixir CB3 (Table 6) | / | 0.5 | mL | [FeO₄S•7H₂O] = 1.8.10⁻⁶M |
| | | | | | [CaCl₂] = 1.4.10⁻⁴M |
| | Ferric citrate (20 mM) | C₆H₅FeO₇ | 10 | mL | 200 μM |
| | Deionized water | H₂O | 1 | L | / |

TABLE 3
Condition 3: 7 different vitamin cocktails tested
(MSR-1 megnetotactic bacteria grown in 50 mL tubes)

| | Name of chemicals | Chemical formula | Quantity | | Concentration |
|---|---|---|---|---|---|
| Pregrowth | Sodium lactate | C₃H₅NaO₃ | 2.6 | g | 2.30.10⁻²M |
| medium | Ammonium chloride | NH₄Cl | 0.4 | g | 7.41.10⁻³M |
| | Magnesium sulfate heptahydrate | MgSO₄•7H₂O | 0.1 | g | 4.06.10⁻⁴M |
| | Potassium phosphate dibasic | K₂HPO₄ | 0.5 | g | 2.87.10⁻³M |
| | Vitamins coktail: Vit1X, Vit5X, Vit10X, Vit5X, | / | 0.1 | mL | 8.2.10⁻¹¹M < [Vit] < 1.1.10⁻⁵M |
| | Vit0.5X, or Vit0.1X Table 8 | | | | |
| | Mineral elixir CB3 (Table 6) | / | 0.5 | mL | [FeO₄S•7H₂O] = 1.8.10⁻⁵M |
| | | | | | [CaCl₂] = 1.4.10⁻³M |
| | Deionized water | H₂O | 1 | L | / |
| Growth | Sodium lactate | C₃H₅NaO₄ | 2.6 | g | 2.30.10⁻²M |
| medium | Ammonium chloride | NH₄Cl | 0.4 | g | 7.41.10⁻³M |
| | Magnesium sulfate heptahydrate | MgSO₄•7H₂O | 0.1 | g | 4.06.10⁻⁴M |
| | Potassium phosphate dibasic | K₂HPO₅ | 0.5 | g | 2.87.10⁻³M |
| | Vitamins coktail: Vit1X, Vit5X, Vit10X, Vit5X, | / | 0.1 | mL | 8.2.10⁻¹¹M < [Vit] < 1.1.10⁻⁵M |
| | Vit0.5X, or Vit0.1X Table 8 | | | | |
| | Mineral elixir CB3 (Table 6) | / | 0.5 | mL | [FeO₄S•7H₂O] = 1.8.10⁻⁶M |
| | | | | | [CaCl₂] = 1.4.10⁻⁴M |
| | Ferric citrate (20 mM) | C₆H₅FeO₇ | 10 | mL | 200 μM |
| | Deionized water | H₂O | 1 | L | / |

TABLE 4
Condition 4: 9 different individual vitamins tested
(MSR-1 megnetotactic bacteria grown in 50 mL tubes)

| | Name of chemicals | Chemical formula | Quantity | | Concentration |
|---|---|---|---|---|---|
| Pregrowth | Sodium lactate | C₃H₅NaO₃ | 2.6 | g | 2.30.10⁻²M |
| medium | Ammonium chloride | NH₄Cl | 0.4 | g | 7.41.10⁻³M |
| | Magnesium sulfate heptahydrate | MgSO₄•7H₂O | 0.1 | g | 4.06.10⁻⁴M |
| | Potassium phosphate dibasic | K₂HPO₄ | 0.5 | g | 2.87.10⁻³M |
| | Individual vitamins: Bt, CP, FA, I, NA, AA, | / | 0.1 | mL | 8.2.10⁻¹⁰M < [Vit] < 1.1.10⁻⁶M |
| | P, R, or T Table 9 | | | | |
| | Mineral elixir CB3 (Table 6) | / | 0.5 | mL | [FeO₄S•7H₂O] = 1.8.10⁻⁶M |
| | | | | | [CaCl₂] = 1.4.10⁻⁴M |
| | Deionized water | H₂O | 1 | L | / |
| Growth | Sodium lactate | C₃H₅NaO₄ | 2.6 | g | 2.30.10⁻²M |
| medium | Ammonium chloride | NH₄Cl | 0.4 | g | 7.41.10⁻³M |
| | Magnesium sulfate heptahydrate | MgSO₄•7H₂O | 0.1 | g | 4.06.10⁻⁴M |
| | Potassium phosphate dibasic | K₂HPO₅ | 0.5 | g | 2.87.10⁻³M |
| | Individual vitamins: Bt, CP, FA, I, NA, AA, | / | 0.1 | mL | 8.2.10⁻¹⁰M < [Vit] < 1.1.10⁻⁶M |
| | P, R, or T Table 9 | | | | |
| | Mineral elixir CB3 (Table 6) | / | 0.5 | mL | [FeO₄S•7H₂O] = 1.8.10⁻⁶M |
| | | | | | [CaCl₂] = 1.4.10⁻⁴M |
| | Ferric citrate (20 mM) | C₆H₅FeO₇ | 10 | mL | 200 μM |
| | Deionized water | H₂O | 1 | L | / |

TABLE 5
Condition 5: Test of 4 different concentrations of the main constituants of the growth media
sodium lactate (SL), Ammonium chloride (A), Magnesium sulfate heptahydrate (MG), Potassium phosphate dibasic (P)
(MSR-1 magnetotactic baacteria grown in 50 mL tubes)

| | | Chemical | | | | | | | | | | | | | | | | | | |
|---|---|---|---|---|---|---|---|---|---|---|---|---|---|---|---|---|---|---|---|---|
| | Name of Chemicals | formula | N | | SL0 | | SL0.5X | | SL0.2X | | SL0.1X | | AC0 | | AC0.5X | | AC0.2X | | AC0.1X | |
| Pregrowth | Sodium lactate | C₃H₅NaO₃ | 2.6 | g | 0 | g | 1.3 | g | 0.52 | g | 0.26 | g | 2.6 | g | 2.6 | g | 2.6 | g | 2.6 | g |
| medium | Ammonium chloride | NH₄Cl | 0.4 | g | 0.4 | g | 0.4 | g | 0.4 | g | 0.4 | g | 0 | g | 0.2 | g | 0.08 | g | 0.04 | g |
| | Magnesium sulfate | MgSO₄•7H₂O | 0.1 | g | 0.1 | g | 0.1 | g | 0.1 | g | 0.1 | g | 0.1 | g | 0.1 | g | 0.1 | g | 0.1 | g |
| | heptahydrate | | | | | | | | | | | | | | | | | | | |
| | Potassium phosphate | K₂HPO₄ | 0.5 | g | 0.5 | g | 0.5 | g | 0.5 | g | 0.5 | g | 0.5 | g | 0.5 | g | 0.5 | g | 0.5 | g |
| | dibasic | | | | | | | | | | | | | | | | | | | |
| | Mineral elixir CB3 | / | 0.5 | ml | 0.5 | ml | 0.5 | ml | 0.5 | ml | 0.5 | ml | 0.5 | ml | 0.5 | ml | 0.5 | ml | 0.5 | ml |
| | (Table 6) | | | | | | | | | | | | | | | | | | | |
| | Vitamins cocktail | / | 0.1 | ml | 0.1 | ml | 0.1 | ml | 0.1 | ml | 0.1 | ml | 0.1 | ml | 0.1 | ml | 0.1 | ml | 0.1 | ml |
| | Vit0.1X (Table 8) | | | | | | | | | | | | | | | | | | | |
| | Deionized water | H₂O | 1 | l | 1 | l | 1 | l | 1 | l | 1 | l | 1 | l | 1 | l | 1 | l | 1 | l |
| Growth | Sodium lactate | C₃H₅NaO₃ | 2.6 | g | 0 | g | 1.3 | g | 0.52 | g | 0.26 | g | 0.26 | g | 2.6 | g | 2.6 | g | 2.6 | g |
| medium | Ammonium chloride | NH₄Cl | 0.4 | g | 0.4 | g | 0.4 | g | 0.4 | g | 0.4 | g | 0 | g | 0.2 | g | 0.08 | g | 0.04 | g |
| | Magnesium sulfate | MgSO₄•7H₂O | 0.1 | g | 0.1 | g | 0.1 | g | 0.1 | g | 0.1 | g | 0.1 | g | 0.1 | g | 0.1 | g | 0.1 | g |
| | heptahydrate | | | | | | | | | | | | | | | | | | | |
| | Potassium phosphate | K₂HPO₄ | 0.5 | g | 0.5 | g | 0.5 | g | 0.5 | g | 0.5 | g | 0.5 | g | 0.5 | g | 0.5 | g | 0.5 | g |
| | dibasic | | | | | | | | | | | | | | | | | | | |
| | Mineral elixir CB3 | / | 0.5 | ml | 0.5 | ml | 0.5 | ml | 0.5 | ml | 0.5 | ml | 0.5 | ml | 0.5 | ml | 0.5 | ml | 0.5 | ml |
| | (Table 6) | | | | | | | | | | | | | | | | | | | |
| | Vitamins cocktail | / | 0.1 | ml | 0.1 | ml | 0.1 | ml | 0.1 | ml | 0.1 | ml | 0.1 | ml | 0.1 | ml | 0.1 | ml | 0.1 | ml |
| | Vit0.1X (Table 8) | | | | | | | | | | | | | | | | | | | |
| | Ferric citrate (20 mM) | C₆H₅FeO₇ | 10 | ml | 10 | ml | 10 | ml | 10 | ml | 10 | ml | 10 | ml | 10 | ml | 10 | ml | 10 | ml |
| | Deionized water | H₂O | 1 | l | 1 | l | 1 | l | 1 | l | 1 | l | 1 | l | 1 | l | 1 | l | 1 | l |

| | Name of Chemicals | MG0 | | MG0.5X | | MG0.2X | | MG0.1X | | P0 | | P0.5X | | P0.2X | | P0.1X | |
|---|---|---|---|---|---|---|---|---|---|---|---|---|---|---|---|---|---|
| Pregrowth | Sodium lactate | 2.6 | g | 2.6 | g | 2.6 | g | 2.6 | g | 2.6 | g | 2.6 | g | 2.6 | g | 2.6 | g |
| medium | Ammonium chloride | 0.04 | g | 0.04 | g | 0.04 | g | 0.04 | g | 0.04 | g | 0.04 | g | 0.04 | g | 0.04 | g |
| | Magnesium sulfate | 0 | g | 0.05 | g | 0.02 | g | 0.01 | g | 0.01 | g | 0.01 | g | 0.01 | g | 0.01 | g |
| | heptahydrate | | | | | | | | | | | | | | | | |
| | Potassium phosphate | 0.5 | g | 0.5 | g | 0.5 | g | 0.5 | g | 0 | g | 0.25 | g | 0.1 | g | 0.05 | g |
| | dibasic | | | | | | | | | | | | | | | | |
| | Mineral elixir CB3 | 0.5 | ml | 0.5 | ml | 0.5 | ml | 0.5 | ml | 0.5 | ml | 0.5 | ml | 0.5 | ml | 0.5 | ml |
| | (Table 6) | | | | | | | | | | | | | | | | |
| | Vitamins cocktail | 0.1 | ml | 0.1 | ml | 0.1 | ml | 0.1 | ml | 0.1 | ml | 0.1 | ml | 0.1 | ml | 0.1 | ml |
| | Vit0.1X (Table 8) | | | | | | | | | | | | | | | | |
| | Deionized water | 1 | l | 1 | l | 1 | l | 1 | l | 1 | l | 1 | l | 1 | l | 1 | l |
| Growth | Sodium lactate | 2.6 | g | 2.6 | g | 2.6 | g | 2.6 | g | 2.6 | g | 2.6 | g | 2.6 | g | | |
| medium | Ammonium chloride | 0.04 | g | 0.04 | g | 0.04 | g | 0.04 | g | 0.04 | g | 0.04 | g | 0.04 | g | 0.04 | g |
| | Magnesium sulfate | 0 | g | 0.05 | g | 0.02 | g | 0.01 | g | 0.01 | g | 0.01 | g | 0.01 | g | 0.01 | g |
| | heptahydrate | | | | | | | | | | | | | | | | |
| | Potassium phosphate | 0.5 | g | 0.5 | g | 0.5 | g | 0.5 | g | 0 | g | 0.25 | g | 0.1 | g | 0.05 | g |
| | dibasic | | | | | | | | | | | | | | | | |
| | Mineral elixir CB3 | 0.5 | ml | 0.5 | ml | 0.5 | ml | 0.5 | ml | 0.5 | ml | 0.5 | ml | 0.5 | ml | 0.5 | ml |
| | (Table 6) | | | | | | | | | | | | | | | | |
| | Vitamins cocktail | 0.1 | ml | 0.1 | ml | 0.1 | ml | 0.1 | ml | 0.1 | ml | 0.1 | ml | 0.1 | ml | 0.1 | ml |
| | Vit0.1X (Table 8) | | | | | | | | | | | | | | | | |
| | Ferric citrate (20 mM) | 10 | ml | 10 | ml | 10 | ml | 10 | ml | 10 | ml | 10 | ml | 10 | ml | 10 | ml |
| | Deionized water | 1 | l | 1 | l | 1 | l | 1 | l | 1 | l | 1 | l | 1 | l | 1 | l |

l: liter;
ml: milliliter

TABLE 6
Compositions of the different tested mineral elixirs:

| Name of Chemicals | Chemical formula | V0 | | CB1 | | V2 | | CB2 | | CB3 | |
|---|---|---|---|---|---|---|---|---|---|---|---|
| Nitrilotriacetic acid | C₆H₅NO₆Na₃ | 15 | g | 0 | g | 15 | g | 0 | g | 0 | g |
| trisodium salt | | | | | | | | | | | |
| Magnesium sulfate | MgSO₄•7H₂O | 3 | g | 0 | g | 3 | g | 3 | g | 0 | g |
| heptahydrate | | | | | | | | | | | |
| Manganese (II) sulfate | MnO₄S•H₂O | 5 | g | 0 | g | 0.002 | g | 0 | g | 0 | g |
| monohydrate | | | | | | | | | | | |
| Sodium Chloride | NaCl | 10 | g | 0 | g | 10 | g | 10 | g | 0 | g |
| Iron(II) sulfate heptahydrate | FeO₄S•7H₂O | 1 | g | 0 | g | 1 | g | 1 | g | 1 | g |
| Cobalt(II) nitrate | CoN₂O₆•6H₂O | 1.8 | g | 0 | g | 0.003 | g | 0 | g | 0 | g |
| hexahydrate | | | | | | | | | | | |
| Calcium chloride | CaCl₂ | 30 | g | 0 | g | 30 | g | 30 | g | 30 | g |
| Zinc sulfate heptahydrate | O₄SZn•7H₂O | 1.8 | g | 0 | g | 0.003 | g | 0 | g | 0 | g |
| Copper(II) sulfate | CuO₄S•5H₂O | 0.1 | g | 0 | g | 0.002 | g | 0 | g | 0 | g |
| pentahydrate | | | | | | | | | | | |
| Aluminum potassium | AlK(SO₄)₂•12H₂O | 0.2 | g | 0 | g | 0.005 | g | 0 | g | 0 | g |
| sulfate dodecahydrate | | | | | | | | | | | |
| Boric acid | H₂BO₃ | 0.1 | | 0 | g | 0.1 | | 0 | g | 0 | g |
| Sodium molybdate | Na₂MoO₄•2H₂O | 0.1 | g | 0 | g | 0.1 | g | 0 | g | 0 | g |
| dihydrate | | | | | | | | | | | |
| Nickle(II) chloride | Cl₂Ni•6H₂O | 0.25 | g | 0 | g | 0.002 | g | 0 | g | 0 | g |
| hexahydrate | | | | | | | | | | | |
| Sodium selenite | Na₂SeO₅•5H₂O | 0.003 | g | 0 | g | 0.003 | g | 0 | g | 0 | g |
| pentahydrate | | | | | | | | | | | |
| EDTA | (HO₂CCH₂)₂NCH₂CH₂N(CH₂CO₂H)₂ | 0 | g | 0 | g | 0 | g | 0 | g | 0 | g |
| Iron(III) oxalate hexahydrate | Fe₂(C₂O₄)₃•6H₂O | 0 | g | 0 | g | 0 | g | 0 | g | 0 | g |
| Protoporphyrin IX | C₃4H₃4NaO₄ | 0 | g | 0 | g | 0 | g | 0 | g | 0 | g |
| Deionized water | H₂O | 1 | L | 1 | L | 1 | L | 1 | L | 1 | L |

| Name of Chemicals | CB4 | | CB5 | | CB7 | | CB9 | | CB10 | | CB11 | | CB12 | | CB13 | |
|---|---|---|---|---|---|---|---|---|---|---|---|---|---|---|---|---|
| Nitrilotriacetic acid | 0 | g | 0 | g | 15 | g | 1.5 · 10⁻⁶ | g | 0 | g | 0 | g | 0 | g | 0 | g |
| trisodium salt | | | | | | | | | | | | | | | | |
| Magnesium sulfate | 3 | g | 3 | g | 3 | g | 3 · 10⁻⁶ | g | 3 | g | 3 | g | 0 | g | 0 | g |
| heptahydrate | | | | | | | | | | | | | | | | |
| Manganese (II) sulfate | 2 · 10⁻⁷ | g | 0 | g | 0 | | 5 · 10⁻⁸ | g | 0 | g | 0 | g | 0 | g | 0 | g |
| monohydrate | | | | | | | | | | | | | | | | |
| Sodium Chloride | 10 | g | 10 | g | 10 | g | 3 · 10⁻⁵ | g | 10 | g | 10 | g | 0 | g | 0 | g |
| Iron(II) sulfate heptahydrate | 1 | g | 1 | g | 1 | g | 3 · 10⁻⁵ | g | 1 | g | 1 | g | 1 | g | 1 | g |
| Cobalt(II) nitrate | 3 · 10⁻¹⁰ | g | 0 | g | 0 | g | 10⁻⁶ | g | 0 | g | 0 | g | 0 | g | 0 | g |
| hexahydrate | | | | | | | | | | | | | | | | |
| Calcium chloride | 30 | g | 30 | g | 30 | g | 2 · 10⁻⁶ | g | 30 | g | 30 | g | 15 | g | 20 | g |
| Zinc sulfate heptahydrate | 0.003 | g | 0 | g | 0 | g | 2 · 10⁻⁶ | g | 0 | g | 0 | g | 0 | g | 0 | g |
| Copper(II) sulfate | 0.002 | g | 0 | g | 0 | g | 10⁻⁷ | g | 0 | g | 0 | g | 0 | g | 0 | g |
| pentahydrate | | | | | | | | | | | | | | | | |
| Aluminum potassium | 0.005 | g | 0 | g | 0 | g | 2 · 10⁻⁷ | g | 0 | g | 0 | g | 0 | g | 0 | g |
| sulfate dodecahydrate | | | | | | | | | | | | | | | | |
| Boric acid | 0 | g | 0 | g | 0.1 | g | 10⁻⁷ | g | 0 | g | 0 | g | 0 | g | 0 | g |
| Sodium molybdate | 0 | g | 0 | g | 0 | g | 10⁻⁷ | g | 0 | g | 0 | g | 0 | g | 0 | g |
| dihydrate | | | | | | | | | | | | | | | | |
| Nickle(II) chloride | 0 | g | 0 | g | 0 | g | 2.5 · 10⁻⁷ | g | 0 | g | 0 | g | 0 | g | 0 | g |
| hexahydrate | | | | | | | | | | | | | | | | |
| Sodium selenite | 0 | g | 0 | g | 0 | g | 3 · 10⁻⁹ | g | 0 | g | 0 | g | 0 | g | 0 | g |
| pentahydrate | | | | | | | | | | | | | | | | |
| EDTA | 0.4 | g | 0.4 | g | 0 | g | 0 | g | 0 | g | 0 | g | 0 | g | 0 | g |
| Iron(III) oxalate hexahydrate | 0 | g | 1 | g | 1 | g | 0 | g | 1 | g | 0 | g | 0 | g | 0 | g |
| Protoporphyrin IX | 0 | g | 0 | g | 0 | g | 0 | g | 0 | g | 0.06 | g | 0 | g | 0 | g |
| Deionized water | 1 | L | 1 | L | 1 | L | 1 | L | 1 | L | 1 | L | 1 | L | 1 | L |

TABLE 7
Compositions of the different tested yeast extract:

| Chemical name | Chemical formula | YE | | YNBWAA | | YNBWoAA | | YNBWoAA.AS | |
|---|---|---|---|---|---|---|---|---|---|
| Yeast extract | / | 0.1 | g | 0 | g | 0 | g | 0 | g |
| Ammonium sulfate | (NH₄)₂SO₄ | 0 | g | 5 | g | 5 | g | 0 | g |
| L-histidine | C₆H₉N₃O₂ | 0 | g | 0.01 | g | 0 | g | 0 | g |
| DL-methionine | CH₃SCH₂CH₂CH(NH₂)COOH | 0 | g | 0.02 | g | 0 | g | 0 | g |
| DL-tryptophan | C₁₁H₁₂N₂O₂ | 0 | g | 0 | g | 0 | g | 0 | g |
| Potassium phosphate | KH₂PO₄ | 0 | g | 1 | g | 1 | g | 1 | g |
| monobasic | | | | | | | | | |
| Magnesium sulfate | MgSO₄ | 0 | g | 0.5 | g | 0.5 | g | 0.5 | g |
| Sodium chloride | NaCl | 0 | g | 0.1 | g | 0.1 | g | 0.1 | g |
| Calcium chloride | CaCl₂ | 0 | g | 0.1 | g | 0.1 | g | 0.1 | g |
| Trace elements | H³BO³, CuO₄S, KI, FeCl³, Na²MoO⁴, O₄SZn | 0 | g | <4.10⁻⁴ | g | <4.10⁻⁴ | g | <4.10⁻⁴ | g |
| Biotin | C₁₀H₁₆N₂O₃S | 0 | g | 2.10⁻⁶ | g | 2.10⁻⁶ | g | 2.10⁻⁶ | g |
| Calcium pantothenate | HOCH₂C(CH₃)₂CH(OH)CONHCH₂CH₂CO₂•1/2Ca | 0 | g | 4.10⁻⁴ | g | 4.10⁻⁴ | g | 4.10⁻⁴ | g |
| Folic acid | C₁₉H₁₉N₇O₆ | 0 | g | 2.10⁻⁶ | g | 2.10⁻⁶ | g | 2.10⁻⁶ | g |
| Inositol | C₆H₁₂O₆ | 0 | g | 2.10⁻³ | g | 2.10⁻³ | g | 2.10⁻³ | g |
| Nicotinic acid | C₆H₅NO₂ | 0 | g | 4.10⁻⁴ | g | 4.10⁻⁴ | g | 4.10⁻⁴ | g |
| p-Aminobenzoic acid | H₂NC₆H₄CO₂H | 0 | g | 2.10⁻⁴ | g | 2.10⁻⁴ | g | 2.10 ⁻⁴ | g |
| Pyridoxine HCl | C₈H₁₁NO₃•HCl | 0 | g | 4.10⁻⁴ | g | 4.10⁻⁴ | g | 4.10⁻⁴ | g |
| Riboflavin | C₁₇H₂₀N₄O₆ | 0 | g | 2.10⁻⁴ | g | 2.10⁻⁴ | g | 2.10⁻⁴ | g |
| Thiamine HCL | C₁₂H₁₇ClN₄OS•HCl | 0 | g | 4.10⁻⁴ | g | 4.10⁻⁴ | g | 4.10⁻⁴ | g |
| Deionized water | H₂O | 1 | L | 1 | L | 1 | L | 1 | L |

TABLE 8
Compositions of the different vitamins cocktails tested:

| Chemical name | Chemical formula | Vit1X | | Vit5X | | Vit10X | | Vit0.5X | | Vit0.1X | |
|---|---|---|---|---|---|---|---|---|---|---|---|
| Biotin | C₁₀H₁₆N₂O₃S | 2 · 10⁻⁶ | g | 10⁻⁶ | g | 2 · 10⁻⁵ | g | 10⁻⁶ | g | 2 · 10⁻⁷ | g |
| Calcium pantothenate | HOCH₂C(CH₃)₂CH(OH)CONHCH₂CH₂CO₂•½Ca | 4 · 10⁻⁴ | g | 2 · 10⁻³ | g | 4 · 10⁻³ | g | 2 · 10⁻⁴ | g | 4 · 10⁻⁶ | g |
| Folic acid | C₁₉H₁₉N₇O₆ | 2 · 10⁻⁶ | g | 2 · 10⁻⁶ | g | 2 · 10⁻⁵ | g | 10⁻⁶ | g | 2 · 10⁻⁷ | g |
| Inositol | C₆H₁₂O₆ | 2 · 10⁻³ | g | 10⁻³ | g | 2 · 10⁻² | g | 10⁻³ | g | 2 · 10⁻⁴ | g |
| Nicotinic acid | C₆H₅NO₂ | 4 · 10⁻⁴ | g | 2 · 10⁻³ | g | 4 · 10⁻³ | g | 2 · 10⁻⁴ | g | 4 · 10⁻⁵ | g |
| p-Aminobenzoic acid | H₂NC₆H₄CO₂H | 2 · 10⁻⁴ | g | 10⁻³ | g | 2 · 10⁻³ | g | 10⁻⁴ | g | 2 · 10⁻⁹ | g |
| Pyridoxine HCl | C₈C₁₁NO₃•HCl | 4 · 10⁻⁴ | g | 2 · 10⁻³ | g | 4 · 10⁻³ | g | 2 · 10⁻⁴ | g | 4 · 10⁻⁵ | g |
| Riboflavin | C₁₇H₂₀N₄O₆ | 2 · 10⁻⁴ | g | 10⁻³ | g | 2 · 10⁻³ | g | 10⁻⁴ | g | 2 · 10⁻⁵ | g |
| Thiamine HCL | C₁₂H₁₇ClN₄OS•HCl | 4 · 10⁻⁴ | g | 2 · 10⁻³ | g | 4 · 10⁻³ | g | 2 · 10⁻⁴ | g | 4 · 10⁻⁵ | g |
| Deionized water | H₂O | 1 | L | 1 | L | 1 | L | 1 | L | 1 | L |

TABLE 9
Compositions of the different individual vitamins tested:

| Chemical name | Chemical formula | Bt | | CP | | FA | |
|---|---|---|---|---|---|---|---|
| Biotin | C₁₀H₁₆O₂O₃S | 2 · 10⁻⁷ | g | 0 | g | 0 | g |
| Calcium | HOCH₂C(CH₃)₂CH(OH)CONHCH₂CH₂CO₂•½Ca | 0 | g | 4 · 10⁻⁵ | g | 0 | g |
| pantothenate | | | | | | | |
| Folic acid | C₁₉H₁₉N₇O₆ | 0 | g | 0 | g | 2 · 10⁻⁷ | g |
| Inositol | C₆H₁₂O₆ | 0 | g | 0 | g | 0 | g |
| Nicotinic acid | C₆H₅NO₂ | 0 | g | 0 | g | 0 | g |
| p-Aminobenzoic | H₂NC₆H₄CO₂H | 0 | g | 0 | g | 0 | g |
| acid | | | | | | | |
| Pyridoxine HCl | C₈H₁₁NO₃•HCl | 0 | g | 0 | g | 0 | g |
| Riboflavin | C₁₇H₂₀N₄O₆ | 0 | g | 0 | g | 0 | g |
| Thiamine HCL | C₁₂H₁₇ClN₄OS•HCl | 0 | g | 0 | g | 0 | g |
| Deionized water | H₂O | 1 | L | 1 | L | 1 | L |

| Chemical name | I | | NA | | AA | | P | | R | | T | |
|---|---|---|---|---|---|---|---|---|---|---|---|---|
| Biotin | 0 | g | 0 | g | 0 | g | 0 | g | 0 | g | 0 | g |
| Calcium | 0 | g | 0 | g | 0 | g | 0 | g | 0 | g | 0 | g |
| pantothenate | | | | | | | | | | | | |
| Folic acid | 0 | g | 0 | g | 0 | g | 0 | g | 0 | g | 0 | g |
| Inositol | 2 · 10⁻⁴ | g | 0 | g | 0 | g | 0 | g | 0 | g | 0 | g |
| Nicotinic acid | 0 | g | 4 · 10⁻⁵ | g | 0 | g | 0 | g | 0 | g | 0 | g |
| p-Aminobenzoic | 0 | g | 0 | g | 2 · 10⁻⁵ | g | 0 | g | 0 | g | 0 | g |
| acid | | | | | | | | | | | | |
| Pyridoxine HCl | 0 | g | 0 | g | 0 | g | 4 · 10⁻⁵ | g | 0 | g | 0 | g |
| Riboflavin | 0 | g | 0 | g | 0 | g | 0 | g | 2 · 10⁻⁵ | g | 0 | g |
| Thiamine HCL | 0 | g | 0 | g | 0 | g | 0 | g | 0 | g | 4 · 10⁻⁶ | g |
| Deionized water | 1 | L | 1 | L | 1 | L | 1 | L | 1 | L | 1 | L |

TABLE 10

| | | Optical | Optical | | |
|---|---|---|---|---|---|
| | | density | density | Growth | |
| | | following | following | ratio | Magnetic |
| | Mineral | pre-growth | growth | (OD_{D13/} | response |
| Condition | elixir | (OD_{D6}) | (OD_{D13}) | OD_{D6}) | % |
| 1 | V0 | 0.03 | 0.26 | 9.1 | >90 |
| 1 | CB1 | 0.03 | 0.12 | 3.6 | 0 |
| 1 | V2 | 0.02 | 0.18 | 9.0 | >90 |
| 1 | CB2 | 0.08 | 0.33 | 4.3 | >90 |
| 1 | CB3 | 0.08 | 0.62 | 7.6 | >90 |
| 1 | CB4 | 0.05 | 0.23 | 4.7 | >90 |
| 1 | CB5 | 0.04 | 0.23 | 5.4 | >90 |
| 1 | CB7 | 0.02 | 0.24 | 10.9 | >90 |
| 1 | CB9 | 0.04 | 0.1 | 2.3 | 0 |
| 1 | CB10 | 0.05 | 0.22 | 4.1 | >90 |
| 1 | CB11 | 0.03 | 0.25 | 10.0 | >90 |
| 1 | CB12 | 0.22 | 1.46 | 6.6 | >90 |
| 1 | CB13 | 0.32 | 1.52 | 4.8 | >90 |

TABLE 11

| | | Optical | Optical | | |
|---|---|---|---|---|---|
| | Yeast | density | density | Growth | |
| | extract/ | following | following | ratio | |
| Con- | Vitamins | pre-growth | growth | (OD_{D13/} | Magnetic |
| dition | cocktails | (OD_{D6}) | (OD_{D13}) | OD_{D6}) | response |
| 2 | YE | 0.32 | 1.1 | 3.4 | >90 |
| 2 | YNBWAA | 0.31 | 0.82 | 2.6 | >90 |
| 2 | YNBWoAA | 0.32 | 0.76 | 2.4 | >90 |
| 2 | YNBWoAA.AS | 0.41 | 1.3 | 3.2 | >90 |
| 3 | Vit1X | 0.34 | 1.79 | 5.3 | >90 |
| 3 | Vit5X | 0 | 0 | | 0 |
| 3 | Vit10X | 0 | 0 | | 0 |
| 3 | Vit0.5X | 0.34 | 1.7 | 5.0 | >90 |
| 3 | Vit0.1X | 0.35 | 1.68 | 4.8 | >90 |

TABLE 12

| | | Optical | Optical | | |
|---|---|---|---|---|---|
| | | density | density | Growth | |
| | | following | following | ratio | Magnetic |
| Con- | | pre-growth | growth | (OD_{D13/} | response |
| dition | Vitamins | (OD_{D6}) | (OD_{D13}) | OD_{D6}) | (%) |
| 4 | Bt | 0.1 | 0.98 | 9.8 | >90 |
| 4 | CP | 0.3 | 0.98 | 3.3 | 5 |
| 4 | FA | 0.4 | 1.14 | 2.9 | 50 |
| 4 | I | 0.33 | 0.8 | 2.4 | 20 |
| 4 | NA | 0.27 | 0.65 | 2.4 | >90 |
| 4 | AA | 0.12 | 0.8 | 6.7 | 20 |
| 4 | P | 0.25 | 0.7 | 2.8 | 5 |
| 4 | R | 0.19 | 0.92 | 4.8 | >90 |
| 4 | T | 0.26 | 1.51 | 5.8 | >90 |

TABLE 13

| | | Optical | Optical | | |
|---|---|---|---|---|---|
| | | density | density | Growth | |
| | | following | following | ratio | Magnetic |
| | | pre-growth | growth | (OD_{D13/} | response |
| Condition | Chemicals | (OD_{D6}) | (OD_{D13}) | OD_{D6}) | (%) |
| 5 | N | 0.7 | 1.1 | 1.6 | >90 |
| 5 | SL0 | 0.12 | 0.1 | 0.8 | 0 |
| 5 | SL0.5X | 0.32 | 0.68 | 2.1 | 20 |
| 5 | SL0.2X | 0.1 | 0.23 | 2.3 | 20 |
| 5 | SL0.1X | 0.04 | 0.08 | 2.0 | 0 |
| 5 | AC0 | 0.15 | 0 | 0 | 0 |
| 5 | AC0.5X | 0.47 | 1.31 | 2.8 | 20 |
| 5 | AC0.2X | 0.27 | 0.99 | 3.7 | 5 |
| 5 | AC0.1X | 0.15 | 0.87 | 5.8 | 5 |
| 5 | MG0 | 0.16 | 0 | 0 | 0 |
| 5 | MG0.5X | 0.3 | 1.7 | 5.7 | 20 |
| 5 | MG0.2X | 0.003 | 0.002 | 0.7 | 5 |
| 5 | MG0.1X | 0.003 | 0.006 | 2.0 | 5 |
| 5 | P0 | 1 | 0 | 0 | 0 |
| 5 | P0.5X | 0.54 | 0.83 | 1.5 | >90 |
| 5 | P0.2X | 0.5 | 1.07 | 2.1 | >90 |
| 5 | P0.1X | 0.55 | 1.16 | 2.1 | 50 |

TABLE 14(a)
Condition 6 in 1L fermenters with non pharmaceutical grade chemicals:

| | | | B1 | | B1 | B4 | | B4 |
|---|---|---|---|---|---|---|---|---|
| | Chemical name | Chemical formula | (quantity) | | (concentration) | (quantity) | | (concentration) |
| Pre-growth medium | Sodium lactate | C₃H₅NaO₃ | 2.6 | g | 2.3 · 10⁻²M | 2.6 | g | 2.3 · 10⁻²M |
| | Ammonium chloride | NH₄Cl | 0.4 | g | 7.4 · 10⁻³M | 0.4 | g | 7.4 · 10⁻³M |
| | Magnesium sulfate heptahydrate | MgSO₄•7H₂O | 0.1 | g | 4.1 · 10⁻⁴M | 0.1 | g | 4.1 · 10⁻⁴M |
| | Potassium phosphate dibasic | K₂HPO₄ | 0.5 | g | 2.9 · 10⁻³M | 0.5 | g | 2.9 · 10⁻³M |
| | Mineral elixir CB3 (Table 6) | / | 0.5 | mL | / | 0.5 | mL | / |
| | Vitamins cocktail Vit0.1X (Table 8) | / | 0.1 | mL | / | 0.1 | mL | / |
| | Deionized water | H₂O | 1 | L | / | 1 | L | / |
| Growth medium | Sodium lactate | C₃H₅NaO₃ | 104 | g | 0.9 · 10⁻¹M | 104 | g | 0.9 · 10⁻¹M |
| (NFG) | Ammonium chloride | NH₄Cl | 16 | g | 0.3 · 10⁻¹M | 16 | g | 0.3 · 10⁻¹M |
| | Magnesium sulfate heptahydrate | MgSO₄•7H₂O | 1.2 | g | 4.9 · 10⁻³M | 1.2 | g | 4.9 · 10⁻³M |
| | Potassium phosphate dibasic | K₂HPO₄ | 2.8 | g | 0.2 · 10⁻²M | 2.8 | g | 0.2 · 10⁻²M |
| | Mineral elixir CB3 (Table 6) | / | 2.8 | mL | / | 2.8 | mL | / |
| | Vitamins cocktail Vit0.1X (Table 8) | / | 3.2 | mL | / | 3.2 | mL | / |
| | Deionized water | H₂O | 1 | L | / | 1 | L | / |
| Fed-batch medium | Lactic acid | CH₃CH(OH)COOH | 100 | g | 0.9 · 10⁻¹M | 100 | g | 0.9 · 10⁻¹M |
| (NFG) | Ammoniac | NH₃ | 4.8 | g | 2.8 · 10⁻¹M | 4.8 | g | 2.8 · 10⁻¹M |
| | Potassium phosphate dibasic | K₂HPO₄ | 6 | g | 3.4 · 10⁻²M | 6 | g | 3.4 · 10⁻²M |
| | Magnesium sulfate heptahydrate | MgSO₄•7H₂O | 2.4 | g | 9.7 · 10⁻³M | 2.4 | g | 9.7 · 10⁻³M |
| | Ferric citrate | C₆H₅FeO₇ | 1.8 | g | 7.4 · 10⁻³M | 0 | g | 0M |
| | Iron III chloride | Cl₃Fe | 0 | g | 0M | 2 | g | 7.4 · 10⁻³M |
| | Mineral elixir CB3 (Table 6) | / | 7 | mL | / | 7 | mL | / |
| | Vitamins cocktail Vit0.1X (Table 8) | / | 1 | mL | / | 1 | mL | / |
| | Deionized water | H₂O | 1 | L | / | 1 | L | / |

TABLE 14(b)
Condition 6 in 1L fermenters with pharmaceutical grade chemicals:

| | | | B2 | | B2 | B3 | | B3 |
|---|---|---|---|---|---|---|---|---|
| | Chemical Name | Chemical formula | (quantity) | | (concentration) | (quantity) | | (concentration) |
| Pre-growth medium | Sodium lactate | C₃H₅NaO₃ | 2.6 | g | 2.3 · 10⁻²M | 2.6 | g | 2.3 · 10⁻²M |
| | Ammonium chloride | NH₄Cl | 0.4 | g | 7.4 · 10⁻³M | 0.4 | g | 7.4 · 10⁻³M |
| | Magnesium sulfate heptahydrate | MgSO₄ •7H₂O | 0.1 | g | 4.1 · 10⁻⁴M | 0.1 | g | 4.1 · 10⁻⁴M |
| | Potassium phosphate dibasic | K₂HPO₄ | 0.5 | g | 2.9 · 10⁻³M | 0.5 | g | 2.9 · 10⁻³M |
| | Mineral elixir CB3 (Table 6) | / | 0.5 | mL | / | 0.5 | mL | / |
| | Vitamins cocktail Vit0.1X (Table 8) | / | 0.1 | mL | / | 0.1 | mL | / |
| | Deionized water | H₂O | 1 | L | / | 1 | L | / |
| Growth medium | Sodium lactate | C₃H₅NaO₃ | 104 | g | 0.9 · 10⁻¹M | 104 | g | 0.9 · 10⁻¹M |
| (FG) | Ammonium chloride | NH₄Cl | 16 | g | 0.3 · 10⁻¹M | 16 | g | 0.3 · 10⁻¹M |
| | Magnesium sulfate heptahydrate | MgSO₄•7H₂O | 1.2 | g | 4.9 · 10⁻³M | 1.2 | g | 4.9 · 10⁻³M |
| | Potassium phosphate dibasic | K₂HPO₄ | 2.8 | g | 0.2 · 10⁻²M | 2.8 | g | 0.2 · 10⁻²M |
| | Mineral elixir CB3 (Table 6) | / | 2.8 | mL | / | 2.8 | mL | / |
| | Vitamins cocktail Vit0.1X (Table 8) | / | 3.2 | mL | / | 3.2 | mL | / |
| | Deionized water | H₂O | 1 | L | / | 1 | L | / |
| Fed-batch | Lactic acid | CH₃CH(OH)COOH | 100 | g | 0.9 · 10⁻¹M | 100 | g | 0.9 · 10⁻¹M |
| medium (FG) | Ammoniac | NH₃ | 4.8 | g | 2.8 · 10⁻¹M | 4.8 | g | 2.8 · 10⁻¹M |
| | Potassium phosphate dibasic | K₂HPO₄ | 6 | g | 3.4 · 10⁻²M | 6 | g | 3.4 · 10⁻²M |
| | Magnesium sulfate heptahydrate | MgSO₄•7H₂O | 2.4 | g | 9.7 · 10⁻³M | 2.4 | g | 9.7 · 10⁻³M |
| | Ferric citrate | C₆H₅FeO₇ | 1.8 | g | 7.4 · 10⁻³M | 0 | g | 0M |
| | Iron III chloride | Cl₃Fe | 0 | g | 0M | 2 | g | 7.4 · 10⁻³M |
| | Mineral elixir CB3 (Table 6) | / | 7 | mL | / | 7 | mL | / |
| | Vitamins cocktail Vit0.1X (Table 8) | / | 1 | mL | / | 1 | mL | / |
| | Deionized water | H₂O | 1 | L | / | 1 | L | / |

TABLE 15

| | | Pre-growth (D0) | | Pre-growth (D8) | | Growth (D9) | | Growth (D10) | | Growth (D11) | | | |
|---|---|---|---|---|---|---|---|---|---|---|---|---|---|
| | | V1 = 250 mL | | V2 = 1.5 L | | V3 = 800 mL | | V3 = 800 mL | | V3 = 800 mL | | | Magnetic |
| Growth | | Optical | Number of | Optical | Number of | Optical | Number of | Optical | Number of | Optical | Number of | Growth ratio | response |
| Conditions | | density | cells/mL | density | cells/mL | density | cells/mL | density | cells/mL | density | cells/mL | OD_{D11}/OD_{D9} | at D11 (%) |
| 6 | B1 | 0.001 | 5 · 10⁶ | 0.4 | 2 · 10⁹ | 0.1 | 5 · 10⁸ | 0.49 | 2.5 · 10⁹ | 1.13 | 5.5 · 10⁹ | 11.3 | 80 |
| 6 | B2 | 0.001 | 5 · 10⁶ | 0.4 | 2 · 10⁹ | 0.1 | 5 · 10⁸ | 0.7 | 3.5 · 10⁹ | 2 | 1 · 10¹⁰ | 20.0 | 80 |
| 6 | B3 | 0.001 | 5 · 10⁶ | 0.4 | 2 · 10⁹ | 0.1 | 5 · 10⁸ | 0.96 | 5 · 10⁹ | 2.68 | 2.4 · 10¹⁰ | 26.8 | 100 |
| 6 | B4 | 0.001 | 5 · 10⁶ | 0.4 | 2 · 10⁹ | 0.1 | 5 · 10⁸ | 0.86 | 4.5 · 10⁹ | 1.75 | 8.5 · 10⁹ | 17.5 | 100 |

TABLE 16

| Growth | | Ag | Al | As | Ba | Cd | Co | Cr | Cu | Mn | Mo |
|---|---|---|---|---|---|---|---|---|---|---|---|
| Conditions | Element | (μg/g) | (μg/g) | (μg/g) | (μg/g) | (μg/g) | (μg/g) | (μg/g) | (μg/g) | (μg/g) | (μg/g) |
| B1 | Elemental | 169 | 307 | 0 | 1283 | 97 | 0 | 227 | 0 | 2751 | 0 |
| (NFG) | impureties | | | | | | | | | | |
| B2 | (μg of | 266 | 628 | 0 | 363 | 104 | 0 | 1627 | 0 | 785 | 0 |
| (FG) | impurity per | | | | | | | | | | |
| B3 | g of iron in | 71 | 441 | 0 | 160 | 97 | 0 | 418 | 0 | 813 | 0 |
| (FG) | nanoparticle | | | | | | | | | | |
| B4 | μg/g) | 179 | 1390 | 0 | 418 | 118 | 0 | 3538 | 0 | 497 | 0 |
| (NFG) | | | | | | | | | | | |

| Growth | Ni | Pb | Sb | Se | Si | Sn | Ti | Tl | W | Zn |
|---|---|---|---|---|---|---|---|---|---|---|
| Conditions | (μg/g) | (μg/g) | (μg/g) | (μg/g) | (μg/g) | (μg/g) | (μg/g) | (μg/g) | (μg/g) | (μg/g) |
| B1 | 473 | 367 | 0 | 0 | 0 | 0 | 625 | 0 | 0 | 0 |
| (NFG) | | | | | | | | | | |
| B2 | 660 | 677 | 0 | 0 | 0 | 12 | 1900 | 43 | 0 | 654 |
| (FG) | | | | | | | | | | |
| B3 | 292 | 408 | 0 | 0 | 0 | 19 | 129 | 18 | 0 | 0 |
| (FG) | | | | | | | | | | |
| B4 | 727 | 379 | 0 | 0 | 0 | 21 | 197 | 48 | 0 | 3208 |
| (NFG) | | | | | | | | | | |

NFG: Non pharmaceutical grade chemicals in growth and fed-batch medium
FG: Pharmaceutical grade chemicals in growth and fed-batch medium

TABLE 17

| | | | After | After | After 0 h of | After 6 h | After 12 h of | After 24 h of | After 48 h of |
|---|---|---|---|---|---|---|---|---|---|
| Growth | | Pre-growth 1 | pre-growth 1 | pre-growth 2 | growth | of growth | growth | growth | growth |
| Conditions | Chemicals | (D0) | (D8) | (D9) | (D9) | (D9) | (D9) | (D10) | (D11) |
| B1 | Iron concentration | 1.8 · 10⁶M | 1.8 · 10⁶M | 1.8 · 10⁶M | 1.8 · 10⁶M | 5.5 · 10⁶M | 1.3 · 10⁴M | 4.8 · 10⁴M | 2.1 · 10⁴M |
| | Fed-batch medium | | | | 0 mL | 0.4 mL | 1.2 mL | 5 mL | 23 mL |
| B2 | Iron concentration | 1.8 · 10⁶M | 1.8 · 10⁶M | 1.8 · 10⁶M | 1.8 · 10⁶M | 8.3 · 10⁶M | 2.2 · 10⁴M | 8.5 · 10⁴M | 3.7 · 10⁴M |
| | Fed-batch medium | | | | 0 mL | 0.7 mL | 2.2 mL | 9 mL | 40 mL |
| B3 | Iron concentration | 1.8 · 10⁶M | 1.8 · 10⁶M | 1.8 · 10⁶M | 1.8 · 10⁶M | 1.1 · 10⁴M | 2.9 · 10⁴M | 1.12 · 10⁴M | 4.9 · 10⁴M |
| | Fed-batch medium | | | | 0 mL | 1 mL | 3 mL | 12 mL | 54 mL |
| B4 | Iron concentration | 1.8 · 10⁶M | 1.8 · 10⁶M | 1.8 · 10⁶M | 1.8 · 10⁶M | 5.5 · 10⁶M | 1.8 · 10⁴M | 7.4 · 10⁴M | 3.2 · 10⁴M |
| | Fed-batch medium | | | | 0 mL | 0.6 mL | 1.9 mL | 7.8 mL | 35 mL |

TABLE 18

| Samples | Lysis | Treatment | Tᵢ | T₁, rᵢ₁, t₁ | T₂, r₁₂, rᵢ₂, t₂ | T₃, r₁₃, r₃₂, t₃ | T₄, r₃₄, t₄ | T_{f}, r_{1f}, r_{2f}, r_{3f}, r_{4f} |
|---|---|---|---|---|---|---|---|---|
| Sample 0 | None | No | 20° C. | NA | NA | NA | NA | 25° C. |
| Sample 1 | NaOH | No | 20° C. | NA | NA | NA | NA | 25° C. |
| Sample 2 | KOH | No | 20° C. | NA | NA | NA | NA | 25° C. |
| Sample 3 | NaOH | Phenol- | 20° C. | NA | NA | NA | NA | 25° C. |
| | | chloroform | | | | | | |
| Sample 4 | KOH | Heat | 20° C. | T₁ = 200° C. | NA | NA | NA | T_{f} = 25° C. |
| | | | | rᵢ₁ = 6° C./min | | | | r_{1f} = 0.3° C./min |
| | | | | t₁ = 1 hour | | | | |
| Sample 5 | KOH | Heat | 20° C. | NA | T₂ = 400° C. | NA | NA | T_{f} = 25° C. |
| | | | | | r₁₂ = 6° C./min | | | r₂ᵣ = 0.3° C./min |
| | | | | | t₂ = 1 hour | | | |
| Sample 6 | KOH | Heat | 20° C. | T₁ = 200° C. | NA | T₃ = 300° C. | NA | T_{f} = 25° C. |
| | | | | rᵢ₁ = 9° C./min | | r₁₃ = 10° C./min | | r_{3f} = 0.4° C./min |
| | | | | t₁ = 30 min | | t₃ = 1 hour | | |
| Sample 7 | KOH | Heat | 20° C. | T₁ = 200° C. | T₂ = 380° C. | T₃ = 300° C. | T₃ = 550° C. | T_{f} = 25° C. |
| | | | | rᵢ₁ = 9° C./min | r₁₂ = 8° C./min | r₃₂ = 10° C./min | r₃₄ = 8.5° C./min | r_{4f} = 0.4° C./min |
| | | | | t₁ = 30 min | t₂ = 1 hour | t₃ = 1 hour | t₄ = 1 hour | |
| Sample 8 | KOH | Heat | 20° C. | T₁ = 200° C. | T₂ = 380° C. | T₃ = 300° C. | NA | T_{f} = 25° C. |
| | | | | rᵢ₁ = 9° C./min | r₁₂ = 8° C./min | r₃₂ = 10° C./min | | r_{3f} = 0.5° C./min |
| | | | | t₁ = 30 min | t₂ = 1 hour | t₃ = 1 hour | | |
| Sample 9 | NaOH | Heat | 20° C. | T₁ = 200° C. | T₂ = 380° C. | T₃ = 300° C. | NA | T_{f} = 25° C. |
| | | | | rᵢ₁ = 9° C./min | r₁₂ = 8° C./min | r₃₂ = 10° C./min | | r_{3f} = 0.5° C./min |
| | | | | t₁ = 30 min | t₂ = 1 hour | t₃ = 1 hour | | |
| Sample 10 | KOH | Heat | 20° C. | T₁ = 200° C. | T₂ = 380° C. | T₃ = 300° C. | NA | T_{f} = 25° C. |
| | | | | rᵢ₁ = 1.2° C./min | r₁₂ = 1° C./min | r₃₂ = 1.25° C./min | | r_{3f} = 0.5° C./min |
| | | | | t₁ = 1 hour | t₂ = 2 hours | t₃ = 2 hours | | |
| Sample 11 | NaOH | Heat | 20° C. | T₁ = 200° C. | T₂ = 380° C. | T₃ = 300° C. | NA | T_{f} = 25° C. |
| | | | | rᵢ₁ = 1.2° C./min | r₁₂ = 81° C./min | r₃₂ = 1.25° C./min | | r_{3f} = 0.5° C./min |
| | | | | t₁ = 1 hour | t₂ = 2 hours | t₃ = 2 hours | | |

Rᵢ: rate of temperature increase to reach Tᵢ
Tᵢ: temperature maintained during step i
NA: Not applicable.

TABLE 19

| Samples | Lysis | Treatment | %C | %N | %Cᵢ | %Nᵢ | Δ%C = %Cᵢ ~ %C | Δ%N = %Nᵢ ~ %N | $\frac{100 * Δ \% C}{\% {C}_{i}}$ | $\frac{100 * Δ \% N}{\% {N}_{i}}$ |
|---|---|---|---|---|---|---|---|---|---|---|
| Sample 0 | None | No | 44 ± 4 | 9.9 ± 0.7 | | | | | | |
| Sample 1 | NaOH | No | 14 ± 6 | 2.5 ± 0.8 | 44 ± 4 | 9.9 ± 0.7 | −30 ± 10 | −7 ± 2 | −65 ± 17 | −75 ± 15 |
| Sample 2 | KOH | No | 7.1 ± 0.7 | 0.8 ± 0.2 | 44 ± 4 | 9.9 ± 0.7 | −37 ± 5 | −9.1 ± 0.9 | −84 ± 4 | −92 ± 3 |
| Sample 3 | NaOH | Phenol | 5 ± 2 | 0.3 ± 0.2 | 14 ± 6 | 2.5 ± 0.9 | −9 ± 8 | −2 ± 1 | −64 ± 30 | −88 ± 6 |
| | | chloroform | | | | | | | | |
| Sample 4 | KOH | Heat | 5 ± 1 | 0.28 ± 0.08 | 7.1 ± 0.7 | 0.8 ± 0.2 | −2 ± 2 | −0.5 ± 0.2 | −30 ± 25 | −65 ± 9 |
| Sample 5 | KOH | Heat | 3 ± 1 | 0.2 ± 0.1 | 7.1 ± 0.7 | 0.8 ± 0.2 | −4 ± 2 | −0.6 ± 0.3 | −56 ± 22 | −75 ± 19 |
| Sample 6 | KOH | Heat | 0.65 ± 0.01 | 0.099 ± 0.001 | 7.1 ± 0.7 | 0.8 ± 0.2 | −6.5 ± 0.7 | −0.7 ± 0.2 | −91 ± 1 | −88 ± 3 |
| Sample 7 | KOH | Heat | 0.3 ± 0.1 | 0.03 ± 0.03 | 7.1 ± 0.7 | 0.8 ± 0.2 | −6.8 ± 0.8 | −0.8 ± 0.2 | −96 ± 2 | −96 ± 1 |
| Sample 8 | KOH | Heat | 0.3 ± 0.1 | 0.03 ± 0.01 | 7.1 ± 0.7 | 0.8 ± 0.2 | −6.8 ± 0.8 | −0.8 ± 0.2 | −96 ± 2 | −96 ± 1 |
| Sample 9 | NaOH | Heat | 1.0 ± 0.3 | 0.4 ± 0.1 | 14 ± 6 | 2.9 ± 0.9 | −13 ± 1 | −2 ± 1 | −93 ± 33 | −64 ± 10 |
| Sample 10 | KOH | Heat | 0.23 ± 0.07 | 0.01 ± 0.01 | 7.1 ± 0.7 | 0.6 ± 0.2 | −6.9 ± 0.7 | −0.8 ± 0.2 | −96.8 ± 0.3 | −98.8 ± 0.3 |
| Sample 11 | NaOH | Heat | 0.8 ± 0.2 | 0.25 ± 0.03 | 14 ± 6 | 2.5 ± 0.9 | −13 ± 1 | −2.3 ± 0.9 | −94 ± 33 | −90 ± 4 |

TABLE 20

| Non reduced medium (VO) | | | | Reduced medium (CB3) | | | | | |
|---|---|---|---|---|---|---|---|---|---|
| | | % w of | | | | relatively | | | |
| | | each | | | | %w of | | | |
| | | metallic | | | | each | | | |
| | | element | | | | metallic | | | |
| | | relatively | | | | element | | | |
| | | to all | | | | to all | | | |
| | | metallic | | | | metallic | | | |
| | | elements | | | | elements | | | |
| | Data in | in the | | | Data in | in the | | | |
| Element | μg | mineral | Data in % | Element | μg | mineral | Data in % | Element | Data in μg |
| μgCd/gFe | 36.6 | % Cd | 3.58 × 10⁻³ | μgCd/gFe | 1.8 | % Cd | 1.74 × 10⁻⁴ | μgCd/μgMg | 6.08 × 10⁻⁵ |
| | (2.34) | | | | (2.6) | | | | |
| μgPb/gFe | 6654 | % Pb | 6.50 × 10⁻¹ | μgPb/gFe | 54 | % Pb | 5.24 × 10⁻³ | μgPb/μgMg | 1.8 × 10⁻³ |
| | (728.8) | | | | (5.6) | | | | |
| μgNi/gFe | 217.2 | % Ni | 2.12 × 10⁻² | μgNi/gFe | 67.8 | % Ni | 6.58 × 10⁻³ | μgNi/μgMg | 2.29 × 10⁻³ |
| | (111.8) | | | | (18.4) | | | | |
| Coμg/gFe | 121 | % Co | 1.18 × 10⁻² | Coμg/gFe | 1 | % Co | 7.71 × 10⁻⁵ | μgCo/μgMg | 3.38 × 10⁻⁵ |
| | (42.8) | | | | (0.2) | | | | |
| μgAl/gFe | 670.8 | % Al | 6.56 × 10⁻² | μgAl/gFe | 112.2 | % Al | 1.09 × 10⁻² | μgAl/μgMg | 3.80 × 10⁻³ |
| | (157) | | | | (74.4) | | | | |
| μgMn/gFe | 14069.2 | % Mn | 1 | μgMn/gFe | 79.8 | % Mn | 7.75 × 10⁻³ | μgMn/μgMg | 2.70 × 10⁻³ |
| | (3311.6) | | | | (18.8) | | | | |
| μgZn/gFe | 567.2 | % Zn | 5.55 × 10⁻² | μgZn/gFe | 121.4 | % Zn | 1.18 × 10⁻² | μgZn/μgMg | 4.11 × 10⁻³ |
| | (155.2) | | | | (128.4) | | | | |
| μgLi/gFe | 60 (23.6) | % Li | 5.87 × 10⁻³ | μgLi/gFe | 8.2 | % Li | 7.96 × 10⁻⁴ | μgLi/μgMg | 2.77 × 10⁻⁴ |
| | | | | | (1.8) | | | | |
| μgCu/gFe | 139 (12) | % Cu | 1.36 × 10⁻² | μgCu/gFe | 50.6 | % Cu | 4.91 × 10⁻³ | μgCu/μgMg | 1.71 × 10⁻³ |
| | | | | | (4.6) | | | | |
| μgMg/gFe | 127.4* | % Mg | N/A | μgMg/gFe | 29560 | % Mg | 2.8 | μgMg/μgMg | N/A |
| | | | | | (240) | | | | |

*Thesis Matthieu AMOR “Signatures chimiques et isotopiques de la magnétite des bactéries magnétotactiques”

TABLE 21

| | | | Total | Total | | |
|---|---|---|---|---|---|---|
| | | | concentration | concentration | Total | Total |
| | | | of each | of each | concentration | concentration of |
| | | | element in | element in | of each | each element in |
| | | | gram per liter | gram per liter | element in | gram per liter of |
| | | | of non- | of non-reduced | gram per liter | reduced Fed- |
| | | | reduced | Fed-batch | of reduced | batch medium |
| | | Elements | medium (V0) | medium (V0) | medium (CB3) | (CB3) |
| ICH-Q3D | Class | As | N/A | N/A | N/A | N/A |
| elements | 1 | Cd | N/A | N/A | N/A | N/A |
| | | Hg | N/A | N/A | N/A | N/A |
| | | Pb | N/A | N/A | N/A | N/A |
| | Class | Co | 9.10⁻⁴ | 1.3.10⁻² | N/A | N/A |
| | 2 | Ni | 1.25.10⁻⁴ | 1.8.10⁻³ | N/A | N/A |
| | | V | N/A | N/A | N/A | N/A |
| | | Ag | N/A | N/A | N/A | N/A |
| | | Au | N/A | N/A | N/A | N/A |
| | | Ir | N/A | N/A | N/A | N/A |
| | | Os | N/A | N/A | N/A | N/A |
| | | Pd | N/A | N/A | N/A | N/A |
| | | Pt | N/A | N/A | N/A | N/A |
| | | Rh | N/A | N/A | N/A | N/A |
| | | Ru | N/A | N/A | N/A | N/A |
| | | Se | 1.5.10⁻⁶ | 2.1.10⁻⁵ | N/A | N/A |
| | | Tl | | | N/A | N/A |
| | Class | Ba | N/A | N/A | N/A | N/A |
| | 3 | Cr | N/A | N/A | N/A | N/A |
| | | Cu | 5.10⁻⁵ | 2.01.10⁻¹ | N/A | N/A |
| | | Li | N/A | N/A | N/A | N/A |
| | | Mo | 5.10⁻⁵ | 2.01.10⁻¹ | N/A | N/A |
| | | Sb | N/A | N/A | N/A | N/A |
| | | Sn | N/A | N/A | N/A | N/A |
| | Other | Al | 1.10⁻⁴ | 1.4.10⁻³ | N/A | N/A |
| | elements | B | N/A | N/A | N/A | N/A |
| | | Ca | 1.5.10⁻² | 2.1.10⁻¹ | 1.3.10⁻² | 1.9.10⁻¹ |
| | | Fe | 5.10⁻⁴ | 2.01 | 5.10⁻⁴ | 2.01 |
| | | K | 0.5 | 6 | 0.5 | 6 |
| | | Mg | 1.10⁻¹ | 2.4 | 1.10⁻¹ | 2.4 |
| | | Mn | 2.5.10⁻³ | 3.5.10⁻² | N/A | N/A |
| | | Na | N/A | N/A | N/A | N/A |
| | | W | N/A | N/A | N/A | N/A |
| | | Zn | 9.10⁻⁴ | 1.3.10⁻² | N/A | N/A |

TABLE 22

| | | | Total | Total | Total | Total |
|---|---|---|---|---|---|---|
| | | | concentration | concentration | concentration | concentration |
| | | | of each | of each | of each | of each |
| | | | element in | element in | element in | element in |
| | | | gram per | gram per liter | gram per | gram per liter |
| | | | liter of non- | of non- | liter of | of reduced |
| | | | reduced | reduced Fed- | reduced | Fed-batch |
| | | | medium | batch medium | medium | medium |
| | | Elements | (V0) | (V0) | (CB3) | (CB3) |
| ICH-Q3D | Class | As | 2.1.10⁻⁷ | 7.3.10⁻⁶ | 2.0.10⁻⁷ | 3.0.10⁻⁶ |
| elements | 1 | Cd | 1.10⁻¹² | 1.10⁻¹² | 1.10⁻¹² | 1.10⁻¹² |
| | | Hg | 1.10⁻¹² | 1.10⁻¹² | 1.10⁻¹² | 1.10⁻¹² |
| | | Pb | 1.10⁻¹² | 1.10⁻¹² | 1.10⁻¹² | 1.10⁻¹² |
| | Class | Co | 2.3.10⁻⁴ | 2.8.10⁻³ | 1.10⁻¹² | 6.0.10⁻⁷ |
| | 2 | Ni | 4.2.10⁻⁵ | 4.7.10⁻⁴ | 1.10⁻¹¹ | 1.9.10⁻⁵ |
| | | V | 1.10⁻¹² | 1.10⁻¹² | 1.10⁻¹² | 1.10⁻¹² |
| | | Ag | 1.10⁻¹² | 1.9.10⁻⁶ | 1.10⁻¹² | 1.1.10⁻⁷ |
| | | Au | 1.10⁻¹² | 1.10⁻¹² | 1.10⁻¹² | 1.10⁻¹² |
| | | Ir | 1.10⁻¹² | 1.10⁻¹² | 1.10⁻¹² | 1.10⁻¹² |
| | | Os | 1.10⁻¹² | 1.10⁻¹² | 1.10⁻¹² | 1.10⁻¹² |
| | | Pd | 1.10⁻¹² | 1.5.10⁻⁵ | 1.10⁻¹² | 1.10⁻¹² |
| | | Pt | 1.10⁻¹² | 1.10⁻¹² | 1.10⁻¹² | 1.10⁻¹² |
| | | Rh | 1.10⁻¹² | 2.6.10⁻¹⁰ | 1.10⁻¹² | 4.5.10⁻⁹ |
| | | Ru | 5.4.10⁻⁹ | 2.7.10⁻⁸ | | |
| | | Se | 8.3.10⁻⁸ | 5.5.10⁻⁶ | 1.7.10⁻⁵ | 4.1.10⁻⁷ |
| | | Tl | 8.0.10⁻¹⁰ | 1.2.10⁻⁷ | 1.10⁻¹² | 1.10⁻¹² |
| | Class | Ba | 1.10⁻¹² | 4.4.10⁻⁶ | 1.3.10⁻⁶ | 4.2.10⁻⁵ |
| | 3 | Cr | 1.10⁻¹² | 1.2.10⁻⁵ | 1.10⁻¹² | 1.3.10⁻⁵ |
| | | Cu | 1.10⁻¹² | 5.7.10⁻⁶ | 1.10⁻¹² | 1.10⁻¹² |
| | | Li | 4.8.10⁻⁸ | 2.3.10⁻⁶ | | 1.3.10⁻⁷ |
| | | Mo | 2.1.10⁻⁵ | 2.6.10⁻⁴ | 1.5.10⁻⁸ | 2.4.10⁻⁷ |
| | | Sb | 1.10⁻¹² | 1.10⁻¹² | 1.10⁻¹² | 1.10⁻¹² |
| | | Sn | 1.10⁻¹² | 1.10⁻¹² | 1.10⁻¹² | 1.10⁻¹² |
| | Other | Al | 1.10⁻¹⁰ | 1.4.10⁻⁴ | 1.10⁻¹⁰ | 1.10⁻¹⁰ |
| | elements | B | | 3.0.10⁻⁴ | | 1.6.10⁻⁴ |
| | | Ca | 4.5.10⁻³ | 6.6.10⁻² | 5.10⁻³ | 7.9.10⁻² |
| | | Fe | 1.10⁻¹¹ | 3.9.10⁻¹ | 7.8.10⁻⁶ | 3.9.10⁻¹ |
| | | K | 2.8.10⁻¹ | 3.3 | 2.2.10⁻¹ | 2.8 |
| | | Mg | 1.5.10⁻² | 2.4.10⁻¹ | 9.3.10⁻³ | 2.4.10⁻¹ |
| | | Mn | 9.6.10⁻⁴ | 1.2.10⁻² | 1.2.10⁻⁶ | 1.4.10⁻⁵ |
| | | Na | 6.3.10⁻¹ | 5.3.10⁻² | 6.2.10⁻¹ | 2.5.10⁻² |
| | | W | 1.10⁻¹¹ | 1.10⁻¹¹ | 1.10⁻¹¹ | 9.9.10⁻⁸ |
| | | Zn | 1.9.10⁻⁴ | 3.5.10⁻³ | 1.10⁻¹¹ | 1.10⁻¹¹ |

1. High purity magnetosomes produced by magnetotactic bacteria, comprising a magnetosome composition and a structure that comprises at least (i) a magnetosome central part or magnetosome mineral or magnetosome core and optionally (ii) a magnetosome coating surrounding the magnetosome mineral or magnetosome central part or magnetosome core;
wherein the magnetosome mineral or magnetosome core or magnetosome central part or magnetosome coating or magnetosome composition comprises a metallic composition, the metallic composition is characterized by at least one property selected from the group consisting of:
i) a mass of Cd comprised in 1 gram of or magnetosome mineral or magnetosome core or magnetosome central part or magnetosome coating or magnetosome composition, is smaller than 35 μg,
ii) a percentage in mass of Cd comprised in the magnetosome mineral or magnetosome core or magnetosome central part or magnetosome coating or magnetosome composition is smaller than 3·10⁻³%,
iii) a mass of Pb comprised in 1 gram of or magnetosome mineral or magnetosome core or magnetosome central part or magnetosome coating or magnetosome composition, is smaller than 5000 μg,
iv) a percentage in mass of Pb comprised in the or magnetosome mineral or magnetosome core or magnetosome central part or magnetosome coating or magnetosome composition is smaller than 5·10⁻¹%,
v) amass of Ni comprised in 1 gram of or magnetosome mineral or magnetosome core or magnetosome central part or magnetosome coating or magnetosome composition is smaller than 200 μg,
vi) a percentage in mass of Ni comprised in the or magnetosome mineral or magnetosome core or magnetosome central part or magnetosome coating or magnetosome composition is smaller than 10⁻²%,
vii) a mass of Co comprised in 1 gram of or magnetosome mineral or magnetosome core or magnetosome central part or magnetosome coating or magnetosome composition is smaller than 100 μg,
viii) a percentage in mass of Co comprised in the or magnetosome mineral or magnetosome core or magnetosome central part or magnetosome coating or magnetosome composition is smaller than 10⁻²%,
ix) a mass of Al comprised in 1 gram of or magnetosome mineral or magnetosome core or magnetosome central part or magnetosome coating or magnetosome composition is smaller than 500 μg,
x) a percentage in mass of Al comprised in the or magnetosome mineral or magnetosome core or magnetosome central part or magnetosome coating or magnetosome composition is smaller than 5·10⁻²%,
xi) a mass of Zn comprised in 1 gram of or magnetosome mineral or magnetosome core or magnetosome central part or magnetosome coating or magnetosome composition is smaller than 500 μg,
xii) a percentage in mass of Zn comprised in the or magnetosome mineral or magnetosome core or magnetosome central part or magnetosome coating or magnetosome composition is smaller than 5·10⁻²%,
xiii) a mass of Mn comprised in 1 gram of or magnetosome mineral or magnetosome core or magnetosome central part or magnetosome coating or magnetosome composition is smaller than 1000 μg,
xiv) a percentage in mass of Mn comprised in or magnetosome mineral or magnetosome core or magnetosome central part or magnetosome coating or magnetosome composition is smaller than 1%,
xv) a mass of Li comprised in 1 gram of magnetosome mineral or magnetosome core or magnetosome central part or magnetosome coating or magnetosome composition is smaller than 50 μg,
xvi) a percentage in mass of Li comprised in the or magnetosome mineral or magnetosome core or magnetosome central part or magnetosome coating or magnetosome composition is smaller than 5·10⁻³%,
xvii) a mass of Cu comprised in 1 gram of or magnetosome mineral or magnetosome core or magnetosome central part or magnetosome coating or magnetosome composition is smaller than 100 μg,
xviii) a percentage in mass of Cu comprised in the or magnetosome mineral or magnetosome core or magnetosome central part or magnetosome coating or magnetosome composition is smaller than 10⁻²%,
and
xix) in or for a given or fixed quantity or mass of Fe comprised in the or magnetosome mineral or magnetosome core or magnetosome central part or magnetosome coating or magnetosome composition, a quantity or mass of at least one metal comprised in the or magnetosome mineral or magnetosome core or magnetosome central part or magnetosome coating or magnetosome composition selected from the group consisting of: Cd, Pb, Ni, Co, Al, Mn, Zn, Li, and Cu, is at least 10 times smaller than a quantity or mass of Mg comprised in the or magnetosome mineral or magnetosome core or magnetosome central part or magnetosome coating or magnetosome composition,
wherein a mass of or magnetosome mineral or magnetosome core or magnetosome central part or magnetosome coating or magnetosome composition is estimated as or corresponds to a mass of Fe comprised in or magnetosome mineral or magnetosome core or magnetosome central part or magnetosome coating or magnetosome composition,
wherein a percentage in mass of at least one compound comprised in the or magnetosome mineral or magnetosome core or magnetosome central part or magnetosome coating or magnetosome composition is estimated relatively to amass of all metals or of Fe and/or Mg comprised in the or magnetosome mineral or magnetosome core or magnetosome central part or magnetosome coating or magnetosome composition,
wherein the at least one metal originates from the pre-growth and/or growth and/or fed-batch medium(a).

2. The high purity magnetosomes according to claim 1, wherein the metallic composition of the magnetosome is characterized by at least one property selected from the group consisting of:
i) the magnetosome central part, magnetosome core or magnetosome mineral part comprises more than 95, 99 or 99.9% of iron, relatively to the mass of iron and at least one other metal comprised in the magnetosome central part, magnetosome core or magnetosome mineral part,
ii) the magnetosome composition comprises more than 90, 95 or 99% of iron and Magnesium.

3. The high purity magnetosomes according to claim 1, wherein the magnetosome composition is such that:
i) the magnetosome composition comprises the magnetosome central part, magnetosome core or magnetosome mineral part,
ii) the magentosome composition does not comprise at least one compound synthesized or produced by at least one magnetotactic bacterium, which is selected from the group consisting of: i) a magnetosome coating, ii) at least one protein, iii) at least one lipid, iv) at least one lipopolysaccharide, v) at least one endotoxin, vi) at least one piece of DNA, vi) at least one piece of RNA, v) at least one biological compound,
and/or
iii) the magnetosome composition comprises a magnetosome coating surrounding the magnetosome central part, magnetosome core or magnetosome mineral part, wherein the magnetosome coating does not comprise at least one compound mentioned in ii).

4. The high purity magnetosomes according to claim 1, wherein the magnetosome composition is such that it comprises three parts:
i) a first part, which is the magnetosome central part or magnetosome mineral or magnetosome central part or magnetosome core;
ii) a second part, which is the magnetosome coating surrounding the magnetosome mineral or magnetosome central part or magnetosome core;
iii) a third part, which is the environment or material or liquid or excipient or water surrounding the magnetosome coating;
wherein the first part serves to enhance the effect of at least one radiation or thermal perturbation applied on the composition to more efficiently destroy or deactivate at least one pathological cell;
wherein the second part serves to stabilize the second part or to yield magnetosome chain arrangement;
wherein the third part serves to enable the administration of the composition to the body art, i.e. without the third part it is not feasible or not easy or not convenient to administer the magnetosome composition to a body part,
wherein the third part serves to preserve partly or fully the first and/or second part(s) before during or after administration of the magnetosome composition in or to a body part.

5. The high purity magnetosomes according to claim 1, wherein the magnetosome composition is such that:
i) the magnetosome composition comprises the magnetosome central part, magnetosome core or magnetosome mineral part,
ii) the magnetosome composition does not comprise at least one compound synthesized or produced by at least one magnetotactic bacterium, which is selected from the group consisting of: i) a magnetosome coating, ii) at least one protein, iii) at least one lipid, iv) at least one lipopolysaccharide, v) at least one endotoxin, vi) at least one piece of DNA, vi) at least one piece of RNA, v) at least one biological compound.

6. The high purity magnetosomes according to claim 1, wherein the magnetosome composition is such that:
i) the magnetosome composition comprises at least one metal of group A selected from the group consisting of: Aluminum, Barium, Calcium, Cerium, Cesium, Chromium, Dysprosium, Erbium, Europium, Gadolinium, Gallium, Hafnium, Holmium, Indium, Iron, Lanthanum, Lutetium, Magnesium, Manganese, Molybdenum, Niobium, Neodymium, Potassium, Praseodymium, Rhenium, Samarium, Scandium, Sodium, Strontium, Technetium, Terbium, Thulium, Titanium, Tungsten, Yttrium, Zinc, and Zirconium,
and
ii) the magnetosome composition does not comprise at least one metal of group B or comprises at least one metal of group B in smaller quantity than at least one metal of group A, where the at least one metal of group B is selected from the group consisting of: Antimony, Arsenic, Cadmium, Cobalt, Copper, Gold, Iridium, Lead, Lithium, Mercury, Nickel, Osmium, Palladium, Platinum, Rhodium, Ruthenium, Selenium, Silver, Thallium, Tin, and Vanadium.

7. The high purity magnetosomes according to claim 1 comprising more than 50, 95 or 99% of iron and Mg, based on a ratio of (M_{FeN}+M_{MgN})/M_{MN}, where M_{FeN} is the mass of iron in the high purity magnetosomes, M_{MgN} is the mass of magnesium in the high purity magnetosomes, and M_{MN} is the mass of iron and metals or metalloids other than iron in the high purity iron oxide nanoparticle(s),
wherein iron is comprised partly or fully in the magnetosome mineral or magnetosome core or magnetosome central part,
wherein magnesium is comprised partly or fully outside of the magnetosome mineral or magnetosome core or magnetosome central part or magnetosome coating,
wherein magnesium is comprised in the magnetosome composition in the form of a magnesium salt.

8. The high purity magnetosomes according to claim 1, wherein the magnetosome composition comprises magnesium at:
i) a percentage in mass that is larger 0.1, 0.5, 1, 2 or 2.5%;
and/or
ii) a percentage in mass or concentration that is larger than the percentage in mass or concentration of Manganese, by a factor of at least 1, 1.1, 2, 5, 10 or 10³;
wherein the percentage in mass is estimated relatively to the mass of iron and at least one other metal comprised in the magnetosome composition,
wherein the large quantity of magnesium in the magnetosome composition is obtained by reducing the concentration of manganese in at least one medium used for magnetosome production and/or by reducing the quantity of manganese in the magnetosome composition.

9. The high purity magnetosomes according to claim 1, wherein the magnetosome comprises a percentage in mass of magnesium or of magnesium and iron that is larger than the percentage in mass of at least 5 different metals selected from the group consisting of: As, Cd, Hg, Pb, Co, Ni, V, Ag, Au, Ir, Os, Pd, Pt, Rh, Ru, Se, Tl, Ba, Cr, Cu, Li, Mo, Sb, Sn, Al, B, Ca, Fe, K, Mg, Mn, Na, W, and Zn.

10. The high purity magnetosomes according to claim 1, wherein the metallic composition is of:
i) the magnetosome core or magnetosome central part or magnetosome mineral,
ii) a region of the magnetosome composition that comprises crystallized iron or iron oxide, with a nanometric size or a size smaller than 500 nm for individual crystallized iron or iron oxide entities or particles, observable by a method such as electron microscopy,
iii) the magnetosome synthesized by magnetotactic bacteria without at least one compound selected from the group consisting of: i) a coating, ii) at least one compound that does not belong to the magnetosome core or magnetosome central part or magnetosome mineral, iii) at least one excipient or water or liquid.

11. The high purity magnetosomes according to claim 1, wherein the magnetosomes composition consists of:
more than 50 or 90 or 99% of iron in the magnetosome core or magnetosome central part or magnetosome mineral;
more than 50 or 90 or 99% of another metal than iron or of a carbonaceous material not originating partly or fully from magnetotactic bacteria in the magnetosome coating surrounding the magnetosome core or magnetosome mineral or magnetosome central part; and/or
more than 50 or 90 or 99% of another metal than iron or of a carbonaceous material not originating partly or fully from magnetotactic bacteria, comprised in the magnetosome coating surrounding the magnetosome core or magnetosome mineral or magnetosome central part.

12. The high purity magnetosomes according to claim 1, wherein the magnetosomes are obtained by cultivating magnetotactic bacteria in a growth medium, which is substantially free of at least one metal or non-metal or comprises less than 1 nano-Molar of at least one metal or non-metal, wherein the at least one metal or non-metal is selected from the group consisting of: 1) cadmium, 2) lead, 3) arsenic, 4) mercury, 5) cobalt, 6) vanadium, 7) nickel, 8) lithium, 9) antimony, 10) copper, 11) Vanadium, 12) Molybdate, 13) Selenium, 14) Barium, 15) Chrome, 16) Strontium, 17) a radioactive chemical element, 18) Beryllium, 19) Rubidium, 20) Ruthenium, 21) Rhodium, 22) Palladium, 23) Promethium, 24) Ytterbium, 25) Tantalum, 26) Osmium, 27) Iridium, 28) Bismuth, 29) Polonium, 30) Francium, 31) Radium, 32) Actinium, 33) Thorium, 34) Protactinium, 35) Uranium, 36) Neptunium, 37) Plutonium, 38) Americium, 39) Curium, 40) Berkelium, 41) Californium, Einsteinium, 42) Fermium, 43) Mendelevium, 44) Nobelium, 45) Lawrencium, 46) Rutherfordium, 47) Dubnium, 48) Seaborgium, 49) Bohrium, 50) Hassium, 51) Meitnerium, 52) Darmstadtium, 53) Roentgenium, 54) Copernicium, 55) Nihonium, 56) Flerovium, 57) Moscovium, 58) Livermorium, 59) Astatine, 60) Tennessine, 61) Oganesson, and 62) derivatives thereof.

13. The high purity magnetosomes according to claim 1, wherein the high purity magentosomes are produced by a method comprising the following steps:
a) a pre-growth step comprising amplifying the magnetotactic bacteria in a pre-growth and/or fed-batch medium/media,
and
b) a growth step comprising amplifying the magnetotactic bacteria originating from the pre-growth step in a growth and/or fed-batch medium/media,
wherein the fed-batch medium in the pre-growth step and the growth step, when present, is a medium that supplements the pre-growth and/or growth medium/media,
wherein the fed-batch medium comprises an iron source, or a higher concentration of iron than the pre-growth and/or growth medium(a), to enable magnetosome production,
wherein more magnetosomes are produced in the growth step than in the pre-growth step.

14. The high purity magnetosomes according to claim 1, wherein the high purity magnetosomes are produced by magnetotactic bacteria amplified in or using a pre-growth and/or growth and/or fed-batch medium(a) comprising at least one compound selected from the group consisting of: As, Cd, Hg, Pb, Co, Ni, V, Ag, Au, Ir, Os, Pd, Pt, Rh, Ru, Se, Tl, Ba, Cr, Cu, Li, Mo, Sb, Sn, Al, B, Ca, Fe, K, Mg, Mn, Na, W, and Zn, at concentration(s) estimated in gram of compound per liter of pre-growth and/or growth and/or fed-batch medium(a) that is or are lower than: 2·10⁻⁷ for As; 10⁻¹² for Cd; 10⁻¹² for Hg; 10⁻¹² for Pb; 2·10⁻⁴ for Co; 10⁻¹² for V; 10⁻¹² for Ag; 10⁻¹² for Au; 10⁻¹² for Ag; 10⁻¹² for Au; 10⁻¹² for Ir; 10⁻¹² for Os; 10⁻¹² for Pd; 10⁻¹² for Pt; 10⁻¹² for Rh; 5·10⁻⁹ for Ru; 10⁻¹² for Ir; 8·10⁻⁸ for Se; 8·10⁻¹⁰ for Ti; 10⁻¹² for Ba; 10⁻¹² for Cr; 10⁻¹² for Cu; 2·10⁻⁵ for Mo; 10⁻¹² for Sb; 10⁻¹² for Sn; 10⁻¹⁰ for Al; 4·10⁻⁴ for Ca; 2·10⁻² for K; 9·10⁻⁴ for Mn; 6·10⁻¹ for W; and 10⁻⁴ for Zn.

15. The high purity magnetosomes according to claim 1, wherein the high purity magnetosomes are produced by magnetotactic bacteria amplified in or using a fed-batch medium(a) comprising at least one compound selected from the group consisting of: As, Cd, Hg, Pb, Co, Ni, V, Ag, Au, Ir, Os, Pd, Pt, Rh, Ru, Se, Tl, Ba, Cr, Cu, Li, Mo, Sb, Sn, Al, B, Ca, Fe, K, Mg, Mn, Na, W, and Zn, at concentration(s) estimated in gram of compound per liter of fed-batch medium that is or are lower than: 7·10⁻⁶ for As; 10⁻¹² for Cd; 10⁻¹² for Hg; 10⁻¹² for Pb; 2·10⁻³ for Co; 4·10⁻⁴ for Ni; 10⁻¹² for V; 10⁻⁶ for Ag; 10⁻¹² for Au; 10⁻¹² for Ir; 10⁻¹² for Os; 10⁻⁵ for Pd; 10⁻¹² for Pt; 10⁻⁸ for Ru; 5·10⁻⁶ for Se; 10⁻⁷ for Tl; 10⁻⁶ for Ba; 10⁻⁵ for Cr; 5·10⁻⁶ for Cu; 2·10⁻⁶ for Li; 2·10⁻⁴ for Mo; 10⁻¹² for Sb; 10⁻¹² for Sn; 10⁻⁴ for Al; 3·10⁻⁴ for Ca; 3 for K; 10⁻² for Mn; and 10⁻³ for Zn.

16. The high purity magnetosomes according to claim 1, the high purity magnetosomes are produced by magnetotactic bacteria amplified in or using a fed-batch medium(a) comprising at least one compound selected from the groups consisting of: Cobalt, Molybdenum, and Manganese, at concentration(s) in the fed-batch medium that is/are:
at least 200, 500 or 1000 times smaller than the concentration of iron for Cobalt;
at least 2000, 5000 or 10000 times smaller than the concentration of iron for Molybdenum;
at least 50, 100 or 1000 times smaller than the concentration of iron for Manganese.

17. High purity nanoparticles produced through a chemical synthesis having at least one property in common with the magnetosome of high purity of claim 1, where this property is selected from the group consisting of: i) a nanoparticle composition, ii) a nanoparticle chain arrangement, and iii) a ferrimagnetic or ferromagnetic behavior.
